SEQUENCE LISTING (137 sequences)

SEQ ID NO: 1 - protein, 22 aa, synthetic construct
  REGION 1..22: note = Synthetic
MDAQTRRRERRAEKQAQWKAAN

SEQ ID NO: 2 - protein, 22 aa, synthetic construct
  REGION 1..22: note = Synthetic
GNARTRRRERRAEKQAQWKAAN

SEQ ID NO: 3 - protein, 21 aa, synthetic construct
  REGION 1..21: note = Synthetic
GNAKTRRHERRRKLAIERDTI

SEQ ID NO: 4 - protein, 17 aa, synthetic construct
  REGION 1..17: note = Synthetic
TRQARRNRRRRWRERQR

SEQ ID NO: 5 - protein, 30 aa, synthetic construct
  REGION 1..30: note = Synthetic
MSTVGTGKLTRAQRRAAARKNKRNTRVVQP

SEQ ID NO: 6 - protein, 6 aa, synthetic construct
  REGION 1..6: note = Synthetic
RRLRRP

SEQ ID NO: 7 - protein, 12 aa, synthetic construct
  REGION 1..12: note = Synthetic
RRKRRKRRKRRK

SEQ ID NO: 8 - protein, 16 aa, synthetic construct
  REGION 1..16: note = Synthetic
RRLRRPRRLRRPRRPR

SEQ ID NO: 9 - protein, 6 aa, synthetic construct
  REGION 1..6: note = Synthetic
GSGGGS

SEQ ID NO: 10 - protein, 7 aa, synthetic construct
  REGION 1..7: note = Synthetic
  VARIANT 2: note = X can be any naturally occurring amino acid
  VARIANT 4: note = X can be any naturally occurring amino acid
  VARIANT 6: note = X can be any naturally occurring amino acid
CXCXCXC

SEQ ID NO: 11 - protein, 13 aa, synthetic construct
  REGION 1..13: note = Synthetic
  VARIANT 3..4: note = X can be any naturally occurring amino acid
  VARIANT 7..8: note = X can be any naturally occurring amino acid
  VARIANT 11..12: note = X can be any naturally occurring amino acid
LIXXGVXXGVXXT

SEQ ID NO: 12 - protein, 4 aa, synthetic construct
  REGION 1..4: note = Synthetic
GALA

SEQ ID NO: 13 - protein, 30 aa, synthetic construct
  REGION 1..30: note = Synthetic
WEAALAEALAEALAEHLAEALAEALEALAA

SEQ ID NO: 14 - protein, 10 aa, synthetic construct
  REGION 1..10: note = Synthetic
  VARIANT 2..4: note = X can be any naturally occurring amino acid
  VARIANT 6..7: note = X can be any naturally occurring amino acid
  VARIANT 9: note = X can be any naturally occurring amino acid
LXXXLXXLXL

SEQ ID NO: 15 - protein, 4 aa, synthetic construct
  REGION 1..4: note = Synthetic
KDEL

SEQ ID NO: 16 - protein, 15 aa, synthetic construct
  REGION 1..15: note = Synthetic
GGGGGGGGGGGGGGG

SEQ ID NO: 17 - protein, 15 aa, synthetic construct
  REGION 1..15: note = Synthetic
GGGGSGGGGSGGGGS

SEQ ID NO: 18 - protein, 5 aa, synthetic construct
  REGION 1..5: note = Synthetic
TEFCA

SEQ ID NO: 19 - protein, 8 aa, synthetic construct
  REGION 1..8: note = Synthetic
GPLGIAGQ

SEQ ID NO: 20 - protein, 7 aa, Simian virus 40
PKKKRKV

SEQ ID NO: 21 - protein, 10 aa, Simian virus 40
PKKKRKVEAS

SEQ ID NO: 22 - protein, 16 aa, synthetic construct
  REGION 1..16: note = Synthetic
KRPAATKKAGQAKKKK

SEQ ID NO: 23 - protein, 9 aa, Homo sapiens
PAAKRVKLD

SEQ ID NO: 24 - protein, 11 aa, Homo sapiens
RQRRNELKRSP

SEQ ID NO: 25 - protein, 38 aa, Homo sapiens
NQSSNFGPMKGGNFGGRSSGPYGGGGQYFAKPRNQGGY

SEQ ID NO: 26 - protein, 42 aa, Homo sapiens
RMRIZFKNKGKDTAELRRRRVEVSVELRKAKKDEQILKRRNV

SEQ ID NO: 27 - protein, 8 aa, Homo sapiens
VSRKRPRP

SEQ ID NO: 28 - protein, 8 aa, Homo sapiens
PPKKARED

SEQ ID NO: 29 - protein, 8 aa, Homo sapiens
PQPKKKPL

SEQ ID NO: 30 - protein, 12 aa, Mus sp.
SALIKKKKKMAP

SEQ ID NO: 31 - protein, 5 aa, Influenza virus
DRLRR

SEQ ID NO: 32 - protein, 7 aa, Influenza virus
PKQKKRK

SEQ ID NO: 33 - protein, 10 aa, Hepatitis D virus
RKLKKKIKKL

SEQ ID NO: 34 - protein, 10 aa, Mus sp.
REKKKFLKRR

SEQ ID NO: 35 - protein, 20 aa, Homo sapiens
KRKGDEVDGVDEVAKKKSKK

SEQ ID NO: 36 - protein, 17 aa, Homo sapiens
RKCLQAGMNLEARKTKK

SEQ ID NO: 37 - protein, 401 aa, synthetic construct
  REGION 1..401: note = Synthetic
MSTDATLIRTTPSHAEADATDTLVATPLMPPRRVISPWPGPGEGQSLMRIPVVDIRGMALMPCTPAKARHLLKSGNARPKRNKLGLFYVQLSYEQEPDNQSLVAGVDPGSKFEGLSVVGTKDTVLNLMVEAPDHVKGAVQTRRTMRRARRQRKWRRPKRFHNRLNRMQRIPPSTRSRWEAKARIVAHLRTILPFTDVVVEDVQAVTRKGKGGTWNGSFSPVQVGKEHLYRLLRAMGLTLHLREGWQTKELREQHGLKKTKSKSKQSFESHAVDSWVLAASISGAEHPTCTRLWYMVPAILHRRQLHRLQASKGGVRKPYGGTRSLGVKRGTLVEHKKYGRCTVGGVDRKRNTISLHEYRTNTRLTQAAKVETCRVLTWLSWRSWLLRGKRTSSKGKGSHSS

SEQ ID NO: 38 - protein, 432 aa, synthetic construct
  REGION 1..432: note = Synthetic
MQPAKQQNWVFQINGDKQPLDMINPGRCRELQNRGKLASFRRFPYVVIQQQTIENPQTKEYILKIDPGSQWTGFAIQCGNDILFRAELNHRGEAIKFDLVKRAWFRRGRRSRNLRYRKKRLNRAKPEGWLAPSIRHRVLTVETWIKRFMRYCPIAWIEIEQVRFDTQKLANPEIDGVEYQQGELQGYEVREYLLQKWGRKCAYCGTENVPLEVEHIQSKSKGGSSRIGNLTLACHVCNVKKGNLDVRDFLAKSPDILNQVLENSTKPLKDAAAVNSTRYAIVKMAKSICENVKCSSGARTKMNRVRQGLEKTHSLDAACVGESGASIRVLTDRPLLITCKGHGSRQSIRVNASGFPAVKNAKTVFTHIAAGDVVRFTIGKDRKKAQAGTYTARVKTPTPKGFEVLIDGARISLSTMSNVVFVHRSDGYGYEL

SEQ ID NO: 39 - protein, 461 aa, synthetic construct
  REGION 1..461: note = Synthetic
MAVFVIDKHKRPLMPCSEKRARLLLERGRAVVHRQVPFVIRLKDRTVQHSAVQPLRVALDPGSRATGMALVREKNTVDTGTGEVYRERIALNLFELVHRGHRIREQLDQRRNFRRRRRGANLRYRAPRFDNRRRPPGWLAPSLQHRVDTTMAWVRRLCRWAPASAIGIETVRFDTQRLQNPEISGVEYQQGALAGCEVREYLLEKWGRKCAYCGAENVPLEIEHIVPKSRGGSDRVSNLALACRACNQAKGNRDVRAFLADQPERLARILAQAKAPLKDAAAVNATRWALYRALVDTGLPVEAGTGGRTKWNRTRLGLPKTHALDALCVGQVDQVRHWRVPVLGIRCAGRGSYRRTRLTRHGFPRGYLTRNKSAFGFQTGDLIRAVVTKGKKAGTYLGRIAIRASGSFNIQTPMGVVQGIHHRFCTLLQRADGYGYFVQPKPTEAALSSPRLKAGVSSAGN

SEQ ID NO: 40 - protein, 439 aa, synthetic construct
  REGION 1..439: note = Synthetic
MTTNVVFVIDTNQKPLQPCSAAVARKLLLRGKAAMFRRYPAVIILKKEVDSVGKPKIELRIDPGSKYTGFALVDSKDNADFIIWGTELEHRGAAICKELTKRSAIRRSRRNRKTRYRKKRFERRKPEGWLAPSLQHRVDTTLTWVKRICKFVPIMSISVEQVKFDLQKLENSDIQGIEYQQGTLAGYTLREALLEHWGRKCAYCDVENVFLEIEHIYPKSKGGSDKFSNLTLACHKCNINKGNKSIDEFLLSDHKRLEQIKLHQKKTLKDAAAVNATRKKLVTTLQEKTFLNVLVSDGASTKMTRLSSSLAKRHWIDAGCVNTTLIVILKTLQPLQVKCNGHGNKQFVTMDAYGFPRKSYEPKKVRKDWKAGDIIRVTKKDGTMLMGRVKKAAKKLVYIPFGGKEASFSSENAKAIHRSDGYRYSFAAIDSELLQKMAT

SEQ ID NO: 41 - protein, 419 aa, synthetic construct
  REGION 1..419: note = Synthetic
MPNKYAFVLDSKGKLLDPTKSKKAWYLIRKGKASLVEEYPLIIKLKREVPKDQVNSDKLILGIDDGTKKVGFALVQKCQTKNKVLFKAVMEQRQDVSKKMEERRGYRRYRRSHKRYRPARFDNRSSSKRKGRIPPSILQKKQAILRVVNKLKKYIRIDKIVLEDVSIDIRKLTEGRELYNWEYQESNRLDENLRKATLYRDDCTCQLCGTTETMLHAHHIMPRRDGGADSIYNLITLCKACHKDKVDNNEYQYKDQFLAIIDSKELSDLKSASHVMQGKTWLRDKLSKIAQLEITSGGNTANKRIDYEIEKSHSNDAICTTGLLPVDNIDDIKEYYIKPLRKKSKAKIKELKCFRQRDLVKYTKRNGETYTGYITSLRIKNNKYNSKVCNFSTLKGKIFRGYGFRNLTLLNRPKGLMIV

SEQ ID NO: 42 - protein, 1408 aa, synthetic construct
  REGION 1..1408: note = Synthetic
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTIDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 43 - protein, 1053 aa, synthetic construct
  REGION 1..1053: note = Synthetic
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 44 - protein, 1365 aa, synthetic construct
  REGION 1..1365: note = Synthetic
KYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 45 - protein, 288 aa, synthetic construct
  REGION 1..288: note = Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 46 - protein, 183 aa, synthetic construct
  REGION 1..183: note = Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 47 - protein, 5 aa, synthetic construct
  REGION 1..5: note = Synthetic
VITYF

SEQ ID NO: 48 - protein, 5 aa, synthetic construct
  REGION 1..5: note = Synthetic
VVVVV

SEQ ID NO: 49 - protein, 7 aa, synthetic construct
  REGION 1..7: note = Synthetic
KPWWPRR

SEQ ID NO: 50 - protein, 9 aa, synthetic construct
  REGION 1..9: note = Synthetic
NIVNVSLVK

SEQ ID NO: 51 - protein, 5 aa, synthetic construct
  REGION 1..5: note = Synthetic
LPFFD

SEQ ID NO: 52 - protein, 4 aa, synthetic construct
  REGION 1..4: note = Synthetic
KLVF

SEQ ID NO: 53 - protein, 7 aa, synthetic construct
  REGION 1..7: note = Synthetic
PGKLVYA

SEQ ID NO: 54 - protein, 7 aa, synthetic construct
  REGION 1..7: note = Synthetic
LVEALYL

SEQ ID NO: 55 - protein, 6 aa, synthetic construct
  REGION 1..6: note = Synthetic
RGFFYT

SEQ ID NO: 56 - protein, 36 aa, synthetic construct
  REGION 1..36: note = Synthetic
GSGGGSGMDAQTRRRERRAEKQAQWKAANGSGGGSG

SEQ ID NO: 57 - protein, 36 aa, synthetic construct
  REGION 1..36: note = Synthetic
GSGGGSGGNARTRRRERRAEKQAQWKAANGSGGGSG

SEQ ID NO: 58 - protein, 35 aa, synthetic construct
  REGION 1..35: note = Synthetic
GSGGGSGGNAKTRRHERRRKLAIERDTIGSGGGSG

SEQ ID NO: 59 - protein, 31 aa, synthetic construct
  REGION 1..31: note = Synthetic
GSGGGSGTRQARRNRRRRWRERQRGSGGGSG

SEQ ID NO: 60 - protein, 44 aa, synthetic construct
  REGION 1..44: note = Synthetic
GSGGGSGMSTVGTGKLTRAQRRAAARKNKRNTRVVQPGSGGGSG

SEQ ID NO: 61 - protein, 142 aa, synthetic construct
  REGION 1..142: note = Synthetic
GSGGGSGWEATLGPDRESLEGLEVAPRPPARITGVGAVPLPASGNSFDARPSQGYRRRRGRGQHRRGGVARAGSRGSRKRKRHTFCYSCGEDGHIRVQCINPSNLLLVKQKKQAAVESGNGNWAWDKSHPKSKAKGSGGGSG

SEQ ID NO: 62 - protein, 26 aa, synthetic construct
  REGION 1..26: note = Synthetic
GSMDAQTRRRERRAEKQAQWKAANGS

SEQ ID NO: 63 - protein, 26 aa, synthetic construct
  REGION 1..26: note = Synthetic
GSGNARTRRRERRAEKQAQWKAANGS

SEQ ID NO: 64 - protein, 25 aa, synthetic construct
  REGION 1..25: note = Synthetic
GSGNAKTRRHERRRKLAIERDTIGS

SEQ ID NO: 65 - protein, 21 aa, synthetic construct
  REGION 1..21: note = Synthetic
GSTRQARRNRRRRWRERQRGS

SEQ ID NO: 66 - protein, 34 aa, synthetic construct
  REGION 1..34: note = Synthetic
GSMSTVGTGKLTRAQRRAAARKNKRNTRVVQPGS

SEQ ID NO: 67 - protein, 132 aa, synthetic construct
  REGION 1..132: note = Synthetic
GSWEATLGPDRESLEGLEVAPRPPARITGVGAVPLPASGNSFDARPSQGYRRRRGRGQHRRGGVARAGSRGSRKRKRHTFCYSCGEDGHIRVQCINPSNLLLVKQKKQAAVESGNGNWAWDKSHPKSKAKGS

SEQ ID NO: 68 - protein, 24 aa, synthetic construct
  REGION 1..24: note = Synthetic
GSMDAQTRRRERRAEKQAQWKAAN

SEQ ID NO: 69 - protein, 24 aa, synthetic construct
  REGION 1..24: note = Synthetic
GSGNARTRRRERRAEKQAQWKAAN

SEQ ID NO: 70 - protein, 25 aa, synthetic construct
  REGION 1..25: note = Synthetic
GSGNAKTRRHERRRKLAIERDTIGS

SEQ ID NO: 71 - protein, 19 aa, synthetic construct
  REGION 1..19: note = Synthetic
GSTRQARRNRRRRWRERQR

SEQ ID NO: 72 - protein, 32 aa, synthetic construct
  REGION 1..32: note = Synthetic
GSMSTVGTGKLTRAQRRAAARKNKRNTRVVQP

SEQ ID NO: 73 - protein, 130 aa, synthetic construct
  REGION 1..130: note = Synthetic
GSWEATLGPDRESLEGLEVAPRPPARITGVGAVPLPASGNSFDARPSQGYRRRRGRGQHRRGGVARAGSRGSRKRKRHTFCYSCGEDGHIRVQCINPSNLLLVKQKKQAAVESGNGNWAWDKSHPKSKAK

SEQ ID NO: 74 - protein, 5 aa, synthetic construct
  REGION 1..5: note = Synthetic
GSGGS

SEQ ID NO: 75 - protein, 53 aa, synthetic construct
  REGION 1..53: note = Synthetic
GSGGGSLEIRAAFLRQRNTALRTEVAELEQEVQRLENEVSQYETRYGPLGGGK

SEQ ID NO: 76 - protein, 53 aa, synthetic construct
  REGION 1..53: note = Synthetic
GSGGGSLEIEAAFLERENTALETRVAELRQRVQRLRNRVSQYRTRYGPLGGGK

SEQ ID NO: 77 - protein, 9 aa, synthetic construct
  REGION 1..9: note = Synthetic
CYGGRGDTP

SEQ ID NO: 78 - protein, 12 aa, synthetic construct
  REGION 1..12: note = Synthetic
WHSDMEWWYLLG

SEQ ID NO: 79 - protein, 53 aa, Homo sapiens
NSDSECPLSHDGYCLHDGVCMYIEALDKYACNCVVGYIGERCQYRDLKWWELR

SEQ ID NO: 80 - protein, 51 aa, Homo sapiens
GSREGCASRCMKYNDELEKCEARMMSMSNTEEDCEQELEDLLYCLDHCHSQ

SEQ ID NO: 81 - protein, 12 aa, Homo sapiens
SFSIIHTPILPL

SEQ ID NO: 82 - protein, 20 aa, synthetic construct
  REGION 1..20: note = Synthetic
AFSWGSLWSGIKNFGSTVKN

SEQ ID NO: 83 - protein, 23 aa, synthetic construct
  REGION 1..23: note = Synthetic
GLFHAIAHFIHGGWHGLIHGWYG

SEQ ID NO: 84 - protein, 11 aa, synthetic construct
  REGION 1..11: note = Synthetic
YGRKKRRQRRR

SEQ ID NO: 85 - protein, 5 aa, synthetic construct
  REGION 1..5: note = Synthetic
RRRRR

SEQ ID NO: 86 - protein, 353 aa, synthetic construct
  REGION 1..353: note = Synthetic
MAMTLLEDWCRGMDVNSQRALLVWGIPVNCDEAEIEETLQAAMPQVSYRMLGRMFWREENAKAALLELTGAVDYAAIPREMPGKGGVWKVLFKPPTSDAEFLERLHLFLAREGWTVQDVARVLGFQNPTPTPGPEMPAEMLNYILDNVIQPLVESIWYKRLTLFSGRDIPGPGEETFDPWLEHTNEVLEEWQVSDVEKRRRLMESLRGPAADVIRILKSNNPAITTAECLKALEQVFGSVESSRDAQIKFLNTYQNPGEKLSAYVIRLEPLLQKVVEKGAIDKDNVNQARLEQVIAGANHSGAIRRQLWLTGAGEGPAPNLFQLLVQIREEEAKEEEEEAEATLLQLGLEGHF

SEQ ID NO: 87 - DNA, 2602 bp, synthetic construct
  misc_feature 1..2602: note = Synthetic
  source 1..2602: mol_type = other DNA
agcagtaacgtcgcggcgggttgcgggtaggactggacgccagagcagccgcgcagcgcctgaaccgctgcgggccgccgcggccgccccttcccaccctcgcctctgctgtctccagcctcgcttctccgactttcctgctcctctgctgccttcgtttctggtcctcggccgtcctcgccgcccgcccagaggagtccccgcgcccgccaagaagccgctttccgctggcccgcagccgccgcgacttcggcacagtttctccctctggctagtctcccaaacggccgctcctcgcccgcgggaagaccaggctgcgaccgcgaacgcccgatcctctccaggagccgcagcgagcgcccggcggccacgccccgcgaccacaccccggcggctctcggcccagcgcgcctgccttcgccgcccgccgtcgctcctcgcccgctgcacgacgacgcgacgcccctgctgcaggcggcggacccgaccggacccagacccagacgcaagatggcgacggccgcgtgactgcctcagcgtccccgagctcggctccgagtgcacctacggactgactgtgggggcagagaagggcgagatcaggactctgtctttgttaatcgtgactgcatgaaggtcgcctccctcgggcctacttggtgggagtgtctggtattgttctaaggccaggagcacggtgagccacagtctgttggtagaatttggcgtcttgatagttgagaaaatggcgatgacactgttggaagactggtgccgggggatggatgtgaactcccagagagctctgttagtctggggcatcccagtgaactgtgatgaggctgaaatcgaagagaccctccaggctgcgatgccccaggtctcctaccgaatgcttgggagaatgttctggagggaagaaaatgcgaaagcagccttattagagctcactggcgctgtagattacgccgcgatccccagggagatgccgggcaaaggaggggtctggaaagtgttatttaagcccccaacttctgatgctgaatttttagaaagattgcacctcttcctagctagagaggggtggaccgtgcaagatgttgcccgtgtccttgggtttcagaaccctactccgaccccgggcccagagatgccagcagagatgctaaactatattttggataatgttattcagcctcttgttgagtccatatggtacaagaggctgacacttttctcggggagggacatcccagggcctggagaggaaacctttgatccctggctggagcacactaatgaggtcctagaggagtggcaggtgtccgatgtagaaaagaggcggcggttgatggagagtcttagaggccccgccgctgatgttattcgcatccttaagtccaacaaccccgcgataaccactgccgaatgcctgaaggcgcttgagcaggtgtttgggagcgttgagagctctagggatgcccagatcaaatttctgaacacttatcagaacccgggagaaaaattgtctgcttatgtcattcgtctggagcctctgctacagaaggtggtagagaagggggccattgataaagataatgtgaaccaggcccgcctagagcaggtcattgccggggccaaccacagcggggccatccgaaggcagctgtggcttaccggggctggggaagggccagccccaaacctctttcagttgctggtgcagatccgtgaggaggaagccaaggaggaggaggaggaggctgaggccacccttctgcagttaggcctggaagggcacttctgagtgccaggaaaggcagctttagtgcagacctagatcacagctacttttcttgtccctgtggggtcttacagatgtgtctctgagtagtaaaggcttagccttgttctgttttgttgttttttggaggggaaggttagtcaggcctgagtattcatgtaacattctaaaattgtgccagcgagcaccgtgaacgactgcaatgcaagcgggtcttgctggctaaaatgccaggtaaagggttggttggacacagcgcttagtgcacgctgtcatcatggacatcataatcagttgtgaaaaacacgcgaacctatgacacttcttattccacactgaatgtgaaattgcatgttcagatgtttactacgaggcctggctcacaggaagtgttcagtaaaagtatgcactgttagattactgataacgcggatagatttttgtttaccataaattgttccagatttatattaatggaaggaagtgtgcatttattagctattactcaactttacaatgcaaacatcttatttctcatctttaaacatgtcgaccagtttaattgaaaagtattctgagactgcaaaatggggtgttaaaaaatactgcagttacggagctgtgtaaaccagtttctcattgcataagatacagatgtaaattgcatggagaggttgatatgcacctgtacagtaattcactcccccatttcacatctttgtcagagaatagttcttgttcatactgagtgttctaaatttgaagttatatatacaaattaaaatattttaaaaattc

SEQ ID NO: 88 - protein, 364 aa, synthetic construct
  REGION 1..364: note = Synthetic
MALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDD

SEQ ID NO: 89 - DNA, 4730 bp, synthetic construct
  misc_feature 1..4730: note = Synthetic
  source 1..4730: mol_type = other DNA
agaccaccagctaatggatgcggagcggagggcccgctgaccgctctccgcgcctggagcagcttggcttggctggagctaagagccagacacaccactgtgtggaggtgggtgatgtcttcctgtgctaaaaggtgaataaataagctcctcacctctcgcggaacactcgggaacacatcaacaggggtccaagccgccctgctgggaggcttctcttcaagagttctgggtcccagagtggaaggcattttcccatcaactggagagagacgaaacatcagagaccaggaggctgtggagaaagcagctgtcccaggtgcctcaactatcagagaagggtcagcgtcacgtggctgccagcatctttgagaaaatcactggcaatcggacttcagagctgcgggcacaggtgtggttagaactgagatacgacctgcccacctgggtcaggcctaaagacaagaagtcctgagttcttgccactgagtaggccagggtcatttgtccagaaaactttgtgactgtctttgagtgacctagtctgggacccattcattggtgggttctaaggttagaagctcatccaggatattttcaatattaagtcagtgcatagctgcaccactaacaaattggtgcctgtagagtcagagtgggtcaattcttaggacaatggcgctggcactgttagaggactggtgcaggataatgagtgtggatgagcagaagtcactgatggttacggggataccggcggactttgaggaggctgagattcaggaggtccttcaggagactttaaagtctctgggcaggtatagactgcttggcaagatattccggaagcaggagaatgccaatgctgtcttactagagcttctggaagatactgatgtctcggccattcccagtgaggtccagggaaaggggggtgtctggaaggtgatctttaagacccctaatcaggacactgagtttcttgaaagattgaacctgtttctagaaaaagaggggcagacggtctcgggtatgtttcgagccctggggcaggagggcgtgtctccagccacagtgccctgcatctcaccagaattactggcccatttgttgggacaggcaatggcacatgcgcctcagcccctgctacccatgagataccggaaactgcgagtattctcagggagtgctgtcccagccccagaggaagagtcctttgaggtctggttggaacaggccacggagatagtcaaagagtggccagtaacagaggcagaaaagaaaaggtggctggcggaaagcctgcggggccctgccctggacctcatgcacatagtgcaggcagacaacccgtccatcagtgtagaagagtgtttggaggcctttaagcaagtgtttgggagcctagagagccgcaggacagcccaggtgaggtatctgaagacctatcaggaggaaggagagaaggtctcagcctatgtgttacggctagaaaccctgctccggagagcggtggagaaacgcgccatccctcggcgtattgcggaccaggtccgcctggagcaggtcatggctggggccactcttaaccagatgctgtggtgccggcttagggagctgaaggatcagggcccgccccccagcttccttgagctaatgaaggtaatacgggaagaagaggaggaagaggcctcctttgagaatgagagtatcgaagagccagaggaacgagatggctatggccgctggaatcatgagggagacgactgaaaaccacctgggggcaggacccacagccagtgggctaagacctttaaaaaatttttttctttaatgtatgggactgaaatcaaaccatgaaagccaattattgaccttccttccttccttccttccctcccttcctccttctctccttctctcctcctctctcctctcctctcctctctttccttccttccttccttttttctttttctctttcttctttatttcttgggtctcactctcatcacccaggctagagtgcagtggcacaaaaatctcggctcactgcagccttgacttcccaggctcaggctcaggtgatcctcacaccttagcctcccaagtacctgggactacaggcacgcaccaccatgcctagctattcttttgtatttttggtagagacagggttttgctgtgttgctcaggctggtctggaacccctaggctcaaatgatgtgcccaactcggcctcccaaagtgctgggattacaggcatgaaccgccatgcctggcccttgatttttctttttaagaaaaaaatatctaggagtttcttagaccctatgtagattattaatgaacaaaagattaaactccaaatattaaatagtaagcctgaaggaatctgaaacacttgtacttccaattttctttaaataatcccaaatagaccagaattggcccataccatagaagaaagaattggcagtcaaaaaaaaaaataccttttgtaatgtttgaaaaataaagctgtttgacttgtcaggtgttttcctttctcaaatcagcaaattctctctgagtgcctggctttgtgagacactgtacaaggagttacaagactacagctataacctgcagttgagcagttataaacctacaaaatgggccctgccctcagagaggttccagtctagatgaggagctgatctagacaggtaaaaggctaactaaccctttgtgtaaataagttcatcaccccagtaaaagtgtcatcacccagtgaataggaccacctctgcctgcagatttttgttgttgttgttgtcattgttgttgttgttttaacctgggaagtgttcttcctgcctttctgctaggtgtcagatagatggtcccagagctaggtgctgtgtcaggccctgaagacacagatgactcaacctaagctttactttccagaggtccacagcctgagaggtgtccccaaagaaagggggacatgaggggactgcatgcttgagagcagggttgtttagggcaggtttggatttagtgagcaggctggtttgcttagagaaggcttttagtggcaacaaaggatgaagaggagagaaaaggaactcacatttattgagggcctactgtgtgcaaagtgtttcatgtatatctcattgaatgtatacagccaccctgttgtggtataattttgctctttataaagagaaagaccgaagctcagatgagttaagtggtctcctcaacaccaaaatgccaagaagtgatggagcctagacagaagcccagaactttctgactcacactagtccatcctctaccatcacgatgactttcaaattgtgctctgcagttctgcagattttctagcagtgccatctccaaaatgtgttttaaactctttatttttttaattattattagtattattttgagactgagtcttgctctatcacccaggctggagtgcagtggtgcaatctcagctcactgcaacctccgcctcccaggttcaagcgatttcgtgcctcagcctcccgagtagctgggattacaggcacccaccaccacgcccagctaatttttgtatttttagtagaaatggggtttcaccatgttggccaggctggtctcgaactcctgacctcaagtgatccactcacctcggcctcccaaagtgctgggattacaggtgtgagccaccatgcctgggctaaactctttaagtctctagtaaatgcagctagattcaaatgggctgataaccaaattttaacacatcagcattcaccaccaggtttacttttattttcagattggctcattttgtgcagaccttagagcaaagtttcctttatggtatctgtgtacgtatccaaacttcttttaattgttcacagattttaaaagcggtagcaccacatggttgtgtagatcagacctgtgtatttagatcagacctgtgtatcacgtaagtgtgtgagtgcagtgcagatgagcaccatttagttatatgtgctaggcaaatctccaacacagttgatgtgtagtcttgtggtagatttgtgcatactgtaagcaaattgcttagcttctctagacatcagtttccacatctgaaaaataagaagatgagagtacacggttgttatgaacaaatgacttaatgctttttaagcacgttgcatgacatctggaacacagaaagccctcaatacattgaagctcttaggattttcacgatgttcctgtctgctcaatgcatgctttctttattgttctgacagttgtgtggtaacaagctaatatgcttccagttgacttccagtctaccctggtgttagaaaccgtttcatctcttattgtaaatttgagtgcttgttgttttttatatttgtgatgactcttccagcagttgttgacaattgttagaggtttgacttttaaataattacttattttttctgattgtggttcagtttaactgaagaatatcctgagattgtaagaaaagcattttttaaaaggtatcacttgtgatcatttatctttctaaattctatttttaatactgttccaccaaagtgatgcagtggttaccatgacaccctaatttcatgtgtttttgtatttatgaaaatagtttcattgtcatttattggcggtatacaaagtaaaatgttataaatgtgaagttataaaataaatatatgctaataaaa

SEQ ID NO: 90 - protein, 463 aa, synthetic construct
  REGION 1..463: note = Synthetic
MPLTLLQDWCRGEHLNTRRCMLILGIPEDCGEDEFEETLQEACRHLGRYRVIGRMFRREENAQAILLELAQDIDYALLPREIPGKGGPWEVIVKPRNSDGEFLNRLNRFLEEERRTVSDMNRVLGSDTNCSAPRVTISPEFWTWAQTLGAAVQPLLEQMLYRELRVFSGNTISIPGALAFDAWLEHTTEMLQMWQVPEGEKRRRLMECLRGPALQVVSGLRASNASITVEECLAALQQVFGPVESHKIAQVKLCKAYQEAGEKVSSFVLRLEPLLQRAVENNVVSRRNVNQTRLKRVLSGATLPDKLRDKLKLMKQRRKPPGFLALVKLLREEEEWEATLGPDRESLEGLEVAPRPPARITGVGAVPLPASGNSFDARPSQGYRRRRGRGQHRRGGVARAGSRGSRKRKRHTFCYSCGEDGHIRVQCINPSNLLLVKQKKQAAVESGNGNWAWDKSHPKSKAK

SEQ ID NO: 91 - DNA, 3682 bp, synthetic construct
  misc_feature 1..3682: note = Synthetic
  source 1..3682: mol_type = other DNA
gcactggcccacgtgctgcgcgagcgagggagagccacagtctgagcgaacgtccgcgctgggagccaggggtgcccgacccccgtccgccgccgccgccgccgccgcgcatagcccccggagagccctctggggaccccgaccagaagggaccttgccctgggagaaggctgtggagacctgggccttctgcgatcaccctaggagttgatccagatatgtgcctcacgccctgatcactccccccaaattagtatccgcagagattcgaggacatgccgttgaccttgttacaggactggtgtcggggggaacacctgaacacccggaggtgcatgctcatcctggggatccccgaggactgtggcgaggatgagtttgaggagacactccaggaggcttgcaggcacctgggcagatacagggtgattggcaggatgtttaggagggaggagaacgcccaggcgattctactggagctggcacaagatatcgactatgctttgctcccaagggaaataccaggaaagggggggccctgggaagtgattgtaaaaccccgtaactcagatggggaatttctcaacagactgaaccgcttcttagaggaggagaggcggaccgtgtcagatatgaaccgagtcctcgggtcggacaccaattgttcggctccaagagtgactatatcaccagagttctggacctgggcccagactctgggggcagcagtgcagcctctgctagaacaaatgttgtaccgagaactaagagtgttttctgggaacaccatatccatcccaggtgcactggcctttgatgcctggcttgagcacaccactgagatgctacagatgtggcaggtgcccgagggggaaaagaggcggaggctgatggaatgcttacggggccctgctctccaggtggtcagtgggctccgggccagcaatgcttccataactgtggaggagtgcctggctgccttgcagcaggtgttcggacctgtggagagccataaaattgcccaggtgaagttgtgtaaagcctatcaggaggcaggagagaaagtatctagctttgtgttacgtttggaacccctgctccaaagagctgtagaaaacaatgtggtatcacgtagaaacgtgaatcagactcgcctgaaacgagtcttaagtggggccacccttcctgacaaactccgagataagcttaagctgatgaaacagcgaaggaagcctcctggtttcctggccctggtgaagctcctgcgtgaggaggaggaatgggaggccactttaggtccagatagggagagtctggaggggctggaagtagccccaaggccacctgccaggatcactggggttggggcagtacctctccctgcctctggcaacagttttgatgcgaggccttcccagggctaccggcgccggaggggcagaggccaacaccgaaggggtggtgtggcaagggctggctctcgaggctcaagaaaacggaaacgccacacattctgctatagctgtggggaagacggccacatcagggtacagtgcatcaacccctccaacctgctcttggtaaagcagaagaaacaggctgcagttgagtcgggaaacgggaactgggcttgggacaagagccatcccaagtccaaggccaagtaggctcgggagaacagggcaacatttcctaccacaggccaaggagacaaaagagatattggaaggaggggaaagagaagcccagacaaacagcagatgagttgagtggggcagagggacagggcagccagaccaaggccaagccttctcacccttggccagctggaagggacttcagcaaccaagaccacctggcaacaggctcagtgggggtcaggtccaggtccccgaagaggtgctggagaggaaagcagggagccactgcatccagcacatggggtgcctgggcctcagatggggaccccaaagaagcagaagctgaagaaggtacggctgggggttctgtcctgctcatccaaccacccctaaatacccaccctgtggactttgagctgaacatgcccactggcccccaggccacatgggacctggaggagcctacctggggcctgcccctgccagcaggtgccagggctggtgaggaagagctggggggcagaggtaaagccctgcaggggaggccacagggtccatcccgtcttcaggatcatctacactgcactaggggagccccaggaaggcagcaccctggaggccctgtgccagtgaggacaggagaccctaaggccccgggagcccagtgccagccagaggttgtgcaggcaaggagaccaaagattgatgagaagacccccagcaggggtactgggtacccggcaggccagtgccctcacagttgacttggaccagggtggctgtgaagggaagtctttgttgcaaaggaggaggaaaagggaggacttggtagggttttgtttcttctgcttgtttctgtacagggccaccagactcctggagagatcaagcaaggagaacctggggctgccatggccaaagcaactcaacagatgccaatgccaattccaaggccagccacaaccctgccaccttggggaatccagcctggaggcatcccctaagcagccagccatggcctgggtggaggcacctgaagacgtctgtcccaaactcccccagccctgagctgggagatgacagggggaaagaggccctctcaagggtgccagatgcctgggtctcccaagaggggtcccccaactcactgttcccgggacaggctgccccctgttccaggaagctcatcctcacctgtgtaggcccctgtagtgacccacgcgtccagcagacgcccacccaccgctagccgttgttcctgtgcaaagtagtgtgctatgcacccacccaggtggccgcctctgggcccaaggcacatgctgtgagcttcctgtgagcccaggctctgctcactgctgtcccgcgtcatgagcaccacctctgctttccctgtgtagatctaggccagtggctgcttgttcttgtggagctgtgtgtgttcttctctgagcagctcctccccggagtcccccagcacagtcccaggagatgacaggaaggaagcaccagggcaaggcggacgctcaccctgtgaccacgatggtgaccgtgactgtgggaggaagaactggacccaggacggagcggggctgccctgcctgaggctcccgaggagctttgtgctttggtgttccacccctgttgttactcatgactcagtttccttaacctggtagggtgttccctgctgtgttttccagtgtcctgtgactgtcctgtgcgggccatagggcagggccctgccccagcagatgggcttgggaggggactccctaaagccagtggacactgccagagtctaccttcctggcaagaggcagaccccggggccctcaggaaggagggagttggcagcgggggctgcagcaggagtaggagcagatgaggcgtcttgccaggaacctcaggaggagggggcccgggacctgtgtgggacctgtgtcctgtggtggccgtttgcagtttctctctgtgttgtgattcccttctcttcaacgttttcagtacgtgtttctcttcaataaacttcattcagtgttcca

SEQ ID NO: 92 - protein, 455 aa, synthetic construct
  REGION 1..455: note = Synthetic
MPLTLLQDWCRGEHLNTRRCMLILGIPEDCGEDEFEETLQEACRHLGRYRVIGRMFRREENAQAILLELAQDIDYALLPREIPGKGGPWEVIVKPRNSDGEFLNRLNRFLEEERRTVSDMNRVLGSDTNCSAPRVTISPEFWTWAQTLGAAVQPLLEQMLYRELRVFSGNTISIPGALAFDAWLEHTTEMLQMWQVPEGEKRRRLMECLRGPALQVVSGLRASNASITVEECLAALQQVFGPVESHKIAQVKLCKAYQEAGEKVSSFVLRLEPLLQRAVENNVVSRRNVNQTRLKRVLSGATLPDKLRDKLKLMKQRRKPPGFLALVKLLREEEEWEATLGPDRESLEGLEVAPRPPARITGVGAVPLPASGNSFDARPSQGYRRRRGRGQHRRGGVARAGSRGSRKRKRHTFCYSCGEDGHIRVQCINPSNLLLAKETKEILEGGEREAQTNSR

SEQ ID NO: 93 - DNA, 3561 bp, synthetic construct
  misc_feature 1..3561: note = Synthetic
  source 1..3561: mol_type = other DNA
gcactggcccacgtgctgcgcgagcgagggagagccacagtctgagcgaacgtccgcgctgggagccaggggtgcccgacccccgtccgccgccgccgccgccgccgcgcatagcccccggagagccctctggggaccccgaccagaagggaccttgccctgggagaaggctgtggagacctgggccttctgcgatcaccctaggagttgatccagatatgtgcctcacgccctgatcactccccccaaattagtatccgcagagattcgaggacatgccgttgaccttgttacaggactggtgtcggggggaacacctgaacacccggaggtgcatgctcatcctggggatccccgaggactgtggcgaggatgagtttgaggagacactccaggaggcttgcaggcacctgggcagatacagggtgattggcaggatgtttaggagggaggagaacgcccaggcgattctactggagctggcacaagatatcgactatgctttgctcccaagggaaataccaggaaagggggggccctgggaagtgattgtaaaaccccgtaactcagatggggaatttctcaacagactgaaccgcttcttagaggaggagaggcggaccgtgtcagatatgaaccgagtcctcgggtcggacaccaattgttcggctccaagagtgactatatcaccagagttctggacctgggcccagactctgggggcagcagtgcagcctctgctagaacaaatgttgtaccgagaactaagagtgttttctgggaacaccatatccatcccaggtgcactggcctttgatgcctggcttgagcacaccactgagatgctacagatgtggcaggtgcccgagggggaaaagaggcggaggctgatggaatgcttacggggccctgctctccaggtggtcagtgggctccgggccagcaatgcttccataactgtggaggagtgcctggctgccttgcagcaggtgttcggacctgtggagagccataaaattgcccaggtgaagttgtgtaaagcctatcaggaggcaggagagaaagtatctagctttgtgttacgtttggaacccctgctccaaagagctgtagaaaacaatgtggtatcacgtagaaacgtgaatcagactcgcctgaaacgagtcttaagtggggccacccttcctgacaaactccgagataagcttaagctgatgaaacagcgaaggaagcctcctggtttcctggccctggtgaagctcctgcgtgaggaggaggaatgggaggccactttaggtccagatagggagagtctggaggggctggaagtagccccaaggccacctgccaggatcactggggttggggcagtacctctccctgcctctggcaacagttttgatgcgaggccttcccagggctaccggcgccggaggggcagaggccaacaccgaaggggtggtgtggcaagggctggctctcgaggctcaagaaaacggaaacgccacacattctgctatagctgtggggaagacggccacatcagggtacagtgcatcaacccctccaacctgctcttggccaaggagacaaaagagatattggaaggaggggaaagagaagcccagacaaacagcagatgagttgagtggggcagagggacagggcagccagaccaaggccaagccttctcacccttggccagctggaagggacttcagcaaccaagaccacctggcaacaggctcagtgggggtcaggtccaggtccccgaagaggtgctggagaggaaagcagggagccactgcatccagcacatggggtgcctgggcctcagatggggaccccaaagaagcagaagctgaagaaggtacggctgggggttctgtcctgctcatccaaccacccctaaatacccaccctgtggactttgagctgaacatgcccactggcccccaggccacatgggacctggaggagcctacctggggcctgcccctgccagcaggtgccagggctggtgaggaagagctggggggcagaggtaaagccctgcaggggaggccacagggtccatcccgtcttcaggatcatctacactgcactaggggagccccaggaaggcagcaccctggaggccctgtgccagtgaggacaggagaccctaaggccccgggagcccagtgccagccagaggttgtgcaggcaaggagaccaaagattgatgagaagacccccagcaggggtactgggtacccggcaggccagtgccctcacagttgacttggaccagggtggctgtgaagggaagtctttgttgcaaaggaggaggaaaagggaggacttggtagggttttgtttcttctgcttgtttctgtacagggccaccagactcctggagagatcaagcaaggagaacctggggctgccatggccaaagcaactcaacagatgccaatgccaattccaaggccagccacaaccctgccaccttggggaatccagcctggaggcatcccctaagcagccagccatggcctgggtggaggcacctgaagacgtctgtcccaaactcccccagccctgagctgggagatgacagggggaaagaggccctctcaagggtgccagatgcctgggtctcccaagaggggtcccccaactcactgttcccgggacaggctgccccctgttccaggaagctcatcctcacctgtgtaggcccctgtagtgacccacgcgtccagcagacgcccacccaccgctagccgttgttcctgtgcaaagtagtgtgctatgcacccacccaggtggccgcctctgggcccaaggcacatgctgtgagcttcctgtgagcccaggctctgctcactgctgtcccgcgtcatgagcaccacctctgctttccctgtgtagatctaggccagtggctgcttgttcttgtggagctgtgtgtgttcttctctgagcagctcctccccggagtcccccagcacagtcccaggagatgacaggaaggaagcaccagggcaaggcggacgctcaccctgtgaccacgatggtgaccgtgactgtgggaggaagaactggacccaggacggagcggggctgccctgcctgaggctcccgaggagctttgtgctttggtgttccacccctgttgttactcatgactcagtttccttaacctggtagggtgttccctgctgtgttttccagtgtcctgtgactgtcctgtgcgggccatagggcagggccctgccccagcagatgggcttgggaggggactccctaaagccagtggacactgccagagtctaccttcctggcaagaggcagaccccggggccctcaggaaggagggagttggcagcgggggctgcagcaggagtaggagcagatgaggcgtcttgccaggaacctcaggaggagggggcccgggacctgtgtgggacctgtgtcctgtggtggccgtttgcagtttctctctgtgttgtgattcccttctcttcaacgttttcagtacgtgtttctcttcaataaacttcattcagtgttcca

SEQ ID NO: 94 - protein, 351 aa, synthetic construct
  REGION 1..351: note = Synthetic
MTLRLLEDWCRGMDMNPRKALLIAGISQSCSVAEIEEALQAGLAPLGEYRLLGRMFRRDENRKVALVGLTAETSHALVPKEIPGKGGIWRVIFKPPDPDNTFLSRLNEFLAGEGMTVGELSRALGHENGSLDPEQGMIPEMWAPMLAQALEALQPALQCLKYKKLRVFSGRESPEPGEEEFGRWMFHTTQMIKAWQVPDVEKRRRLLESLRGPALDVIRVLKINNPLITVDECLQALEEVFGVTDNPRELQVKYLTTYQKDEEKLSAYVLRLEPLLQKLVQRGAIERDAVNQARLDQVIAGAVHKTIRRELNLPEDGPAPGFLQLLVLIKDYEAAEEEEALLQAILEGNFT

SEQ ID NO: 95 - DNA, 2407 bp, synthetic construct
  misc_feature 1..2407: note = Synthetic
  source 1..2407: mol_type = other DNA
gcgcagcttccccgagcgagaccaaaacaggtggaatccgggctggagccggagctccggcggcgcgggtggcggcacgtccctccagacagtaccacaggcacctggagtaccggcatcggtcgctgtggcccccgagtgtccgtcagagcctaggggagcctgccctcccgcgcctcgtcggggcccggccaggcaccttggccgccggcgcacggacgcgggcacgagcactagatcacggctgctggacctcggcacgttgacaagatttctctggggtaccgcggaggattactttgaatttcggtggtcgcctgtggtctggcatatttagaacttaagtctattatttcgggcaccatgactttgaggcttttagaagactggtgcagggggatggacatgaaccctcggaaagcgctattgattgccggcatctcccagagctgcagtgtggcagaaatcgaggaggctctgcaggctggtttagctcccttgggggagtacagactgcttggaaggatgttcaggagggatgagaacaggaaagtagccttagtagggcttactgcggagactagtcacgccctggtccctaaggagataccgggaaaagggggtatctggagagtgatctttaagccccctgacccagataatacatttttaagcagattaaatgaatttttagcgggagagggcatgacagtgggtgagttgagcagagctcttggacatgaaaatggctccttagacccagagcagggcatgatcccggaaatgtgggcccctatgttggcacaggcattagaggctcttcagcctgccctgcaatgcttgaagtataaaaagctgagagtgttctcgggcagggagtctccagaaccaggagaagaagaatttggacgctggatgtttcatactactcagatgataaaggcgtggcaggtgccagatgtagagaagagaaggcgattgctagagagccttcgaggcccagcacttgatgttattcgtgtcctcaagataaacaatcctttaattactgtcgatgaatgtctgcaggctcttgaggaggtatttggggttacagataatcctagggagttgcaggtcaaatatctaaccacttaccagaaggatgaggaaaagttgtcggcttatgtactaaggctggagcctttgttacagaagctggtacagagaggagcaattgagagagatgctgtgaatcaggcccgcctagaccaagtcattgctggggcagtccacaaaacaattcgcagagagcttaatctgccagaggatggcccagcccctggtttcttgcagttattggtactaataaaggattatgaggcagctgaggaggaggaggcccttctccaggcaatattggaaggtaatttcacctgagtctcagggaaccacgaagggatatggcaatgagtagagcatgaaggtagaacagtctatatactcttgtgacacatacaatccctaccttgtgctgccaagtaactcatttttgtgcaattctcagtataagccctttgtcgtttctgtgcctatttaaagtctcctaaaggtgtaattgactaggaaggatgtagttctacactgccatttacctatttaaattcatccttgtgaatatctttgttgttgttgttgagacagagtctcgctctgtcacccaggctggagtgcagtggcgtgatcttggctcactgcatcctccgccttccaggtttaagctattctcctgcctcagttgcccgagtagctgggactacaggcatgtgccaccacgcccagctaagttttgcattatcagtagagacggggtttcaccatgttggccaggctagtttcgaactcctgacctcaagtgatccacctgcttcggcctctgaaagtgctgggattacaggcgtgagacactgcgcccagcctcatccctgtgaatttcttattgtacacaagttctttcactatctgtgtgcagtgctctgaggggacagacaaggcttgggtgtatatgccaaccagatctcattgaagtatcagcttgtttggtactaggtgcaagtgtagcatgtcacatgtgaccatgttggatcattgacaatttttaggtatgtactgacctacatttatgatgaagatcctgagcggaggttaagatattaagttattttccatatgaatcagaattatattgattctgtgcaatcaaaacaaaaggcagaatagaatgctgagattggttaagtttgcaatgaccatcttgaaccacagatttctgctatgtgtcatcaaaacatctagttctgagtaacattttcacgattgttataaaattataggtgtgaacttctaaaataaaggaatgctaataaaa

SEQ ID NO: 96 - protein, 448 aa, synthetic construct
  REGION 1..448: note = Synthetic
MALTLLEDWCKGMDMDPRKALLIVGIPMECSEVEIQDTVKAGLQPLCAYRVLGRMFRREDNAKAVFIELADTVNYTTLPSHIPGKGGSWEVVVKPRNPDDEFLSRLNYFLKDEGRSMTDVARALGCCSLPAESLDAEVMPQVRSPPLEPPKESMWYRKLKVFSGTASPSPGEETFEDWLEQVTEIMPIWQVSEVEKRRRLLESLRGPALSIMRVLQANNDSITVEQCLDALKQIFGDKEDFRASQFRFLQTSPKIGEKVSTFLLRLEPLLQKAVHKSPLSVRSTDMIRLKHLLARVAMTPALRGKLELLDQRGCPPNFLELMKLIRDEEEWENTEAVMKNKEKPSGRGRGASGRQARAEASVSAPQATVQARSFSDSSPQTIQGGLPPLVKRRRLLGSESTRGEDHGQATYPKAENQTPGREGPQAAGEELGNEAGAGAMSHPKPWET

SEQ ID NO: 97 - DNA, 3118 bp, synthetic construct
  misc_feature 1..3118: note = Synthetic
  source 1..3118: mol_type = other DNA
tctcattgcattgcgcagagctcagccatctttctcatccgtcaggcattcctgagaaagagggccactctacctctcttgctgccagccttcactccagcaaggagggtgctgggtgacctgagcccacatagccccgagcacccgaagcacacacagcagagtcctctggactttgaggaagaacccacagcaggaggaagtcagcagggagtggctgtgtgaaacctgggaccacttctgccttcctacgtggcagtggctcagagttatttgagtgctgtcaaactgagctgattgctgccctagtattagatcagtccatagaaagtgggagcaatggcactgacactgctagaggattggtgcaaggggatggacatggaccccagaaaggccctgctgattgtaggcatccccatggagtgtagtgaggtggaaattcaggacactgtgaaggcaggcttacagcccctgtgcgcatacagggtcctagggagaatgttcaggagggaagacaatgccaaggcagtcttcattgaactggctgacactgtcaattacactactctgcccagtcacataccaggaaagggtggctcctgggaagtggtggtaaaaccccgtaacccagatgatgagtttctcagtagactgaactacttcctgaaagatgagggccgaagtatgacagatgtggccagagccctgggatgttgcagcctccctgccgagagcctggatgcagaggtcatgccccaagttagatccccacctttagagcctccgaaagaaagtatgtggtacaggaaactgaaagtgttttcgggaactgcttcccctagcccaggcgaagagacctttgaagactggctagagcaggtcactgagataatgcccatatggcaagtgtctgaggtggagaagaggcggcgtttgctggagagcttacgtgggcctgctctgtcaatcatgcgggtgctccaggccaacaatgactccataactgtggagcagtgccttgacgccctaaagcagatctttggggataaagaggactttagagcctctcagtttaggtttctgcagacctctccgaagattggagagaaagtctccactttcctgctgcgcttagagcccctgctgcagaaagccgtgcacaagagccccttgtcagtgcgcagcacagacatgattcgtctgaaacatctcttagctcgggtcgccatgacccccgccctcaggggcaagctggagctcttggatcagcgagggtgtcctcccaattttctggagttaatgaagctcattcgagatgaagaagagtgggagaacactgaggcagtgatgaagaataaggagaagccatcagggagaggccggggggcctccggtagacaggcaagagcagaagccagtgtctctgcacctcaggccaccgtgcaggcaaggtcttttagcgacagcagcccccagaccatacagggaggcttacccccattagtgaaacgcaggcggctgttaggcagtgaaagcactaggggagaagaccatggccaggccacgtatcccaaggctgaaaaccagactccaggcagggaggggccacaggctgcaggagaggagttgggaaacgaggcaggggctggggccatgagccatcccaagccctgggaaacataggctcagaagaccgaagcactccttcccctggcaggcagcagggacatttgaacaaggggaaggggcagcttacactaacagggaacttgactggggcagagggacagggaagccaggtcgagatcaagcccccttactttccaccagctggaagggacttcaccaaccaagaccaaatagcatctggttcagtgggggccgggcccatagcccctaacaggtacaggagacacaagcggggaaccactacacttagcacatggggtgcctgggcttcagatggggaccccagcgaagcaggggctgaagaaggtgtggctgggggctctggcctggtcctctggacacccccaaatatctaccctgtggactttgagctgagcatgcccactggcacccaggccacgtgggacctggaggagcctgcctggggcctgcctctgccagaaggagccagggctgctgaggaagagctgtggggcagaagtgaagccccgcaggggaggccacagggtccagcctgtctttaggatcatctacactgcatgaggggagccccaggaaggcagcattcataagcccctgcaccagtgaggaagagaaagaggaggagaattaagaagaggatgatcgggaggaggaagaggagaataaggaggagcaggaggaagagggggaggagaaggaggaagagggggaggaggaagaggggaaggaggaggaagaagtgtaagaagagaagagaggatgtgttgggggtgggtagttgtttttggtttgcgtcatcatcaggcccgggctctgctcactgctgttccgtgtcaagaactccacctctgctttgccggtgtagatctcggccagtggctgcttgttctcttggggatgtgtgtgtttttatctgagcagctcctttccagagtaccccagcgcagttccaggagacggtgggaaggaggcatggcagacgctcaccttgtgactgtgacctcgactgcaggaggaccaaccagacacgggaaggagcagggctgcctgaggctccccaggaagctttgtgctttggcgttccacccctgctgttactcgtgactcagtttcctcaacttggtggggtgttccctgctatgttttccagcatcctgtgactgtcctgtgcagtctatagggcagggccctgccccagcgggtgggctcaggagggggttccctgaagcgagtacacattgccagagcccaccatcctggcaagaggtggatcctggggccctatggaaggagggatgtggtggggggggccacactgagggaggggccggggacttgtgacctgtagtggctgtttgcagtttctctctgtgttagattcccttttcttcagcagtttcagttcatgtttctcttcagtaaattttgttcagtgttccaaaaa

SEQ ID NO: 98 - protein, 448 aa, synthetic construct; identical to SEQ ID NO: 96
  REGION 1..448: note = Synthetic

SEQ ID NO: 99 - DNA, 3218 bp, synthetic construct
  misc_feature 1..3218: note = Synthetic
  source 1..3218: mol_type = other DNA
tctcattgcattgcgcagagctcagccatctttctcatccgtcaggcattcctgagaaagagggccactctacctctcttgctgccagccttcactccagcaaggagggtgctgggtgacctgagcccacatagccccgagtgagcagtgaggctgtctcctgccctcttctgcctggagggctcgtcagtgtccccaggtgtcaggccctgcctcctgacgttggccttttcactacaggcacccgaagcacacacagcagagtcctctggactttgaggaagaacccacagcaggaggaagtcagcagggagtggctgtgtgaaacctgggaccacttctgccttcctacgtggcagtggctcagagttatttgagtgctgtcaaactgagctgattgctgccctagtattagatcagtccatagaaagtgggagcaatggcactgacactgctagaggattggtgcaaggggatggacatggaccccagaaaggccctgctgattgtaggcatccccatggagtgtagtgaggtggaaattcaggacactgtgaaggcaggcttacagcccctgtgcgcatacagggtcctagggagaatgttcaggagggaagacaatgccaaggcagtcttcattgaactggctgacactgtcaattacactactctgcccagtcacataccaggaaagggtggctcctgggaagtggtggtaaaaccccgtaacccagatgatgagtttctcagtagactgaactacttcctgaaagatgagggccgaagtatgacagatgtggccagagccctgggatgttgcagcctccctgccgagagcctggatgcagaggtcatgccccaagttagatccccacctttagagcctccgaaagaaagtatgtggtacaggaaactgaaagtgttttcgggaactgcttcccctagcccaggcgaagagacctttgaagactggctagagcaggtcactgagataatgcccatatggcaagtgtctgaggtggagaagaggcggcgtttgctggagagcttacgtgggcctgctctgtcaatcatgcgggtgctccaggccaacaatgactccataactgtggagcagtgccttgacgccctaaagcagatctttggggataaagaggactttagagcctctcagtttaggtttctgcagacctctccgaagattggagagaaagtctccactttcctgctgcgcttagagcccctgctgcagaaagccgtgcacaagagccccttgtcagtgcgcagcacagacatgattcgtctgaaacatctcttagctcgggtcgccatgacccccgccctcaggggcaagctggagctcttggatcagcgagggtgtcctcccaattttctggagttaatgaagctcattcgagatgaagaagagtgggagaacactgaggcagtgatgaagaataaggagaagccatcagggagaggccggggggcctccggtagacaggcaagagcagaagccagtgtctctgcacctcaggccaccgtgcaggcaaggtcttttagcgacagcagcccccagaccatacagggaggcttacccccattagtgaaacgcaggcggctgttaggcagtgaaagcactaggggagaagaccatggccaggccacgtatcccaaggctgaaaaccagactccaggcagggaggggccacaggctgcaggagaggagttgggaaacgaggcaggggctggggccatgagccatcccaagccctgggaaacataggctcagaagaccgaagcactccttcccctggcaggcagcagggacatttgaacaaggggaaggggcagcttacactaacagggaacttgactggggcagagggacagggaagccaggtcgagatcaagcccccttactttccaccagctggaagggacttcaccaaccaagaccaaatagcatctggttcagtgggggccgggcccatagcccctaacaggtacaggagacacaagcggggaaccactacacttagcacatggggtgcctgggcttcagatggggaccccagcgaagcaggggctgaagaaggtgtggctgggggctctggcctggtcctctggacacccccaaatatctaccctgtggactttgagctgagcatgcccactggcacccaggccacgtgggacctggaggagcctgcctggggcctgcctctgccagaaggagccagggctgctgaggaagagctgtggggcagaagtgaagccccgcaggggaggccacagggtccagcctgtctttaggatcatctacactgcatgaggggagccccaggaaggcagcattcataagcccctgcaccagtgaggaagagaaagaggaggagaattaagaagaggatgatcgggaggaggaagaggagaataaggaggagcaggaggaagagggggaggagaaggaggaagagggggaggaggaagaggggaaggaggaggaagaagtgtaagaagagaagagaggatgtgttgggggtgggtagttgtttttggtttgcgtcatcatcaggcccgggctctgctcactgctgttccgtgtcaagaactccacctctgctttgccggtgtagatctcggccagtggctgcttgttctcttggggatgtgtgtgtttttatctgagcagctcctttccagagtaccccagcgcagttccaggagacggtgggaaggaggcatggcagacgctcaccttgtgactgtgacctcgactgcaggaggaccaaccagacacgggaaggagcagggctgcctgaggctccccaggaagctttgtgctttggcgttccacccctgctgttactcgtgactcagtttcctcaacttggtggggtgttccctgctatgttttccagcatcctgtgactgtcctgtgcagtctatagggcagggccctgccccagcgggtgggctcaggagggggttccctgaagcgagtacacattgccagagcccaccatcctggcaagaggtggatcctggggccctatggaaggagggatgtggtggggggggccacactgagggaggggccggggacttgtgacctgtagtggctgtttgcagtttctctctgtgttagattcccttttcttcagcagtttcagttcatgtttctcttcagtaaattttgttcagtgttccaaaaa

SEQ ID NO: 100 - protein, 448 aa, synthetic construct; identical to SEQ ID NO: 96
  REGION 1..448: note = Synthetic

SEQ ID NO: 101 - DNA, 3196 bp, synthetic construct
  misc_feature 1..3196: note = Synthetic
  source 1..3196: mol_type = other DNA
attgcattgcgcagagctcagccatctttctcatccgtcaggcattcctgagaaagagggccactctacctctcttgctgccagccttcactccagcaaggagggtgctgggtgacctgagcccacatagccccgagtgagcagtgaggctgtctcctgccctcttctgcctggagggctcgtcagtgtccccaggtgtcaggccctgcctcctgacgttggccttttcactacaggcacccgaagcacacacagcagagtcctctggactttgaggaagaacccacagcaggaggaagctgtgtgaaacctgggaccacttctgccttcctacgtggcagtggctcagagttatttgagtgctgtcaaactgagctgattgctgccctagtattagatcagtccatagaaagtgggagcaatggcactgacactgctagaggattggtgcaaggggatggacatggaccccagaaaggccctgctgattgtaggcatccccatggagtgtagtgaggtggaaattcaggacactgtgaaggcaggcttacagcccctgtgcgcatacagggtcctagggagaatgttcaggagggaagacaatgccaaggcagtcttcattgaactggctgacactgtcaattacactactctgcccagtcacataccaggaaagggtggctcctgggaagtggtggtaaaaccccgtaacccagatgatgagtttctcagtagactgaactacttcctgaaagatgagggccgaagtatgacagatgtggccagagccctgggatgttgcagcctccctgccgagagcctggatgcagaggtcatgccccaagttagatccccacctttagagcctccgaaagaaagtatgtggtacaggaaactgaaagtgttttcgggaactgcttcccctagcccaggcgaagagacctttgaagactggctagagcaggtcactgagataatgcccatatggcaagtgtctgaggtggagaagaggcggcgtttgctggagagcttacgtgggcctgctctgtcaatcatgcgggtgctccaggccaacaatgactccataactgtggagcagtgccttgacgccctaaagcagatctttggggataaagaggactttagagcctctcagtttaggtttctgcagacctctccgaagattggagagaaagtctccactttcctgctgcgcttagagcccctgctgcagaaagccgtgcacaagagccccttgtcagtgcgcagcacagacatgattcgtctgaaacatctcttagctcgggtcgccatgacccccgccctcaggggcaagctggagctcttggatcagcgagggtgtcctcccaattttctggagttaatgaagctcattcgagatgaagaagagtgggagaacactgaggcagtgatgaagaataaggagaagccatcagggagaggccggggggcctccggtagacaggcaagagcagaagccagtgtctctgcacctcaggccaccgtgcaggcaaggtcttttagcgacagcagcccccagaccatacagggaggcttacccccattagtgaaacgcaggcggctgttaggcagtgaaagcactaggggagaagaccatggccaggccacgtatcccaaggctgaaaaccagactccaggcagggaggggccacaggctgcaggagaggagttgggaaacgaggcaggggctggggccatgagccatcccaagccctgggaaacataggctcagaagaccgaagcactccttcccctggcaggcagcagggacatttgaacaaggggaaggggcagcttacactaacagggaacttgactggggcagagggacagggaagccaggtcgagatcaagcccccttactttccaccagctggaagggacttcaccaaccaagaccaaatagcatctggttcagtgggggccgggcccatagcccctaacaggtacaggagacacaagcggggaaccactacacttagcacatggggtgcctgggcttcagatggggaccccagcgaagcaggggctgaagaaggtgtggctgggggctctggcctggtcctctggacacccccaaatatctaccctgtggactttgagctgagcatgcccactggcacccaggccacgtgggacctggaggagcctgcctggggcctgcctctgccagaaggagccagggctgctgaggaagagctgtggggcagaagtgaagccccgcaggggaggccacagggtccagcctgtctttaggatcatctacactgcatgaggggagccccaggaaggcagcattcataagcccctgcaccagtgaggaagagaaagaggaggagaattaagaagaggatgatcgggaggaggaagaggagaataaggaggagcaggaggaagagggggaggagaaggaggaagagggggaggaggaagaggggaaggaggaggaagaagtgtaagaagagaagagaggatgtgttgggggtgggtagttgtttttggtttgcgtcatcatcaggcccgggctctgctcactgctgttccgtgtcaagaactccacctctgctttgccggtgtagatctcggccagtggctgcttgttctcttggggatgtgtgtgtttttatctgagcagctcctttccagagtaccccagcgcagttccaggagacggtgggaaggaggcatggcagacgctcaccttgtgactgtgacctcgactgcaggaggaccaaccagacacgggaaggagcagggctgcctgaggctccccaggaagctttgtgctttggcgttccacccctgctgttactcgtgactcagtttcctcaacttggtggggtgttccctgctatgttttccagcatcctgtgactgtcctgtgcagtctatagggcagggccctgccccagcgggtgggctcaggagggggttccctgaagcgagtacacattgccagagcccaccatcctggcaagaggtggatcctggggccctatggaaggagggatgtggtggggggggccacactgagggaggggccggggacttgtgacctgtagtggctgtttgcagtttctctctgtgttagattcccttttcttcagcagtttcagttcatgtttctcttcagtaaattttgttcagtgttcca

SEQ ID NO: 102 - protein, 448 aa, synthetic construct; identical to SEQ ID NO: 96
  REGION 1..448: note = Synthetic

SEQ ID NO: 103 - DNA, 3188 bp, synthetic construct
  misc_feature 1..3188: note = Synthetic
  source 1..3188: mol_type = other DNA
gcagtcagcacggaggcggcagccgccatagacgtgtggtggtcgcgctgcgcgggaactccggcttggaggcacctccggcagatccagtgaagcaggacccatcacgcctgagtcaggaactgcctaactggatagagttgaaaagcagagcgagtctgaagtggctgtgttattgcagcatccgcgctgccagcagggccaacccagcaaggcacccgaagcacacacagcagagtcctctggactttgaggaagaacccacagcaggaggaagtcagcagggagtggctgtgtgaaacctgggaccacttctgccttcctacgtggcagtggctcagagttatttgagtgctgtcaaactgagctgattgctgccctagtattagatcagtccatagaaagtgggagcaatggcactgacactgctagaggattggtgcaaggggatggacatggaccccagaaaggccctgctgattgtaggcatccccatggagtgtagtgaggtggaaattcaggacactgtgaaggcaggcttacagcccctgtgcgcatacagggtcctagggagaatgttcaggagggaagacaatgccaaggcagtcttcattgaactggctgacactgtcaattacactactctgcccagtcacataccaggaaagggtggctcctgggaagtggtggtaaaaccccgtaacccagatgatgagtttctcagtagactgaactacttcctgaaagatgagggccgaagtatgacagatgtggccagagccctgggatgttgcagcctccctgccgagagcctggatgcagaggtcatgccccaagttagatccccacctttagagcctccgaaagaaagtatgtggtacaggaaactgaaagtgttttcgggaactgcttcccctagcccaggcgaagagacctttgaagactggctagagcaggtcactgagataatgcccatatggcaagtgtctgaggtggagaagaggcggcgtttgctggagagcttacgtgggcctgctctgtcaatcatgcgggtgctccaggccaacaatgactccataactgtggagcagtgccttgacgccctaaagcagatctttggggataaagaggactttagagcctctcagtttaggtttctgcagacctctccgaagattggagagaaagtctccactttcctgctgcgcttagagcccctgctgcagaaagccgtgcacaagagccccttgtcagtgcgcagcacagacatgattcgtctgaaacatctcttagctcgggtcgccatgacccccgccctcaggggcaagctggagctcttggatcagcgagggtgtcctcccaattttctggagttaatgaagctcattcgagatgaagaagagtgggagaacactgaggcagtgatgaagaataaggagaagccatcagggagaggccggggggcctccggtagacaggcaagagcagaagccagtgtctctgcacctcaggccaccgtgcaggcaaggtcttttagcgacagcagcccccagaccatacagggaggcttacccccattagtgaaacgcaggcggctgttaggcagtgaaagcactaggggagaagaccatggccaggccacgtatcccaaggctgaaaaccagactccaggcagggaggggccacaggctgcaggagaggagttgggaaacgaggcaggggctggggccatgagccatcccaagccctgggaaacataggctcagaagaccgaagcactccttcccctggcaggcagcagggacatttgaacaaggggaaggggcagcttacactaacagggaacttgactggggcagagggacagggaagccaggtcgagatcaagcccccttactttccaccagctggaagggacttcaccaaccaagaccaaatagcatctggttcagtgggggccgggcccatagcccctaacaggtacaggagacacaagcggggaaccactacacttagcacatggggtgcctgggcttcagatggggaccccagcgaagcaggggctgaagaaggtgtggctgggggctctggcctggtcctctggacacccccaaatatctaccctgtggactttgagctgagcatgcccactggcacccaggccacgtgggacctggaggagcctgcctggggcctgcctctgccagaaggagccagggctgctgaggaagagctgtggggcagaagtgaagccccgcaggggaggccacagggtccagcctgtctttaggatcatctacactgcatgaggggagccccaggaaggcagcattcataagcccctgcaccagtgaggaagagaaagaggaggagaattaagaagaggatgatcgggaggaggaagaggagaataaggaggagcaggaggaagagggggaggagaaggaggaagagggggaggaggaagaggggaaggaggaggaagaagtgtaagaagagaagagaggatgtgttgggggtgggtagttgtttttggtttgcgtcatcatcaggcccgggctctgctcactgctgttccgtgtcaagaactccacctctgctttgccggtgtagatctcggccagtggctgcttgttctcttggggatgtgtgtgtttttatctgagcagctcctttccagagtaccccagcgcagttccaggagacggtgggaaggaggcatggcagacgctcaccttgtgactgtgacctcgactgcaggaggaccaaccagacacgggaaggagcagggctgcctgaggctccccaggaagctttgtgctttggcgttccacccctgctgttactcgtgactcagtttcctcaacttggtggggtgttccctgctatgttttccagcatcctgtgactgtcctgtgcagtctatagggcagggccctgccccagcgggtgggctcaggagggggttccctgaagcgagtacacattgccagagcccaccatcctggcaagaggtggatcctggggccctatggaaggagggatgtggtggggggggccacactgagggaggggccggggacttgtgacctgtagtggctgtttgcagtttctctctgtgttagattcccttttcttcagcagtttcagttcatgtttctcttcagtaaattttgttcagtgttcca

SEQ ID NO: 104 - protein, 399 aa, synthetic construct
  REGION 1..399: note = Synthetic
MAVTMLQDWCRWMGVNARRGLLILGIPEDCDDAEFQESLEAALRPMGHFTVLGKAFREEDNATAALVELDREVNYALVPREIPGTGGPWNVVFVPRCSGEEFLGLGRVFHFPEQEGQMVESVAGALGVGLRRVCWLRSIGQAVQPWVEAVRCQSLGVFSGRDQPAPGEESFEVWLDHTTEMLHVWQGVSERERRRRLLEGLRGTALQLVHALLAENPARTAQDCLAALAQVFGDNESQATIRVKCLTAQQQSGERLSAFVLRLEVLLQKAMEKEALARASADRVRLRQMLTRAHLTEPLDEALRKLRMAGRSPSFLEMLGLVRESEAWEASLARSVRAQTQEGAGARAGAQAVARASTKVEAVPGGPGREPEGLLQAGGQEAEELLQEGLKPVLEECDN

SEQ ID NO: 105 - DNA, 2238 bp, synthetic construct
  misc_feature 1..2238: note = Synthetic
  source 1..2238: mol_type = other DNA
ccaaacgccgagcgagcgagggagaggcacagtcagagggaacgcccgcgcggggagccaggggcgcccgaccccgccgccgccgcagcggcgcgcagcccccgacgcgccctgtggggacccggaccaggagggaccctgccccgggaaaaggctgtggagacctgggctccgaccccagttcatccccccacacccccgccgccccgtgccaccctggtccgcgctgggaaccctatcctgcccctcgtgtcagcccggcactggccagaatcgcgggcatggcggtgaccatgctgcaggactggtgccggtggatgggggtcaacgctcgcaggggcctgctcatcctgggcatcccggaggactgtgatgatgccgaattccaagagtccctcgaggctgccctgaggcctatgggacactttacagtgctaggcaaagcgtttcgagaggaggataatgccaccgcggccctggtcgagctcgaccgggaagtcaactatgctttggtccccagggaaatccccggcactgggggcccgtggaacgtggtctttgtgccccgttgctcaggcgaggagtttctcggtctcggtcgcgtgttccacttcccggagcaagaggggcagatggtggagagcgtggccggcgccctgggtgtggggctgcgcagggtgtgctggctgcgatccatcggtcaggcggtccagccctgggtggaggccgtgaggtgccagagcctgggcgtgttttccgggagggaccagccagccccaggggaggagtcctttgaggtctggctagaccacaccaccgaaatgctgcatgtgtggcagggggtctcggaaagggagaggaggaggaggctgctggaaggcttgcgtgggaccgccctgcagctcgtgcacgcgctcctggcggagaaccccgccaggacggcgcaggactgtctggcggccctggcccaggtgtttggagacaacgagtcccaggcgaccatccgggtgaagtgtctgaccgctcagcagcagtcaggcgagcgtctctcagctttcgtgttgcggctggaagtgctgctgcagaaggccatggagaaggaggccctggccagagcatccgccgaccgcgtgcgcctgaggcagatgctcaccagggcccaccttactgagcctctggatgaagcactgaggaagctgagaatggccgggaggtctccaagtttcttggagatgctggggctcgttcgggagtctgaggcatgggaggccagtctagccaggagcgtgagagcccagacacaggaaggggccggtgcccgggctggtgcccaggctgttgccagagccagcactaaagtagaggcggtcccaggaggtcctggtcgggagccagagggcctcctccaggcaggaggccaggaggctgaggagctcctccaggaggggctcaagcccgtcctggaggaatgtgataactaggttggggctggggaggcagcccagcgcgagtcctccccgggcaaataggctccgagggccccggggcctcctctcctcctctcaggcagcagggccctggagacaggcggaggcggggccagggccggtccctcaccccacatcgggatcggggccccccacttccccccaaggggccctgcccaccaccaccttcccggtgacccagatgaccacatttaataccaaatggggtggggggaggcgcccctccagtgccaggggcacgtgctgtgagcttcctgggagcccaggttgtgctcactgctctcccgtatcgtgagcaccacctctgctttccctgcgtagatctaggccaggggctgcttgtttttgtggagccgtgtgtgttcttctctgagcagctcctccccagaggaccccagcgcagtcccgggagatggcggaaagaaggcaccagggcacagtggacactcatcccgtgacagcgatggtgaccatgactgtgggagaaagaacaggacccgggatggagtggggctgtctgagtttccccagtgaactttgtgctttggcgttccacccctgttgttacttgttactgagtttcctagacctggggtgggtgttcccccaggaggaggggggtcccgacctgtgtcctgtggtggccatttgcagcttctgtgttgtgattcccttctcttcaacggtttcagtacatatctctcttcaataaatttcattcagtgttcca

SEQ ID NO: 106 - protein, 369 aa, synthetic construct
  REGION 1..369: note = Synthetic
MALAMLRDWCRWMGANAERSLLILGIPDDCKEHEFQEAVRAALSPLGRREQPGCEEESFESWVEHAKDMLQLWCHASEREKKRWLLESLGGPALEVVSGLLEEDTNLSALDCLAALGQVFRNQDTRMTSRLKFLTCTQGPQEGLFAFVVRLEGLLQRAVEKGAVCPALANYLRLQQVLSWARPSEALQDTLRGMQLEKRPPGFLGLLRLIREMEAWAAFPARSQQGVAWAAAPVESEDPAAAQASPAQGNASEAGPGAEDAAEAASATKEAARGAPAAGEGESAPAGPEGLGQARPIEVPWGSSPARMSSAVWVFPRGLSWGPEGLIQVRGQEARKPPLEGLQTILEEPENEDEDGAGDEGQPKSSQGK

SEQ ID NO: 107 - DNA, 2555 bp, synthetic construct
  misc_feature 1..2555: note = Synthetic
  source 1..2555: mol_type = other DNA
acacgcttccacgctgcggagctgaggtgatctcttcagacccttcggagagacccagatatctgacttcccactgagagacccggaattgcccagagtttgagaaagctgtgtcggagactgcaacagtcagcacacattcctgattgatcaggctccctccctcaagtcctctgcgatggctctggcgatgcttcgggactggtgcaggtggatgggtgcgaacgcagagcgctccctgctcatcctgggtatccctgatgactgcaaggaacatgagttccaggaggccgtgcgggctgccctgtcgcccctgggcaggagggagcagccaggctgcgaggaagagtcctttgagagctgggtggagcatgccaaggatatgctgcagctgtggtgccatgcgtcggaaagggagaagaagaggtggctgctggagagcttgggcggcccggccctggaagtcgtgagcggcctcctggaggaagataccaacttgtccgcgctggactgcctggcggcgctggggcaggtatttaggaaccaggacactcgaatgacttcgaggctgaagttcctgacctgcacgcaggggccccaggaggggctgtttgccttcgtggtgcgcctggaaggcctgctgcagagggctgtggagaagggggccgtctgcccagccttggccaattacctgcgactacagcaggtgctgtcttgggcccgccccagcgaggcactccaggataccctgagagggatgcagctggagaagaggccacctggcttcctggggctgctccggctcatccgggagatggaggcatgggcagccttcccagcgaggagccagcagggtgtggcctgggcagcggccccagtggagagtgaagacccagctgctgcccaggcctccccagcccaggggaatgccagcgaggctggtcccggagcagaagatgctgccgaggccgcttctgccaccaaagaggctgcaaggggagcccctgccgctggggaaggtgaaagtgcccctgcaggccccgaaggcctaggtcaggcaaggcccatagaggtcccctggggctcctccccagcccggatgagcagtgctgtctgggtgttcccaagaggtcttagctggggtccagagggcctcatccaggtgagaggccaggaagccaggaaacccccactggaggggctccagaccatcttggaggagccggaaaacgaggatgaggatggggccggggacgagggccagcccaagtcctcccagggcaaataggctcctagggccccggggcctcctctcctctcaggcagcagcgccttggaggcagacagaggccaggccagggccagtccctcaccccacattcaggagtaagggccccccacctcccccaaggggctctggccaccacccccattccttccctgtgacccggatgaccacgtttgatacaaaatggggtggggagaggcgcccccccgctcccttgcacccagcacacccagccccagccccaaaccctgccgccacggggggctggcctggagggagccctgagtgggcagctgtggcctgggtgggggcacctgaagatgtctgcccccaacccaggccgtgagttgggagagacagggggaaagaggccctctcaaggttgccagctgcctgggtctctcaagaggggtcagcccacttgccatctccggggcaggctgccctctgttccgggaagctcaccctcacctgtgtgacccacgcgcccagcagacgcccacccactgctagccatcatttctgcgaaaagtcatgtactgtgcgcccatgcaggcggccacctctgggcccggggcacgtgctgtgagcttcctgcgagcccaggctctgcttgctgctgtcctgcatcgtgagcaccacctctgctttcctggcgtagatctaggccaggggctgcttgttctcgtggagctgcgtgtgttcttctctgagcagcctctccccggagacccccagcgcagtcccaggagatggcgggaaggaggcaccagggcacggcggacgctcaccccgtgaccacgatggtgaccctgactgcgggaagaagaaccggacccggggcacagcggggctgcgtgaggatccacaagaactatgctttggcgtttcacccctgttgttacttgtgactcagtttcttcagcctggtggggtgttccctggtgtttcccagtgttctgtgactgtcctgtgaaggccatagggatgggctcaggagggggctccctgaagccagtggacactgccagagtccactgtcctggcaaaaggcagaccctggggccctcgggaaggagggaggtggcagcgggggcagcagcgggagggggagcagatgacgtgccttgccaggaaccccaggaggagggggcctgggacctgtgtcctgtggtggctgtttacagtttctctctgtattgtggtttccttctcttcaatagtttcagtatatgtttctcttcaataaatttcattccgtgttcca

SEQ ID NO: 108 - protein, 369 aa, synthetic construct; identical to SEQ ID NO: 106
  REGION 1..369: note = Synthetic

SEQ ID NO: 109 - DNA, 2475 bp, synthetic construct
  misc_feature 1..2475: note = Synthetic
  source 1..2475: mol_type = other DNA
ggtgccgtggtcaggtcctagggctgggctgtgtcggagactgcaacagtcagcacacattcctgattgatcaggctccctccctcaagtcctctgcgatggctctggcgatgcttcgggactggtgcaggtggatgggtgcgaacgcagagcgctccctgctcatcctgggtatccctgatgactgcaaggaacatgagttccaggaggccgtgcgggctgccctgtcgcccctgggcaggagggagcagccaggctgcgaggaagagtcctttgagagctgggtggagcatgccaaggatatgctgcagctgtggtgccatgcgtcggaaagggagaagaagaggtggctgctggagagcttgggcggcccggccctggaagtcgtgagcggcctcctggaggaagataccaacttgtccgcgctggactgcctggcggcgctggggcaggtatttaggaaccaggacactcgaatgacttcgaggctgaagttcctgacctgcacgcaggggccccaggaggggctgtttgccttcgtggtgcgcctggaaggcctgctgcagagggctgtggagaagggggccgtctgcccagccttggccaattacctgcgactacagcaggtgctgtcttgggcccgccccagcgaggcactccaggataccctgagagggatgcagctggagaagaggccacctggcttcctggggctgctccggctcatccgggagatggaggcatgggcagccttcccagcgaggagccagcagggtgtggcctgggcagcggccccagtggagagtgaagacccagctgctgcccaggcctccccagcccaggggaatgccagcgaggctggtcccggagcagaagatgctgccgaggccgcttctgccaccaaagaggctgcaaggggagcccctgccgctggggaaggtgaaagtgcccctgcaggccccgaaggcctaggtcaggcaaggcccatagaggtcccctggggctcctccccagcccggatgagcagtgctgtctgggtgttcccaagaggtcttagctggggtccagagggcctcatccaggtgagaggccaggaagccaggaaacccccactggaggggctccagaccatcttggaggagccggaaaacgaggatgaggatggggccggggacgagggccagcccaagtcctcccagggcaaataggctcctagggccccggggcctcctctcctctcaggcagcagcgccttggaggcagacagaggccaggccagggccagtccctcaccccacattcaggagtaagggccccccacctcccccaaggggctctggccaccacccccattccttccctgtgacccggatgaccacgtttgatacaaaatggggtggggagaggcgcccccccgctcccttgcacccagcacacccagccccagccccaaaccctgccgccacggggggctggcctggagggagccctgagtgggcagctgtggcctgggtgggggcacctgaagatgtctgcccccaacccaggccgtgagttgggagagacagggggaaagaggccctctcaaggttgccagctgcctgggtctctcaagaggggtcagcccacttgccatctccggggcaggctgccctctgttccgggaagctcaccctcacctgtgtgacccacgcgcccagcagacgcccacccactgctagccatcatttctgcgaaaagtcatgtactgtgcgcccatgcaggcggccacctctgggcccggggcacgtgctgtgagcttcctgcgagcccaggctctgcttgctgctgtcctgcatcgtgagcaccacctctgctttcctggcgtagatctaggccaggggctgcttgttctcgtggagctgcgtgtgttcttctctgagcagcctctccccggagacccccagcgcagtcccaggagatggcgggaaggaggcaccagggcacggcggacgctcaccccgtgaccacgatggtgaccctgactgcgggaagaagaaccggacccggggcacagcggggctgcgtgaggatccacaagaactatgctttggcgtttcacccctgttgttacttgtgactcagtttcttcagcctggtggggtgttccctggtgtttcccagtgttctgtgactgtcctgtgaaggccatagggatgggctcaggagggggctccctgaagccagtggacactgccagagtccactgtcctggcaaaaggcagaccctggggccctcgggaaggagggaggtggcagcgggggcagcagcgggagggggagcagatgacgtgccttgccaggaaccccaggaggagggggcctgggacctgtgtcctgtggtggctgtttacagtttctctctgtattgtggtttccttctcttcaatagtttcagtatatgtttctcttcaataaatttcattccgtgttcca

SEQ ID NO: 110 - protein, 647 aa, synthetic construct
  REGION 1..647: note = Synthetic
MALAMLRDWCRWMGANAERSLLILGIPDDCKEHEFQEAVRAALSPLGRYRVLTKHFRKELGAKAALVEFAEYLNRSLIPHQIPGNGGPWKVIFLPQVPVIEFQDMPSFPAQPQGQAVAKAAGEGGGAGEAGGVGEVGAAGEAGGTGEAGATGEAGAAGEAGGAGEAGGVGEAGAAGEAGGAGEAGAAGEGGAAGEAGGAGEAGGVGEAGAAGEAGGAGEAGGVGEAGAAGEAGGAGEAGAAGEAGGAGEGRAAGEAGAAGEAGAVGEAGAAGEAGAVGEAGAAGEAGAVGEAGGTNVTKAWVQPWRCTLQPVLENRAYRELRPFSRREQPGCEEESFESWVEHAKDMLQLWCHASEREKKRWLLESLGGPALEVVSGLLEEDTNLSALDCLAALGQVFRNQDTRMTSRLKFLTCTQGPQEGLFAFVVRLEGLLQRAVEKGAVCPALANYLRLQQVLSWARPSEALQDTLRGMQLEKRPPGFLGLLRLIREMEAWAAFPARSQQGVAWAAAPVESEDPAAAQASPAQGNASEAGPGAEDAAEAASATKEAARGAPAAGEGESAPAGPEGLGQARPIEVPWGSSPARMSSAVWVFPRGLSWGPEGLIQVRGQEARKPPLEGLQTILEEPENEDEDGAGDEGQPKSSQGK

SEQ ID NO: 111 - DNA, 3389 bp, synthetic construct
  misc_feature 1..3389: note = Synthetic
  source 1..3389: mol_type = other DNA
acacgcttccacgctgcggagctgaggtgatctcttcagacccttcggagagacccagatatctgacttcccactgagagacccggaattgcccagagtttgagaaagctgtgtcggagactgcaacagtcagcacacattcctgattgatcaggctccctccctcaagtcctctgcgatggctctggcgatgcttcgggactggtgcaggtggatgggtgcgaacgcagagcgctccctgctcatcctgggtatccctgatgactgcaaggaacatgagttccaggaggccgtgcgggctgccctgtcgcccctgggcaggtaccgagtactcaccaagcacttcagaaaggagctcggggccaaggcagccttggtggagttcgctgagtatttaaaccgaagcttgattccccatcaaataccaggcaatggggggccctggaaagtgatcttcctgccccaagtacctgttattgagtttcaggatatgcccagttttcctgcacagccccagggtcaagcagtagcaaaagctgcaggtgagggaggaggcgcaggtgaggcaggaggtgtaggtgaggtaggagcagcaggtgaggcaggaggcacaggtgaggcaggagcaacaggtgaggcaggagcagcaggtgaggcaggaggcgcaggtgaggcaggaggtgtaggtgaggcaggagcagcaggtgaggcaggaggcgcaggtgaggcaggagcagcaggtgagggaggagcagcaggtgaggcaggaggcgcaggtgaggcaggaggtgtaggtgaggcaggagcagcaggtgaggcaggaggcgcaggtgaggcaggaggtgtaggtgaggcaggagcagcaggtgaggcaggaggcgcaggtgaggcaggagcagcaggtgaggcaggaggcgcaggtgagggaagagcagcaggtgaggcaggagcagcaggtgaggcaggagctgtgggtgaggcaggagcagcaggtgaggcaggagctgtgggtgaggcaggagctgcaggtgaggcaggagctgtgggtgaagcaggagggacaaatgtaacaaaagcctgggtccagccttggcgctgcaccctacagcctgtgctggaaaacagggcctaccgggaattgagacccttttccaggagggagcagccaggctgcgaggaagagtcctttgagagctgggtggagcatgccaaggatatgctgcagctgtggtgccatgcgtcggaaagggagaagaagaggtggctgctggagagcttgggcggcccggccctggaagtcgtgagcggcctcctggaggaagataccaacttgtccgcgctggactgcctggcggcgctggggcaggtatttaggaaccaggacactcgaatgacttcgaggctgaagttcctgacctgcacgcaggggccccaggaggggctgtttgccttcgtggtgcgcctggaaggcctgctgcagagggctgtggagaagggggccgtctgcccagccttggccaattacctgcgactacagcaggtgctgtcttgggcccgccccagcgaggcactccaggataccctgagagggatgcagctggagaagaggccacctggcttcctggggctgctccggctcatccgggagatggaggcatgggcagccttcccagcgaggagccagcagggtgtggcctgggcagcggccccagtggagagtgaagacccagctgctgcccaggcctccccagcccaggggaatgccagcgaggctggtcccggagcagaagatgctgccgaggccgcttctgccaccaaagaggctgcaaggggagcccctgccgctggggaaggtgaaagtgcccctgcaggccccgaaggcctaggtcaggcaaggcccatagaggtcccctggggctcctccccagcccggatgagcagtgctgtctgggtgttcccaagaggtcttagctggggtccagagggcctcatccaggtgagaggccaggaagccaggaaacccccactggaggggctccagaccatcttggaggagccggaaaacgaggatgaggatggggccggggacgagggccagcccaagtcctcccagggcaaataggctcctagggccccggggcctcctctcctctcaggcagcagcgccttggaggcagacagaggccaggccagggccagtccctcaccccacattcaggagtaagggccccccacctcccccaaggggctctggccaccacccccattccttccctgtgacccggatgaccacgtttgatacaaaatggggtggggagaggcgcccccccgctcccttgcacccagcacacccagccccagccccaaaccctgccgccacggggggctggcctggagggagccctgagtgggcagctgtggcctgggtgggggcacctgaagatgtctgcccccaacccaggccgtgagttgggagagacagggggaaagaggccctctcaaggttgccagctgcctgggtctctcaagaggggtcagcccacttgccatctccggggcaggctgccctctgttccgggaagctcaccctcacctgtgtgacccacgcgcccagcagacgcccacccactgctagccatcatttctgcgaaaagtcatgtactgtgcgcccatgcaggcggccacctctgggcccggggcacgtgctgtgagcttcctgcgagcccaggctctgcttgctgctgtcctgcatcgtgagcaccacctctgctttcctggcgtagatctaggccaggggctgcttgttctcgtggagctgcgtgtgttcttctctgagcagcctctccccggagacccccagcgcagtcccaggagatggcgggaaggaggcaccagggcacggcggacgctcaccccgtgaccacgatggtgaccctgactgcgggaagaagaaccggacccggggcacagcggggctgcgtgaggatccacaagaactatgctttggcgtttcacccctgttgttacttgtgactcagtttcttcagcctggtggggtgttccctggtgtttcccagtgttctgtgactgtcctgtgaaggccatagggatgggctcaggagggggctccctgaagccagtggacactgccagagtccactgtcctggcaaaaggcagaccctggggccctcgggaaggagggaggtggcagcgggggcagcagcgggagggggagcagatgacgtgccttgccaggaaccccaggaggagggggcctgggacctgtgtcctgtggtggctgtttacagtttctctctgtattgtggtttccttctcttcaatagtttcagtatatgtttctcttcaataaatttcattccgtgttcca

SEQ ID NO: 112 - protein, 578 aa, synthetic construct
  REGION 1..578: note = Synthetic
MLQDWCRRMGVNAERSLLILDIPDDCEEHEFQEAVRAALSPLGRYRVLIKVFRKELGARAALVEFAEGLNQSLIPRQIAGKGGPWKVISLPQALDAEFQDIPSFPAQPQGQAVARGAGEAGAAGEAGSVGEAGGVNEERSAGEDEAGGIGEAGGVGEAGAAGEAGAAGEAGAAGEAGGAGEAGGAGEAGGAGEEGGTGEEGGAGEAGGAGEEGGEDEAGAAGEAVGAGVVEAWTQSWRQTLRPLVKTMAYRELRPFSGREQPGCVEESFESWLEDAKDMLQLWCHASERERRRRLLDSLDGLALDIVSGLLEEDPDFSAQDCLTALGQVFRSRDTWMTSRMKFLTCTQGPQEGLFAFVVRLEGLLQKAVEKGAVHPAMANHLRLRQVLSRARPSEALQDTLRRMQLERRPPDFLRLLRLIRDMEAWAASLARSQQGVAWAAAPVESEDPAAAQASPAQGDASEADPGAEDADEAASTTKEAARVAPATGEDENAPAGLEGLGQGRSPDAPGGLPARMGSAVDMAPGGPSWEPEGLVQVGGQEAEEPPQEGLKPILEESENEDEDGAGEAGKPKSPPGK

SEQ ID NO: 113 - DNA, 3294 bp, synthetic construct
  misc_feature 1..3294: note = Synthetic
  source 1..3294: mol_type = other DNA
ctcatctacctggggcttctgggagaagcgggaagccctgtagtcttagcacagcgggctttggagacccctgcgctgcgcagcgcggccgcgtgactgaggcgagatcttccaccctcctgctccagggcctctaggtggcctccagagtgggctccacagcttgaaggcggcgtttttccgcgggtggtcgcagcgtccgcagagtcggagaaagctgcgtcagagactgcaacagtcagcacacattcctgtctgatcaggctccctccctcaagtcctctgcgatagctctggcgatgcttcaggactggtgcaggaggatgggtgtgaacgcagagcgctctctgctcatcctggatatccctgacgactgcgaggaacatgagttccaggaggccgtgcgggctgccctgtcgcccctgggcaggtaccgagtgctcatcaaggtcttcagaaaggagctcggggccagggcagccttggtggaattcgctgagggtttaaaccaaagcttgattccccgccaaatagcaggcaaagggggaccctggaaagtgatctccctgccccaggcccttgatgctgagtttcaggatatacccagtttccctgcacagccccaggggcaagcagtggccagaggtgcaggtgaagcaggagctgcaggtgaggcaggatctgtaggtgaggcaggaggtgtgaatgaggaaagatctgcaggtgaagatgaggcaggaggtataggtgaggcaggaggtgtaggtgaggcaggagcagcaggtgaggcaggagcagcaggtgaggcaggagcagcaggtgaggcaggaggtgcaggtgaggcaggaggtgcaggtgaggcaggaggtgcaggtgaggaaggaggtacaggtgaggaaggaggtgcaggtgaggcaggaggcgcaggtgaggaaggaggtgaagatgaggcaggagctgcaggtgaggcagtaggtgcaggtgtggtggaggcctggacccagtcatggcgccagaccctgcggcctctggtgaaaactatggcctaccgggaactgagacccttttccgggagggagcagccaggctgcgtggaagagtcctttgagagctggttggaggacgccaaggatatgctgcagctgtggtgccacgcgtcggaaagggagagaaggaggcggctgctggacagcttggatggcctggccctggatatcgtgagcggcctcctggaggaagatccagacttctctgcgcaggactgcctgacggcgctggggcaggtgtttaggagccgggatacatggatgacctcgcggatgaagttcctgacctgcacgcaggggccccaggaggggctgtttgccttcgtggtgcgcctggaaggcctgctgcagaaggccgtggagaagggggcggtccacccagccatggccaaccacctgcgactgcggcaggtgctgtctcgggcccgccccagcgaggcactccaggataccctgaggaggatgcagctggagaggaggccacctgacttcctgaggctgctgcggctcatccgggacatggaggcgtgggcagcctccctagcaaggagccagcagggtgtggcctgggcagcggccccagtggagagtgaagacccagctgctgcccaggcctccccggcccagggggacgccagcgaggctgatcccggagcagaagatgctgatgaggctgcttccaccaccaaagaggccgccagggttgcccctgccactggggaagatgaaaatgcccctgcaggccttgaaggcctaggtcagggaaggtcccccgacgcccctgggggcctcccagccaggatgggcagtgcagtcgacatggccccaggaggtcccagctgggagccagagggccttgtccaggttggaggccaggaggctgaggagcctccccaggaggggctcaagcccatcttagaggagtcagaaaacgaggatgaggatggggctggggaggcgggcaagcccaagtcccccccgggcaaataggctctgagggccctgagggctcctctcctctcaggcagcagcaccctggaggcagacagaggccaggccagggccggtccctcaccccatgttgggagctgtgcctcccacctcccctaatgggtcctggccaccaccaccagcccttcccagtgactcagatgaccacgtttgataccaaatggggagctggcctggagggagctctgagcggccagcctgaagatgtctgccccccaccccttgtcctgtgttgggagagatgggggaaagaggccctctcaagggtgacagctgcctacatgtcccaagaggtggcccccacttgccatccctggggcagactgccccccgttccgggaagcccaccctcacctctgtaggccccatagtgacccacgcgtccgaacagcagacgcccactcacccaccgctagccattgttcctacacaaagttgtatgctgtgcgcccatcggtgcggctgcctctgggcctggggcacatgctgtgagcttcctgctagcccaggctctgctagctgctacctgtcccttgtcgtgagcaccacctctgctttcctggtgtagatctaggccaggggctgcttgttctcgtggagctgtgtgtgttcttctctgagcagctcctccccggaggcccccagcacagtcctgggagatggtggaaggaggcgccagggcacgatggacgctcatcctgtgactgctatggtgagcatgaccacgatggtgcccgtgaccatgatggtgaccatgactgtgtgaggaagaaccggacctgggacagagcagagctgccctgcctgaggctccccggggagctttgtgctttggtgttctattcctgttgttacttgtgactcagtttccttgacttgggggggtgttccctgctgtgttttcagtgtcctgtgagggctatagggcagggccctgccccagcggatgggctcgggagggggctccctgaagccagtggacactgccagagtccaccatcctggcaagaggcggaccctggggccctcaggaaggagggagttggcagcgggggctgcagcaggagtaggagcagatgaggcgtcttgccaggaaccccaggaggagggggcccgggacctgtgtgggacctgtgtcctgtggtggcaatttgcagtttctctctgtgttgtgattcccttctcttcaatggtttcagtacatgtttctcttcaataaacttcattcagtgttcca

SEQ ID NO: 114 - protein, 439 aa, synthetic construct
  REGION 1..439: note = Synthetic
MSKTMAMNLLEDWCRGMEVDIHRSLLVTGIPEDCGQAEIEETLNGVLSPLGPYRVLNKIFVREENVKAALIEVGEGVNLSTIPREFPGRGGVWRVVCRDPTQDAEFLKNLNEFLDAEGRTWEDVVRLLQLNHPTLSQNQHQPPENWAEALGVLLGAVVQIIFCMDAEIRSREEARAQEAAEFEEMAAWALAAGRKVKKEPGLAAEVGSALKAETPNNWNATEDQHEPTKPLVRRAGAKSRSRRKKQKKNSRQEAVPWKKPKGINSNSTANLEDPEVGDAESMAISEPIKGSRKPCVNKEELALKKPMAKCAWKGPREPPQDARAEAESPGGASESDQDGGHESPPKKKAVAWVSAKNPAPMRKKKKVSLGPVSYVLVDSEDGRKKPVMPKKGPGSRREASDQKAPRGQQPAEATASTSRGPKAKPEGSPRRATNESRKV

SEQ ID NO: 115 - DNA, 3684 bp, synthetic construct
  misc_feature 1..3684: note = Synthetic
  source 1..3684: mol_type = other DNA
agtgcgcaggcgcgcgctgcgacgccaacctcggcttctcgccgctaacggcatcgagtctggacgccccgtgacccgcctgggccggagcgggggcggacggcgccttcttggccctttctgcctctagcagcggcgccggggtagccggagccagcgactgggaaacggctgcattccactgcgtctccttggcctggctgggcggtcggaggctgatctgccaagattactgtttggagaccgctgcagcccacgtccacctgatagactatttactacacatagtaggttcagagccgctaaatgtccaagaccatggcgatgaaccttctggaggattggtgcaggggaatggaagtggacatccacaggtccttgttggtcacaggcatcccagaggactgtgggcaggcagaaattgaggagaccttgaatggggtcctctccccactgggcccgtaccgcgtgctcaacaagatttttgtgagggaagagaatgttaaagctgccctcattgaggttggtgaaggtgtgaatctgagcaccatcccccgtgaattcccaggaaggggtggtgtctggagagtggtctgtagagaccctacccaggatgccgagtttttaaaaaatctgaatgaattcctggatgccgaggggcgcacctgggaggatgtggtccgcctgctccagctcaaccaccccaccctgtcccagaaccagcatcagcccccagagaactgggcagaagctctgggggtgcttctgggagcagtggtgcagatcatcttctgcatggatgccgagatccgcagccgggaggaagccagggcccaggaggccgctgaattcgaggagatggcagcctgggctttagcagcagggaggaaggtgaagaaagaaccggggcttgcagcagaggtgggttctgccttaaaggcagagacccccaacaactggaatgccacggaagaccagcatgagcctaccaaacctttggttcgcagggctggagctaagtctcgctccaggagaaagaagcagaagaagaactccaggcaggaagcagtgccctggaaaaaacccaaaggcatcaattccaacagcacagctaacttggaggatcctgaggtgggtgatgctgaaagcatggcgatctcagagccgatcaagggcagcagaaagccctgtgtgaataaggaggagttggctttgaagaagcccatggcgaaatgtgcctggaagggtcccagagagccacctcaggatgcccgggcagaagccgagagcccaggaggcgcctctgagtcagaccaagatggtggccatgaaagcccaccaaagaagaaggccgtggcctgggtgtctgccaagaaccccgctcccatgaggaagaagaagaaggtgagcttgggccctgtctcctacgtcttggttgactcagaagatggcaggaagaagccggtgatgccaaagaaagggccaggctcaagaagggaggcatcagatcagaaggcccctcggggccagcagcctgccgaggcaacagcctcaacctctaggggtccgaaggccaagccagaaggctctcctcggcgtgccaccaatgaatccagaaaggtttgatctgggggaccacccatactgaggagttgaaagaacaaggaagaagtaccaagtcaaccaagttctctcttgtcactgaataagactttggactctcttagggcccctgttgatagagatctggccctgaggtaaacgataggtgaggtcttgggtgggagggagttggggaagggaggtggatctctatgcccttttctctaccaggcctgccgtttcaccgccttctctactcaccttctcttcggaggcaggagagttggtaagaggattgggaaaattctagaacattcattcccctttatgcatgagcagggtcactgttacactcatccatgttcagcttttctcccccacgccttgctccctcctcggaatggtcagcgacctctgcaggccctggcatttggaagagctgggtggccctgccatattcttcctcccgccttcctctcgtgtaaccagcggggtaagttaagccaggaccttcgctgcaaacctggttttattgctccttcagtctccagcttccatcctccagttatctagccaggaggtcccaagagttagttttagggaaaaagaatgtctgcctagacctcaaagtctttgagttttagagtctgttagtaaaaatggcactttgattcccatttgggatgagccctctcaggaatcctgtggggaaggggggtgattatagggaaggacgcaggcattcctaggtccccagctctaattccatccatctacccaactgtcaccatctttgcaccaaactgtcaccatctttgcagcagaaggtcactactcacattatagtaagaggggaaaaaaatcttttaaaacttggctgttggccgggcacggtggctcacgcctgtaatcccagcactttgggaggctgaggcaggtcgatcacgaggtcaggcgtttaagaccagcttgaccaacatggtgaaaccccatctctacaaaaattagctgggcgtggtggcgcgcgcctgtaatcccagctactcaggaggctgaggcagatgaattgcttgaatccaggaggcagaggttgcaatgagccgagattgtgccacggcactccagcctgggcagcagagtgagactctctctcaaaaaaacaaaacttggctgttaatgtctgccctctgaattcagacacactatattagaccagaccaccatgtgtcattgtgtgtgtgtgtgtgtgtgtttgcgtgcgcatgtgtagaggagagagcagggtccctgagataatggtttccaaactgtatcgtagccgtccacaaagatgtaatccaaatctatttttctcacgtgtttaaaaaactgaaaagtgactactcagatatgttggaagtcacatcgaagactatcagaatattaccttgtgttcataggttaacttgtttttgtacacgtcttagttatttcaggcatctctttgcttaaaattgagtttcttgtaaatgtgactgatgagcgaggttagaagtggaagaaattcctgtgcatgttctataatctgacaccctgaaagcaagtttcctttcgtcattcacatgctcttgttctgccgtgactgttcaggtgtatggtagtaagtaaatgtattaacatggtgaacagtagtaatattctatcatagagtattagcccttgcaagttttcagggcgtcttttccgacttcagtttttgtgataaagaatgtgaacagttgttagatgttctcagtgattcaactttaaaacaaatttctcgtgatgattcatttcaaaatcctgagtgagtctgactgaaaaatacgagagaaaagagagtggtttccgtttgcagctacacagctgtgtgcatcgacgttctcctggggtgtgtgccaagcgaaacccaggggtgaattggattcttgaagagaccaaagcctgtaactgtccagcttctaatttcaaaacgggtccattagggcttcgttgtgttaacaagttgacaccatgactagtaaatgtaaacgtgtatgtataaaataaagtttagcaaattaa

SEQ ID NO: 116 - protein, 378 aa, synthetic construct
  REGION 1..378: note = Synthetic
MSKTMAMNLLEDWCRGMEVDIHRSLLVTGIPEDCGQAEIEETLNGVLSPLGPYRVLNKIFVREENVKAALIEVGEGVNLSTIPREFPGRGGVWRVVCRDPTQDAEFLKNLNEFLDAEGRTWEDVVRLLQLNHPTLSQNQHQPPENWAEALGVLLGAVVQIIFCMDAEIRSREEARAQEAAEFEEMAAWALAAGRKVKKEPGLAAEVGSALKAETPNNWNATEDQHEPTKPLVRRAGAKSRSRRKKQKKNSRQEAVPWKKPKGINSNSTANLEDPEVGDAESMAISEPIKGSRKPCVNKEELALKKPMAKCAWKGPREPPQDARAEAESPGGASESDQDGGHESPPKKKAVAWVSAKNPAPMRKKKKNPERFDLGDHPY

SEQ ID NO: 117 - DNA, 3479 bp, synthetic construct
  misc_feature 1..3479: note = Synthetic
  source 1..3479: mol_type = other DNA
agtgcgcaggcgcgcgctgcgacgccaacctcggcttctcgccgctaacggcatcgagtctggacgccccgtgacccgcctgggccggagcgggggcggacggcgccttcttggccctttctgcctctagcagcggcgccggggtagccggagccagcgactgggaaacggctgcattccactgcgtctccttggcctggctgggcggtcggaggctgatctgccaagattactgtttggagaccgctgcagcccacgtccacctgatagactatttactacacatagtaggttcagagccgctaaatgtccaagaccatggcgatgaaccttctggaggattggtgcaggggaatggaagtggacatccacaggtccttgttggtcacaggcatcccagaggactgtgggcaggcagaaattgaggagaccttgaatggggtcctctccccactgggcccgtaccgcgtgctcaacaagatttttgtgagggaagagaatgttaaagctgccctcattgaggttggtgaaggtgtgaatctgagcaccatcccccgtgaattcccaggaaggggtggtgtctggagagtggtctgtagagaccctacccaggatgccgagtttttaaaaaatctgaatgaattcctggatgccgaggggcgcacctgggaggatgtggtccgcctgctccagctcaaccaccccaccctgtcccagaaccagcatcagcccccagagaactgggcagaagctctgggggtgcttctgggagcagtggtgcagatcatcttctgcatggatgccgagatccgcagccgggaggaagccagggcccaggaggccgctgaattcgaggagatggcagcctgggctttagcagcagggaggaaggtgaagaaagaaccggggcttgcagcagaggtgggttctgccttaaaggcagagacccccaacaactggaatgccacggaagaccagcatgagcctaccaaacctttggttcgcagggctggagctaagtctcgctccaggagaaagaagcagaagaagaactccaggcaggaagcagtgccctggaaaaaacccaaaggcatcaattccaacagcacagctaacttggaggatcctgaggtgggtgatgctgaaagcatggcgatctcagagccgatcaagggcagcagaaagccctgtgtgaataaggaggagttggctttgaagaagcccatggcgaaatgtgcctggaagggtcccagagagccacctcaggatgcccgggcagaagccgagagcccaggaggcgcctctgagtcagaccaagatggtggccatgaaagcccaccaaagaagaaggccgtggcctgggtgtctgccaagaaccccgctcccatgaggaagaagaagaagaatccagaaaggtttgatctgggggaccacccatactgaggagttgaaagaacaaggaagaagtaccaagtcaaccaagttctctcttgtcactgaataagactttggactctcttagggcccctgttgatagagatctggccctgaggtaaacgataggtgaggtcttgggtgggagggagttggggaagggaggtggatctctatgcccttttctctaccaggcctgccgtttcaccgccttctctactcaccttctcttcggaggcaggagagttggtaagaggattgggaaaattctagaacattcattcccctttatgcatgagcagggtcactgttacactcatccatgttcagcttttctcccccacgccttgctccctcctcggaatggtcagcgacctctgcaggccctggcatttggaagagctgggtggccctgccatattcttcctcccgccttcctctcgtgtaaccagcggggtaagttaagccaggaccttcgctgcaaacctggttttattgctccttcagtctccagcttccatcctccagttatctagccaggaggtcccaagagttagttttagggaaaaagaatgtctgcctagacctcaaagtctttgagttttagagtctgttagtaaaaatggcactttgattcccatttgggatgagccctctcaggaatcctgtggggaaggggggtgattatagggaaggacgcaggcattcctaggtccccagctctaattccatccatctacccaactgtcaccatctttgcaccaaactgtcaccatctttgcagcagaaggtcactactcacattatagtaagaggggaaaaaaatcttttaaaacttggctgttggccgggcacggtggctcacgcctgtaatcccagcactttgggaggctgaggcaggtcgatcacgaggtcaggcgtttaagaccagcttgaccaacatggtgaaaccccatctctacaaaaattagctgggcgtggtggcgcgcgcctgtaatcccagctactcaggaggctgaggcagatgaattgcttgaatccaggaggcagaggttgcaatgagccgagattgtgccacggcactccagcctgggcagcagagtgagactctctctcaaaaaaacaaaacttggctgttaatgtctgccctctgaattcagacacactatattagaccagaccaccatgtgtcattgtgtgtgtgtgtgtgtgtgtttgcgtgcgcatgtgtagaggagagagcagggtccctgagataatggtttccaaactgtatcgtagccgtccacaaagatgtaatccaaatctatttttctcacgtgtttaaaaaactgaaaagtgactactcagatatgttggaagtcacatcgaagactatcagaatattaccttgtgttcataggttaacttgtttttgtacacgtcttagttatttcaggcatctctttgcttaaaattgagtttcttgtaaatgtgactgatgagcgaggttagaagtggaagaaattcctgtgcatgttctataatctgacaccctgaaagcaagtttcctttcgtcattcacatgctcttgttctgccgtgactgttcaggtgtatggtagtaagtaaatgtattaacatggtgaacagtagtaatattctatcatagagtattagcccttgcaagttttcagggcgtcttttccgacttcagtttttgtgataaagaatgtgaacagttgttagatgttctcagtgattcaactttaaaacaaatttctcgtgatgattcatttcaaaatcctgagtgagtctgactgaaaaatacgagagaaaagagagtggtttccgtttgcagctacacagctgtgtgcatcgacgttctcctggggtgtgtgccaagcgaaacccaggggtgaattggattcttgaagagaccaaagcctgtaactgtccagcttctaatttcaaaacgggtccattagggcttcgttgtgttaacaagttgacaccatgactagtaaatgtaaacgtgtatgtataaaataaagtttagcaaattaa

SEQ ID NO: 118 - protein, 635 aa, synthetic construct
  REGION 1..635: note = Synthetic
MAMSLLQDWCRSLDVDAHRALLVTGIPEGLEQADVEAVLQPTLLPLGTFRLRHMKALMNEKAQAALVEFVEDVNHAAIPREIPGKDGVWRVLWKDRAQDTRVLRQMRRLLLDDGPTQAAEAGTPGEAPTPPASETQAQDSGEVTGQAGSLLGAARNPRRGRRGRRNRTRRNRLTQKGKKRSRGGRPSAPARSEAEDSSDESLGIVIEEIDQGDLSGEEDQSALYATLQAAARELVRQWAPCNSEGEEDGPREFLALVTVTDKSKKEEAEKEPAGAESIRLNTKEDKNGVPDLVALLAVRDTPDEEPVDSDTSESDSQESGDQETEELDNPEFVAIVAYTDPSDPWAREEMLKIASVIESLGWSDEKDKRDPLRQVLSVMSKDTNGTRVKVEEAGREVDAVVLRKAGDDGDLRECISTLAQPDLPPQAKKAGRGLFGGWSEHREDEGGLLELVALLAAQDMAEVMKEEKENAWEGGKYKYPKGKLGEVLALLAARENMGSNEGSEEASDEQSEEESEDTESEASEPEDRASRKPRAKRARTAPRGLTPAGAPPTASGARKTRAGGRGRGRGVTPEKKAGSRGSAQDDAAGSRKKKGSAGAGAHARAGEAKGQAPTGSKAARGKKARRGRRLPPKCR

SEQ ID NO: 119 - DNA, 4677 bp, synthetic construct
  misc_feature 1..4677: note = Synthetic
  source 1..4677: mol_type = other DNA
agacgcggcgcgagcgccaggcaagctgcggctgctacctcccacgcctctccaggtgcactcggcgccgcccccctgcacctggctgcggtgccgagtcactcaggcctgtgtcagggagagagggagggagctgtcctggaaagcagacacgtaagccccccgcggatcctcagacagctctggagaggggtcccgggggaaggtcactgcgtccagccggccagcaggcagctagagcccccgagccccgagccccactccagccttgccacattcaccggaaccgggactctaagccctgcaagtggctttctagggttgcattgacaccgtgcgctgcagcccacccctatctcgggctccctgctgccccaagatcagcgccaagggggctgcaccatggccatgagccttttgcaggactggtgccggagcctggacgtggacgcgcacagggccctgctggtcaccggcatcccggagggcctggagcaggcagacgtcgaagccgtcctgcagccgaccctcctgcccctgggcacgttcaggttgcgacacatgaaggctttgatgaacgagaaggcccaggccgccctggtggagtttgtggaggacgtcaatcacgctgccattcccagggagatcccaggcaaggatggggtctggagggttctgtggaaggaccgtgcgcaggacacgagggtcctgaggcagatgagacgcctgctgctggatgacgggcccacgcaggccgcggaggctgggacccccggggaggcacccacccctcccgcttcggagacgcaggcccaggattctggggaggtaacagggcaggctggctcgcttcttggggcagccaggaacccaaggaggggccgtcggggtcgcagaaacagaaccagacgcaacaggttgacccagaagggcaagaagagaagccgaggaggacggccgtctgctcccgcgaggagtgaggccgaggactcttccgacgagagcctgggcatcgtgatcgaggagatcgaccagggcgacctgagcggagaagaggaccagagcgcgctgtacgccacgctgcaggccgctgccagggagctggttaggcagtgggcgccctgcaactccgagggggaagaagacggtccccgcgagttcttggctctggtcaccgtcaccgacaaatcgaagaaagaagaggcagagaaggagccagctggggccgaatccatccgcttgaacaccaaagaagacaaaaatggtgtccccgacttagtggccctgctggctgtgagagacaccccggacgaggagccggtggacagcgacacttcggagagcgactcgcaggaaagtggggaccaagaaacagaggagttggataatcctgagttcgtggccattgtggcctataccgacccgtcggacccctgggcccgggaggagatgttgaaaatcgcttctgttatcgagtcgctgggctggagcgacgagaaagacaagcgagaccccctccgacaggtcttgtccgtcatgtccaaggacactaacgggacccgcgtgaaggtggaagaggcgggccgcgaggtggacgccgtggtcctgcgcaaggccggggatgacggggacctccgggagtgcatttccaccttggcgcagccggatctccctccccaggcgaagaaggctgggcgtggcctcttcgggggctggagcgagcaccgtgaggacgaagggggtcttctggagctggtggcgctcctggctgcccaggacatggcggaggtgatgaaggaggaaaaagaaaacgcctgggaaggcgggaagtacaaataccccaaaggcaaactgggggaggtattggcgctcctggccgcccgggagaacatggggtccaacgaggggtcggaggaggcttcggacgaacagtccgaggaggagtcggaggacaccgagagcgaggcgtcggagccggaggacagggcatccaggaagccccgggccaagagggcgcgcacggcccccaggggcctgactccggccggcgcgcctcccaccgcttccggggcccgcaaaacccgcgcgggcggccgaggccgaggccggggcgtcactcccgagaagaaagccgggagccggggctcggcccaggacgacgccgcaggaagcaggaagaagaaggggagcgcgggcgccggggcccatgccagggcaggcgaggccaagggccaggcgcccactggatccaaggccgcgcgcgggaagaaggcccgtcggggccggaggctgccccctaaatgccgctagtggccccccaagaagccgcccaggctgcgagcaggccccgcagggcacccgcccgcctgtggcccccgccctcccctcccctcttcctgtcctccgcagacgcaatctcctcgcttcacagcgcgcccgggccgcgttttgccagcgtcacgttcccctctcgggccctcgcaggccgggggcgccagcgatcccgacggaggaagcccggatgggaggaggaaagagaagtgggcgcccgaggcagcagcgcagggccgagatggggacgcgccaagtggaccaggattgggggcccgggttgcccccggagggggtgtgtgtgtggacgccgggcacctgcagaggcgagcagggctcttcgtggcgctctcggggcctgcgcctggcaggtgctgtaggccgctgtcgcccctaccccagtctgactgggccctgggtctgtggtggaggctcagtcaccagccgcgcagcgcgtgtcagggcgcaactctcagccaggggaggccccagctcccagccaggggaagagatgattccagaaaggaaagtctgagagatagaaggcggttgggaaggggaggaggaggaaaggggagaggaacggtgggagaagggaaagaggaggaggagggggaggggggagcagagggaagacacatgccagccctgcctactggggcgcccctgataacaaaggaaccagccccaggccaaggccacctgcccctgaccacaagttgaatttgtcactcagactgcagtgtttcccaacattctaattatttgcagaggtgttcaatttggggtaattcacttaaaatccagttttggttcttctgggctgagtgggccctggcccctcccataggctgtggctcccctgggtgccccctctccagtggagctgacccaccgctcagcgctggccttgcagcccttactaaaagacttgaaagtccctgggttcaccccctgagtgaattaaaggccagaggggccccgaagggcactgtgagggacagaggctcacctgggcagtgcagaagccggccgcgtgtccctccttacaggggatgaaatgacctggggaggaaaccccagccctgccctggaggttccagagtaggcgggccggtgctgtgaggcttcacaacctgctgtcccaagcacgcttgagttgtatgtgagtctgtgccgtgccgtgccgtatgcttcagctcctgcaaccccggctgagctcgatttttacctaaatatcagtctccacgggaccccaccttcattcatgccttcttgtccctggggcaatgtgtgtgcttcctcgtcccaatttccattccctggcagtgaggagcccatcgtgccagggggccctgccccacttgtccctgggaaggaataggagggtttgggtgtgacctcacagtccagaccagactgtcccagtcctatgtcagggacacccagatgtagaagctgactgagacctgctgcagggcgtgggtgctcccctctgcttggaggctgtccctggacagtgacccacccactgaggaccaggctgggtgtaccttgagctgggcacagcagcctgtggtgttgcctgtgggtggggagggccccaggtgtgcttctcccgtagcagtcctaggcttctctccctgtgccctgtgtcacctggatcctccagtaaagtgaaattcagcactgtactctctctgtgctctgggcagtggggcaggcggggtgtgggagcgtgggccacagatgtccacggtcttgactgtggtttgcccagaatacctgggaactgtcctgtcactggtttacatacactgtcctttgctgcttcgggatccctgcctggctctccctaccccccagcatcatctcaccccttgcagatctgagccagcttccactcccacccctgatgcctccccacttccagcctcagctccgaagcccctggacacccatggagaccccgcccagccaatccccaccctagcttccacccagatacactctgccaggccacagctgcaggcactctccccccagcctccacccctcacctgtgccctggacctcagactcagctttccatcctacctgagttttctgcctccctccatcctgtgtccccccaccatacatggctgccagagacgtcctcttagaagtcacacctggggtctgattgcgtccctcgcctccccagatcccccaaggtctccttcctgtgccgtcatatctgcagttcttaggactgtctagacatgctttgttcaactaggtaatcacacggggtaaattggatttaaatgtaattaagattaaataaaaatacacatgcatgtc

SEQ ID NO: 120 - protein, 204 aa, synthetic construct
  REGION 1..204: note = Synthetic
MLFGVKDIALLEHGCKALEVDSYKSLMILGIPEDCNHEEFEEIIRLPLKPLGKFEVAGKAYLEEDKSKAAIIQLTEDINYAVVPREIKGKGGVWRVVYMPRKQDIEFLTKLNLFLQSEGRTVEDMARVLRQELCPPATGPRELPARKCSVPGLGEKPEAGATVQMDVVPPLDSSEKESKAGVGKRGKRKNKKNRRRHHASDKKL

SEQ ID NO: 121 - DNA, 4240 bp, synthetic construct
  misc_feature 1..4240: note = Synthetic
  source 1..4240: mol_type = other DNA
gacgtcaccgcgggagcaagtgcagcggccacatcctcgtcctagtccggcgacagggcactgagtgcaacctctgggccagcggtgagggacgcgcctccctgcctgccagggccgcgcctacgacactctgttggcagttgtcccagggagatcagcagtcccagatccggagagaccgttggggcctgtgagaccttcggaggccacgccaagaaaggggaagcatcatctttagaccctccttggtgcagccttgcccaggacccgggagccacgagtcaggtggcaaacaccgccttctgcttctccagcacgcgtaagtccctgggaatcttcgagcccactcagagggagccacagaagccccgacgttgcacagccctgcaggcaggggctgggggcatccttcactgagctggacgaaggggtcctgggagcggcctccggcccctgaggcctcactctctcgtagcctttgggaaggaatcgcggcatcctggataatttcgtggattcccgaggcagctcaggtgtccaccagcgggcatactgctgcctcaaagagttgggcaagatgctgttcggggtgaaggacattgcactgttggagcacgggtgcaaggccctggaggtggacagttacaagtccctgatgatcctggggatcccggaggactgcaaccacgaggaattcgaagagattattcggctgcccctcaaacctctaggcaagttcgaagtggctgggaaggcctatctggaagaagataaatccaaggcggccatcattcagctgacggaggacatcaattacgccgtggtccccagggagatcaagggcaagggcggtgtgtggagagtggtctacatgccccggaagcaggacattgaattcctgaccaagctgaacctctttctgcagagcgagggcaggacggtggaggatatggcccgggtcctgaggcaggaactgtgtcccccagccacgggccccagagagctgcctgcaagaaagtgctctgtgcctgggctgggggagaaaccggaggctggggccaccgtccagatggacgtggtgccacctctggactcctccgagaaggagagcaaggctggagttggcaagaggggcaaaaggaagaacaagaaaaaccgccggcggcatcacgcctcagacaagaagctgtgaggtggtggcatcgggtgggcgtggggagtgtctgagggggtgtgggtgatgactgtgggagggtaggtaccacctcactacgtttaaggagtgcgagtgagtgtgggtaatgcaagggagggtgggaatgactctagtgagtgggtaactttggagtaagaatgggtatcatttggatagcacaggtaacatctgactagtgcgaaagaggtggatatctttatctgagttaactaacttgtgggttacatctgagtgggtgtggttaacgtctgggtgcccgtggctcatttattcatcttacagatatttattgcatacctactatgttccaggagctggggatatggaagggagtaaactcaataaaaatccctgccttcatggcactcagagtaagtgggtaatatttgaattacttggagacatcagagtgagcgtaggtagcatttgagtacatgtgggtaacagctgaatgatcataaaatctggttgagtgtggattgctcattcatccatgtatccatgcaaccaacaaaatttcctgaggacctacagtgtgccaggcactggggatacagaaggggaataaacagaccaaaaccttgcccgtgactctcatagtagctatttgagtaggtgcgggtaacatttgggttacttgaatacacgttagtgagtgtgggtagcattagagtgtgtgggtggcatccgcgtataattgaaagtagttgtgggtaatatttgagagtgtgcgggtaacacctgaatatgtgtatgggtatcatttgagtagatgagagtaatttctaagcgaggaagggtctcaaagacccacaaatatcttccgctactcgggagaccccgcgggaccagccgcgcaggcgcagtcagagccagaacctagcagcgcgtccgaccgttgctatggagaccacaggctgaccccaggcgcctgcgccctttgcagaaaatggaggaggtggaaacggtcctggattccaaggttttgcagatctcttactggagttgaagaaaactcacgcagacccaggaggaacgtgtcagggagatccctactcgggagaagggccgccatcctccttcggctgtcacccacggcctgattctggggacagcctctcttgctggggggaggcgggcggcacggacccctctagctttgcgccctcctggaaatcctgttattgcaaagtctagagccgttttctgtgtttcaaaagcatgtaccttgaatgtacctctggcacaaacttgttcggagtgggccaccctgttttgcgcatgtcaagcagaaaccatggctgagagtgagttgcctgctccccacctctgaggaactcactgatctgtaagcggctcttgaaaagctgtcgtctttctctacacgctgagtctctcttacagaagtggtcagaagcgggacccttctgccccgattggcgcctggggtgagggtgtgagtctccccaagttaaattccagaagctctggcttggtgaggaggagtgaactccactctctaaagaagaggcaaggcttctgggcaatggtccaaaaggaggtaaatcattaagaggccattttcaagagttaccaatgaacgacgttaacactgaggtgaagaagaattttaaaaatcaggtaaaatccaaatacatgtgagtaggcagatcttttgctcagagacaacggacctgtcaatttgcaaacttgttagttcaaggatctgcaaaggtttgactgaatctcttttcaaaaatgtaacttacacacaacacaaagactcagcctaccaaattgtaatgtcagagactcctaacgtcttttttttttgagacagagtctcgctctgtcaccaggctggagtgcagaggtgcaatctcagctcactgcaacctctgactccctggtccaagcgattctcctgcctcagcctccctagtagctaggattacaggcatgaaccaccacgtccagctaaattttgtatttttactagagaaagggtttcaccatgttggccaggatggtctcaatctcccgccttggtctcccaaagtgctgggattgcaggtgtaagccactgcgcccagcttctctgttgattctttttttttttttttttttttttttgaggtggagtctccttctgtagcccaggctggagtgcagtggcacgatcttggctcactgcaacctctgcttcctgggttcaagtgattctcctgcctcagcctcctgagtatctgggactacaggtgcgcaccaccacgcccagctaatttttgtatttttagtagagacagggtttcaccgtattagccaggatggtctcaatctcctgaccttgtgatccgcccacttcggcctcccaaagtgttgggattacaggcgtgagccaccacgcccggcctgatttttctattccttgtaaatgcccagcctgggcatacccactcgcagaatattgtactactgtgttcgttattgcatcccagagtctgagcttgaataaatgctacgaaagcctccagcgtgtttgttgcatgctgctctcccatcaaggcataatcgttgtataataagggatagtgtgacatttcttatcatattgcagcatgaagactgtattttgtgcttattgtggatttcaggggtataacatttatatttgtgatacacaacatgcgtggttgttcaatgttgcccatgaaatcaaaatcctaccacttctctttcaagattttctggaccaaagtgtatcctgggactccaatgcaagttgtcatgcaaaactgtcaccatttgaagctacttagctgagtgaaaacatcactggtagggcaaatcccaagcaaaaattcacaagtaaattgaatgtgccagctgttggagggctgcatctggcttctgtaaacccctgatttcaaaagggggttcagctaaaactcttcattgttctttgtgattgagatgctcttttgtacaaatatgaactcataaaataaatgtatgctaatgaaa

SEQ ID NO: 122 - protein, 538 aa, synthetic construct
  REGION 1..538: note = Synthetic
MLQIGEDVDYLLIPREVRLAGGVWRVISKPATKEAEFRERLTQFLEEEGRTLEDVARIMEKSTPHPPQPPKKPKEPRVRRRVQQMVTPPPRLVVGTYDSSNASDSEFSDFETSRDKSRQGPRRGKKVRKMPVSYLGSKFLGSDLESEDDEELVEAFLRRQEKQPSAPPARRRVNLPVPMFEDNLGPQLSKADRWREYVSQVSWGKLKRRVKGWAPRAGPGVGEARLASTAVESAGVSSAPEGTSPGDRLGNAGDVCVPQASPRRWRPKINWASFRRRRKEQTAPTGQGADIEADQGGEAADSQREEAIADQREGAAGNQRAGAPADQGAEAADNQREEAADNQRAGAPAEEGAEAADNQREEAADNQRAEAPADQRSQGTDNHREEAADNQRAEAPADQGSEVTDNQREEAVHDQRERAPAVQGADNQRAQARAGQRAEAAHNQRAGAPGIQEAEVSAAQGTTGTAPGARARKQVKTVRFQTPGRFSWFCKRRRAFWHTPRLPTLPKRVPRAGEARNLRVLRAEARAEAEQGEQEDQL

SEQ ID NO: 123 - DNA, 3236 bp, synthetic construct
  misc_feature 1..3236: note = Synthetic
  source 1..3236: mol_type = other DNA
gcagagctctaagcgcgcgggctggcaggctgcggcgcgtcaaggtcagcctggagctgggtggcggcctgcctgggggcgggggaccctactggaggcccgggctggggcctcccagcgcctcggccatattgaatagcttcgactggaccgtctttgtctgcgaagtcctgtcccaagttccagccgcgtccctggggcctggggcaggaagagtcgctggcagcccgcgcgccccaacttggagctgggacaccacgtttccagcttggagtgggccttgagccttgggactgacctcgcccccggctcacgtaggcatcctggaaattgattcccccaagtccttggtgggggagccggacttggtcaagactgtacttgttgcaggcgaagagattggaggcgtttggctcgtccctggctagggaggtgagactctccggtcagcgttgctggaactccccccatccagtccctccctcaagactaagggctacagtagtttgttggggctcattgccccctcaccccagatatcaccctggagatcttaaagactctcgagaaaagccacgtggggggctggttcccctggggcttcctgccgtcccccgactgcctcattctttggagcgtccccgatgtctgcaaagatgtggatttggacgtcctcgtggaagccctaaagcccgtggggacatttaagaagatcggcaaggtgttccgcaaggaggaggactccacggtggggatgctgcagatcggggaggacgtcgactatttgctcatcccccgggaggtcaggctggctgggggcgtctggagagtcatctctaagcccgccaccaaggaagcagaatttcgggagcggctgacccagttcctggaagaagagggccgcaccctggaggacgtggcccgcatcatggagaagagcaccccgcacccgccccagccccccaaaaagcccaaggagccccgagtgaggaggagagtgcagcagatggtgactcctccgccccggctggtcgtgggcacgtacgacagcagcaacgccagcgacagcgagttcagcgacttcgagacctccagagacaagagccgccagggcccgcggcggggcaagaaggtgcgcaaaatgcccgtcagctacctgggcagcaagttcctgggaagcgacctggagagtgaggatgatgaggaactggtcgaggccttcctccggcgacaggagaagcagcccagcgcgccgcctgcccgccgccgcgtcaacctgccagtgcccatgtttgaggacaacctggggcctcagctgtccaaagcggacaggtggcgggagtatgtcagccaggtgtcctgggggaagctgaagcggagggtgaagggttgggcgccgagggcgggccccggggtgggcgaggcccggctggcctccaccgcagtggagagcgcaggggtatcatcggcgccagagggcaccagcccgggggatcgcttgggaaacgcgggagatgtttgtgtgccccaggcttcccctaggcgatggaggcccaagatcaactgggcctcctttcggcgccgcaggaaggagcagacagcacccacaggtcagggggcagacatcgaggctgatcaggggggagaggctgcagatagtcaaagggaagaggccatagctgaccagcgggaaggggctgcaggtaatcagagggcaggggccccagctgaccagggggcagaggctgcagataatcagagggaagaggctgcagataatcagagggcaggggccccagctgaggagggggcagaggctgcagataaccagagggaagaggctgcagataatcagagggcagaggccccagctgaccagaggtcacagggcacagataaccacagggaagaggctgcagataatcagagggcggaggccccagctgaccaggggtcagaggttacagataatcaaagggaagaggccgtacatgaccagagggaaagggccccagctgtccagggtgcagataatcagagggcacaggcccgggctggccagagggcagaggctgcacataatcagagggcaggggccccaggtatccaggaagctgaagtctcagctgcccaagggaccacaggaacagctccaggagccagggcccggaaacaggtcaagacagtgaggttccagacccctggacgcttttcgtggttttgcaagcgccggagagccttctggcacactccccggttgccaaccctgcccaagagagtccccagggcaggagaggccaggaacctcagggtgctgagggccgaggccagagcagaagctgagcagggagagcaagaagaccagctgtgaggtgagggctagagacagcccacgggccctccctccaagtgtgggagggagagatgctctgcctctgaacttcaaagtggaggtggagtgctggccacgtctccacctaacaaccctctttattctcttgttaaagttttgttcatgctttgatttttttttaaattttttagagacagggtctcactctgttgcccaggctggagtgcagtggcatgatcataactcactgcagcctcaaacttctggcctcaagtgatcctcctgcctcggcctcccaaaatgctgggattacagatgtgagccaccacacacaccatctgattaaaaaaaaaaaatactgattccttgtagcaacccaaaaaatgtaaagaaagaattcgacacaatggggttgcgtcacctatagttttcatttcctcaaatgcaactcaaggtgctcagcttgataaagggagagagggagggcaggggagaaactgactcctttttccatctcttttttgcctgcacacttaatgtggactgcctccttggacttaaaaagagttgagccaacatgcaaatcaaaaaacatttatgaactgatttcttagctttggacttgacaactgacgaatgctctgggggtgacctggcccttgctgtccgttccactcccatccttgatgatggagacacctgtgtttatgttgtcacttcgcttgccatcctcatgccccgggctctgctggtttggaggggacagggcccttggtttgcacacctcacccgatcttggttataaggtacagtaaaagcgtctgaaaaatgcattcaaaacacagattctggggccctcggcaccagaggtgtggatttgcta

SEQ ID NO: 124 - protein, 402 aa, synthetic construct
  REGION 1..402: note = Synthetic
MASIIARVGNSRRLNAPLPPWAHSMLRSLGRSLGPIMASMADRNMKLFSGRVVPAQGEETFENWLTQVNGVLPDWNMSEEEKLKRLMKTLRGPAREVMRVLQATNPNLSVADFLRAMKLVFGESESSVTAHGKFFNTLQAQGEKASLYVIRLEVQLQNAIQAGIIAEKDANRTRLQQLLLGGELSRDLRLRLKDFLRMYANEQERLPNFLELIRMVREEEDWDDAFIKRKRPKRSESMVERAVSPVAFQGSPPIVIGSADCNVIEIDDTLDDSDEDVILVESQDPPLPSWGAPPLRDRARPQDEVLVIDSPHNSRAQFPSTSGGSGYKNNGPGEMRRARKRKHTIRCSYCGEEGHSKETCDNESDKAQVFENLIITLQELTHTEMERSRVAPGEYNDFSEPL

SEQ ID NO: 125 - DNA, 2197 bp, synthetic construct
  misc_feature 1..2197: note = Synthetic
  source 1..2197: mol_type = other DNA
ggcgctgcctcgtctctgctacccctggttgggcggccctgcgaagcagctccttcgggcagccccgggtcgcttagcggccaaggaggcttcagttctttgccgcctgcaaggcggagaccagaaggcggaatccacagctggcgacgcgggagcatctgctgtccaccagcggagcacaggccatcaaagccgcatctgaacttgaattctgtgcagctgattgcagagctggacccggatctgcgaccccctgtggacagaggttgaccgtaccccggagaggagctttctcacggagggcactggttgcagaggctggaagtgaaataaagacgcgctcttgtttcagagttcgtcccctgctgagataggaaggcagagccacctcctctcctctcccacctgcagattaagcttttctaaaaagcctaggcatcttcttatattcagataccctatcgtcgtcagtcatggctagcatcattgcacgtgtcggtaacagccggcggctgaatgcacccttgccgccttgggcccattccatgctgaggtccctggggagaagtctcggtcctataatggccagcatggcagacagaaacatgaagttgttctcggggagggtggtgccagcccaaggggaagaaacctttgaaaactggctgacccaagtcaatggcgtcctgccagattggaatatgtctgaggaggaaaagctcaagcgcttgatgaaaacccttaggggccctgcccgcgaggtcatgcgtgtgcttcaggcgaccaaccctaacctaagtgtggcagatttcttgcgagccatgaaattggtgtttggggagtctgaaagcagtgtgactgcccatggtaaattttttaacaccctacaagctcaaggggagaaagcctccctttatgtgatccgtttagaggtgcagctccagaacgctattcaggcaggcattatagctgagaaagatgcaaaccggactcgcttgcagcagctccttttaggcggtgagctgagtagggacctccgactcagacttaaggattttctcaggatgtatgcaaatgagcaggagcggcttcccaactttctggagttaatcagaatggtaagggaggaagaggattgggatgatgcttttattaaacggaagcgtccaaaaaggtctgagtcaatggtggagagggcagtcagccctgtggcatttcagggctccccaccgatagtgatcggcagtgctgactgcaatgtgatagagatagatgataccctcgacgactccgatgaggatgtgatcctggtggagtctcaggaccctccacttccatcctggggtgcccctcccctcagagacagggccagacctcaggatgaagtgctggtcattgattccccccacaattccagggctcagtttccttccaccagtggtggttctggctataagaataacggtcctggggagatgcgtagagccaggaagcgaaaacacacaatccgctgttcgtattgtggtgaggaaggccactcaaaagaaacctgtgacaacgagagtgacaaggcccaggtttttgagaatttgatcatcactctccaggagctgacccatactgagatggagaggtcaagagtggcccctggcgaatacaatgacttctctgagccactgtaagggaccacccccaggtttcagtgaacccttacctatattcagcatccagtagtgggaaaactggggtgggggtgggggtgggacttctaactgcatgaattaatccacaaagcggctatcttttggggtggagtagaaagggtcttggataccagcacattggagggagatagcctgacctctgtccttgctccttctccctgcagcctacgggtctgttttctgtgtgtgcccatttccttgacagctttattctttgtgaaagtggtataatttattgttaaatatttgaacaataaaaaaggtacaaaaagtgaagtacaaattacccaaatctctccacccttatataatcattgtcaaccctttgatgagtgatatttccctatacctatgtacccagatagatatatgcatagataaaagtgatgaaatataagtgctgttctatctgtattttttcaccaaacaatatatgttgtgagcttctatgtcaataaatatatatatcagca

SEQ ID NO: 126 - protein, 402 aa, synthetic construct; identical to SEQ ID NO: 124
  REGION 1..402: note = Synthetic

SEQ ID NO: 127 - DNA, 2145 bp, synthetic construct
  misc_feature 1..2145: note = Synthetic
  source 1..2145: mol_type = other DNA
ggcgctgcctcgtctctgctacccctggttgggcggccctgcgaagcagctccttcgggcagccccgggtcgcttagcggccaaggaggcttcagttctttgccgcctgcaaggcggagaccagaaggcggaatccacagctggcgacgcgggagcatctgctgtccaccagcggagcacaggacccggatctgcgaccccctgtggacagaggttgaccgtaccccggagaggagctttctcacggagggcactggttgcagaggctggaagtgaaataaagacgcgctcttgtttcagagttcgtcccctgctgagataggaaggcagagccacctcctctcctctcccacctgcagattaagcttttctaaaaagcctaggcatcttcttatattcagataccctatcgtcgtcagtcatggctagcatcattgcacgtgtcggtaacagccggcggctgaatgcacccttgccgccttgggcccattccatgctgaggtccctggggagaagtctcggtcctataatggccagcatggcagacagaaacatgaagttgttctcggggagggtggtgccagcccaaggggaagaaacctttgaaaactggctgacccaagtcaatggcgtcctgccagattggaatatgtctgaggaggaaaagctcaagcgcttgatgaaaacccttaggggccctgcccgcgaggtcatgcgtgtgcttcaggcgaccaaccctaacctaagtgtggcagatttcttgcgagccatgaaattggtgtttggggagtctgaaagcagtgtgactgcccatggtaaattttttaacaccctacaagctcaaggggagaaagcctccctttatgtgatccgtttagaggtgcagctccagaacgctattcaggcaggcattatagctgagaaagatgcaaaccggactcgcttgcagcagctccttttaggcggtgagctgagtagggacctccgactcagacttaaggattttctcaggatgtatgcaaatgagcaggagcggcttcccaactttctggagttaatcagaatggtaagggaggaagaggattgggatgatgcttttattaaacggaagcgtccaaaaaggtctgagtcaatggtggagagggcagtcagccctgtggcatttcagggctccccaccgatagtgatcggcagtgctgactgcaatgtgatagagatagatgataccctcgacgactccgatgaggatgtgatcctggtggagtctcaggaccctccacttccatcctggggtgcccctcccctcagagacagggccagacctcaggatgaagtgctggtcattgattccccccacaattccagggctcagtttccttccaccagtggtggttctggctataagaataacggtcctggggagatgcgtagagccaggaagcgaaaacacacaatccgctgttcgtattgtggtgaggaaggccactcaaaagaaacctgtgacaacgagagtgacaaggcccaggtttttgagaatttgatcatcactctccaggagctgacccatactgagatggagaggtcaagagtggcccctggcgaatacaatgacttctctgagccactgtaagggaccacccccaggtttcagtgaacccttacctatattcagcatccagtagtgggaaaactggggtgggggtgggggtgggacttctaactgcatgaattaatccacaaagcggctatcttttggggtggagtagaaagggtcttggataccagcacattggagggagatagcctgacctctgtccttgctccttctccctgcagcctacgggtctgttttctgtgtgtgcccatttccttgacagctttattctttgtgaaagtggtataatttattgttaaatatttgaacaataaaaaaggtacaaaaagtgaagtacaaattacccaaatctctccacccttatataatcattgtcaaccctttgatgagtgatatttccctatacctatgtacccagatagatatatgcatagataaaagtgatgaaatataagtgctgttctatctgtattttttcaccaaacaatatatgttgtgagcttctatgtcaataaatatatatatcagca

SEQ ID NO: 128 - protein, 403 aa, synthetic construct
  REGION 1..403: note = Synthetic
MASITACVGNSRQQNAPLPPWAHSMLRSLGRSLCPLVVKMAERNMKLFSGRVVPAQGKETFENWLIQVNEVLPDWSMSEEEKLKRLMKTLRGPAREVMRLLQAANPNLSVADFLRAMKLVFGESESSVTAHGKFFNTLQAQGEKASLYVIRLEVQLQNAIQAGILAEKDANQTRLQQLLLGAELNRDLRFRLKHLLRMYANKQERLPNFLELIKMIREEEDWDDAFIKRKRPKRSEPIMERAASPVAFQGAQPIAISSADCNCNVIEIDDTLDDSDEDVILVVSLYPSLTPTGAPPFRGRARPLDQVLVIDSPNNSGAQSLSTSGGSGYKNDGPGNIRRARKRKYTTRCSYCGEEGHSKETCDNESNKAQVFENLIITLQELTHTEERSKEVPGEHSDASEPQ

SEQ ID NO: 129 - DNA, 2940 bp, synthetic construct
  misc_feature 1..2940: note = Synthetic
  source 1..2940: mol_type = other DNA
gctcttttgtctcagtcgccagagactgaaagcaaccgcggctgcccggagggccgaactggagggtgggcgcagccttggctgccttgggaaccgtgctgcctcgactcggctacccctcgctgggcggtcgtgcgcactcgctaggtcgggcagccccgggtcgcttagcggccgaggaggcggcagagatcccgttcccctgcagagctacggggaccggaaggcgaaacagagctgtggacgcgggagcacatgctgcccaccagtggagcacaagtaaagaaagaacgcgcgattgcccggcgcggagaggggtgggggaagccccgccaggactgggcggcgcccggcttggagaatcagtaactgcgaggcaggggatggagcactcccttggcctggttgcgctgaggaaggagttgtggttcatctggatgctgccgactggataggatttaggatttgcctggattaaggtcatcaaagccgaatccgaacttgaattctgtgcggcggattgcagagctgtcttgaggccctgaggactgggatctgcgacacccagtggacaaaagtcggccgtaccccagaggctaattttctcacggcaggcacctgttgcagaggctgcacgtaaaataaaggcgaacgctagtttccgagctcatgtaagaatctgagaaataacgggggaaacgttgacaagggggtgtgattgagacctgaggccatgaggttaatggtttcttattatcataaatctgtgacacacaaccattgagttccgttttgagggcccagtatcagctcccaacactatgtcatctagaattagaagcaacagttgtcgcccctcctggagtacaggcaggaggggatggatgtggctctgaaaaactacctactagcccagggacacctgctagctctggaatgttgtaattgccagtctcctgcagggtattgttaaggcactgggcagatataaaatgcactgcaaggctattcaggaagatagagaatgctactgcagactgcttccccacagcccactatcttattaaccttttttactttccttagaatccctactgaaatataaggcaggcacagcccaggaacgttgctttggagaatcctgcagataaggcttttccaaaaagcgcgagcatcttgttgtattcagataccctatcgtcgtcagtcatggctagcatcactgcgtgtgtgggtaacagcaggcagcagaatgcacctttgccgccttgggcccattccatgttgaggtctctggggaggagtctctgtcctttagtggtcaaaatggcagagagaaacatgaagttgttctcaggaagagtggtgccagcccaggggaaagaaacctttgaaaactggctgatccaagtcaatgaggtcctgccagattggagtatgtctgaggaggaaaaactcaagcgcttgatgaaaacacttaggggccctgcccgggaggtcatgcgtttgcttcaggcggccaaccccaacctaagtgtagcagatttcttgcgggcaatgaaattggtgtttggggagtctgaaagcagtgtgactgcccatggtaaattttttaacaccctgcaggcacaaggggagaaagcctccctttatgtgatccgtttagaggtgcagctccagaatgctattcaggcaggcatcctagctgagaaagatgcaaaccagactcgcttgcaacagcttcttttaggcgctgagctgaatagggacctgcgcttcaggcttaagcatcttctcaggatgtatgcaaataagcaggagcggcttcccaatttcctggagttaatcaagatgataagggaggaagaggattgggatgatgcttttattaaacggaagcggccgaaaaggtctgagccaataatggagagggcagccagccctgtggcatttcagggcgcccagccaatagcaatcagcagtgctgactgtaactgcaacgtgatagaaatagatgatacccttgatgactctgatgaggatgtgatcctggtggtgtctctgtacccttcactgacacctacaggtgcccctcccttcagaggaagagccagacctctggatcaagtgctggttattgattcccccaacaattctggggctcagtctctttctaccagtggtggttctgggtataagaatgatggtcctgggaatattcgtagagccaggaagcgaaaatacacaacccgctgctcatattgtggggaggagggccactcaaaagaaacctgtgacaatgagagcaacaaggcccaggtttttgagaatctgatcatcaccctgcaggagctgacacatacagaggagaggtcaaaagaggtccctggagaacacagtgatgcttctgagccacagtaaggatctagtccagccctaaatgagtccttgactgtattcagagtctggtaatgggaataacaggagaggggggtgggtttctaactgcatgaattaatccacaaagcagttttcctttgggaaggagaagaggtcttgcataccagcacagtggatggggaatggtgtgacctctgtccctgctctcttctctgctggcttaagggtctattctccatgtgtctatttctttgatgactttatactttttatgaaaggagcatcttttcattaaaccttcaaaaataaaggaagatacaaaaactaaagtacaaattacctgaaactcagcacccttttgtaaccattgtcagccctttggtgaatgtccttccagatatctccctgtacctgtgtacccagatagatatatgtatagataagagtgaccaaatataagtgctgttctatactgtgtatttttcaccaaataatgtatctcgtggacttctatgtcgatgaatatatataaatatctaa

SEQ ID NO: 130 - protein, 8 aa, synthetic construct
  REGION 1..8: note = Synthetic
EEEEEEAS

SEQ ID NO: 131 - DNA, 20 bp, synthetic construct
  misc_feature 1..20: note = Synthetic
  source 1..20: mol_type = other DNA
cccagcttccttgagctaat

SEQ ID NO: 132 - DNA, 21 bp, synthetic construct
  misc_feature 1..21: note = Synthetic
  source 1..21: mol_type = other DNA
gttcctctggctcttcgatac

SEQ ID NO: 133 - DNA, 19 bp, synthetic construct
  misc_feature 1..19: note = Synthetic
  source 1..19: mol_type = other DNA
cgatgcaacgagtgatgag

SEQ ID NO: 134 - DNA, 18 bp, synthetic construct
  misc_feature 1..18: note = Synthetic
  source 1..18: mol_type = other DNA
gcaaacggactgaagcat

SEQ ID NO: 135 - DNA, 42 bp, synthetic construct
  misc_feature 1..42: note = Synthetic
  source 1..42: mol_type = other DNA
ggctcgttgggaggtcccgggttgaaatcccggacgagcccg

SEQ ID NO: 136 - DNA, 100 bp, Homo sapiens
  source 1..100: mol_type = other DNA
tagccaaatgcctcgtcatctaattagtgacgcgcatgaatggatgaacgagattcccactgtccctacctactatccagcgaaaccacagccaagggaa

SEQ ID NO: 137 - DNA, 125 bp, Bombyx mori
  source 1..125: mol_type = other DNA
tagccaaatgcctcgtcatctaattagtgacgcgcatgaatggattaacgagattcccactgtccctatctactatctagcgaaaccacagccaagggaacgggcttgggagaatcagcggggaa

(19) United States
(12) Patent Application Publication
(10) Pub. No.: US 2025/0064976 A1
(43) Pub. Date: Feb. 27, 2025

(54) ENGINEERED PNMA PROTEINS AND DELIVERY SYSTEMS THEREOF

(71) Applicants: The Broad Institute, Inc., Cambridge, MA (US); Massachusetts Institute of Technology, Cambridge, MA (US)

(72) Inventors: Feng Zhang, Cambridge, MA (US); Victoria Madigan, Cambridge, MA (US); Yugang Zhang, Cambridge, MA (US); Rumya Raghavan, Cambridge, MA (US); Max Wilkinson, Cambridge, MA (US); Guilhem Faure, Cambridge, MA (US); Rhiannon Macrae, Cambridge, MA (US)

(21) Appl. No.: 18/948,601

(22) Filed: Nov. 15, 2024

Related U.S. Application Data
(63) Continuation of application No. PCT/US2023/067069, filed on May 16, 2023.
(60) Provisional application No. 63/342,401, filed on May 16, 2022.
(60) Provisional application No. 63/437,894, filed on Jan. 9, 2023.

(51) Int. Cl. A61K 48/00; C12N 7/00; C12N 9/22; C12N 15/11; C12N 15/86

(52) U.S. Cl. CPC A61K 48/005; C12N 7/00; C12N 9/22; C12N 15/11; C12N 15/86; C12N 2310/20; C12N 2740/15023; C12N 2740/15042; C12N 2740/15052

(57) ABSTRACT

Described herein are engineered paraneoplastic Ma protein (PNMA) capable of forming a capsid. In some embodiments, the engineered PNMA proteins comprise one or more modifications that enhance binding or loading of a cargo into the capsid, one or more modifications that modify cell-specificity of the capsid, one or more modifications that enhance intracellular delivery of the capsid, or a combination thereof. Also described herein are delivery systems comprising capsids comprising an engineered PNMA protein and a cargo.

### CROSS-REFERENCE TO RELATED APPLICATIONS

This application is a continuation filed under 35 U.S.C. § 111 of International Application No. PCT/US2023/067069 filed May 16, 2023, which claims the benefit of and priority to U.S. Provisional Patent Application No. 63/342,401, filed on May 16, 2022, and U.S. Provisional Patent application No. 63/437,894, filed on Jan. 9, 2023, the contents of which are incorporated by reference herein in their entireties.

### STATEMENT REGARDING FEDERALLY SPONSORED RESEARCH

This invention was made with government support under Grant No. HL141201 and HG009761 awarded by the National Institutes of Health. The government has certain rights in the invention.

### SEQUENCE LISTING

This application contains a sequence listing filed in electronic form as an xml file entitled BROD-5575US_ST26.xml, created on Nov. 6, 2024, and having a size of 248,000 bytes. The content of the sequence listing is incorporated herein in its entirety.

### TECHNICAL FIELD

The subject matter disclosed herein is generally directed to engineered delivery compositions, systems, and uses thereof.

### BACKGROUND

Gene editing technologies, such as CRISPR-Cas, and RNA-based therapeutics, including mRNA-based vaccines, have the potential to be deployed in a wide range of disease contexts. To achieve this potential, however, a suite of delivery vehicles that can efficiently package and safely deliver therapeutic oligonucleotide cargoes to specific tissues is needed. Several delivery modalities have already been developed, including non-viral approaches such as lipid nanoparticles (LNPs), which have been successfully developed for oligonucleotide and mRNA therapeutics, and viral vectors such as adeno-associated virus (AAV) (1). However, the broad applicability of these approaches is limited due to a combination of factors including packaging constraints, immunogenicity, difficulty in achieving tissue-specific targeting, and costly large-scale manufacturability. Thus, there continues to be a need for new delivery approaches.

Citation or identification of any document in this application is not an admission that such a document is available as prior art to the present invention.

### SUMMARY

Described in certain example embodiments herein are engineered paraneoplastic Ma proteins (PNMA) capable of forming a capsid and comprising one or more modifications that enhance binding or loading of a cargo into the capsid, one or more modifications that modify cell-specificity of the capsid, one or more modifications that enhance intracellular delivery of the capsid, or a combination thereof.

In certain example embodiments, the one or more modifications that enhance binding or loading of the cargo comprise addition of a peptide comprising charged residues; addition of a polynucleotide binding domain; addition of a polypeptide binding domain; or a combination thereof.

In certain example embodiments, the peptide comprising charged residues is (a) inserted between any two consecutive amino acids in a loop domain of the PNMA; (b) an addition to the C- or N-terminus of the PNMA; (c) an addition to a C- or N-terminally truncated PNMA; or (a) combined with (b) or (c).

In certain example embodiments, the peptide comprising charged residues is inserted into a loop domain at amino acids 170-180, amino acids 256-263, or amino acids 302-305 of PNMA2, or a position in another PNMA corresponding thereto.

In certain example embodiments, the peptide comprising charged residues is inserted between amino acids 175 and 176, 261 and 262, 303 and 304 of PNMA2, or at a position of another PNMA corresponding thereto.

In certain example embodiments, wherein the size of the C-terminus truncation of the C-terminally truncated PNMA is 1 to 31 amino acids.

In certain example embodiments, the peptide comprising charged residues about 20 to about 150 amino acids in size.

In certain example embodiments, the peptide comprising charged residues comprises an arginine, lysine, and/or proline rich motif. In certain example embodiments, the peptide comprising charged residues comprises two or more RKK repeats or two or more RRLRRP (SEQ ID NO: 6) repeats. In certain example embodiments, the peptide comprising charged residues is RRKRRKRRKRRK (SEQ ID NO: 7). In certain example embodiments, the peptide comprising charged residues is RRLRRPRRLRRPRRPR (SEQ ID NO: 8).

In certain example embodiments, polynucleotide binding domain is (a) inserted between any two consecutive amino acids in a loop domain of the PNMA; (b) inserted in place of at least a portion of a zinc finger region; (c) an extension of a C- or N-terminus of the PNMA; (d) an extension of a C- or N-terminally truncated PNMA; or (a) combined with (b); or (a) and (b) combined with (c) or (d); or (b) combined with (c) or (d).

In certain example embodiments, the polynucleotide binding domain is inserted between amino acids 256-263 or 302-305 of PNMA2 or at a position corresponding thereto.

In certain example embodiments, the polynucleotide binding domain is inserted between amino acids 261 and 262, or amino acid 303 and 304 of PNMA2 or an amino acid position in another PNMA corresponding thereto.

In certain example embodiments, the polynucleotide binding domain replaces amino acids 412 to 429 of PNMA3 or an amino acid position of another PNMA corresponding thereto.

In certain example embodiments, the polynucleotide binding domain is inserted between M1 and P2 of the N-terminus of PNMA3 or a position in another PNMA corresponding thereto.

In certain example embodiments, the polynucleotide binding domain comprises or consists of a PNMA RNA recognition motif, a λN polypeptide, a P22N polypeptide, a MS2 polypeptide, an R17 polypeptide, a retroviral or lentiviral Rev polypeptide, polynucleotide binding domain of a nuclease, a Zinc Finger domain, a 14-3-3 polypeptide, a STAR-family polypeptide, a toll-like receptor polypeptide, CCMV N-terminal sequence, an arginine, lysine and/or proline rich motif, or any combination thereof.

In certain example embodiments, the λN comprises SEQ ID NO: 1 or 2. In certain example embodiments, the P22N comprises SEQ ID NO: 3. In certain example embodiments, the Rev polypeptide comprises SEQ ID NO: 4.

In certain example embodiments, the protein binding domain is added to a C- or N-terminus of the PNMA. In certain example embodiments, the protein binding domain is a dimerization domain, optionally a leucine zipper.

In certain example embodiments, the one or more modifications that modify cell-specificity comprise insertion of a cell surface binding peptide, cell penetrating peptide, monobody, nanobody, or antibody or fragment thereof, in the N-terminus of the PNMA, optionally wherein the one or more modifications are inserted between amino acid residues P27-E31, G125 and S138, P196 and T198, D224 and S229, G319 and S323, or any combination thereof with reference to PNMA2 or PNMA3 or a position in another PNMA corresponding thereto.

In certain example embodiments, the cell surface binding peptide is an integrin binding peptide, a VEGFR-1 ligand, an EGF peptide, a human transferrin receptor binding peptide, a hepatocellular carcinoma targeting peptide, a monobody capable of specifically binding a cell surface or molecule thereon, or a nanobody capable of specifically binding a cell surface or molecule thereon.

In certain example embodiments, the one or more modifications that enhance intracellular delivery are capable of enhancing cell entry, endosomal escape or both, and optionally wherein the one or more modifications comprise or consist of. In certain example embodiments, the endosomal escape peptides are selected from the group consisting of pVI, H5WYG, HIV tat, R5, and LAH4.

Described in certain example embodiments herein are polynucleotides encoding the engineered PNMA of any one of the preceding paragraphs and as described elsewhere herein.

Described in certain example embodiments herein are vectors encoding the engineered PNMA of any one of the preceding paragraphs and as described elsewhere herein.

Described in certain example embodiments herein are delivery systems comprising a capsid comprising the engineered PNMA of any one of the preceding paragraphs and as described elsewhere herein; and a cargo captured by, or packaged within, the capsid.

Described in certain example embodiments herein are methods for cellular delivery of cargos, comprising delivering the delivery system of the preceding paragraph and elsewhere herein to a cell or population of cells in vitro or in vivo.

Described in certain example embodiments herein are methods of in vitro packaging a cargo in a capsid comprising one or more engineered paraneoplastic Ma proteins (PNMA) described herein comprising combining a cargo and a plurality of engineered PNMA protein monomers in an assembly solution comprising an amount of a salt and an amount of calcium chloride thereby promoting assembly of the capsid and packaging of the cargo in the capsid. In some embodiments, the one or more PNMA is PNMA2 and/or PMNA3.

In certain example embodiments, the amount of salt in the assembly solution is about 100 mM to about 600 mM, wherein the amount of calcium chloride in the assembly solution is about 5 to about 100 mM, or both. In some embodiments, the amount of salt in the assembly solution is about 500 mM, wherein the amount of calcium chloride in the assembly solution is about 10 mM, or both.

In certain example embodiments, the method further comprises generating the plurality of engineered PNMA monomers prior to combining, wherein generating the plurality of engineered PNMA monomers comprises disassembling one or more capsids comprising a plurality of engineered PNMAs by exposing the capsid comprising one or more engineered PNMAs to a disassembly solution thereby generating the plurality of PNMA monomers.

In certain example embodiments, the disassembly solution comprises an amount of a salt or an amount of urea effective to disassembly the one or more capsids comprising a plurality of engineered PNMAs. In certain example embodiments, the disassembly solution comprises an amount of salt or an amount of urea effective to promote disassembly of the one or more capsids. In certain example embodiments, the disassembly solution comprises about 5 mM to about 50 mM salt or about 6 M urea. In certain example embodiments, the disassembly solution does not contain calcium chloride.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1B—PNMA family members are derived from retrotransposons and contain GAG domains. The phylogenetic tree shown in FIG. 1A shows human PNMA genes as well as marsupial PNMA ancestor (mPNMA) and Gypsy (PNMA ancestor).

FIG. 2—PNMA2 forms capsids in vivo and in vitro but does not package an RNA genome.

FIG. 3—PNMA2 forms icosahedral capsids.

FIG. 4A-4B—PNMA2 inner surface is negatively charged with poly glutamate (E) stretches extending into the capsid.

FIG. 5—Diagram and supporting TEM images of in vitro PNMA2 capsid formation from PNMA2 monomers. PNMA2 capsid assembly is ionic strength dependent.

FIG. 6A-6B—Representative PNMA2 constructs for in vitro capsid formation and RNA packaging. Adding CCMV peptide to PNMA2 allows for RNA packaging. FIG. 6A shows a construct diagram for adding a CCMV peptide to residues 1-340 of PNMA2. FIG. 6B shows packaging of RNA by PNMA2 monomers from the construct of FIG. 6A.

FIG. 7—PNMA2 with a C-terminal CCMV can package and deliver functional mRNA.

FIG. 8—A PNMA2 construct with a PNMA2 having a N-terminal cell penetrating peptide (CPP) and a C-terminal CCMV.

FIG. 9A-9B—(FIG. 9A) Chromosomal location of human PNMA genes. (FIG. 9B) Phylogenetic tree of PNMA family including all humans PNMAs (PNMA1-8 and CCDC8), the marsupial PNMA (MePNMA), and a turtle Gypsy (see Methods, Example 3) from which the tree is rooted. Domain architecture of each protein is deduced from the structural models (see Methods, Example 3) and is shown adjacent to each leaf Domain architecture encompasses an RRM-like fold domain (pink), dimerization domain that forms only upon interaction (in orange), and capsid domain in light blue for N-terminal capsid domain and dark blue for C-terminal capsid domain. Additional domains predicted to fold are shown in gray. Zinc fingers are shown in yellow, and regions with a high concentration of K-R are shown in green.

FIG. 10A-10E—(FIG. 10A) Schematic of isolation of cell lysate and VLP fraction. (FIG. 10B) Western blot showing PNMA protein expression in HEK293FT cells in either cell lysate (top) or the VLP fraction (bottom). (FIG. 10C) TEM micrographs of HA-immunoprecipitated VLP fraction from negative control cells (top panels) or cells overexpressing HA-tagged PNMA2 (bottom panels). (FIG. 10D) (Top) Expression of PNMA2 or a mutant of PNMA2 lacking the start codon in whole-cell lysate or the VLP fraction. (Bottom) Quantification of PNMA2 mRNA (cDNA) in either the whole-cell lysate or VLP fraction in cells expressing either wild-type PNMA2 (black) or a mutant lacking the start codon (gray). (FIG. 10E) (Top) Schematic of experimental procedure to identify mRNA packaged in PNMA2 capsids. (Bottom) Volcano plots showing differential mRNA expression of PNMA2 CRISPRa samples versus nontargeting guide control in either the cell lysate (left) or VLP fraction (right).

FIG. 11A-11C—(FIG. 11A) TEM micrograph of PNMA2 purified from E. coli. (FIG. 11B) Size-exclusion chromatography (SEC) trace of PNMA2 particles purified from E. coli. (FIG. 11C) Schematic of workflow for in vitro production of PNMA2 capsids with representative TEM images below.

FIG. 12A-12E—(FIG. 12A) Cryo-EM density of PNMA2 with 14 symmetry imposed, colored by radial distance from the center of the capsid. (FIG. 12B) Model of the PNMA2 capsid with a protein monomer outlined. (FIG. 12C) Details of the interactions of a PNMA2 monomer (pink highlight) with adjacent monomers, with symmetry axes indicated. (FIG. 12D) Electrostatic potential of the inside of the PNMA2 capsid. Red indicates negative charge. (FIG. 12E) Central slice of the PNMA2 cryo-EM density. The projected positions of the modeled N- and C-termini are shown as blue and red circles.

FIG. 13A-13F—(FIG. 13A) Schematic of ePNMA2 protein and TEM micrograph of ePNMA2 capsids. (FIG. 13B) Quantification of genomes per capsid packaged by ePNMA2 during reassembly across a range of salt conditions following RNaseA treatment.

(FIG. 13C) Immunofluorescence showing ePNMA2 entry into Neuro2A cells with either no co-treatment or LAH4. Scale bar is 1 um. (FIG. 13D) Schematic of workflow for in vitro production of ePNMA2 capsids. (FIG. 13E) Quantification of GFP positive cells by flow cytometry following delivery by ePNMA2 of Cre mRNA to N2A-loxP-GFP recipient cells. (FIG. 13F) White light (WL) and GFP fluorescence images of N2A-loxP-GFP recipient cells 6 hours after ePNMA2-mediated delivery of Cre mRNA. Scale bar is 10 um.

FIG. 14—Pentamer assembly of human PNMA proteins using AlphaFold2 multimer.

FIG. 15—Western blot of cell lysate (left) and VLP fractions (right) from HeLa, U87, Neuro2A and U20S cells transfected with either PNMA2-HA or mCherry-HA control plasmid.

FIG. 16—Immunofluorescence images of U20S cells transfected with a CMV-driven PNMA2 overexpression plasmid or negative control empty vector plasmid.

FIG. 17A-17C—(FIG. 17A) Schematic of disassembly conditions for the PNMA2 capsid (top) and resulting size exclusion chromatography trace, compared to treatment of ePNMA2 with the same disassembly conditions (bottom). (FIG. 17B) Transmission electron micrographs of PNMA2(340)-CCMV(30) under different screening conditions for disassembly. (FIG. 17C) Schematic of PNMA2(340)-CCMV(30) reassembly conditions and electron micrograph of resulting capsids.

FIG. 18—Flow cytometric quantification of GFP fluorescence of Neuro2A-loxP-GFP reporter cells transduced with various doses of RNAse treated ePNMA2(Cre) and an equal dose of Cre mRNA with and without RNAse treatment. Capsids and RNAs were treated with LAH4 before being added to Neuro2A cells. Error bars represent SEM of technical triplicate, n=3.

FIG. 19A-19C—(FIG. 19A) TEM micrographs of human PNMA3v1, PNMA3v2, PNMA5, PNMA7b and PNMA8av1, as well as MePNMA. Scale bar is 100 nm. (FIG. 19B) Expression of human PNMA transcripts across human tissues. (FIG. 19C) Expression of human PNMA proteins across human tissues.

FIG. 20A-20F—(FIG. 20A) Example cryo-EM micrograph of PNMA2. (FIG. 20B) Representative 2D class averages. (FIG. 20C) Cryo-EM data processing workflow. (FIG. 20D) Gold-standard half-map Fourier Shell Correlation (FSC) curves. (FIG. 20E) FSC between the final map and the built model. (FIG. 20F) Cryo-EM density (C5 reconstruction) masked around a single PNMA2 monomer.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, the term “specific binding” refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide engineered paraneoplastic Ma protein (PNMA) capable of forming a capsid, such as via self-assembly, that is capable of capturing or otherwise packaging a cargo. The engineered PNMA (ePNMAs) proteins are engineered to contain one or more modifications that enhance binding or loading of a cargo into the capsid formed from the engineered PNMA proteins, one or more modifications that modify cell-specificity of the capsid, one or more modifications that enhance intracellular delivery of the capsid, or any combination thereof. The engineered PNMA capsids can be used to deliver one or more cargos (e.g., nucleic acids, proteins, etc.) to a cell or cell population. Delivery can be in vitro, in vivo, or ex vivo. Given then the ePNMAs are derived from PNMA genes that occur in the human genome, the ePNMAs are expected to have reduced immunogenicity when used as a delivery modality in humans. Many PNMA family members are also highly expressed in the central nervous system (CNS), raising the possibility that the ePNMA versions thereof may be harnessed for delivery of cargos to the brain and/or other parts of the CNS.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Engineered Pnma Proteins

Described in certain example embodiments herein are engineered paraneoplastic Ma (PNMA) polypeptides and engineered PNMA capsids formed from the engineered PNMA polypeptides. In some embodiments, the engineered PNMA capsids contain one or more cargos. In certain example embodiments, the engineered PNMA polypeptides are capable of forming a capsid. In certain example embodiments, the engineered PNMA polypeptides are engineered to include one or more modifications that enhance binding and/or loading of a cargo into the capsid, one or more modifications that modify cell-specificity of the capsid, one or more modifications that enhance intracellular delivery of the capsid, or any combination thereof. Without being bound by theory, one or more modifications that enhance intracellular delivery can do so by enhancing cell entry and/or endosomal escape.

In certain example embodiments the engineered PNMA polypeptide is modified relative to a wild-type PNMA1, PNMA2, PNMA3, PNMA3_i2, PNMA5, PNMA5_i2, PNMA5_i3, PNMA5_i4, PNMA6A, PNMA6E, PNMA6E_i2, PNMA6E_i3, PNMA6F, PNMA8A, PNMA8A_i2, PNMA8B, PNMA8B_i2, PNMA8C, CCDC8, ZCCHC12 (PNMA7A), ZCCHC12_i2 (PNMA7A_i2), ZCCHC12_i3 (PNMA7A_i3), ZCCHC18, or MOAP1 (PNMA4) or a functional domain thereof. Thus, in some embodiments, the engineered PNMA polypeptide is an engineered PNMA1, PNMA2, PNMA3, PNMA3_i2, PNMA5, PNMA5_i2, PNMA5 i3, PNMA5_i4, PNMA6A, PNMA6E, PNMA6E_i2, PNMA6E_i3, PNMA6F, PNMA8A, PNMA8A_i2, PNMA8B, PNMA8B_i2, PNMA8C, CCDC8, ZCCHC12 (PNMA7A), ZCCHC12_i2 (PNMA7A_i2), ZCCHC12_i3 (PNMA7A_i3), ZCCHC18, or MOAP1 (PNMA4) polypeptide or a functional domain thereof. Exemplary reference wild-type PNMAs are set forth in Table 1. Other reference wild-type PNMAs (e.g., variants, homologues and orthologues) will be instantly appreciated by one of ordinary skill in the art in view of the description herein.

TABLE 1
Exemplary Reference wild-type PNMA Sequences

| | GenBank | | |
|---|---|---|---|
| PNMA | Accession | | |
| (homo | No./Version No. | | |
| sapiens) | for Protein | Protein Sequence | Encoding Polynucleotide Sequence |
| PNMA1 | NM 006029/ | MAMTLLEDWCRGM | AGCAGTAACGTCGCGGCGGGTTGCGGGTAGGA |
| | NM 006029.5 | DVNSQRALLVWGIPV | CTGGACGCCAGAGCAGCCGCGCAGCGCCTGAA |
| | (encoding | NCDEAEIEETLQAAM | CCGCTGCGGGCCGCCGCGGCCGCCCCTTCCCAC |
| | polynucleotide) | PQVSYRMLGRMFWR | CCTCGCCTCTGCTGTCTCCAGCCTCGCTTCTCCG |
| | NP_006020/ | EENAKAALLELTGAV | ACTTTCCTGCTCCTCTGCTGCCTTCGTTTCTGGT |
| | NP_006020.4 | DYAAIPREMPGKGG | CCTCGGCCGTCCTCGCCGCCCGCCCAGAGGAGT |
| | (protein) | VWKVLFKPPTSDAEF | CCCCGCGCCCGCCAAGAAGCCGCTTTCCGCTGG |
| | | LERLHLFLAREGWTV | CCCGCAGCCGCCGCGACTTCGGCACAGTTTCTC |
| | | QDVARVLGFQNPTPT | CCTCTGGCTAGTCTCCCAAACGGCCGCTCCTCG |
| | | PGPEMPAEMLNYILD | CCCGCGGGAAGACCAGGCTGCGACCGCGAACG |
| | | NVIQPLVESIWYKRL | CCCGATCCTCTCCAGGAGCCGCAGCGAGCGCCC |
| | | TLFSGRDIPGPGEETF | GGCGGCCACGCCCCGCGACCACACCCCGGCGGC |
| | | DPWLEHTNEVLEEW | TCTCGGCCCAGCGCGCCTGCCTTCGCCGCCCGC |
| | | QVSDVEKRRRLMESL | CGTCGCTCCTCGCCCGCTGCACGACGACGCGAC |
| | | RGPAADVIRILKSNNP | GCCCCTGCTGCAGGCGGCGGACCCGACCGGACC |
| | | AITTAECLKALEQVF | CAGACCCAGACGCAAGATGGCGACGGCCGCGT |
| | | GSVESSRDAQIKFLN | GACTGCCTCAGCGTCCCCGAGCTCGGCTCCGAG |
| | | TYQNPGEKLSAYVIR | TGCACCTACGGACTGACTGTGGGGGCAGAGAA |
| | | LEPLLQKVVEKGAID | GGGCGAGATCAGGACTCTGTCTTTGTTAATCGT |
| | | KDNVNQARLEQVIA | GACTGCATGAAGGTCGCCTCCCTCGGGCCTACT |
| | | GANHSGAIRRQLWLT | TGGTGGGAGTGTCTGGTATTGTTCTAAGGCCAG |
| | | GAGEGPAPNLFQLLV | GAGCACGGTGAGCCACAGTCTGTTGGTAGAATT |
| | | QIREEEAKEEEEEAEA | TGGCGTCTTGATAGTTGAGAAAATGGCGATGAC |
| | | TLLQLGLEGHF (SEQ | ACTGTTGGAAGACTGGTGCCGGGGGATGGATGT |
| | | ID NO: 86) | GAACTCCCAGAGAGCTCTGTTAGTCTGGGGCAT |
| | | | CCCAGTGAACTGTGATGAGGCTGAAATCGAAGA |
| | | | GACCCTCCAGGCTGCGATGCCCCAGGTCTCCTA |
| | | | CCGAATGCTTGGGAGAATGTTCTGGAGGGAAGA |
| | | | AAATGCGAAAGCAGCCTTATTAGAGCTCACTGG |
| | | | CGCTGTAGATTACGCCGCGATCCCCAGGGAGAT |
| | | | GCCGGGCAAAGGAGGGGTCTGGAAAGTGTTATT |
| | | | TAAGCCCCCAACTTCTGATGCTGAATTTTTAGA |
| | | | AAGATTGCACCTCTTCCTAGCTAGAGAGGGGTG |
| | | | GACCGTGCAAGATGTTGCCCGTGTCCTTGGGTT |
| | | | TCAGAACCCTACTCCGACCCCGGGCCCAGAGAT |
| | | | GCCAGCAGAGATGCTAAACTATATTTTGGATAA |
| | | | TGTTATTCAGCCTCTTGTTGAGTCCATATGGTAC |
| | | | AAGAGGCTGACACTTTTCTCGGGGAGGGACATC |
| | | | CCAGGGCCTGGAGAGGAAACCTTTGATCCCTGG |
| | | | CTGGAGCACACTAATGAGGTCCTAGAGGAGTGG |
| | | | CAGGTGTCCGATGTAGAAAAGAGGCGGCGGTT |
| | | | GATGGAGAGTCTTAGAGGCCCCGCCGCTGATGT |
| | | | TATTCGCATCCTTAAGTCCAACAACCCCGCGAT |
| | | | AACCACTGCCGAATGCCTGAAGGCGCTTGAGCA |
| | | | GGTGTTTGGGAGCGTTGAGAGCTCTAGGGATGC |
| | | | CCAGATCAAATTTCTGAACACTTATCAGAACCC |
| | | | GGGAGAAAAATTGTCTGCTTATGTCATTCGTCT |
| | | | GGAGCCTCTGCTACAGAAGGTGGTAGAGAAGG |
| | | | GGGCCATTGATAAAGATAATGTGAACCAGGCCC |
| | | | GCCTAGAGCAGGTCATTGCCGGGGCCAACCACA |
| | | | GCGGGGCCATCCGAAGGCAGCTGTGGCTTACCG |
| | | | GGGCTGGGGAAGGGCCAGCCCCAAACCTCTTTC |
| | | | AGTTGCTGGTGCAGATCCGTGAGGAGGAAGCCA |
| | | | AGGAGGAGGAGGAGGAGGCTGAGGCCACCCTT |
| | | | CTGCAGTTAGGCCTGGAAGGGCACTTCTGAGTG |
| | | | CCAGGAAAGGCAGCTTTAGTGCAGACCTAGATC |
| | | | ACAGCTACTTTTCTTGTCCCTGTGGGGTCTTACA |
| | | | GATGTGTCTCTGAGTAGTAAAGGCTTAGCCTTG |
| | | | TTCTGTTTTGTTGTTTTTTGGAGGGGAAGGTTAG |
| | | | TCAGGCCTGAGTATTCATGTAACATTCTAAAAT |
| | | | TGTGCCAGCGAGCACCGTGAACGACTGCAATGC |
| | | | AAGCGGGTCTTGCTGGCTAAAATGCCAGGTAAA |
| | | | GGGTTGGTTGGACACAGCGCTTAGTGCACGCTG |
| | | | TCATCATGGACATCATAATCAGTTGTGAAAAAC |
| | | | ACGCGAACCTATGACACTTCTTATTCCACACTG |
| | | | AATGTGAAATTGCATGTTCAGATGTTTACTACG |
| | | | AGGCCTGGCTCACAGGAAGTGTTCAGTAAAAGT |
| | | | ATGCACTGTTAGATTACTGATAACGCGGATAGA |
| | | | TTTTTGTTTACCATAAATTGTTCCAGATTTATAT |
| | | | TAATGGAAGGAAGTGTGCATTTATTAGCTATTA |
| | | | CTCAACTTTACAATGCAAACATCTTATTTCTCAT |
| | | | CTTTAAACATGTCGACCAGTTTAATTGAAAAGT |
| | | | ATTCTGAGACTGCAAAATGGGGTGTTAAAAAAT |
| | | | ACTGCAGTTACGGAGCTGTGTAAACCAGTTTCT |
| | | | CATTGCATAAGATACAGATGTAAATTGCATGGA |
| | | | GAGGTTGATATGCACCTGTACAGTAATTCACTC |
| | | | CCCCATTTCACATCTTTGTCAGAGAATAGTTCTT |
| | | | GTTCATACTGAGTGTTCTAAATTTGAAGTTATAT |
| | | | ATACAAATTAAAATATTTTAAAAATTC (SEQ ID |
| | | | NO: 87) |
| PNMA2 | NM_007257/ | MALALLEDWCRIMS | AGACCACCAGCTAATGGATGCGGAGCGGAGGG |
| | NM_007257.6 | VDEQKSLMVTGIPAD | CCCGCTGACCGCTCTCCGCGCCTGGAGCAGCTT |
| | (encoding | FEEAEIQEVLQETLKS | GGCTTGGCTGGAGCTAAGAGCCAGACACACCAC |
| | polynucleotide) | LGRYRLLGKIFRKQE | TGTGTGGAGGTGGGTGATGTCTTCCTGTGCTAA |
| | NP_009188/ | NANAVLLELLEDTDV | AAGGTGAATAAATAAGCTCCTCACCTCTCGCGG |
| | NP_009188.1 | SAIPSEVQGKGGVWK | AACACTCGGGAACACATCAACAGGGGTCCAAG |
| | (protein) | VIFKTPNQDTEFLERL | CCGCCCTGCTGGGAGGCTTCTCTTCAAGAGTTCT |
| | | NLFLEKEGQTVSGMF | GGGTCCCAGAGTGGAAGGCATTTTCCCATCAAC |
| | | RALGQEGVSPATVPC | TGGAGAGAGACGAAACATCAGAGACCAGGAGG |
| | | ISPELLAHLLGQAMA | CTGTGGAGAAAGCAGCTGTCCCAGGTGCCTCAA |
| | | HAPQPLLPMRYRKLR | CTATCAGAGAAGGGTCAGCGTCACGTGGCTGCC |
| | | VFSGSAVPAPEEESFE | AGCATCTTTGAGAAAATCACTGGCAATCGGACT |
| | | VWLEQATEIVKEWP | TCAGAGCTGCGGGCACAGGTGTGGTTAGAACTG |
| | | VTEAEKKRWLAESLR | AGATACGACCTGCCCACCTGGGTCAGGCCTAAA |
| | | GPALDLMHIVQADNP | GACAAGAAGTCCTGAGTTCTTGCCACTGAGTAG |
| | | SISVEECLEAFKQVFG | GCCAGGGTCATTTGTCCAGAAAACTTTGTGACT |
| | | SLESRRTAQVRYLKT | GTCTTTGAGTGACCTAGTCTGGGACCCATTCATT |
| | | YQEEGEKVSAYVLRL | GGTGGGTTCTAAGGTTAGAAGCTCATCCAGGAT |
| | | ETLLRRAVEKRAIPR | ATTTTCAATATTAAGTCAGTGCATAGCTGCACC |
| | | RIADQVRLEQVMAG | ACTAACAAATTGGTGCCTGTAGAGTCAGAGTGG |
| | | ATLNQMLWCRLREL | GTCAATTCTTAGGACAATGGCGCTGGCACTGTT |
| | | KDQGPPPSFLELMKV | AGAGGACTGGTGCAGGATAATGAGTGTGGATG |
| | | IREEEEEEASFENESIE | AGCAGAAGTCACTGATGGTTACGGGGATACCGG |
| | | EPEERDGY GRWNHE | CGGACTTTGAGGAGGCTGAGATTCAGGAGGTCC |
| | | GDD (SEQ ID NO: 88) | TTCAGGAGACTTTAAAGTCTCTGGGCAGGTATA |
| | | | GACTGCTTGGCAAGATATTCCGGAAGCAGGAGA |
| | | | ATGCCAATGCTGTCTTACTAGAGCTTCTGGAAG |
| | | | ATACTGATGTCTCGGCCATTCCCAGTGAGGTCC |
| | | | AGGGAAAGGGGGGTGTCTGGAAGGTGATCTTTA |
| | | | AGACCCCTAATCAGGACACTGAGTTTCTTGAAA |
| | | | GATTGAACCTGTTTCTAGAAAAAGAGGGGCAGA |
| | | | CGGTCTCGGGTATGTTTCGAGCCCTGGGGCAGG |
| | | | AGGGCGTGTCTCCAGCCACAGTGCCCTGCATCT |
| | | | CACCAGAATTACTGGCCCATTTGTTGGGACAGG |
| | | | CAATGGCACATGCGCCTCAGCCCCTGCTACCCA |
| | | | TGAGATACCGGAAACTGCGAGTATTCTCAGGGA |
| | | | GTGCTGTCCCAGCCCCAGAGGAAGAGTCCTTTG |
| | | | AGGTCTGGTTGGAACAGGCCACGGAGATAGTCA |
| | | | AAGAGTGGCCAGTAACAGAGGCAGAAAAGAAA |
| | | | AGGTGGCTGGCGGAAAGCCTGCGGGGCCCTGCC |
| | | | CTGGACCTCATGCACATAGTGCAGGCAGACAAC |
| | | | CCGTCCATCAGTGTAGAAGAGTGTTTGGAGGCC |
| | | | TTTAAGCAAGTGTTTGGGAGCCTAGAGAGCCGC |
| | | | AGGACAGCCCAGGTGAGGTATCTGAAGACCTAT |
| | | | CAGGAGGAAGGAGAGAAGGTCTCAGCCTATGT |
| | | | GTTACGGCTAGAAACCCTGCTCCGGAGAGCGGT |
| | | | GGAGAAACGCGCCATCCCTCGGCGTATTGCGGA |
| | | | CCAGGTCCGCCTGGAGCAGGTCATGGCTGGGGC |
| | | | CACTCTTAACCAGATGCTGTGGTGCCGGCTTAG |
| | | | GGAGCTGAAGGATCAGGGCCCGCCCCCCAGCTT |
| | | | CCTTGAGCTAATGAAGGTAATACGGGAAGAAG |
| | | | AGGAGGAAGAGGCCTCCTTTGAGAATGAGAGT |
| | | | ATCGAAGAGCCAGAGGAACGAGATGGCTATGG |
| | | | CCGCTGGAATCATGAGGGAGACGACTGAAAAC |
| | | | CACCTGGGGGCAGGACCCACAGCCAGTGGGCT |
| | | | AAGACCTTTAAAAAATTTTTTTCTTTAATGTATG |
| | | | GGACTGAAATCAAACCATGAAAGCCAATTATTG |
| | | | ACCTTCCTTCCTTCCTTCCTTCCCTCCCTTCCTCC |
| | | | TTCTCTCCTTCTCTCCTCCTCTCTCCTCTCCTCTC |
| | | | CTCTCTTTCCTTCCTTCCTTCCTTTTTTCTTTTTCT |
| | | | CTTTCTTCTTTATTTCTTGGGTCTCACTCTCATCA |
| | | | CCCAGGCTAGAGTGCAGTGGCACAAAAATCTCG |
| | | | GCTCACTGCAGCCTTGACTTCCCAGGCTCAGGC |
| | | | TCAGGTGATCCTCACACCTTAGCCTCCCAAGTA |
| | | | CCTGGGACTACAGGCACGCACCACCATGCCTAG |
| | | | CTATTCTTTTGTATTTTTGGTAGAGACAGGGTTT |
| | | | TGCTGTGTTGCTCAGGCTGGTCTGGAACCCCTA |
| | | | GGCTCAAATGATGTGCCCAACTCGGCCTCCCAA |
| | | | AGTGCTGGGATTACAGGCATGAACCGCCATGCC |
| | | | TGGCCCTTGATTTTTCTTTTTAAGAAAAAAATAT |
| | | | CTAGGAGTTTCTTAGACCCTATGTAGATTATTAA |
| | | | TGAACAAAAGATTAAACTCCAAATATTAAATAG |
| | | | TAAGCCTGAAGGAATCTGAAACACTTGTACTTC |
| | | | CAATTTTCTTTAAATAATCCCAAATAGACCAGA |
| | | | ATTGGCCCATACCATAGAAGAAAGAATTGGCAG |
| | | | TCAAAAAAAAAAATACCTTTTGTAATGTTTGAA |
| | | | AAATAAAGCTGTTTGACTTGTCAGGTGTTTTCCT |
| | | | TTCTCAAATCAGCAAATTCTCTCTGAGTGCCTGG |
| | | | CTTTGTGAGACACTGTACAAGGAGTTACAAGAC |
| | | | TACAGCTATAACCTGCAGTTGAGCAGTTATAAA |
| | | | CCTACAAAATGGGCCCTGCCCTCAGAGAGGTTC |
| | | | CAGTCTAGATGAGGAGCTGATCTAGACAGGTAA |
| | | | AAGGCTAACTAACCCTTTGTGTAAATAAGTTCA |
| | | | TCACCCCAGTAAAAGTGTCATCACCCAGTGAAT |
| | | | AGGACCACCTCTGCCTGCAGATTTTTGTTGTTGT |
| | | | TGTTGTCATTGTTGTTGTTGTTTTAACCTGGGAA |
| | | | GTGTTCTTCCTGCCTTTCTGCTAGGTGTCAGATA |
| | | | GATGGTCCCAGAGCTAGGTGCTGTGTCAGGCCC |
| | | | TGAAGACACAGATGACTCAACCTAAGCTTTACT |
| | | | TTCCAGAGGTCCACAGCCTGAGAGGTGTCCCCA |
| | | | AAGAAAGGGGGACATGAGGGGACTGCATGCTT |
| | | | GAGAGCAGGGTTGTTTAGGGCAGGTTTGGATTT |
| | | | AGTGAGCAGGCTGGTTTGCTTAGAGAAGGCTTT |
| | | | TAGTGGCAACAAAGGATGAAGAGGAGAGAAAA |
| | | | GGAACTCACATTTATTGAGGGCCTACTGTGTGC |
| | | | AAAGTGTTTCATGTATATCTCATTGAATGTATAC |
| | | | AGCCACCCTGTTGTGGTATAATTTTGCTCTTTAT |
| | | | AAAGAGAAAGACCGAAGCTCAGATGAGTTAAG |
| | | | TGGTCTCCTCAACACCAAAATGCCAAGAAGTGA |
| | | | TGGAGCCTAGACAGAAGCCCAGAACTTTCTGAC |
| | | | TCACACTAGTCCATCCTCTACCATCACGATGACT |
| | | | TTCAAATTGTGCTCTGCAGTTCTGCAGATTTTCT |
| | | | AGCAGTGCCATCTCCAAAATGTGTTTTAAACTC |
| | | | TTTATTTTTTTAATTATTATTAGTATTATTTTGAG |
| | | | ACTGAGTCTTGCTCTATCACCCAGGCTGGAGTG |
| | | | CAGTGGTGCAATCTCAGCTCACTGCAACCTCCG |
| | | | CCTCCCAGGTTCAAGCGATTTCGTGCCTCAGCCT |
| | | | CCCGAGTAGCTGGGATTACAGGCACCCACCACC |
| | | | ACGCCCAGCTAATTTTTGTATTTTTAGTAGAAAT |
| | | | GGGGTTTCACCATGTTGGCCAGGCTGGTCTCGA |
| | | | ACTCCTGACCTCAAGTGATCCACTCACCTCGGC |
| | | | CTCCCAAAGTGCTGGGATTACAGGTGTGAGCCA |
| | | | CCATGCCTGGGCTAAACTCTTTAAGTCTCTAGTA |
| | | | AATGCAGCTAGATTCAAATGGGCTGATAACCAA |
| | | | ATTTTAACACATCAGCATTCACCACCAGGTTTA |
| | | | CTTTTATTTTCAGATTGGCTCATTTTGTGCAGAC |
| | | | CTTAGAGCAAAGTTTCCTTTATGGTATCTGTGTA |
| | | | CGTATCCAAACTTCTTTTAATTGTTCACAGATTT |
| | | | TAAAAGCGGTAGCACCACATGGTTGTGTAGATC |
| | | | AGACCTGTGTATTTAGATCAGACCTGTGTATCA |
| | | | CGTAAGTGTGTGAGTGCAGTGCAGATGAGCACC |
| | | | ATTTAGTTATATGTGCTAGGCAAATCTCCAACA |
| | | | CAGTTGATGTGTAGTCTTGTGGTAGATTTGTGCA |
| | | | TACTGTAAGCAAATTGCTTAGCTTCTCTAGACAT |
| | | | CAGTTTCCACATCTGAAAAATAAGAAGATGAGA |
| | | | GTACACGGTTGTTATGAACAAATGACTTAATGC |
| | | | TTTTTAAGCACGTTGCATGACATCTGGAACACA |
| | | | GAAAGCCCTCAATACATTGAAGCTCTTAGGATT |
| | | | TTCACGATGTTCCTGTCTGCTCAATGCATGCTTT |
| | | | CTTTATTGTTCTGACAGTTGTGTGGTAACAAGCT |
| | | | AATATGCTTCCAGTTGACTTCCAGTCTACCCTGG |
| | | | TGTTAGAAACCGTTTCATCTCTTATTGTAAATTT |
| | | | GAGTGCTTGTTGTTTTTTATATTTGTGATGACTC |
| | | | TTCCAGCAGTTGTTGACAATTGTTAGAGGTTTG |
| | | | ACTTTTAAATAATTACTTATTTTTTCTGATTGTG |
| | | | GTTCAGTTTAACTGAAGAATATCCTGAGATTGT |
| | | | AAGAAAAGCATTTTTTAAAAGGTATCACTTGTG |
| | | | ATCATTTATCTTTCTAAATTCTATTTTTAATACT |
| | | | GTTCCACCAAAGTGATGCAGTGGTTACCATGAC |
| | | | ACCCTAATTTCATGTGTTTTTGTATTTATGAAAA |
| | | | TAGTTTCATTGTCATTTATTGGCGGTATACAAAG |
| | | | TAAAATGTTATAAATGTGAAGTTATAAAATAAA |
| | | | TATATGCTAATAAAA (SEQ ID NO: 89) |
| PNMA3 | NM_013364/ | MPLTLLQDWCRGEH | GCACTGGCCCACGTGCTGCGCGAGCGAGGGAG |
| | NM_013364.6 | LNTRRCMLILGIPEDC | AGCCACAGTCTGAGCGAACGTCCGCGCTGGGAG |
| | (encoding | GEDEFEETLQEACRH | CCAGGGGTGCCCGACCCCCGTCCGCCGCCGCCG |
| | polynucleotide) | LGRYRVIGRMFRREE | CCGCCGCCGCGCATAGCCCCCGGAGAGCCCTCT |
| | NP_037496/ | NAQAILLELAQDIDY | GGGGACCCCGACCAGAAGGGACCTTGCCCTGG |
| | NP_037496.4 | ALLPREIPGKGGPWE | GAGAAGGCTGTGGAGACCTGGGCCTTCTGCGAT |
| | (protein) | VIVKPRNSDGEFLNR | CACCCTAGGAGTTGATCCAGATATGTGCCTCAC |
| | | LNRFLEEERRTVSDM | GCCCTGATCACTCCCCCCAAATTAGTATCCGCA |
| | | NRVLGSDTNCSAPRV | GAGATTCGAGGACATGCCGTTGACCTTGTTACA |
| | | TISPEFWTWAQTLGA | GGACTGGTGTCGGGGGGAACACCTGAACACCC |
| | | AVQPLLEQMLYRELR | GGAGGTGCATGCTCATCCTGGGGATCCCCGAGG |
| | | VFSGNTISIPGALAFD | ACTGTGGCGAGGATGAGTTTGAGGAGACACTCC |
| | | AWLEHTTEMLQMW | AGGAGGCTTGCAGGCACCTGGGCAGATACAGG |
| | | QVPEGEKRRRLMECL | GTGATTGGCAGGATGTTTAGGAGGGAGGAGAA |
| | | RGPALQVVSGLRASN | CGCCCAGGCGATTCTACTGGAGCTGGCACAAGA |
| | | ASITVEECLAALQQV | TATCGACTATGCTTTGCTCCCAAGGGAAATACC |
| | | FGPVESHKIAQVKLC | AGGAAAGGGGGGGCCCTGGGAAGTGATTGTAA |
| | | KAYQEAGEKVSSFVL | AACCCCGTAACTCAGATGGGGAATTTCTCAACA |
| | | RLEPLLQRAVENNVV | GACTGAACCGCTTCTTAGAGGAGGAGAGGCGG |
| | | SRRNVNQTRLKRVLS | ACCGTGTCAGATATGAACCGAGTCCTCGGGTCG |
| | | GATLPDKLRDKLKL | GACACCAATTGTTCGGCTCCAAGAGTGACTATA |
| | | MKQRRKPPGFLALV | TCACCAGAGTTCTGGACCTGGGCCCAGACTCTG |
| | | KLLREEEEWEATLGP | GGGGCAGCAGTGCAGCCTCTGCTAGAACAAATG |
| | | DRESLEGLEVAPRPP | TTGTACCGAGAACTAAGAGTGTTTTCTGGGAAC |
| | | ARITGVGAVPLPASG | ACCATATCCATCCCAGGTGCACTGGCCTTTGAT |
| | | NSFDARPSQGYRRRR | GCCTGGCTTGAGCACACCACTGAGATGCTACAG |
| | | GRGQHRRGGVARAG | ATGTGGCAGGTGCCCGAGGGGGAAAAGAGGCG |
| | | SRGSRKRKRHTFCYS | GAGGCTGATGGAATGCTTACGGGGCCCTGCTCT |
| | | CGEDGHIRVQCINPS | CCAGGTGGTCAGTGGGCTCCGGGCCAGCAATGC |
| | | NLLLVKQKKQAAVE | TTCCATAACTGTGGAGGAGTGCCTGGCTGCCTT |
| | | SGNGNWAWDKSHPK | GCAGCAGGTGTTCGGACCTGTGGAGAGCCATAA |
| | | SKAK (SEQ ID NO: 90) | AATTGCCCAGGTGAAGTTGTGTAAAGCCTATCA |
| | | | GGAGGCAGGAGAGAAAGTATCTAGCTTTGTGTT |
| | | | ACGTTTGGAACCCCTGCTCCAAAGAGCTGTAGA |
| | | | AAACAATGTGGTATCACGTAGAAACGTGAATCA |
| | | | GACTCGCCTGAAACGAGTCTTAAGTGGGGCCAC |
| | | | CCTTCCTGACAAACTCCGAGATAAGCTTAAGCT |
| | | | GATGAAACAGCGAAGGAAGCCTCCTGGTTTCCT |
| | | | GGCCCTGGTGAAGCTCCTGCGTGAGGAGGAGG |
| | | | AATGGGAGGCCACTTTAGGTCCAGATAGGGAG |
| | | | AGTCTGGAGGGGCTGGAAGTAGCCCCAAGGCC |
| | | | ACCTGCCAGGATCACTGGGGTTGGGGCAGTACC |
| | | | TCTCCCTGCCTCTGGCAACAGTTTTGATGCGAG |
| | | | GCCTTCCCAGGGCTACCGGCGCCGGAGGGGCAG |
| | | | AGGCCAACACCGAAGGGGTGGTGTGGCAAGGG |
| | | | CTGGCTCTCGAGGCTCAAGAAAACGGAAACGCC |
| | | | ACACATTCTGCTATAGCTGTGGGGAAGACGGCC |
| | | | ACATCAGGGTACAGTGCATCAACCCCTCCAACC |
| | | | TGCTCTTGGTAAAGCAGAAGAAACAGGCTGCAG |
| | | | TTGAGTCGGGAAACGGGAACTGGGCTTGGGAC |
| | | | AAGAGCCATCCCAAGTCCAAGGCCAAGTAGGCT |
| | | | CGGGAGAACAGGGCAACATTTCCTACCACAGGC |
| | | | CAAGGAGACAAAAGAGATATTGGAAGGAGGGG |
| | | | AAAGAGAAGCCCAGACAAACAGCAGATGAGTT |
| | | | GAGTGGGGCAGAGGGACAGGGCAGCCAGACCA |
| | | | AGGCCAAGCCTTCTCACCCTTGGCCAGCTGGAA |
| | | | GGGACTTCAGCAACCAAGACCACCTGGCAACA |
| | | | GGCTCAGTGGGGGTCAGGTCCAGGTCCCCGAAG |
| | | | AGGTGCTGGAGAGGAAAGCAGGGAGCCACTGC |
| | | | ATCCAGCACATGGGGTGCCTGGGCCTCAGATGG |
| | | | GGACCCCAAAGAAGCAGAAGCTGAAGAAGGTA |
| | | | CGGCTGGGGGTTCTGTCCTGCTCATCCAACCAC |
| | | | CCCTAAATACCCACCCTGTGGACTTTGAGCTGA |
| | | | ACATGCCCACTGGCCCCCAGGCCACATGGGACC |
| | | | TGGAGGAGCCTACCTGGGGCCTGCCCCTGCCAG |
| | | | CAGGTGCCAGGGCTGGTGAGGAAGAGCTGGGG |
| | | | GGCAGAGGTAAAGCCCTGCAGGGGAGGCCACA |
| | | | GGGTCCATCCCGTCTTCAGGATCATCTACACTG |
| | | | CACTAGGGGAGCCCCAGGAAGGCAGCACCCTG |
| | | | GAGGCCCTGTGCCAGTGAGGACAGGAGACCCT |
| | | | AAGGCCCCGGGAGCCCAGTGCCAGCCAGAGGT |
| | | | TGTGCAGGCAAGGAGACCAAAGATTGATGAGA |
| | | | AGACCCCCAGCAGGGGTACTGGGTACCCGGCA |
| | | | GGCCAGTGCCCTCACAGTTGACTTGGACCAGGG |
| | | | TGGCTGTGAAGGGAAGTCTTTGTTGCAAAGGAG |
| | | | GAGGAAAAGGGAGGACTTGGTAGGGTTTTGTTT |
| | | | CTTCTGCTTGTTTCTGTACAGGGCCACCAGACTC |
| | | | CTGGAGAGATCAAGCAAGGAGAACCTGGGGCT |
| | | | GCCATGGCCAAAGCAACTCAACAGATGCCAATG |
| | | | CCAATTCCAAGGCCAGCCACAACCCTGCCACCT |
| | | | TGGGGAATCCAGCCTGGAGGCATCCCCTAAGCA |
| | | | GCCAGCCATGGCCTGGGTGGAGGCACCTGAAG |
| | | | ACGTCTGTCCCAAACTCCCCCAGCCCTGAGCTG |
| | | | GGAGATGACAGGGGGAAAGAGGCCCTCTCAAG |
| | | | GGTGCCAGATGCCTGGGTCTCCCAAGAGGGGTC |
| | | | CCCCAACTCACTGTTCCCGGGACAGGCTGCCCC |
| | | | CTGTTCCAGGAAGCTCATCCTCACCTGTGTAGG |
| | | | CCCCTGTAGTGACCCACGCGTCCAGCAGACGCC |
| | | | CACCCACCGCTAGCCGTTGTTCCTGTGCAAAGT |
| | | | AGTGTGCTATGCACCCACCCAGGTGGCCGCCTC |
| | | | TGGGCCCAAGGCACATGCTGTGAGCTTCCTGTG |
| | | | AGCCCAGGCTCTGCTCACTGCTGTCCCGCGTCA |
| | | | TGAGCACCACCTCTGCTTTCCCTGTGTAGATCTA |
| | | | GGCCAGTGGCTGCTTGTTCTTGTGGAGCTGTGT |
| | | | GTGTTCTTCTCTGAGCAGCTCCTCCCCGGAGTCC |
| | | | CCCAGCACAGTCCCAGGAGATGACAGGAAGGA |
| | | | AGCACCAGGGCAAGGCGGACGCTCACCCTGTG |
| | | | ACCACGATGGTGACCGTGACTGTGGGAGGAAG |
| | | | AACTGGACCCAGGACGGAGCGGGGCTGCCCTG |
| | | | CCTGAGGCTCCCGAGGAGCTTTGTGCTTTGGTG |
| | | | TTCCACCCCTGTTGTTACTCATGACTCAGTTTCC |
| | | | TTAACCTGGTAGGGTGTTCCCTGCTGTGTTTTCC |
| | | | AGTGTCCTGTGACTGTCCTGTGCGGGCCATAGG |
| | | | GCAGGGCCCTGCCCCAGCAGATGGGCTTGGGAG |
| | | | GGGACTCCCTAAAGCCAGTGGACACTGCCAGAG |
| | | | TCTACCTTCCTGGCAAGAGGCAGACCCCGGGGC |
| | | | CCTCAGGAAGGAGGGAGTTGGCAGCGGGGGCT |
| | | | GCAGCAGGAGTAGGAGCAGATGAGGCGTCTTG |
| | | | CCAGGAACCTCAGGAGGAGGGGGCCCGGGACC |
| | | | TGTGTGGGACCTGTGTCCTGTGGTGGCCGTTTGC |
| | | | AGTTTCTCTCTGTGTTGTGATTCCCTTCTCTTCA |
| | | | ACGTTTTCAGTACGTGTTTCTCTTCAATAAACTT |
| | | | CATTCAGTGTTCCA (SEQ ID NO: 91) |
| PNMA3_i2 | NM_00128253/ | MPLTLLQDWCRGEH | GCACTGGCCCACGTGCTGCGCGAGCGAGGGAG |
| | NM_001282535.2 | LNTRRCMLILGIPEDC | AGCCACAGTCTGAGCGAACGTCCGCGCTGGGAG |
| | (encoding | GEDEFEETLQEACRH | CCAGGGGTGCCCGACCCCCGTCCGCCGCCGCCG |
| | polynucleotide) | LGRYRVIGRMFRREE | CCGCCGCCGCGCATAGCCCCCGGAGAGCCCTCT |
| | NP_001269464/ | NAQAILLELAQDIDY | GGGGACCCCGACCAGAAGGGACCTTGCCCTGG |
| | NP_001269464.1 | ALLPREIPGKGGPWE | GAGAAGGCTGTGGAGACCTGGGCCTTCTGCGAT |
| | (protein) | VIVKPRNSDGEFLNR | CACCCTAGGAGTTGATCCAGATATGTGCCTCAC |
| | | LNRFLEEERRTVSDM | GCCCTGATCACTCCCCCCAAATTAGTATCCGCA |
| | | NRVLGSDTNCSAPRV | GAGATTCGAGGACATGCCGTTGACCTTGTTACA |
| | | TISPEFWTWAQTLGA | GGACTGGTGTCGGGGGGAACACCTGAACACCC |
| | | AVQPLLEQMLYRELR | GGAGGTGCATGCTCATCCTGGGGATCCCCGAGG |
| | | VFSGNTISIPGALAFD | ACTGTGGCGAGGATGAGTTTGAGGAGACACTCC |
| | | AWLEHTTEMLQMW | AGGAGGCTTGCAGGCACCTGGGCAGATACAGG |
| | | QVPEGEKRRRLMECL | GTGATTGGCAGGATGTTTAGGAGGGAGGAGAA |
| | | RGPALQVVSGLRASN | CGCCCAGGCGATTCTACTGGAGCTGGCACAAGA |
| | | ASITVEECLAALQQV | TATCGACTATGCTTTGCTCCCAAGGGAAATACC |
| | | FGPVESHKIAQVKLC | AGGAAAGGGGGGGCCCTGGGAAGTGATTGTAA |
| | | KAYQEAGEKVSSFVL | AACCCCGTAACTCAGATGGGGAATTTCTCAACA |
| | | RLEPLLQRAVENNVV | GACTGAACCGCTTCTTAGAGGAGGAGAGGCGG |
| | | SRRNVNQTRLKRVLS | ACCGTGTCAGATATGAACCGAGTCCTCGGGTCG |
| | | GATLPDKLRDKLKL | GACACCAATTGTTCGGCTCCAAGAGTGACTATA |
| | | MKQRRKPPGFLALV | TCACCAGAGTTCTGGACCTGGGCCCAGACTCTG |
| | | KLLREEEEWEATLGP | GGGGCAGCAGTGCAGCCTCTGCTAGAACAAATG |
| | | DRESLEGLEVAPRPP | TTGTACCGAGAACTAAGAGTGTTTTCTGGGAAC |
| | | ARITGVGAVPLPASG | ACCATATCCATCCCAGGTGCACTGGCCTTTGAT |
| | | NSFDARPSQGYRRRR | GCCTGGCTTGAGCACACCACTGAGATGCTACAG |
| | | GRGQHRRGGVARAG | ATGTGGCAGGTGCCCGAGGGGGAAAAGAGGCG |
| | | SRGSRKRKRHTFCYS | GAGGCTGATGGAATGCTTACGGGGCCCTGCTCT |
| | | CGEDGHIRVQCINPS | CCAGGTGGTCAGTGGGCTCCGGGCCAGCAATGC |
| | | NLLLAKETKEILEGG | TTCCATAACTGTGGAGGAGTGCCTGGCTGCCTT |
| | | EREAQTNSR (SEQ ID | GCAGCAGGTGTTCGGACCTGTGGAGAGCCATAA |
| | | NO: 92) | AATTGCCCAGGTGAAGTTGTGTAAAGCCTATCA |
| | | | GGAGGCAGGAGAGAAAGTATCTAGCTTTGTGTT |
| | | | ACGTTTGGAACCCCTGCTCCAAAGAGCTGTAGA |
| | | | AAACAATGTGGTATCACGTAGAAACGTGAATCA |
| | | | GACTCGCCTGAAACGAGTCTTAAGTGGGGCCAC |
| | | | CCTTCCTGACAAACTCCGAGATAAGCTTAAGCT |
| | | | GATGAAACAGCGAAGGAAGCCTCCTGGTTTCCT |
| | | | GGCCCTGGTGAAGCTCCTGCGTGAGGAGGAGG |
| | | | AATGGGAGGCCACTTTAGGTCCAGATAGGGAG |
| | | | AGTCTGGAGGGGCTGGAAGTAGCCCCAAGGCC |
| | | | ACCTGCCAGGATCACTGGGGTTGGGGCAGTACC |
| | | | TCTCCCTGCCTCTGGCAACAGTTTTGATGCGAG |
| | | | GCCTTCCCAGGGCTACCGGCGCCGGAGGGGCAG |
| | | | AGGCCAACACCGAAGGGGTGGTGTGGCAAGGG |
| | | | CTGGCTCTCGAGGCTCAAGAAAACGGAAACGCC |
| | | | ACACATTCTGCTATAGCTGTGGGGAAGACGGCC |
| | | | ACATCAGGGTACAGTGCATCAACCCCTCCAACC |
| | | | TGCTCTTGGCCAAGGAGACAAAAGAGATATTGG |
| | | | AAGGAGGGGAAAGAGAAGCCCAGACAAACAGC |
| | | | AGATGAGTTGAGTGGGGCAGAGGGACAGGGCA |
| | | | GCCAGACCAAGGCCAAGCCTTCTCACCCTTGGC |
| | | | CAGCTGGAAGGGACTTCAGCAACCAAGACCAC |
| | | | CTGGCAACAGGCTCAGTGGGGGTCAGGTCCAGG |
| | | | TCCCCGAAGAGGTGCTGGAGAGGAAAGCAGGG |
| | | | AGCCACTGCATCCAGCACATGGGGTGCCTGGGC |
| | | | CTCAGATGGGGACCCCAAAGAAGCAGAAGCTG |
| | | | AAGAAGGTACGGCTGGGGGTTCTGTCCTGCTCA |
| | | | TCCAACCACCCCTAAATACCCACCCTGTGGACT |
| | | | TTGAGCTGAACATGCCCACTGGCCCCCAGGCCA |
| | | | CATGGGACCTGGAGGAGCCTACCTGGGGCCTGC |
| | | | CCCTGCCAGCAGGTGCCAGGGCTGGTGAGGAA |
| | | | GAGCTGGGGGGCAGAGGTAAAGCCCTGCAGGG |
| | | | GAGGCCACAGGGTCCATCCCGTCTTCAGGATCA |
| | | | TCTACACTGCACTAGGGGAGCCCCAGGAAGGCA |
| | | | GCACCCTGGAGGCCCTGTGCCAGTGAGGACAGG |
| | | | AGACCCTAAGGCCCCGGGAGCCCAGTGCCAGCC |
| | | | AGAGGTTGTGCAGGCAAGGAGACCAAAGATTG |
| | | | ATGAGAAGACCCCCAGCAGGGGTACTGGGTAC |
| | | | CCGGCAGGCCAGTGCCCTCACAGTTGACTTGGA |
| | | | CCAGGGTGGCTGTGAAGGGAAGTCTTTGTTGCA |
| | | | AAGGAGGAGGAAAAGGGAGGACTTGGTAGGGT |
| | | | TTTGTTTCTTCTGCTTGTTTCTGTACAGGGCCAC |
| | | | CAGACTCCTGGAGAGATCAAGCAAGGAGAACC |
| | | | TGGGGCTGCCATGGCCAAAGCAACTCAACAGAT |
| | | | GCCAATGCCAATTCCAAGGCCAGCCACAACCCT |
| | | | GCCACCTTGGGGAATCCAGCCTGGAGGCATCCC |
| | | | CTAAGCAGCCAGCCATGGCCTGGGTGGAGGCAC |
| | | | CTGAAGACGTCTGTCCCAAACTCCCCCAGCCCT |
| | | | GAGCTGGGAGATGACAGGGGGAAAGAGGCCCT |
| | | | CTCAAGGGTGCCAGATGCCTGGGTCTCCCAAGA |
| | | | GGGGTCCCCCAACTCACTGTTCCCGGGACAGGC |
| | | | TGCCCCCTGTTCCAGGAAGCTCATCCTCACCTGT |
| | | | GTAGGCCCCTGTAGTGACCCACGCGTCCAGCAG |
| | | | ACGCCCACCCACCGCTAGCCGTTGTTCCTGTGC |
| | | | AAAGTAGTGTGCTATGCACCCACCCAGGTGGCC |
| | | | GCCTCTGGGCCCAAGGCACATGCTGTGAGCTTC |
| | | | CTGTGAGCCCAGGCTCTGCTCACTGCTGTCCCG |
| | | | CGTCATGAGCACCACCTCTGCTTTCCCTGTGTAG |
| | | | ATCTAGGCCAGTGGCTGCTTGTTCTTGTGGAGCT |
| | | | GTGTGTGTTCTTCTCTGAGCAGCTCCTCCCCGGA |
| | | | GTCCCCCAGCACAGTCCCAGGAGATGACAGGA |
| | | | AGGAAGCACCAGGGCAAGGCGGACGCTCACCC |
| | | | TGTGACCACGATGGTGACCGTGACTGTGGGAGG |
| | | | AAGAACTGGACCCAGGACGGAGCGGGGCTGCC |
| | | | CTGCCTGAGGCTCCCGAGGAGCTTTGTGCTTTG |
| | | | GTGTTCCACCCCTGTTGTTACTCATGACTCAGTT |
| | | | TCCTTAACCTGGTAGGGTGTTCCCTGCTGTGTTT |
| | | | TCCAGTGTCCTGTGACTGTCCTGTGCGGGCCAT |
| | | | AGGGCAGGGCCCTGCCCCAGCAGATGGGCTTGG |
| | | | GAGGGGACTCCCTAAAGCCAGTGGACACTGCCA |
| | | | GAGTCTACCTTCCTGGCAAGAGGCAGACCCCGG |
| | | | GGCCCTCAGGAAGGAGGGAGTTGGCAGCGGGG |
| | | | GCTGCAGCAGGAGTAGGAGCAGATGAGGCGTC |
| | | | TTGCCAGGAACCTCAGGAGGAGGGGGCCCGGG |
| | | | ACCTGTGTGGGACCTGTGTCCTGTGGTGGCCGT |
| | | | TTGCAGTTTCTCTCTGTGTTGTGATTCCCTTCTCT |
| | | | TCAACGTTTTCAGTACGTGTTTCTCTTCAATAAA |
| | | | CTTCATTCAGTGTTCCA (SEQ ID NO: 93) |
| MOAP1 | NM_022151/ | MTLRLLEDWCRGMD | GCGCAGCTTCCCCGAGCGAGACCAAAACAGGT |
| (PNMA4) | NM_022151.5 | MNPRKALLIAGISQS | GGAATCCGGGCTGGAGCCGGAGCTCCGGCGGC |
| | (encoding | CSVAEIEEALQAGLA | GCGGGTGGCGGCACGTCCCTCCAGACAGTACCA |
| | polynucleotide) | PLGEYRLLGRMFRRD | CAGGCACCTGGAGTACCGGCATCGGTCGCTGTG |
| | NP_071434/ | ENRKVALVGLTAETS | GCCCCCGAGTGTCCGTCAGAGCCTAGGGGAGCC |
| | NP_071434.2 | HALVPKEIPGKGGIW | TGCCCTCCCGCGCCTCGTCGGGGCCCGGCCAGG |
| | (protein) | RVIFKPPDPDNTFLSR | CACCTTGGCCGCCGGCGCACGGACGCGGGCACG |
| | | LNEFLAGEGMTVGEL | AGCACTAGATCACGGCTGCTGGACCTCGGCACG |
| | | SRALGHENGSLDPEQ | TTGACAAGATTTCTCTGGGGTACCGCGGAGGAT |
| | | GMIPEMWAPMLAQA | TACTTTGAATTTCGGTGGTCGCCTGTGGTCTGGC |
| | | LEALQPALQCLKYKK | ATATTTAGAACTTAAGTCTATTATTTCGGGCACC |
| | | LRVFSGRESPEPGEEE | ATGACTTTGAGGCTTTTAGAAGACTGGTGCAGG |
| | | FGRWMFHTTQMIKA | GGGATGGACATGAACCCTCGGAAAGCGCTATTG |
| | | WQVPDVEKRRRLLE | ATTGCCGGCATCTCCCAGAGCTGCAGTGTGGCA |
| | | SLRGPALDVIRVLKIN | GAAATCGAGGAGGCTCTGCAGGCTGGTTTAGCT |
| | | NPLITVDECLQALEE | CCCTTGGGGGAGTACAGACTGCTTGGAAGGATG |
| | | VFGVTDNPRELQVK | TTCAGGAGGGATGAGAACAGGAAAGTAGCCTT |
| | | YLTTYQKDEEKLSAY | AGTAGGGCTTACTGCGGAGACTAGTCACGCCCT |
| | | VLRLEPLLQKLVQRG | GGTCCCTAAGGAGATACCGGGAAAAGGGGGTA |
| | | AIERDAVNQARLDQ | TCTGGAGAGTGATCTTTAAGCCCCCTGACCCAG |
| | | VIAGAVHKTIRRELN | ATAATACATTTTTAAGCAGATTAAATGAATTTTT |
| | | LPEDGPAPGFLQLLV | AGCGGGAGAGGGCATGACAGTGGGTGAGTTGA |
| | | LIKDYEAAEEEEALL | GCAGAGCTCTTGGACATGAAAATGGCTCCTTAG |
| | | QAILEGNFT (SEQ ID | ACCCAGAGCAGGGCATGATCCCGGAAATGTGG |
| | | NO: 94) | GCCCCTATGTTGGCACAGGCATTAGAGGCTCTT |
| | | | CAGCCTGCCCTGCAATGCTTGAAGTATAAAAAG |
| | | | CTGAGAGTGTTCTCGGGCAGGGAGTCTCCAGAA |
| | | | CCAGGAGAAGAAGAATTTGGACGCTGGATGTTT |
| | | | CATACTACTCAGATGATAAAGGCGTGGCAGGTG |
| | | | CCAGATGTAGAGAAGAGAAGGCGATTGCTAGA |
| | | | GAGCCTTCGAGGCCCAGCACTTGATGTTATTCG |
| | | | TGTCCTCAAGATAAACAATCCTTTAATTACTGTC |
| | | | GATGAATGTCTGCAGGCTCTTGAGGAGGTATTT |
| | | | GGGGTTACAGATAATCCTAGGGAGTTGCAGGTC |
| | | | AAATATCTAACCACTTACCAGAAGGATGAGGAA |
| | | | AAGTTGTCGGCTTATGTACTAAGGCTGGAGCCT |
| | | | TTGTTACAGAAGCTGGTACAGAGAGGAGCAATT |
| | | | GAGAGAGATGCTGTGAATCAGGCCCGCCTAGAC |
| | | | CAAGTCATTGCTGGGGCAGTCCACAAAACAATT |
| | | | CGCAGAGAGCTTAATCTGCCAGAGGATGGCCCA |
| | | | GCCCCTGGTTTCTTGCAGTTATTGGTACTAATAA |
| | | | AGGATTATGAGGCAGCTGAGGAGGAGGAGGCC |
| | | | CTTCTCCAGGCAATATTGGAAGGTAATTTCACC |
| | | | TGAGTCTCAGGGAACCACGAAGGGATATGGCA |
| | | | ATGAGTAGAGCATGAAGGTAGAACAGTCTATAT |
| | | | ACTCTTGTGACACATACAATCCCTACCTTGTGCT |
| | | | GCCAAGTAACTCATTTTTGTGCAATTCTCAGTAT |
| | | | AAGCCCTTTGTCGTTTCTGTGCCTATTTAAAGTC |
| | | | TCCTAAAGGTGTAATTGACTAGGAAGGATGTAG |
| | | | TTCTACACTGCCATTTACCTATTTAAATTCATCC |
| | | | TTGTGAATATCTTTGTTGTTGTTGTTGAGACAGA |
| | | | GTCTCGCTCTGTCACCCAGGCTGGAGTGCAGTG |
| | | | GCGTGATCTTGGCTCACTGCATCCTCCGCCTTCC |
| | | | AGGTTTAAGCTATTCTCCTGCCTCAGTTGCCCGA |
| | | | GTAGCTGGGACTACAGGCATGTGCCACCACGCC |
| | | | CAGCTAAGTTTTGCATTATCAGTAGAGACGGGG |
| | | | TTTCACCATGTTGGCCAGGCTAGTTTCGAACTCC |
| | | | TGACCTCAAGTGATCCACCTGCTTCGGCCTCTG |
| | | | AAAGTGCTGGGATTACAGGCGTGAGACACTGCG |
| | | | CCCAGCCTCATCCCTGTGAATTTCTTATTGTACA |
| | | | CAAGTTCTTTCACTATCTGTGTGCAGTGCTCTGA |
| | | | GGGGACAGACAAGGCTTGGGTGTATATGCCAAC |
| | | | CAGATCTCATTGAAGTATCAGCTTGTTTGGTACT |
| | | | AGGTGCAAGTGTAGCATGTCACATGTGACCATG |
| | | | TTGGATCATTGACAATTTTTAGGTATGTACTGAC |
| | | | CTACATTTATGATGAAGATCCTGAGCGGAGGTT |
| | | | AAGATATTAAGTTATTTTCCATATGAATCAGAA |
| | | | TTATATTGATTCTGTGCAATCAAAACAAAAGGC |
| | | | AGAATAGAATGCTGAGATTGGTTAAGTTTGCAA |
| | | | TGACCATCTTGAACCACAGATTTCTGCTATGTGT |
| | | | CATCAAAACATCTAGTTCTGAGTAACATTTTCA |
| | | | CGATTGTTATAAAATTATAGGTGTGAACTTCTA |
| | | | AAATAAAGGAATGCTAATAAAA (SEQ ID NO: 95) |
| PNMA5 | NM_001103151/ | MALTLLEDWCKGMD | TCTCATTGCATTGCGCAGAGCTCAGCCATCTTTC |
| | NM_001103151.1 | MDPRKALLIVGIPME | TCATCCGTCAGGCATTCCTGAGAAAGAGGGCCA |
| | (encoding | CSEVEIQDTVKAGLQ | CTCTACCTCTCTTGCTGCCAGCCTTCACTCCAGC |
| | polynucleotide) | PLCAYRVLGRMFRRE | AAGGAGGGTGCTGGGTGACCTGAGCCCACATA |
| | NP_001096621/ | DNAKAVFIELADTVN | GCCCCGAGCACCCGAAGCACACACAGCAGAGT |
| | NP_001096621.1 | YTTLPSHIPGKGGSW | CCTCTGGACTTTGAGGAAGAACCCACAGCAGGA |
| | (protein) | EVVVKPRNPDDEFLS | GGAAGTCAGCAGGGAGTGGCTGTGTGAAACCT |
| | | RLNYFLKDEGRSMT | GGGACCACTTCTGCCTTCCTACGTGGCAGTGGC |
| | | DVARALGCCSLPAES | TCAGAGTTATTTGAGTGCTGTCAAACTGAGCTG |
| | | LDAEVMPQVRSPPLE | ATTGCTGCCCTAGTATTAGATCAGTCCATAGAA |
| | | PPKESMWYRKLKVF | AGTGGGAGCAATGGCACTGACACTGCTAGAGG |
| | | SGTASPSPGEETFED | ATTGGTGCAAGGGGATGGACATGGACCCCAGA |
| | | WLEQVTEIMPIWQVS | AAGGCCCTGCTGATTGTAGGCATCCCCATGGAG |
| | | EVEKRRRLLESLRGP | TGTAGTGAGGTGGAAATTCAGGACACTGTGAAG |
| | | ALSIMRVLQANNDSI | GCAGGCTTACAGCCCCTGTGCGCATACAGGGTC |
| | | TVEQCLDALKQIFGD | CTAGGGAGAATGTTCAGGAGGGAAGACAATGC |
| | | KEDFRASQFRFLQTS | CAAGGCAGTCTTCATTGAACTGGCTGACACTGT |
| | | PKIGEKVSTFLLRLEP | CAATTACACTACTCTGCCCAGTCACATACCAGG |
| | | LLQKAVHKSPLSVRS | AAAGGGTGGCTCCTGGGAAGTGGTGGTAAAAC |
| | | TDMIRLKHLLARVA | CCCGTAACCCAGATGATGAGTTTCTCAGTAGAC |
| | | MTPALRGKLELLDQR | TGAACTACTTCCTGAAAGATGAGGGCCGAAGTA |
| | | GCPPNFLELMKLIRD | TGACAGATGTGGCCAGAGCCCTGGGATGTTGCA |
| | | EEEWENTEAVMKNK | GCCTCCCTGCCGAGAGCCTGGATGCAGAGGTCA |
| | | EKPSGRGRGASGRQA | TGCCCCAAGTTAGATCCCCACCTTTAGAGCCTC |
| | | RAEASVSAPQATVQA | CGAAAGAAAGTATGTGGTACAGGAAACTGAAA |
| | | RSFSDSSPQTIQGGLP | GTGTTTTCGGGAACTGCTTCCCCTAGCCCAGGC |
| | | PLVKRRRLLGSESTR | GAAGAGACCTTTGAAGACTGGCTAGAGCAGGTC |
| | | GEDHGQATYPKAEN | ACTGAGATAATGCCCATATGGCAAGTGTCTGAG |
| | | QTPGREGPQAAGEEL | GTGGAGAAGAGGCGGCGTTTGCTGGAGAGCTTA |
| | | GNEAGAGAMSHPKP | CGTGGGCCTGCTCTGTCAATCATGCGGGTGCTC |
| | | WET (SEQ ID NO: 96) | CAGGCCAACAATGACTCCATAACTGTGGAGCAG |
| | | | TGCCTTGACGCCCTAAAGCAGATCTTTGGGGAT |
| | | | AAAGAGGACTTTAGAGCCTCTCAGTTTAGGTTT |
| | | | CTGCAGACCTCTCCGAAGATTGGAGAGAAAGTC |
| | | | TCCACTTTCCTGCTGCGCTTAGAGCCCCTGCTGC |
| | | | AGAAAGCCGTGCACAAGAGCCCCTTGTCAGTGC |
| | | | GCAGCACAGACATGATTCGTCTGAAACATCTCT |
| | | | TAGCTCGGGTCGCCATGACCCCCGCCCTCAGGG |
| | | | GCAAGCTGGAGCTCTTGGATCAGCGAGGGTGTC |
| | | | CTCCCAATTTTCTGGAGTTAATGAAGCTCATTCG |
| | | | AGATGAAGAAGAGTGGGAGAACACTGAGGCAG |
| | | | TGATGAAGAATAAGGAGAAGCCATCAGGGAGA |
| | | | GGCCGGGGGGCCTCCGGTAGACAGGCAAGAGC |
| | | | AGAAGCCAGTGTCTCTGCACCTCAGGCCACCGT |
| | | | GCAGGCAAGGTCTTTTAGCGACAGCAGCCCCCA |
| | | | GACCATACAGGGAGGCTTACCCCCATTAGTGAA |
| | | | ACGCAGGCGGCTGTTAGGCAGTGAAAGCACTA |
| | | | GGGGAGAAGACCATGGCCAGGCCACGTATCCC |
| | | | AAGGCTGAAAACCAGACTCCAGGCAGGGAGGG |
| | | | GCCACAGGCTGCAGGAGAGGAGTTGGGAAACG |
| | | | AGGCAGGGGCTGGGGCCATGAGCCATCCCAAG |
| | | | CCCTGGGAAACATAGGCTCAGAAGACCGAAGC |
| | | | ACTCCTTCCCCTGGCAGGCAGCAGGGACATTTG |
| | | | AACAAGGGGAAGGGGCAGCTTACACTAACAGG |
| | | | GAACTTGACTGGGGCAGAGGGACAGGGAAGCC |
| | | | AGGTCGAGATCAAGCCCCCTTACTTTCCACCAG |
| | | | CTGGAAGGGACTTCACCAACCAAGACCAAATA |
| | | | GCATCTGGTTCAGTGGGGGCCGGGCCCATAGCC |
| | | | CCTAACAGGTACAGGAGACACAAGCGGGGAAC |
| | | | CACTACACTTAGCACATGGGGTGCCTGGGCTTC |
| | | | AGATGGGGACCCCAGCGAAGCAGGGGCTGAAG |
| | | | AAGGTGTGGCTGGGGGCTCTGGCCTGGTCCTCT |
| | | | GGACACCCCCAAATATCTACCCTGTGGACTTTG |
| | | | AGCTGAGCATGCCCACTGGCACCCAGGCCACGT |
| | | | GGGACCTGGAGGAGCCTGCCTGGGGCCTGCCTC |
| | | | TGCCAGAAGGAGCCAGGGCTGCTGAGGAAGAG |
| | | | CTGTGGGGCAGAAGTGAAGCCCCGCAGGGGAG |
| | | | GCCACAGGGTCCAGCCTGTCTTTAGGATCATCT |
| | | | ACACTGCATGAGGGGAGCCCCAGGAAGGCAGC |
| | | | ATTCATAAGCCCCTGCACCAGTGAGGAAGAGAA |
| | | | AGAGGAGGAGAATTAAGAAGAGGATGATCGGG |
| | | | AGGAGGAAGAGGAGAATAAGGAGGAGCAGGA |
| | | | GGAAGAGGGGGAGGAGAAGGAGGAAGAGGGG |
| | | | GAGGAGGAAGAGGGGAAGGAGGAGGAAGAAG |
| | | | TGTAAGAAGAGAAGAGAGGATGTGTTGGGGGT |
| | | | GGGTAGTTGTTTTTGGTTTGCGTCATCATCAGGC |
| | | | CCGGGCTCTGCTCACTGCTGTTCCGTGTCAAGA |
| | | | ACTCCACCTCTGCTTTGCCGGTGTAGATCTCGGC |
| | | | CAGTGGCTGCTTGTTCTCTTGGGGATGTGTGTGT |
| | | | TTTTATCTGAGCAGCTCCTTTCCAGAGTACCCCA |
| | | | GCGCAGTTCCAGGAGACGGTGGGAAGGAGGCA |
| | | | TGGCAGACGCTCACCTTGTGACTGTGACCTCGA |
| | | | CTGCAGGAGGACCAACCAGACACGGGAAGGAG |
| | | | CAGGGCTGCCTGAGGCTCCCCAGGAAGCTTTGT |
| | | | GCTTTGGCGTTCCACCCCTGCTGTTACTCGTGAC |
| | | | TCAGTTTCCTCAACTTGGTGGGGTGTTCCCTGCT |
| | | | ATGTTTTCCAGCATCCTGTGACTGTCCTGTGCAG |
| | | | TCTATAGGGCAGGGCCCTGCCCCAGCGGGTGGG |
| | | | CTCAGGAGGGGGTTCCCTGAAGCGAGTACACAT |
| | | | TGCCAGAGCCCACCATCCTGGCAAGAGGTGGAT |
| | | | CCTGGGGCCCTATGGAAGGAGGGATGTGGTGG |
| | | | GGGGGGCCACACTGAGGGAGGGGCCGGGGACT |
| | | | TGTGACCTGTAGTGGCTGTTTGCAGTTTCTCTCT |
| | | | GTGTTAGATTCCCTTTTCTTCAGCAGTTTCAGTT |
| | | | CATGTTTCTCTTCAGTAAATTTTGTTCAGTGTTC |
| | | | CAAAAA (SEQ ID NO: 97) |
| PNMA5_i2 | NM_001103150/ | MALTLLEDWCKGMD | TCTCATTGCATTGCGCAGAGCTCAGCCATCTTTC |
| | NM_001103150.1 | MDPRKALLIVGIPME | TCATCCGTCAGGCATTCCTGAGAAAGAGGGCCA |
| | (encoding | CSEVEIQDTVKAGLQ | CTCTACCTCTCTTGCTGCCAGCCTTCACTCCAGC |
| | polynucleotide) | PLCAYRVLGRMFRRE | AAGGAGGGTGCTGGGTGACCTGAGCCCACATA |
| | NP_001096620/ | DNAKAVFIELADTVN | GCCCCGAGTGAGCAGTGAGGCTGTCTCCTGCCC |
| | NP_001096620.1 | YTTLPSHIPGKGGSW | TCTTCTGCCTGGAGGGCTCGTCAGTGTCCCCAG |
| | (protein) | EVVVKPRNPDDEFLS | GTGTCAGGCCCTGCCTCCTGACGTTGGCCTTTTC |
| | | RLNYFLKDEGRSMT | ACTACAGGCACCCGAAGCACACACAGCAGAGT |
| | | DVARALGCCSLPAES | CCTCTGGACTTTGAGGAAGAACCCACAGCAGGA |
| | | LDAEVMPQVRSPPLE | GGAAGTCAGCAGGGAGTGGCTGTGTGAAACCT |
| | | PPKESMWYRKLKVF | GGGACCACTTCTGCCTTCCTACGTGGCAGTGGC |
| | | SGTASPSPGEETFED | TCAGAGTTATTTGAGTGCTGTCAAACTGAGCTG |
| | | WLEQVTEIMPIWQVS | ATTGCTGCCCTAGTATTAGATCAGTCCATAGAA |
| | | EVEKRRRLLESLRGP | AGTGGGAGCAATGGCACTGACACTGCTAGAGG |
| | | ALSIMRVLQANNDSI | ATTGGTGCAAGGGGATGGACATGGACCCCAGA |
| | | TVEQCLDALKQIFGD | AAGGCCCTGCTGATTGTAGGCATCCCCATGGAG |
| | | KEDFRASQFRFLQTS | TGTAGTGAGGTGGAAATTCAGGACACTGTGAAG |
| | | PKIGEKVSTFLLRLEP | GCAGGCTTACAGCCCCTGTGCGCATACAGGGTC |
| | | LLQKAVHKSPLSVRS | CTAGGGAGAATGTTCAGGAGGGAAGACAATGC |
| | | TDMIRLKHLLARVA | CAAGGCAGTCTTCATTGAACTGGCTGACACTGT |
| | | MTPALRGKLELLDQR | CAATTACACTACTCTGCCCAGTCACATACCAGG |
| | | GCPPNFLELMKLIRD | AAAGGGTGGCTCCTGGGAAGTGGTGGTAAAAC |
| | | EEEWENTEAVMKNK | CCCGTAACCCAGATGATGAGTTTCTCAGTAGAC |
| | | EKPSGRGRGASGRQA | TGAACTACTTCCTGAAAGATGAGGGCCGAAGTA |
| | | RAEASVSAPQATVQA | TGACAGATGTGGCCAGAGCCCTGGGATGTTGCA |
| | | RSFSDSSPQTIQGGLP | GCCTCCCTGCCGAGAGCCTGGATGCAGAGGTCA |
| | | PLVKRRRLLGSESTR | TGCCCCAAGTTAGATCCCCACCTTTAGAGCCTC |
| | | GEDHGQATYPKAEN | CGAAAGAAAGTATGTGGTACAGGAAACTGAAA |
| | | QTPGREGPQAAGEEL | GTGTTTTCGGGAACTGCTTCCCCTAGCCCAGGC |
| | | GNEAGAGAMSHPKP | GAAGAGACCTTTGAAGACTGGCTAGAGCAGGTC |
| | | WET (SEQ ID NO: 98) | ACTGAGATAATGCCCATATGGCAAGTGTCTGAG |
| | | | GTGGAGAAGAGGCGGCGTTTGCTGGAGAGCTTA |
| | | | CGTGGGCCTGCTCTGTCAATCATGCGGGTGCTC |
| | | | CAGGCCAACAATGACTCCATAACTGTGGAGCAG |
| | | | TGCCTTGACGCCCTAAAGCAGATCTTTGGGGAT |
| | | | AAAGAGGACTTTAGAGCCTCTCAGTTTAGGTTT |
| | | | CTGCAGACCTCTCCGAAGATTGGAGAGAAAGTC |
| | | | TCCACTTTCCTGCTGCGCTTAGAGCCCCTGCTGC |
| | | | AGAAAGCCGTGCACAAGAGCCCCTTGTCAGTGC |
| | | | GCAGCACAGACATGATTCGTCTGAAACATCTCT |
| | | | TAGCTCGGGTCGCCATGACCCCCGCCCTCAGGG |
| | | | GCAAGCTGGAGCTCTTGGATCAGCGAGGGTGTC |
| | | | CTCCCAATTTTCTGGAGTTAATGAAGCTCATTCG |
| | | | AGATGAAGAAGAGTGGGAGAACACTGAGGCAG |
| | | | TGATGAAGAATAAGGAGAAGCCATCAGGGAGA |
| | | | GGCCGGGGGGCCTCCGGTAGACAGGCAAGAGC |
| | | | AGAAGCCAGTGTCTCTGCACCTCAGGCCACCGT |
| | | | GCAGGCAAGGTCTTTTAGCGACAGCAGCCCCCA |
| | | | GACCATACAGGGAGGCTTACCCCCATTAGTGA |
| | | | AACGCAGGCGGCTGTTAGGCAGTGAAAGCACT |
| | | | AGGGGAGAAGACCATGGCCAGGCCACGTATCC |
| | | | CAAGGCTGAAAACCAGACTCCAGGCAGGGAGG |
| | | | GGCCACAGGCTGCAGGAGAGGAGTTGGGAAAC |
| | | | GAGGCAGGGGCTGGGGCCATGAGCCATCCCAA |
| | | | GCCCTGGGAAACATAGGCTCAGAAGACCGAAG |
| | | | CACTCCTTCCCCTGGCAGGCAGCAGGGACATTT |
| | | | GAACAAGGGGAAGGGGCAGCTTACACTAACAG |
| | | | GGAACTTGACTGGGGCAGAGGGACAGGGAAGC |
| | | | CAGGTCGAGATCAAGCCCCCTTACTTTCCACCA |
| | | | GCTGGAAGGGACTTCACCAACCAAGACCAAAT |
| | | | AGCATCTGGTTCAGTGGGGGCCGGGCCCATAGC |
| | | | CCCTAACAGGTACAGGAGACACAAGCGGGGAA |
| | | | CCACTACACTTAGCACATGGGGTGCCTGGGCTT |
| | | | CAGATGGGGACCCCAGCGAAGCAGGGGCTGAA |
| | | | GAAGGTGTGGCTGGGGGCTCTGGCCTGGTCCTC |
| | | | TGGACACCCCCAAATATCTACCCTGTGGACTTT |
| | | | GAGCTGAGCATGCCCACTGGCACCCAGGCCACG |
| | | | TGGGACCTGGAGGAGCCTGCCTGGGGCCTGCCT |
| | | | CTGCCAGAAGGAGCCAGGGCTGCTGAGGAAGA |
| | | | GCTGTGGGGCAGAAGTGAAGCCCCGCAGGGGA |
| | | | GGCCACAGGGTCCAGCCTGTCTTTAGGATCATC |
| | | | TACACTGCATGAGGGGAGCCCCAGGAAGGCAG |
| | | | CATTCATAAGCCCCTGCACCAGTGAGGAAGAGA |
| | | | AAGAGGAGGAGAATTAAGAAGAGGATGATCGG |
| | | | GAGGAGGAAGAGGAGAATAAGGAGGAGCAGG |
| | | | AGGAAGAGGGGGAGGAGAAGGAGGAAGAGGG |
| | | | GGAGGAGGAAGAGGGGAAGGAGGAGGAAGAA |
| | | | GTGTAAGAAGAGAAGAGAGGATGTGTTGGGGG |
| | | | TGGGTAGTTGTTTTTGGTTTGCGTCATCATCAGG |
| | | | CCCGGGCTCTGCTCACTGCTGTTCCGTGTCAAG |
| | | | AACTCCACCTCTGCTTTGCCGGTGTAGATCTCGG |
| | | | CCAGTGGCTGCTTGTTCTCTTGGGGATGTGTGTG |
| | | | TTTTTATCTGAGCAGCTCCTTTCCAGAGTACCCC |
| | | | AGCGCAGTTCCAGGAGACGGTGGGAAGGAGGC |
| | | | ATGGCAGACGCTCACCTTGTGACTGTGACCTCG |
| | | | ACTGCAGGAGGACCAACCAGACACGGGAAGGA |
| | | | GCAGGGCTGCCTGAGGCTCCCCAGGAAGCTTTG |
| | | | TGCTTTGGCGTTCCACCCCTGCTGTTACTCGTGA |
| | | | CTCAGTTTCCTCAACTTGGTGGGGTGTTCCCTGC |
| | | | TATGTTTTCCAGCATCCTGTGACTGTCCTGTGCA |
| | | | GTCTATAGGGCAGGGCCCTGCCCCAGCGGGTGG |
| | | | GCTCAGGAGGGGGTTCCCTGAAGCGAGTACACA |
| | | | TTGCCAGAGCCCACCATCCTGGCAAGAGGTGGA |
| | | | TCCTGGGGCCCTATGGAAGGAGGGATGTGGTGG |
| | | | GGGGGGCCACACTGAGGGAGGGGCCGGGGACT |
| | | | TGTGACCTGTAGTGGCTGTTTGCAGTTTCTCTCT |
| | | | GTGTTAGATTCCCTTTTCTTCAGCAGTTTCAGTT |
| | | | CATGTTTCTCTTCAGTAAATTTTGTTCAGTGTTC |
| | | | CAAAAA (SEQ ID NO: 99) |
| PNMA5_i3 | NM_052926/ | MALTLLEDWCKGMD | ATTGCATTGCGCAGAGCTCAGCCATCTTTCTCAT |
| | NM_052926.3 | MDPRKALLIVGIPME | CCGTCAGGCATTCCTGAGAAAGAGGGCCACTCT |
| | (encoding | CSEVEIQDTVKAGLQ | ACCTCTCTTGCTGCCAGCCTTCACTCCAGCAAG |
| | polynucleotide) | PLCAYRVLGRMFRRE | GAGGGTGCTGGGTGACCTGAGCCCACATAGCCC |
| | NP_443158/ | DNAKAVFIELADTVN | CGAGTGAGCAGTGAGGCTGTCTCCTGCCCTCTT |
| | NP_443158.1 | YTTLPSHIPGKGGSW | CTGCCTGGAGGGCTCGTCAGTGTCCCCAGGTGT |
| | (protein) | EVVVKPRNPDDEFLS | CAGGCCCTGCCTCCTGACGTTGGCCTTTTCACTA |
| | | RLNYFLKDEGRSMT | CAGGCACCCGAAGCACACACAGCAGAGTCCTCT |
| | | DVARALGCCSLPAES | GGACTTTGAGGAAGAACCCACAGCAGGAGGAA |
| | | LDAEVMPQVRSPPLE | GCTGTGTGAAACCTGGGACCACTTCTGCCTTCCT |
| | | PPKESMWYRKLKVF | ACGTGGCAGTGGCTCAGAGTTATTTGAGTGCTG |
| | | SGTASPSPGEETFED | TCAAACTGAGCTGATTGCTGCCCTAGTATTAGA |
| | | WLEQVTEIMPIWQVS | TCAGTCCATAGAAAGTGGGAGCAATGGCACTGA |
| | | EVEKRRRLLESLRGP | CACTGCTAGAGGATTGGTGCAAGGGGATGGAC |
| | | ALSIMRVLQANNDSI | ATGGACCCCAGAAAGGCCCTGCTGATTGTAGGC |
| | | TVEQCLDALKQIFGD | ATCCCCATGGAGTGTAGTGAGGTGGAAATTCAG |
| | | KEDFRASQFRFLQTS | GACACTGTGAAGGCAGGCTTACAGCCCCTGTGC |
| | | PKIGEKVSTFLLRLEP | GCATACAGGGTCCTAGGGAGAATGTTCAGGAG |
| | | LLQKAVHKSPLSVRS | GGAAGACAATGCCAAGGCAGTCTTCATTGAACT |
| | | TDMIRLKHLLARVA | GGCTGACACTGTCAATTACACTACTCTGCCCAG |
| | | MTPALRGKLELLDQR | TCACATACCAGGAAAGGGTGGCTCCTGGGAAGT |
| | | GCPPNFLELMKLIRD | GGTGGTAAAACCCCGTAACCCAGATGATGAGTT |
| | | EEEWENTEAVMKNK | TCTCAGTAGACTGAACTACTTCCTGAAAGATGA |
| | | EKPSGRGRGASGRQA | GGGCCGAAGTATGACAGATGTGGCCAGAGCCCT |
| | | RAEASVSAPQATVQA | GGGATGTTGCAGCCTCCCTGCCGAGAGCCTGGA |
| | | RSFSDSSPQTIQGGLP | TGCAGAGGTCATGCCCCAAGTTAGATCCCCACC |
| | | PLVKRRRLLGSESTR | TTTAGAGCCTCCGAAAGAAAGTATGTGGTACAG |
| | | GEDHGQATYPKAEN | GAAACTGAAAGTGTTTTCGGGAACTGCTTCCCC |
| | | QTPGREGPQAAGEEL | TAGCCCAGGCGAAGAGACCTTTGAAGACTGGCT |
| | | GNEAGAGAMSHPKP | AGAGCAGGTCACTGAGATAATGCCCATATGGCA |
| | | WET (SEQ ID NO: 100) | AGTGTCTGAGGTGGAGAAGAGGCGGCGTTTGCT |
| | | | GGAGAGCTTACGTGGGCCTGCTCTGTCAATCAT |
| | | | GCGGGTGCTCCAGGCCAACAATGACTCCATAAC |
| | | | TGTGGAGCAGTGCCTTGACGCCCTAAAGCAGAT |
| | | | CTTTGGGGATAAAGAGGACTTTAGAGCCTCTCA |
| | | | GTTTAGGTTTCTGCAGACCTCTCCGAAGATTGG |
| | | | AGAGAAAGTCTCCACTTTCCTGCTGCGCTTAGA |
| | | | GCCCCTGCTGCAGAAAGCCGTGCACAAGAGCCC |
| | | | CTTGTCAGTGCGCAGCACAGACATGATTCGTCT |
| | | | GAAACATCTCTTAGCTCGGGTCGCCATGACCCC |
| | | | CGCCCTCAGGGGCAAGCTGGAGCTCTTGGATCA |
| | | | GCGAGGGTGTCCTCCCAATTTTCTGGAGTTAAT |
| | | | GAAGCTCATTCGAGATGAAGAAGAGTGGGAGA |
| | | | ACACTGAGGCAGTGATGAAGAATAAGGAGAAG |
| | | | CCATCAGGGAGAGGCCGGGGGGCCTCCGGTAG |
| | | | ACAGGCAAGAGCAGAAGCCAGTGTCTCTGCACC |
| | | | TCAGGCCACCGTGCAGGCAAGGTCTTTTAGCGA |
| | | | CAGCAGCCCCCAGACCATACAGGGAGGCTTACC |
| | | | CCCATTAGTGAAACGCAGGCGGCTGTTAGGCAG |
| | | | TGAAAGCACTAGGGGAGAAGACCATGGCCAGG |
| | | | CCACGTATCCCAAGGCTGAAAACCAGACTCCAG |
| | | | GCAGGGAGGGGCCACAGGCTGCAGGAGAGGAG |
| | | | TTGGGAAACGAGGCAGGGGCTGGGGCCATGAG |
| | | | CCATCCCAAGCCCTGGGAAACATAGGCTCAGAA |
| | | | GACCGAAGCACTCCTTCCCCTGGCAGGCAGCAG |
| | | | GGACATTTGAACAAGGGGAAGGGGCAGCTTAC |
| | | | ACTAACAGGGAACTTGACTGGGGCAGAGGGAC |
| | | | AGGGAAGCCAGGTCGAGATCAAGCCCCCTTACT |
| | | | TTCCACCAGCTGGAAGGGACTTCACCAACCAAG |
| | | | ACCAAATAGCATCTGGTTCAGTGGGGGCCGGGC |
| | | | CCATAGCCCCTAACAGGTACAGGAGACACAAG |
| | | | CGGGGAACCACTACACTTAGCACATGGGGTGCC |
| | | | TGGGCTTCAGATGGGGACCCCAGCGAAGCAGG |
| | | | GGCTGAAGAAGGTGTGGCTGGGGGCTCTGGC |
| | | | CTGGTCCTCTGGACACCCCCAAATATCTACCCT |
| | | | GTGGACTTTGAGCTGAGCATGCCCACTGGCACC |
| | | | CAGGCCACGTGGGACCTGGAGGAGCCTGCCTGG |
| | | | GGCCTGCCTCTGCCAGAAGGAGCCAGGGCTGCT |
| | | | GAGGAAGAGCTGTGGGGCAGAAGTGAAGCCCC |
| | | | GCAGGGGAGGCCACAGGGTCCAGCCTGTCTTTA |
| | | | GGATCATCTACACTGCATGAGGGGAGCCCCAGG |
| | | | AAGGCAGCATTCATAAGCCCCTGCACCAGTGAG |
| | | | GAAGAGAAAGAGGAGGAGAATTAAGAAGAGG |
| | | | ATGATCGGGAGGAGGAAGAGGAGAATAAGGAG |
| | | | GAGCAGGAGGAAGAGGGGGAGGAGAAGGAGG |
| | | | AAGAGGGGGAGGAGGAAGAGGGGAAGGAGGA |
| | | | GGAAGAAGTGTAAGAAGAGAAGAGAGGATGTG |
| | | | TTGGGGGTGGGTAGTTGTTTTTGGTTTGCGTCAT |
| | | | CATCAGGCCCGGGCTCTGCTCACTGCTGTTCCGT |
| | | | GTCAAGAACTCCACCTCTGCTTTGCCGGTGTAG |
| | | | ATCTCGGCCAGTGGCTGCTTGTTCTCTTGGGGAT |
| | | | GTGTGTGTTTTTATCTGAGCAGCTCCTTTCCAGA |
| | | | GTACCCCAGCGCAGTTCCAGGAGACGGTGGGA |
| | | | AGGAGGCATGGCAGACGCTCACCTTGTGACTGT |
| | | | GACCTCGACTGCAGGAGGACCAACCAGACACG |
| | | | GGAAGGAGCAGGGCTGCCTGAGGCTCCCCAGG |
| | | | AAGCTTTGTGCTTTGGCGTTCCACCCCTGCTGTT |
| | | | ACTCGTGACTCAGTTTCCTCAACTTGGTGGGGT |
| | | | GTTCCCTGCTATGTTTTCCAGCATCCTGTGACTG |
| | | | TCCTGTGCAGTCTATAGGGCAGGGCCCTGCCCC |
| | | | AGCGGGTGGGCTCAGGAGGGGGTTCCCTGAAG |
| | | | CGAGTACACATTGCCAGAGCCCACCATCCTGGC |
| | | | AAGAGGTGGATCCTGGGGCCCTATGGAAGGAG |
| | | | GGATGTGGTGGGGGGGGCCACACTGAGGGAGG |
| | | | GGCCGGGGACTTGTGACCTGTAGTGGCTGTTTG |
| | | | CAGTTTCTCTCTGTGTTAGATTCCCTTTTCTTCA |
| | | | GCAGTTTCAGTTCATGTTTCTCTTCAGTAAATTT |
| | | | TGTTCAGTGTTCCA (SEQ ID NO: 101) |
| PNMA5_i4 | NM_001184924/ | MALTLLEDWCKGMD | GCAGTCAGCACGGAGGCGGCAGCCGCCATAGA |
| | NM_001184924.2 | MDPRKALLIVGIPME | CGTGTGGTGGTCGCGCTGCGCGGGAACTCCGGC |
| | (encoding | CSEVEIQDTVKAGLQ | TTGGAGGCACCTCCGGCAGATCCAGTGAAGCAG |
| | polynucleotide) | PLCAYRVLGRMFRRE | GACCCATCACGCCTGAGTCAGGAACTGCCTAAC |
| | NP_001171853/ | DNAKAVFIELADTVN | TGGATAGAGTTGAAAAGCAGAGCGAGTCTGAA |
| | NP_001171853.1 | YTTLPSHIPGKGGSW | GTGGCTGTGTTATTGCAGCATCCGCGCTGCCAG |
| | (protein) | EVVVKPRNPDDEFLS | CAGGGCCAACCCAGCAAGGCACCCGAAGCACA |
| | | RLNYFLKDEGRSMT | CACAGCAGAGTCCTCTGGACTTTGAGGAAGAAC |
| | | DVARALGCCSLPAES | CCACAGCAGGAGGAAGTCAGCAGGGAGTGGCT |
| | | LDAEVMPQVRSPPLE | GTGTGAAACCTGGGACCACTTCTGCCTTCCTAC |
| | | PPKESMWYRKLKVF | GTGGCAGTGGCTCAGAGTTATTTGAGTGCTGTC |
| | | SGTASPSPGEETFED | AAACTGAGCTGATTGCTGCCCTAGTATTAGATC |
| | | WLEQVTEIMPIWQVS | AGTCCATAGAAAGTGGGAGCAATGGCACTGAC |
| | | EVEKRRRLLESLRGP | ACTGCTAGAGGATTGGTGCAAGGGGATGGACAT |
| | | ALSIMRVLQANNDSI | GGACCCCAGAAAGGCCCTGCTGATTGTAGGCAT |
| | | TVEQCLDALKQIFGD | CCCCATGGAGTGTAGTGAGGTGGAAATTCAGGA |
| | | KEDFRASQFRFLQTS | CACTGTGAAGGCAGGCTTACAGCCCCTGTGCGC |
| | | PKIGEKVSTFLLRLEP | ATACAGGGTCCTAGGGAGAATGTTCAGGAGGG |
| | | LLQKAVHKSPLSVRS | AAGACAATGCCAAGGCAGTCTTCATTGAACTGG |
| | | TDMIRLKHLLARVA | CTGACACTGTCAATTACACTACTCTGCCCAGTC |
| | | MTPALRGKLELLDQR | ACATACCAGGAAAGGGTGGCTCCTGGGAAGTG |
| | | GCPPNFLELMKLIRD | GTGGTAAAACCCCGTAACCCAGATGATGAGTTT |
| | | EEEWENTEAVMKNK | CTCAGTAGACTGAACTACTTCCTGAAAGATGAG |
| | | EKPSGRGRGASGRQA | GGCCGAAGTATGACAGATGTGGCCAGAGCCCTG |
| | | RAEASVSAPQATVQA | GGATGTTGCAGCCTCCCTGCCGAGAGCCTGGAT |
| | | RSFSDSSPQTIQGGLP | GCAGAGGTCATGCCCCAAGTTAGATCCCCACCT |
| | | PLVKRRRLLGSESTR | TTAGAGCCTCCGAAAGAAAGTATGTGGTACAGG |
| | | GEDHGQATYPKAEN | AAACTGAAAGTGTTTTCGGGAACTGCTTCCCCT |
| | | QTPGREGPQAAGEEL | AGCCCAGGCGAAGAGACCTTTGAAGACTGGCTA |
| | | GNEAGAGAMSHPKP | GAGCAGGTCACTGAGATAATGCCCATATGGCAA |
| | | WET (SEQ ID NO: 102) | GTGTCTGAGGTGGAGAAGAGGCGGCGTTTGCTG |
| | | | GAGAGCTTACGTGGGCCTGCTCTGTCAATCATG |
| | | | CGGGTGCTCCAGGCCAACAATGACTCCATAACT |
| | | | GTGGAGCAGTGCCTTGACGCCCTAAAGCAGATC |
| | | | TTTGGGGATAAAGAGGACTTTAGAGCCTCTCAG |
| | | | TTTAGGTTTCTGCAGACCTCTCCGAAGATTGGA |
| | | | GAGAAAGTCTCCACTTTCCTGCTGCGCTTAGAG |
| | | | CCCCTGCTGCAGAAAGCCGTGCACAAGAGCCCC |
| | | | TTGTCAGTGCGCAGCACAGACATGATTCGTCTG |
| | | | AAACATCTCTTAGCTCGGGTCGCCATGACCCCC |
| | | | GCCCTCAGGGGCAAGCTGGAGCTCTTGGATCAG |
| | | | CGAGGGTGTCCTCCCAATTTTCTGGAGTTAATG |
| | | | AAGCTCATTCGAGATGAAGAAGAGTGGGAGAA |
| | | | CACTGAGGCAGTGATGAAGAATAAGGAGAAGC |
| | | | CATCAGGGAGAGGCCGGGGGGCCTCCGGTAGA |
| | | | CAGGCAAGAGCAGAAGCCAGTGTCTCTGCACCT |
| | | | CAGGCCACCGTGCAGGCAAGGTCTTTTAGCGAC |
| | | | AGCAGCCCCCAGACCATACAGGGAGGCTTACCC |
| | | | CCATTAGTGAAACGCAGGCGGCTGTTAGGCAGT |
| | | | GAAAGCACTAGGGGAGAAGACCATGGCCAGGC |
| | | | CACGTATCCCAAGGCTGAAAACCAGACTCCAGG |
| | | | CAGGGAGGGGCCACAGGCTGCAGGAGAGGAGT |
| | | | TGGGAAACGAGGCAGGGGCTGGGGCCATGAGC |
| | | | CATCCCAAGCCCTGGGAAACATAGGCTCAGAAG |
| | | | ACCGAAGCACTCCTTCCCCTGGCAGGCAGCAGG |
| | | | GACATTTGAACAAGGGGAAGGGGCAGCTTACA |
| | | | CTAACAGGGAACTTGACTGGGGCAGAGGGACA |
| | | | GGGAAGCCAGGTCGAGATCAAGCCCCCTTACTT |
| | | | TCCACCAGCTGGAAGGGACTTCACCAACCAAGA |
| | | | CCAAATAGCATCTGGTTCAGTGGGGGCCGGGCC |
| | | | CATAGCCCCTAACAGGTACAGGAGACACAAGC |
| | | | GGGGAACCACTACACTTAGCACATGGGGTGCCT |
| | | | GGGCTTCAGATGGGGACCCCAGCGAAGCAGGG |
| | | | GCTGAAGAAGGTGTGGCTGGGGGCTCTGGCCTG |
| | | | GTCCTCTGGACACCCCCAAATATCTACCCTGTG |
| | | | GACTTTGAGCTGAGCATGCCCACTGGCACCCAG |
| | | | GCCACGTGGGACCTGGAGGAGCCTGCCTGGGGC |
| | | | CTGCCTCTGCCAGAAGGAGCCAGGGCTGCTGAG |
| | | | GAAGAGCTGTGGGGCAGAAGTGAAGCCCCGCA |
| | | | GGGGAGGCCACAGGGTCCAGCCTGTCTTTAGGA |
| | | | TCATCTACACTGCATGAGGGGAGCCCCAGGAAG |
| | | | GCAGCATTCATAAGCCCCTGCACCAGTGAGGAA |
| | | | GAGAAAGAGGAGGAGAATTAAGAAGAGGATGA |
| | | | TCGGGAGGAGGAAGAGGAGAATAAGGAGGAGC |
| | | | AGGAGGAAGAGGGGGAGGAGAAGGAGGAAGA |
| | | | GGGGGAGGAGGAAGAGGGGAAGGAGGAGGAA |
| | | | GAAGTGTAAGAAGAGAAGAGAGGATGTGTTGG |
| | | | GGGTGGGTAGTTGTTTTTGGTTTGCGTCATCATC |
| | | | AGGCCCGGGCTCTGCTCACTGCTGTTCCGTGTC |
| | | | AAGAACT |
| | | | CCACCTCTGCTTTGCCGGTGTAGATCTCGGCCA |
| | | | GTGGCTGCTTGTTCTCTTGGGGATGTGTGTGTTT |
| | | | TTATCTGAGCAGCTCCTTTCCAGAGTACCCCAG |
| | | | CGCAGTTCCAGGAGACGGTGGGAAGGAGGCAT |
| | | | GGCAGACGCTCACCTTGTGACTGTGACCTCGAC |
| | | | TGCAGGAGGACCAACCAGACACGGGAAGGAGC |
| | | | AGGGCTGCCTGAGGCTCCCCAGGAAGCTTTGTG |
| | | | CTTTGGCGTTCCACCCCTGCTGTTACTCGTGACT |
| | | | CAGTTTCCTCAACTTGGTGGGGTGTTCCCTGCTA |
| | | | TGTTTTCCAGCATCCTGTGACTGTCCTGTGCAGT |
| | | | CTATAGGGCAGGGCCCTGCCCCAGCGGGTGGGC |
| | | | TCAGGAGGGGGTTCCCTGAAGCGAGTACACATT |
| | | | GCCAGAGCCCACCATCCTGGCAAGAGGTGGATC |
| | | | CTGGGGCCCTATGGAAGGAGGGATGTGGTGGG |
| | | | GGGGGCCACACTGAGGGAGGGGCCGGGGACTT |
| | | | GTGACCTGTAGTGGCTGTTTGCAGTTTCTCTCTG |
| | | | TGTTAGATTCCCTTTTCTTCAGCAGTTTCAGTTC |
| | | | ATGTTTCTCTTCAGTAAATTTTGTTCAGTGTTCC |
| | | | A (SEQ ID NO: 103) |
| PNMA6A | NM_032882/ | MAVTMLQDWCRWM | CCAAACGCCGAGCGAGCGAGGGAGAGGCACAG |
| | NM_032882.6 | GVNARRGLLILGIPED | TCAGAGGGAACGCCCGCGCGGGGAGCCAGGGG |
| | (encoding | CDDAEFQESLEAALR | CGCCCGACCCCGCCGCCGCCGCAGCGGCGCGCA |
| | polynucleotide) | PMGHFTVLGKAFREE | GCCCCCGACGCGCCCTGTGGGGACCCGGACCAG |
| | NP_116271/ | DNATAALVELDREV | GAGGGACCCTGCCCCGGGAAAAGGCTGTGGAG |
| | NP_116271.3 | NYALVPREIPGTGGP | ACCTGGGCTCCGACCCCAGTTCATCCCCCCACA |
| | (protein) | WNVVFVPRCSGEEFL | CCCCCGCCGCCCCGTGCCACCCTGGTCCGCGCT |
| | | GLGRVFHFPEQEGQ | GGGAACCCTATCCTGCCCCTCGTGTCAGCCCGG |
| | | MVESVAGALGVGLR | CACTGGCCAGAATCGCGGGCATGGCGGTGACCA |
| | | RVCWLRSIGQAVQP | TGCTGCAGGACTGGTGCCGGTGGATGGGGGTCA |
| | | WVEAVRCQSLGVFS | ACGCTCGCAGGGGCCTGCTCATCCTGGGCATCC |
| | | GRDQPAPGEESFEVW | CGGAGGACTGTGATGATGCCGAATTCCAAGAGT |
| | | LDHTTEMLHVWQGV | CCCTCGAGGCTGCCCTGAGGCCTATGGGACACT |
| | | SERERRRRLLEGLRG | TTACAGTGCTAGGCAAAGCGTTTCGAGAGGAGG |
| | | TALQLVHALLAENPA | ATAATGCCACCGCGGCCCTGGTCGAGCTCGACC |
| | | RTAQDCLAALAQVF | GGGAAGTCAACTATGCTTTGGTCCCCAGGGAAA |
| | | GDNESQATIRVKCLT | TCCCCGGCACTGGGGGCCCGTGGAACGTGGTCT |
| | | AQQQSGERLSAFVLR | TTGTGCCCCGTTGCTCAGGCGAGGAGTTTCTCG |
| | | LEVLLQKAMEKEAL | GTCTCGGTCGCGTGTTCCACTTCCCGGAGCAAG |
| | | ARASADVRLRQML | AGGGGCAGATGGTGGAGAGCGTGGCCGGCGCC |
| | | TRAHLTEPLDEALRK | CTGGGTGTGGGGCTGCGCAGGGTGTGCTGGCTG |
| | | LRMAGRSPSFLEMLG | CGATCCATCGGTCAGGCGGTCCAGCCCTGGGTG |
| | | LVRESEAWEASLARS | GAGGCCGTGAGGTGCCAGAGCCTGGGCGTGTTT |
| | | VRAQTQEGAGARAG | TCCGGGAGGGACCAGCCAGCCCCAGGGGAGGA |
| | | AQAVARASTKVEAV | GTCCTTTGAGGTCTGGCTAGACCACACCACCGA |
| | | PGGPGREPEGLLQAG | AATGCTGCATGTGTGGCAGGGGGTCTCGGAAAG |
| | | GQEAEELLQEGLKPV | GGAGAGGAGGAGGAGGCTGCTGGAAGGCTTGC |
| | | LEECDN (SEQ ID NO: | GTGGGACCGCCCTGCAGCTCGTGCACGCGCTCC |
| | | 104) | TGGCGGAGAACCCCGCCAGGACGGCGCAGGAC |
| | | | TGTCTGGCGGCCCTGGCCCAGGTGTTTGGAGAC |
| | | | AACGAGTCCCAGGCGACCATCCGGGTGAAGTGT |
| | | | CTGACCGCTCAGCAGCAGTCAGGCGAGCGTCTC |
| | | | TCAGCTTTCGTGTTGCGGCTGGAAGTGCTGCTG |
| | | | CAGAAGGCCATGGAGAAGGAGGCCCTGGCCAG |
| | | | AGCATCCGCCGACCGCGTGCGCCTGAGGCAGAT |
| | | | GCTCACCAGGGCCCACCTTACTGAGCCTCTGGA |
| | | | TGAAGCACTGAGGAAGCTGAGAATGGCCGGGA |
| | | | GGTCTCCAAGTTTCTTGGAGATGCTGGGGCTCG |
| | | | TTCGGGAGTCTGAGGCATGGGAGGCCAGTCTAG |
| | | | CCAGGAGCGTGAGAGCCCAGACACAGGAAGGG |
| | | | GCCGGTGCCCGGGCTGGTGCCCAGGCTGTTGCC |
| | | | AGAGCCAGCACTAAAGTAGAGGCGGTCCCAGG |
| | | | AGGTCCTGGTCGGGAGCCAGAGGGCCTCCTCCA |
| | | | GGCAGGAGGCCAGGAGGCTGAGGAGCTCCTCC |
| | | | AGGAGGGGCTCAAGCCCGTCCTGGAGGAATGT |
| | | | GATAACTAGGTTGGGGCTGGGGAGGCAGCCCA |
| | | | GCGCGAGTCCTCCCCGGGCAAATAGGCTCCGAG |
| | | | GGCCCCGGGGCCTCCTCTCCTCCTCTCAGGCAG |
| | | | CAGGGCCCTGGAGACAGGCGGAGGCGGGGCCA |
| | | | GGGCCGGTCCCTCACCCCACATCGGGATCGGGG |
| | | | CCCCCCACTTCCCCCCAAGGGGCCCTGCCCACC |
| | | | ACCACCTTCCCGGTGACCCAGATGACCACATTT |
| | | | AATACCAAATGGGGTGGGGGGAGGCGCCCCTC |
| | | | CAGTGCCAGGGGCACGTGCTGTGAGCTTCCTGG |
| | | | GAGCCCAGGTTGTGCTCACTGCTCTCCCGTATC |
| | | | GTGAGCACCACCTCTGCTTTCCCTGCGTAGATCT |
| | | | AGGCCAGGGGCTGCTTGTTTTTGTGGAGCCGTG |
| | | | TGTGTTCTTCTCTGAGCAGCTCCTCCCCAGAGGA |
| | | | CCCCAGCGCAGTCCCGGGAGATGGCGGAAAGA |
| | | | AGGCACCAGGGCACAGTGGACACTCATCCCGTG |
| | | | ACAGCGATGGTGACCATGACTGTGGGAGAAAG |
| | | | AACAGGACCCGGGATGGAGTGGGGCTGTCTGA |
| | | | GTTTCCCCAGTGAACTTTGTGCTTTGGCGTTCCA |
| | | | CCCCTGTTGTTACTTGTTACTGAGTTTCCTAGAC |
| | | | CTGGGGTGGGTGTTCCCCCAGGAGGAGGGGGGT |
| | | | CCCGACCTGTGTCCTGTGGTGGCCATTTGCAGCT |
| | | | TCTGTGTTGTGATTCCCTTCTCTTCAACGGTTTC |
| | | | AGTACATATCTCTCTTCAATAAATTTCATTCAGT |
| | | | GTTCCA (SEQ ID NO: 105) |
| PNMA6E | NM_001351293/ | MALAMLRDWCRWM | ACACGCTTCCACGCTGCGGAGCTGAGGTGATCT |
| | NM_001351293.2 | GANAERSLLILGIPDD | CTTCAGACCCTTCGGAGAGACCCAGATATCTGA |
| | (encoding | CKEHEFQEAVRAALS | CTTCCCACTGAGAGACCCGGAATTGCCCAGAGT |
| | polynucleotide) | PLGRREQPGCEEESFE | TTGAGAAAGCTGTGTCGGAGACTGCAACAGTCA |
| | NP_001338222/ | SWVEHAKDMLQLW | GCACACATTCCTGATTGATCAGGCTCCCTCCCTC |
| | NP_001338222.1 | CHASEREKKRWLLES | AAGTCCTCTGCGATGGCTCTGGCGATGCTTCGG |
| | (protein) | LGGPALEVVSGLLEE | GACTGGTGCAGGTGGATGGGTGCGAACGCAGA |
| | | DTNLSALDCLAALGQ | GCGCTCCCTGCTCATCCTGGGTATCCCTGATGAC |
| | | VFRNQDTRMTSRLKF | TGCAAGGAACATGAGTTCCAGGAGGCCGTGCG |
| | | LTCTQGPQEGLFAFV | GGCTGCCCTGTCGCCCCTGGGCAGGAGGGAGCA |
| | | VRLEGLLQRAVEKG | GCCAGGCTGCGAGGAAGAGTCCTTTGAGAGCTG |
| | | AVCPALANYLRLQQ | GGTGGAGCATGCCAAGGATATGCTGCAGCTGTG |
| | | VLSWARPSEALQDTL | GTGCCATGCGTCGGAAAGGGAGAAGAAGAGGT |
| | | RGMQLEKRPPGFLGL | GGCTGCTGGAGAGCTTGGGCGGCCCGGCCCTGG |
| | | LRLIREMEAWAAFPA | AAGTCGTGAGCGGCCTCCTGGAGGAAGATACCA |
| | | RSQQGVAWAAAPVE | ACTTGTCCGCGCTGGACTGCCTGGCGGCGCTGG |
| | | SEDPAAAQASPAQGN | GGCAGGTATTTAGGAACCAGGACACTCGAATGA |
| | | ASEAGPGAEDAAEA | CTTCGAGGCTGAAGTTCCTGACCTGCACGCAGG |
| | | ASATKEAARGAPAA | GGCCCCAGGAGGGGCTGTTTGCCTTCGTGGTGC |
| | | GEGESAPAGPEGLGQ | GCCTGGAAGGCCTGCTGCAGAGGGCTGTGGAG |
| | | ARPIEVPWGSSPARM | AAGGGGGCCGTCTGCCCAGCCTTGGCCAATTAC |
| | | SSAVWVFPRGLSWG | CTGCGACTACAGCAGGTGCTGTCTTGGGCCCGC |
| | | PEGLIQVRGQEARKP | CCCAGCGAGGCACTCCAGGATACCCTGAGAGG |
| | | PLEGLQTILEEPENED | GATGCAGCTGGAGAAGAGGCCACCTGGCTTCCT |
| | | EDGAGDEGQPKSSQ | GGGGCTGCTCCGGCTCATCCGGGAGATGGAGGC |
| | | GK (SEQ ID NO: 106) | ATGGGCAGCCTTCCCAGCGAGGAGCCAGCAGG |
| | | | GTGTGGCCTGGGCAGCGGCCCCAGTGGAGAGTG |
| | | | AAGACCCAGCTGCTGCCCAGGCCTCCCCAGCCC |
| | | | AGGGGAATGCCAGCGAGGCTGGTCCCGGAGCA |
| | | | GAAGATGCTGCCGAGGCCGCTTCTGCCACCAAA |
| | | | GAGGCTGCAAGGGGAGCCCCTGCCGCTGGGGA |
| | | | AGGTGAAAGTGCCCCTGCAGGCCCCGAAGGCCT |
| | | | AGGTCAGGCAAGGCCCATAGAGGTCCCCTGGG |
| | | | GCTCCTCCCCAGCCCGGATGAGCAGTGCTGTCT |
| | | | GGGTGTTCCCAAGAGGTCTTAGCTGGGGTCCAG |
| | | | AGGGCCTCATCCAGGTGAGAGGCCAGGAAGCC |
| | | | AGGAAACCCCCACTGGAGGGGCTCCAGACCATC |
| | | | TTGGAGGAGCCGGAAAACGAGGATGAGGATGG |
| | | | GGCCGGGGACGAGGGCCAGCCCAAGTCCTCCC |
| | | | AGGGCAAATAGGCTCCTAGGGCCCCGGGGCCTC |
| | | | CTCTCCTCTCAGGCAGCAGCGCCTTGGAGGCAG |
| | | | ACAGAGGCCAGGCCAGGGCCAGTCCCTCACCCC |
| | | | ACATTCAGGAGTAAGGGCCCCCCACCTCCCCCA |
| | | | AGGGGCTCTGGCCACCACCCCCATTCCTTCCCT |
| | | | GTGACCCGGATGACCACGTTTGATACAAAATGG |
| | | | GGTGGGGAGAGGCGCCCCCCCGCTCCCTTGCAC |
| | | | CCAGCACACCCAGCCCCAGCCCCAAACCCTGCC |
| | | | GCCACGGGGGGCTGGCCTGGAGGGAGCCCTGA |
| | | | GTGGGCAGCTGTGGCCTGGGTGGGGGCACCTGA |
| | | | AGATGTCTGCCCCCAACCCAGGCCGTGAGTTGG |
| | | | GAGAGACAGGGGGAAAGAGGCCCTCTCAAGGT |
| | | | TGCCAGCTGCCTGGGTCTCTCAAGAGGGGTCAG |
| | | | CCCACTTGCCATCTCCGGGGCAGGCTGCCCTCT |
| | | | GTTCCGGGAAGCTCACCCTCACCTGTGTGACCC |
| | | | ACGCGCCCAGCAGACGCCCACCCACTGCTAGCC |
| | | | ATCATTTCTGCGAAAAGTCATGTACTGTGCGCC |
| | | | CATGCAGGCGGCCACCTCTGGGCCCGGGGCACG |
| | | | TGCTGTGAGCTTCCTGCGAGCCCAGGCTCTGCTT |
| | | | GCTGCTGTCCTGCATCGTGAGCACCACCTCTGCT |
| | | | TTCCTGGCGTAGATCTAGGCCAGGGGCTGCTTG |
| | | | TTCTCGTGGAGCTGCGTGTGTTCTTCTCTGAGCA |
| | | | GCCTCTCCCCGGAGACCCCCAGCGCAGTCCCAG |
| | | | GAGATGGCGGGAAGGAGGCACCAGGGCACGGC |
| | | | GGACGCTCACCCCGTGACCACGATGGTGACCCT |
| | | | GACTGCGGGAAGAAGAACCGGACCCGGGGCAC |
| | | | AGCGGGGCTGCGTGAGGATCCACAAGAACTAT |
| | | | GCTTTGGCGTTTCACCCCTGTTGTTACTTGTGAC |
| | | | TCAGTTTCTTCAGCCTGGTGGGGTGTTCCCTGGT |
| | | | GTTTCCCAGTGTTCTGTGACTGTCCTGTGAAGGC |
| | | | CATAGGGATGGGCTCAGGAGGGGGCTCCCTGA |
| | | | AGCCAGTGGACACTGCCAGAGTCCACTGTCCTG |
| | | | GCAAAAGGCAGACCCTGGGGCCCTCGGGAAGG |
| | | | AGGGAGGTGGCAGCGGGGGCAGCAGCGGGAGG |
| | | | GGGAGCAGATGACGTGCCTTGCCAGGAACCCCA |
| | | | GGAGGAGGGGGCCTGGGACCTGTGTCCTGTGGT |
| | | | GGCTGTTTACAGTTTCTCTCTGTATTGTGGTTTC |
| | | | CTTCTCTTCAATAGTTTCAGTATATGTTTCTCTTC |
| | | | AATAAATTTCATTCCGTGTTCCA (SEQ ID NO: |
| | | | 107) |
| PNMA6E_i2 | NM_001351294/ | MALAMLRDWCRWM | GGTGCCGTGGTCAGGTCCTAGGGCTGGGCTGTG |
| | NM_001351294.2 | GANAERSLLILGIPDD | TCGGAGACTGCAACAGTCAGCACACATTCCTGA |
| | (encoding | CKEHEFQEAVRAALS | TTGATCAGGCTCCCTCCCTCAAGTCCTCTGCGAT |
| | polynucleotide) | PLGRREQPGCEEESFE | GGCTCTGGCGATGCTTCGGGACTGGTGCAGGTG |
| | NP_001338223/ | SWVEHAKDMLQLW | GATGGGTGCGAACGCAGAGCGCTCCCTGCTCAT |
| | NP_001338223.1 | CHASEREKKRWLLES | CCTGGGTATCCCTGATGACTGCAAGGAACATGA |
| | (protein) | LGGPALEVVSGLLEE | GTTCCAGGAGGCCGTGCGGGCTGCCCTGTCGCC |
| | | DTNLSALDCLAALGQ | CCTGGGCAGGAGGGAGCAGCCAGGCTGCGAGG |
| | | VFRNQDTRMTSRLKF | AAGAGTCCTTTGAGAGCTGGGTGGAGCATGCCA |
| | | LTCTQGPQEGLFAFV | AGGATATGCTGCAGCTGTGGTGCCATGCGTCGG |
| | | VRLEGLLQRAVEKG | AAAGGGAGAAGAAGAGGTGGCTGCTGGAGAGC |
| | | AVCPALANYLRLQQ | TTGGGCGGCCCGGCCCTGGAAGTCGTGAGCGGC |
| | | VLSWARPSEALQDTL | CTCCTGGAGGAAGATACCAACTTGTCCGCGCTG |
| | | RGMQLEKRPPGFLGL | GACTGCCTGGCGGCGCTGGGGCAGGTATTTAGG |
| | | LRLIREMEAWAAFPA | AACCAGGACACTCGAATGACTTCGAGGCTGAAG |
| | | RSQQGVAWAAAPVE | TTCCTGACCTGCACGCAGGGGCCCCAGGAGGGG |
| | | SEDPAAAQASPAQGN | CTGTTTGCCTTCGTGGTGCGCCTGGAAGGCCTG |
| | | ASEAGPGAEDAAEA | CTGCAGAGGGCTGTGGAGAAGGGGGCCGTCTG |
| | | ASATKEAARGAPAA | CCCAGCCTTGGCCAATTACCTGCGACTACAGCA |
| | | GEGESAPAGPEGLGQ | GGTGCTGTCTTGGGCCCGCCCCAGCGAGGCACT |
| | | ARPIEVPWGSSPARM | CCAGGATACCCTGAGAGGGATGCAGCTGGAGA |
| | | SSAVWVFPRGLSWG | AGAGGCCACCTGGCTTCCTGGGGCTGCTCCGGC |
| | | PEGLIQVRGQEARKP | TCATCCGGGAGATGGAGGCATGGGCAGCCTTCC |
| | | PLEGLQTILEEPENED | CAGCGAGGAGCCAGCAGGGTGTGGCCTGGGCA |
| | | EDGAGDEGQPKSSQ | GCGGCCCCAGTGGAGAGTGAAGACCCAGCTGCT |
| | | GK (SEQ ID NO: 108) | GCCCAGGCCTCCCCAGCCCAGGGGAATGCCAGC |
| | | | GAGGCTGGTCCCGGAGCAGAAGATGCTGCCGA |
| | | | GGCCGCTTCTGCCACCAAAGAGGCTGCAAGGGG |
| | | | AGCCCCTGCCGCTGGGGAAGGTGAAAGTGCCCC |
| | | | TGCAGGCCCCGAAGGCCTAGGTCAGGCAAGGC |
| | | | CCATAGAGGTCCCCTGGGGCTCCTCCCCAGCCC |
| | | | GGATGAGCAGTGCTGTCTGGGTGTTCCCAAGAG |
| | | | GTCTTAGCTGGGGTCCAGAGGGCCTCATCCAGG |
| | | | TGAGAGGCCAGGAAGCCAGGAAACCCCCACTG |
| | | | GAGGGGCTCCAGACCATCTTGGAGGAGCCGGA |
| | | | AAACGAGGATGAGGATGGGGCCGGGGACGAGG |
| | | | GCCAGCCCAAGTCCTCCCAGGGCAAATAGGCTC |
| | | | CTAGGGCCCCGGGGCCTCCTCTCCTCTCAGGCA |
| | | | GCAGCGCCTTGGAGGCAGACAGAGGCCAGGCC |
| | | | AGGGCCAGTCCCTCACCCCACATTCAGGAGTAA |
| | | | GGGCCCCCCACCTCCCCCAAGGGGCTCTGGCCA |
| | | | CCACCCCCATTCCTTCCCTGTGACCCGGATGACC |
| | | | ACGTTTGATACAAAATGGGGTGGGGAGAGGCG |
| | | | CCCCCCCGCTCCCTTGCACCCAGCACACCCAGC |
| | | | CCCAGCCCCAAACCCTGCCGCCACGGGGGGCTG |
| | | | GCCTGGAGGGAGCCCTGAGTGGGCAGCTGTGGC |
| | | | CTGGGTGGGGGCACCTGAAGATGTCTGCCCCCA |
| | | | ACCCAGGCCGTGAGTTGGGAGAGACAGGGGGA |
| | | | AAGAGGCCCTCTCAAGGTTGCCAGCTGCCTGGG |
| | | | TCTCTCAAGAGGGGTCAGCCCACTTGCCATCTC |
| | | | CGGGGCAGGCTGCCCTCTGTTCCGGGAAGCTCA |
| | | | CCCTCACCTGTGTGACCCACGCGCCCAGCAGAC |
| | | | GCCCACCCACTGCTAGCCATCATTTCTGCGAAA |
| | | | AGTCATGTACTGTGCGCCCATGCAGGCGGCCAC |
| | | | CTCTGGGCCCGGGGCACGTGCTGTGAGCTTCCT |
| | | | GCGAGCCCAGGCTCTGCTTGCTGCTGTCCTGCA |
| | | | TCGTGAGCACCACCTCTGCTTTCCTGGCGTAGAT |
| | | | CTAGGCCAGGGGCTGCTTGTTCTCGTGGAGCTG |
| | | | CGTGTGTTCTTCTCTGAGCAGCCTCTCCCCGGAG |
| | | | ACCCCCAGCGCAGTCCCAGGAGATGGCGGGAA |
| | | | GGAGGCACCAGGGCACGGCGGACGCTCACCCC |
| | | | GTGACCACGATGGTGACCCTGACTGCGGGAAGA |
| | | | AGAACCGGACCCGGGGCACAGCGGGGCTGCGT |
| | | | GAGGATCCACAAGAACTATGCTTTGGCGTTTCA |
| | | | CCCCTGTTGTTACTTGTGACTCAGTTTCTTCAGC |
| | | | CTGGTGGGGTGTTCCCTGGTGTTTCCCAGTGTTC |
| | | | TGTGACTGTCCTGTGAAGGCCATAGGGATGGGC |
| | | | TCAGGAGGGGGCTCCCTGAAGCCAGTGGACACT |
| | | | GCCAGAGTCCACTGTCCTGGCAAAAGGCAGACC |
| | | | CTGGGGCCCTCGGGAAGGAGGGAGGTGGCAGC |
| | | | GGGGGCAGCAGCGGGAGGGGGAGCAGATGACG |
| | | | TGCCTTGCCAGGAACCCCAGGAGGAGGGGGCCT |
| | | | GGGACCTGTGTCCTGTGGTGGCTGTTTACAGTTT |
| | | | CTCTCTGTATTGTGGTTTCCTTCTCTTCAATAGTT |
| | | | TCAGTATATGTTTCTCTTCAATAAATTTCATTCC |
| | | | GTGTTCCA (SEQ ID NO: 109) |
| PNMA6E_i3 | NM_001367770/ | MALAMLRDWCRWM | ACACGCTTCCACGCTGCGGAGCTGAGGTGATCT |
| | NM_001367770.1 | GANAERSLLILGIPDD | CTTCAGACCCTTCGGAGAGACCCAGATATCTGA |
| | (encoding | CKEHEFQEAVRAALS | CTTCCCACTGAGAGACCCGGAATTGCCCAGAGT |
| | polynucleotide) | PLGRYRVLTKHFRKE | TTGAGAAAGCTGTGTCGGAGACTGCAACAGTCA |
| | NP_001354699/ | LGAKAALVEFAEYLN | GCACACATTCCTGATTGATCAGGCTCCCTCCCTC |
| | NP_001354699.1 | RSLIPHQIPGNGGPW | AAGTCCTCTGCGATGGCTCTGGCGATGCTTCGG |
| | (protein) | KVIFLPQVPVIEFQDM | GACTGGTGCAGGTGGATGGGTGCGAACGCAGA |
| | | PSFPAQPQGQAVAKA | GCGCTCCCTGCTCATCCTGGGTATCCCTGATGAC |
| | | AGEGGGAGEAGGVG | TGCAAGGAACATGAGTTCCAGGAGGCCGTGCG |
| | | EVGAAGEAGGTGEA | GGCTGCCCTGTCGCCCCTGGGCAGGTACCGAGT |
| | | GATGEAGAAGEAGG | ACTCACCAAGCACTTCAGAAAGGAGCTCGGGGC |
| | | AGEAGGVGEAGAAG | CAAGGCAGCCTTGGTGGAGTTCGCTGAGTATTT |
| | | EAGGAGEAGAAGEG | AAACCGAAGCTTGATTCCCCATCAAATACCAGG |
| | | GAAGEAGGAGEAGG | CAATGGGGGGCCCTGGAAAGTGATCTTCCTGCC |
| | | VGEAGAAGEAGGAG | CCAAGTACCTGTTATTGAGTTTCAGGATATGCC |
| | | EAGGVGEAGAAGEA | CAGTTTTCCTGCACAGCCCCAGGGTCAAGCAGT |
| | | GGAGEAGAAGEAGG | AGCAAAAGCTGCAGGTGAGGGAGGAGGCGCAG |
| | | AGEGRAAGEAGAAG | GTGAGGCAGGAGGTGTAGGTGAGGTAGGAGCA |
| | | EAGAVGEAGAAGEA | GCAGGTGAGGCAGGAGGCACAGGTGAGGCAGG |
| | | GAVGEAGAAGEAGA | AGCAACAGGTGAGGCAGGAGCAGCAGGTGAGG |
| | | VGEAGGTNVTKAWV | CAGGAGGCGCAGGTGAGGCAGGAGGTGTAGGT |
| | | QPWRCTLQPVLENR | GAGGCAGGAGCAGCAGGTGAGGCAGGAGGCGC |
| | | AYRELRPFSRREQPG | AGGTGAGGCAGGAGCAGCAGGTGAGGGAGGAG |
| | | CEEESFESWVEHAKD | CAGCAGGTGAGGCAGGAGGCGCAGGTGAGGCA |
| | | MLQLWCHASEREKK | GGAGGTGTAGGTGAGGCAGGAGCAGCAGGTGA |
| | | RWLLESLGGPALEVV | GGCAGGAGGCGCAGGTGAGGCAGGAGGTGTAG |
| | | SGLLEEDTNLSALDC | GTGAGGCAGGAGCAGCAGGTGAGGCAGGAGGC |
| | | LAALGQVFRNQDTR | GCAGGTGAGGCAGGAGCAGCAGGTGAGGCAGG |
| | | MTSRLKFLTCTQGPQ | AGGCGCAGGTGAGGGAAGAGCAGCAGGTGAGG |
| | | EGLFAFVVRLEGLLQ | CAGGAGCAGCAGGTGAGGCAGGAGCTGTGGGT |
| | | RAVEKGAVCPALAN | GAGGCAGGAGCAGCAGGTGAGGCAGGAGCTGT |
| | | YLRLQQVLSWARPSE | GGGTGAGGCAGGAGCTGCAGGTGAGGCAGGAG |
| | | ALQDTLRGMQLEKR | CTGTGGGTGAAGCAGGAGGGACAAATGTAACA |
| | | PPGFLGLLRLIREMEA | AAAGCCTGGGTCCAGCCTTGGCGCTGCACCCTA |
| | | WAAFPARSQQGVAW | CAGCCTGTGCTGGAAAACAGGGCCTACCGGGA |
| | | AAAPVESEDPAAAQ | ATTGAGACCCTTTTCCAGGAGGGAGCAGCCAGG |
| | | ASPAQGNASEAGPGA | CTGCGAGGAAGAGTCCTTTGAGAGCTGGGTGGA |
| | | EDAAEAASATKEAA | GCATGCCAAGGATATGCTGCAGCTGTGGTGCCA |
| | | RGAPAAGEGESAPAG | TGCGTCGGAAAGGGAGAAGAAGAGGTGGCTGC |
| | | PEGLGQARPIEVPWG | TGGAGAGCTTGGGCGGCCCGGCCCTGGAAGTCG |
| | | SSPARMSSAVWVFPR | TGAGCGGCCTCCTGGAGGAAGATACCAACTTGT |
| | | GLSWGPEGLIQVRGQ | CCGCGCTGGACTGCCTGGCGGCGCTGGGGCAGG |
| | | EARKPPLEGLQTILEE | TATTTAGGAACCAGGACACTCGAATGACTTCGA |
| | | PENEDEDGAGDEGQP | GGCTGAAGTTCCTGACCTGCACGCAGGGGCCCC |
| | | KSSQGK (SEQ ID NO: | AGGAGGGGCTGTTTGCCTTCGTGGTGCGCCTGG |
| | | 110) | AAGGCCTGCTGCAGAGGGCTGTGGAGAAGGGG |
| | | | GCCGTCTGCCCAGCCTTGGCCAATTACCTGCGA |
| | | | CTACAGCAGGTGCTGTCTTGGGCCCGCCCCAGC |
| | | | GAGGCACTCCAGGATACCCTGAGAGGGATGCA |
| | | | GCTGGAGAAGAGGCCACCTGGCTTCCTGGGGCT |
| | | | GCTCCGGCTCATCCGGGAGATGGAGGCATGGGC |
| | | | AGCCTTCCCAGCGAGGAGCCAGCAGGGTGTGGC |
| | | | CTGGGCAGCGGCCCCAGTGGAGAGTGAAGACC |
| | | | CAGCTGCTGCCCAGGCCTCCCCAGCCCAGGGGA |
| | | | ATGCCAGCGAGGCTGGTCCCGGAGCAGAAGAT |
| | | | GCTGCCGAGGCCGCTTCTGCCACCAAAGAGGCT |
| | | | GCAAGGGGAGCCCCTGCCGCTGGGGAAGGTGA |
| | | | AAGTGCCCCTGCAGGCCCCGAAGGCCTAGGTCA |
| | | | GGCAAGGCCCATAGAGGTCCCCTGGGGCTCCTC |
| | | | CCCAGCCCGGATGAGCAGTGCTGTCTGGGTGTT |
| | | | CCCAAGAGGTCTTAGCTGGGGTCCAGAGGGCCT |
| | | | CATCCAGGTGAGAGGCCAGGAAGCCAGGAAAC |
| | | | CCCCACTGGAGGGGCTCCAGACCATCTTGGAGG |
| | | | AGCCGGAAAACGAGGATGAGGATGGGGCCGGG |
| | | | GACGAGGGCCAGCCCAAGTCCTCCCAGGGCAA |
| | | | ATAGGCTCCTAGGGCCCCGGGGCCTCCTCTCCT |
| | | | CTCAGGCAGCAGCGCCTTGGAGGCAGACAGAG |
| | | | GCCAGGCCAGGGCCAGTCCCTCACCCCACATTC |
| | | | AGGAGTAAGGGCCCCCCACCTCCCCCAAGGGGC |
| | | | TCTGGCCACCACCCCCATTCCTTCCCTGTGACCC |
| | | | GGATGACCACGTTTGATACAAAATGGGGTGGGG |
| | | | AGAGGCGCCCCCCCGCTCCCTTGCACCCAGCAC |
| | | | ACCCAGCCCCAGCCCCAAACCCTGCCGCCACGG |
| | | | GGGGCTGGCCTGGAGGGAGCCCTGAGTGGGCA |
| | | | GCTGTGGCCTGGGTGGGGGCACCTGAAGATGTC |
| | | | TGCCCCCAACCCAGGCCGTGAGTTGGGAGAGAC |
| | | | AGGGGGAAAGAGGCCCTCTCAAGGTTGCCAGCT |
| | | | GCCTGGGTCTCTCAAGAGGGGTCAGCCCACTTG |
| | | | CCATCTCCGGGGCAGGCTGCCCTCTGTTCCGGG |
| | | | AAGCTCACCCTCACCTGTGTGACCCACGCGCCC |
| | | | AGCAGACGCCCACCCACTGCTAGCCATCATTTC |
| | | | TGCGAAAAGTCATGTACTGTGCGCCCATGCAGG |
| | | | CGGCCACCTCTGGGCCCGGGGCACGTGCTGTGA |
| | | | GCTTCCTGCGAGCCCAGGCTCTGCTTGCTGCTGT |
| | | | CCTGCATCGTGAGCACCACCTCTGCTTTCCTGGC |
| | | | GTAGATCTAGGCCAGGGGCTGCTTGTTCTCGTG |
| | | | GAGCTGCGTGTGTTCTTCTCTGAGCAGCCTCTCC |
| | | | CCGGAGACCCCCAGCGCAGTCCCAGGAGATGG |
| | | | CGGGAAGGAGGCACCAGGGCACGGCGGACGCT |
| | | | CACCCCGTGACCACGATGGTGACCCTGACTGCG |
| | | | GGAAGAAGAACCGGACCCGGGGCACAGCGGGG |
| | | | CTGCGTGAGGATCCACAAGAACTATGCTTTGGC |
| | | | GTTTCACCCCTGTTGTTACTTGTGACTCAGTTTC |
| | | | TTCAGCCTGGTGGGGTGTTCCCTGGTGTTTCCCA |
| | | | GTGTTCTGTGACTGTCCTGTGAAGGCCATAGGG |
| | | | ATGGGCTCAGGAGGGGGCTCCCTGAAGCCAGTG |
| | | | GACACTGCCAGAGTCCACTGTCCTGGCAAAAGG |
| | | | CAGACCCTGGGGCCCTCGGGAAGGAGGGAGGT |
| | | | GGCAGCGGGGGCAGCAGCGGGAGGGGGAGCAG |
| | | | ATGACGTGCCTTGCCAGGAACCCCAGGAGGAG |
| | | | GGGGCCTGGGACCTGTGTCCTGTGGTGGCTGTT |
| | | | TACAGTTTCTCTCTGTATTGTGGTTTCCTTCTCTT |
| | | | CAATAGTTTCAGTATATGTTTCTCTTCAATAAAT |
| | | | TTCATTCCGTGTTCCA (SEQ ID NO: 111) |
| PNMA6F | NM_001354980/ | MLQDWCRRMGVNA | CTCATCTACCTGGGGCTTCTGGGAGAAGCGGGA |
| | NM_001354980.2 | ERSLLILDIPDDCEEH | AGCCCTGTAGTCTTAGCACAGCGGGCTTTGGAG |
| | (encoding | EFQEAVRAALSPLGR | ACCCCTGCGCTGCGCAGCGCGGCCGCGTGACTG |
| | polynucleotide) | YRVLIKVFRKELGAR | AGGCGAGATCTTCCACCCTCCTGCTCCAGGGCC |
| | | AALVEFAEGLNQSLI | TCTAGGTGGCCTCCAGAGTGGGCTCCACAGCTT |
| | | PRQIAGKGGPWKVIS | GAAGGCGGCGTTTTTCCGCGGGTGGTCGCAGCG |
| | | LPQALDAEFQDIPSFP | TCCGCAGAGTCGGAGAAAGCTGCGTCAGAGACT |
| | | AQPQGQAVARGAGE | GCAACAGTCAGCACACATTCCTGTCTGATCAGG |
| | | AGAAGEAGSVGEAG | CTCCCTCCCTCAAGTCCTCTGCGATAGCTCTGGC |
| | | GVNEERSAGEDEAG | GATGCTTCAGGACTGGTGCAGGAGGATGGGTGT |
| | | GIGEAGGVGEAGAA | GAACGCAGAGCGCTCTCTGCTCATCCTGGATAT |
| | | GEAGAAGEAGAAGE | CCCTGACGACTGCGAGGAACATGAGTTCCAGGA |
| | | AGGAGEAGGAGEAG | GGCCGTGCGGGCTGCCCTGTCGCCCCTGGGCAG |
| | | GAGEEGGTGEEGGA | GTACCGAGTGCTCATCAAGGTCTTCAGAAAGGA |
| | | GEAGGAGEEGGEDE | GCTCGGGGCCAGGGCAGCCTTGGTGGAATTCGC |
| | | AGAAGEAVGAGVVE | TGAGGGTTTAAACCAAAGCTTGATTCCCCGCCA |
| | | AWTQSWRQTLRPLV | AATAGCAGGCAAAGGGGGACCCTGGAAAGTGA |
| | | KTMAYRELRPFSGRE | TCTCCCTGCCCCAGGCCCTTGATGCTGAGTTTCA |
| | | QPGCVEESFESWLED | GGATATACCCAGTTTCCCTGCACAGCCCCAGGG |
| | | AKDMLQLWCHASER | GCAAGCAGTGGCCAGAGGTGCAGGTGAAGCAG |
| | | ERRRRLLDSLDGLAL | GAGCTGCAGGTGAGGCAGGATCTGTAGGTGAG |
| | | DIVSGLLEEDPDFSAQ | GCAGGAGGTGTGAATGAGGAAAGATCTGCAGG |
| | | DCLTALGQVFRSRDT | TGAAGATGAGGCAGGAGGTATAGGTGAGGCAG |
| | | WMTSRMKFLTCTQG | GAGGTGTAGGTGAGGCAGGAGCAGCAGGTGAG |
| | | PQEGLFAFVVRLEGL | GCAGGAGCAGCAGGTGAGGCAGGAGCAGCAGG |
| | | LQKAVEKGAVHPAM | TGAGGCAGGAGGTGCAGGTGAGGCAGGAGGTG |
| | | ANHLRLRQVLSRARP | CAGGTGAGGCAGGAGGTGCAGGTGAGGAAGGA |
| | | SEALQDTLRRMQLER | GGTACAGGTGAGGAAGGAGGTGCAGGTGAGGC |
| | | RPPDFLRLLRLIRDME | AGGAGGCGCAGGTGAGGAAGGAGGTGAAGATG |
| | | AWAASLARSQQGVA | AGGCAGGAGCTGCAGGTGAGGCAGTAGGTGCA |
| | | WAAAPVESEDPAAA | GGTGTGGTGGAGGCCTGGACCCAGTCATGGCGC |
| | | QASPAQGDASEADPG | CAGACCCTGCGGCCTCTGGTGAAAACTATGGCC |
| | | AEDADEAASTTKEAA | TACCGGGAACTGAGACCCTTTTCCGGGAGGGAG |
| | | RVAPATGEDENAPA | CAGCCAGGCTGCGTGGAAGAGTCCTTTGAGAGC |
| | | GLEGLGQGRSPDAPG | TGGTTGGAGGACGCCAAGGATATGCTGCAGCTG |
| | | GLPARMGSAVDMAP | TGGTGCCACGCGTCGGAAAGGGAGAGAAGGAG |
| | | GGPSWEPEGLVQVG | GCGGCTGCTGGACAGCTTGGATGGCCTGGCCCT |
| | | GQEAEEPPQEGLKPIL | GGATATCGTGAGCGGCCTCCTGGAGGAAGATCC |
| | | EESENEDEDGAGEAG | AGACTTCTCTGCGCAGGACTGCCTGACGGCGCT |
| | | KPKSPPGK (SEQ ID | GGGGCAGGTGTTTAGGAGCCGGGATACATGGAT |
| | | NO: 112) | GACCTCGCGGATGAAGTTCCTGACCTGCACGCA |
| | | | GGGGCCCCAGGAGGGGCTGTTTGCCTTCGTGGT |
| | | | GCGCCTGGAAGGCCTGCTGCAGAAGGCCGTGG |
| | | | AGAAGGGGGCGGTCCACCCAGCCATGGCCAAC |
| | | | CACCTGCGACTGCGGCAGGTGCTGTCTCGGGCC |
| | | | CGCCCCAGCGAGGCACTCCAGGATACCCTGAGG |
| | | | AGGATGCAGCTGGAGAGGAGGCCACCTGACTTC |
| | | | CTGAGGCTGCTGCGGCTCATCCGGGACATGGAG |
| | | | GCGTGGGCAGCCTCCCTAGCAAGGAGCCAGCA |
| | | | GGGTGTGGCCTGGGCAGCGGCCCCAGTGGAGA |
| | | | GTGAAGACCCAGCTGCTGCCCAGGCCTCCCCGG |
| | | | CCCAGGGGGACGCCAGCGAGGCTGATCCCGGA |
| | | | GCAGAAGATGCTGATGAGGCTGCTTCCACCACC |
| | | | AAAGAGGCCGCCAGGGTTGCCCCTGCCACTGGG |
| | | | GAAGATGAAAATGCCCCTGCAGGCCTTGAAGGC |
| | | | CTAGGTCAGGGAAGGTCCCCCGACGCCCCTGGG |
| | | | GGCCTCCCAGCCAGGATGGGCAGTGCAGTCGAC |
| | | | ATGGCCCCAGGAGGTCCCAGCTGGGAGCCAGA |
| | | | GGGCCTTGTCCAGGTTGGAGGCCAGGAGGCTGA |
| | | | GGAGCCTCCCCAGGAGGGGCTCAAGCCCAT |
| | | | CTTAGAGGAGTCAGAAAACGAGGATGAGGATG |
| | | | GGGCTGGGGAGGCGGGCAAGCCCAAGTCCCCC |
| | | | CCGGGCAAATAGGCTCTGAGGGCCCTGAGGGCT |
| | | | CCTCTCCTCTCAGGCAGCAGCACCCTGGAGGCA |
| | | | GACAGAGGCCAGGCCAGGGCCGGTCCCTCACCC |
| | | | CATGTTGGGAGCTGTGCCTCCCACCTCCCCTAAT |
| | | | GGGTCCTGGCCACCACCACCAGCCCTTCCCAGT |
| | | | GACTCAGATGACCACGTTTGATACCAAATGGGG |
| | | | AGCTGGCCTGGAGGGAGCTCTGAGCGGCCAGCC |
| | | | TGAAGATGTCTGCCCCCCACCCCTTGTCCTGTGT |
| | | | TGGGAGAGATGGGGGAAAGAGGCCCTCTCAAG |
| | | | GGTGACAGCTGCCTACATGTCCCAAGAGGTGGC |
| | | | CCCCACTTGCCATCCCTGGGGCAGACTGCCCCC |
| | | | CGTTCCGGGAAGCCCACCCTCACCTCTGTAGGC |
| | | | CCCATAGTGACCCACGCGTCCGAACAGCAGACG |
| | | | CCCACTCACCCACCGCTAGCCATTGTTCCTACAC |
| | | | AAAGTTGTATGCTGTGCGCCCATCGGTGCGGCT |
| | | | GCCTCTGGGCCTGGGGCACATGCTGTGAGCTTC |
| | | | CTGCTAGCCCAGGCTCTGCTAGCTGCTACCTGTC |
| | | | CCTTGTCGTGAGCACCACCTCTGCTTTCCTGGTG |
| | | | TAGATCTAGGCCAGGGGCTGCTTGTTCTCGTGG |
| | | | AGCTGTGTGTGTTCTTCTCTGAGCAGCTCCTCCC |
| | | | CGGAGGCCCCCAGCACAGTCCTGGGAGATGGTG |
| | | | GAAGGAGGCGCCAGGGCACGATGGACGCTCAT |
| | | | CCTGTGACTGCTATGGTGAGCATGACCACGATG |
| | | | GTGCCCGTGACCATGATGGTGACCATGACTGTG |
| | | | TGAGGAAGAACCGGACCTGGGACAGAGCAGAG |
| | | | CTGCCCTGCCTGAGGCTCCCCGGGGAGCTTTGT |
| | | | GCTTTGGTGTTCTATTCCTGTTGTTACTTGTGAC |
| | | | TCAGTTTCCTTGACTTGGGGGGGTGTTCCCTGCT |
| | | | GTGTTTTCAGTGTCCTGTGAGGGCTATAGGGCA |
| | | | GGGCCCTGCCCCAGCGGATGGGCTCGGGAGGG |
| | | | GGCTCCCTGAAGCCAGTGGACACTGCCAGAGTC |
| | | | CACCATCCTGGCAAGAGGCGGACCCTGGGGCCC |
| | | | TCAGGAAGGAGGGAGTTGGCAGCGGGGGCTGC |
| | | | AGCAGGAGTAGGAGCAGATGAGGCGTCTTGCC |
| | | | AGGAACCCCAGGAGGAGGGGGCCCGGGACCTG |
| | | | TGTGGGACCTGTGTCCTGTGGTGGCAATTTGCA |
| | | | GTTTCTCTCTGTGTTGTGATTCCCTTCTCTTCAAT |
| | | | GGTTTCAGTACATGTTTCTCTTCAATAAACTTCA |
| | | | TTCAGTGTTCCA (SEQ ID NO: 113) |
| PNMA8A | NM_018215/ | MSKTMAMNLLEDW | AGTGCGCAGGCGCGCGCTGCGACGCCAACCTCG |
| | NM_018215.4 | CRGMEVDIHRSLLVT | GCTTCTCGCCGCTAACGGCATCGAGTCTGGACG |
| | (encoding | GIPEDCGQAEIEETLN | CCCCGTGACCCGCCTGGGCCGGAGCGGGGGCG |
| | polynucleotide) | GVLSPLGPYRVLNKI | GACGGCGCCTTCTTGGCCCTTTCTGCCTCTAGCA |
| | NP_060685/ | FVREENVKAALIEVG | GCGGCGCCGGGGTAGCCGGAGCCAGCGACTGG |
| | NP_060685.2 | EGVNLSTIPREFPGRG | GAAACGGCTGCATTCCACTGCGTCTCCTTGGCC |
| | (protein) | GVWRVVCRDPTQDA | TGGCTGGGCGGTCGGAGGCTGATCTGCCAAGAT |
| | | EFLKNLNEFLDAEGR | TACTGTTTGGAGACCGCTGCAGCCCACGTCCAC |
| | | TWEDVVRLLQLNHP | CTGATAGACTATTTACTACACATAGTAGGTTCA |
| | | TLSQNQHQPPENWA | GAGCCGCTAAATGTCCAAGACCATGGCGATGAA |
| | | EALGVLLGAVVQIIFC | CCTTCTGGAGGATTGGTGCAGGGGAATGGAAGT |
| | | MDAEIRSREEARAQE | GGACATCCACAGGTCCTTGTTGGTCACAGGCAT |
| | | AAEFEEMAAWALAA | CCCAGAGGACTGTGGGCAGGCAGAAATTGAGG |
| | | GRKVKKEPGLAAEV | AGACCTTGAATGGGGTCCTCTCCCCACTGGGCC |
| | | GSALKAETPNNWNA | CGTACCGCGTGCTCAACAAGATTTTTGTGAGGG |
| | | TEDQHEPTKPLVRRA | AAGAGAATGTTAAAGCTGCCCTCATTGAGGTTG |
| | | GAKSRSRRKKQKKN | GTGAAGGTGTGAATCTGAGCACCATCCCCCGTG |
| | | SRQEAVPWKKPKGIN | AATTCCCAGGAAGGGGTGGTGTCTGGAGAGTGG |
| | | SNSTANLEDPEVGDA | TCTGTAGAGACCCTACCCAGGATGCCGAGTTTT |
| | | ESMAISEPIKGSRKPC | TAAAAAATCTGAATGAATTCCTGGATGCCGAGG |
| | | VNKEELALKKPMAK | GGCGCACCTGGGAGGATGTGGTCCGCCTGCTCC |
| | | CAWKGPREPPQDAR | AGCTCAACCACCCCACCCTGTCCCAGAACCAGC |
| | | AEAESPGGASESDQD | ATCAGCCCCCAGAGAACTGGGCAGAAGCTCTGG |
| | | GGHESPPKKKAVAW | GGGTGCTTCTGGGAGCAGTGGTGCAGATCATCT |
| | | VSAKNPAPMRKKKK | TCTGCATGGATGCCGAGATCCGCAGCCGGGAGG |
| | | VSLGPVSYVLVDSED | AAGCCAGGGCCCAGGAGGCCGCTGAATTCGAG |
| | | GRKKPVMPKKGPGS | GAGATGGCAGCCTGGGCTTTAGCAGCAGGGAG |
| | | RREASDQKAPRGQQP | GAAGGTGAAGAAAGAACCGGGGCTTGCAGCAG |
| | | AEATASTSRGPKAKP | AGGTGGGTTCTGCCTTAAAGGCAGAGACCCCCA |
| | | EGSPRRATNESRKV | ACAACTGGAATGCCACGGAAGACCAGCATGAG |
| | | (SEQ ID NO: 114) | CCTACCAAACCTTTGGTTCGCAGGGCTGGAGCT |
| | | | AAGTCTCGCTCCAGGAGAAAGAAGCAGAAGAA |
| | | | GAACTCCAGGCAGGAAGCAGTGCCCTGGAAAA |
| | | | AACCCAAAGGCATCAATTCCAACAGCACAGCTA |
| | | | ACTTGGAGGATCCTGAGGTGGGTGATGCTGAAA |
| | | | GCATGGCGATCTCAGAGCCGATCAAGGGCAGC |
| | | | AGAAAGCCCTGTGTGAATAAGGAGGAGTTGGCT |
| | | | TTGAAGAAGCCCATGGCGAAATGTGCCTGGAAG |
| | | | GGTCCCAGAGAGCCACCTCAGGATGCCCGGGCA |
| | | | GAAGCCGAGAGCCCAGGAGGCGCCTCTGAGTC |
| | | | AGACCAAGATGGTGGCCATGAAAGCCCACCAA |
| | | | AGAAGAAGGCCGTGGCCTGGGTGTCTGCCAAG |
| | | | AACCCCGCTCCCATGAGGAAGAAGAAGAAGGT |
| | | | GAGCTTGGGCCCTGTCTCCTACGTCTTGGTTGAC |
| | | | TCAGAAGATGGCAGGAAGAAGCCGGTGATGCC |
| | | | AAAGAAAGGGCCAGGCTCAAGAAGGGAGGCAT |
| | | | CAGATCAGAAGGCCCCTCGGGGCCAGCAGCCTG |
| | | | CCGAGGCAACAGCCTCAACCTCTAGGGGTCCGA |
| | | | AGGCCAAGCCAGAAGGCTCTCCTCGGCGTGCCA |
| | | | CCAATGAATCCAGAAAGGTTTGATCTGGGGGAC |
| | | | CACCCATACTGAGGAGTTGAAAGAACAAGGAA |
| | | | GAAGTACCAAGTCAACCAAGTTCTCTCTTGTCA |
| | | | CTGAATAAGACTTTGGACTCTCTTAGGGCCCCT |
| | | | GTTGATAGAGATCTGGCCCTGAGGTAAACGATA |
| | | | GGTGAGGTCTTGGGTGGGAGGGAGTTGGGGAA |
| | | | GGGAGGTGGATCTCTATGCCCTTTTCTCTACCAG |
| | | | GCCTGCCGTTTCACCGCCTTCTCTACTCACCTTC |
| | | | TCTTCGGAGGCAGGAGAGTTGGTAAGAGGATTG |
| | | | GGAAAATTCTAGAACATTCATTCCCCTTTATGC |
| | | | ATGAGCAGGGTCACTGTTACACTCATCCATGTT |
| | | | CAGCTTTTCTCCCCCACGCCTTGCTCCCTCCTCG |
| | | | GAATGGTCAGCGACCTCTGCAGGCCCTGGCATT |
| | | | TGGAAGAGCTGGGTGGCCCTGCCATATTCTTCC |
| | | | TCCCGCCTTCCTCTCGTGTAACCAGCGGGGTAA |
| | | | GTTAAGCCAGGACCTTCGCTGCAAACCTGGTTT |
| | | | TATTGCTCCTTCAGTCTCCAGCTTCCATCCTCCA |
| | | | GTTATCTAGCCAGGAGGTCCCAAGAGTTAGTTT |
| | | | TAGGGAAAAAGAATGTCTGCCTAGACCTCAAAG |
| | | | TCTTTGAGTTTTAGAGTCTGTTAGTAAAAATGGC |
| | | | ACTTTGATTCCCATTTGGGATGAGCCCTCTCAGG |
| | | | AATCCTGTGGGGAAGGGGGGTGATTATAGGGA |
| | | | AGGACGCAGGCATTCCTAGGTCCCCAGCTCTAA |
| | | | TTCCATCCATCTACCCAACTGTCACCATCTTTGC |
| | | | ACCAAACTGTCACCATCTTTGCAGCAGAAGGTC |
| | | | ACTACTCACATTATAGTAAGAGGGGAAAAAAAT |
| | | | CTTTTAAAACTTGGCTGTTGGCCGGGCACGGTG |
| | | | GCTCACGCCTGTAATCCCAGCACTTTGGGAGGC |
| | | | TGAGGCAGGTCGATCACGAGGTCAGGCGTTTAA |
| | | | GACCAGCTTGACCAACATGGTGAAACCCCATCT |
| | | | CTACAAAAATTAGCTGGGCGTGGTGGCGCGCGC |
| | | | CTGTAATCCCAGCTACTCAGGAGGCTGAGGCAG |
| | | | ATGAATTGCTTGAATCCAGGAGGCAGAGGTTGC |
| | | | AATGAGCCGAGATTGTGCCACGGCACTCCAGCC |
| | | | TGGGCAGCAGAGTGAGACTCTCTCTCAAAAAAA |
| | | | CAAAACTTGGCTGTTAATGTCTGCCCTCTGAATT |
| | | | CAGACACACTATATTAGACCAGACCACCATGTG |
| | | | TCATTGTGTGTGTGTGTGTGTGTGTTTGCGTGCG |
| | | | CATGTGTAGAGGAGAGAGCAGGGTCCCTGAGA |
| | | | TAATGGTTTCCAAACTGTATCGTAGCCGTCCAC |
| | | | AAAGATGTAATCCAAATCTATTTTTCTCACGTGT |
| | | | TTAAAAAACTGAAAAGTGACTACTCAGATATGT |
| | | | TGGAAGTCACATCGAAGACTATCAGAATATTAC |
| | | | CTTGTGTTCATAGGTTAACTTGTTTTTGTACACG |
| | | | TCTTAGTTATTTCAGGCATCTCTTTGCTTAAAAT |
| | | | TGAGTTTCTTGTAAATGTGACTGATGAGCGAGG |
| | | | TTAGAAGTGGAAGAAATTCCTGTGCATGTTCTA |
| | | | TAATCTGACACCCTGAAAGCAAGTTTCCTTTCGT |
| | | | CATTCACATGCTCTTGTTCTGCCGTGACTGTTCA |
| | | | GGTGTATGGTAGTAAGTAAATGTATTAACATGG |
| | | | TGAACAGTAGTAATATTCTATCATAGAGTATTA |
| | | | GCCCTTGCAAGTTTTCAGGGCGTCTTTTCCGACT |
| | | | TCAGTTTTTGTGATAAAGAATGTGAACAGTTGT |
| | | | TAGATGTTCTCAGTGATTCAACTTTAAAACAAA |
| | | | TTTCTCGTGATGATTCATTTCAAAATCCTGAGTG |
| | | | AGTCTGACTGAAAAATACGAGAGAAAAGAGAG |
| | | | TGGTTTCCGTTTGCAGCTACACAGCTGTGTGCAT |
| | | | CGACGTTCTCCTGGGGTGTGTGCCAAGCGAAAC |
| | | | CCAGGGGTGAATTGGATTCTTGAAGAGACCAAA |
| | | | GCCTGTAACTGTCCAGCTTCTAATTTCAAAACG |
| | | | GGTCCATTAGGGCTTCGTTGTGTTAACAAGTTG |
| | | | ACACCATGACTAGTAAATGTAAACGTGTATGTA |
| | | | TAAAATAAAGTTTAGCAAATTAA (SEQ ID NO: |
| | | | 115) |
| PNMA8A_i2 | NM_001103149/ | MSKTMAMNLLEDW | AGTGCGCAGGCGCGCGCTGCGACGCCAACCTCG |
| | NM_001103149.2 | CRGMEVDIHRSLLVT | GCTTCTCGCCGCTAACGGCATCGAGTCTGGACG |
| | (encoding | GIPEDCGQAEIEETLN | CCCCGTGACCCGCCTGGGCCGGAGCGGGGGCG |
| | polynucleotide) | GVLSPLGPYRVLNKI | GACGGCGCCTTCTTGGCCCTTTCTGCCTCTAGCA |
| | NP_001096619/ | FVREENVKAALIEVG | GCGGCGCCGGGGTAGCCGGAGCCAGCGACTGG |
| | NP_001096619.1 | EGVNLSTIPREFPGRG | GAAACGGCTGCATTCCACTGCGTCTCCTTGGCC |
| | (protein) | GVWRVVCRDPTQDA | TGGCTGGGCGGTCGGAGGCTGATCTGCCAAGAT |
| | | EFLKNLNEFLDAEGR | TACTGTTTGGAGACCGCTGCAGCCCACGTCCAC |
| | | TWEDVVRLLQLNHP | CTGATAGACTATTTACTACACATAGTAGGTTCA |
| | | TLSQNQHQPPENWA | GAGCCGCTAAATGTCCAAGACCATGGCGATGAA |
| | | EALGVLLGAVVQIIFC | CCTTCTGGAGGATTGGTGCAGGGGAATGGAAGT |
| | | MDAEIRSREEARAQE | GGACATCCACAGGTCCTTGTTGGTCACAGGCAT |
| | | AAEFEEMAAWALAA | CCCAGAGGACTGTGGGCAGGCAGAAATTGAGG |
| | | GRKVKKEPGLAAEV | AGACCTTGAATGGGGTCCTCTCCCCACTGGGCC |
| | | GSALKAETPNNWNA | CGTACCGCGTGCTCAACAAGATTTTTGTGAGGG |
| | | TEDQHEPTKPLVRRA | AAGAGAATGTTAAAGCTGCCCTCATTGAGGTTG |
| | | GAKSRSRRKKQKKN | GTGAAGGTGTGAATCTGAGCACCATCCCCCGTG |
| | | SRQEAVPWKKPKGIN | AATTCCCAGGAAGGGGTGGTGTCTGGAGAGTGG |
| | | SNSTANLEDPEVGDA | TCTGTAGAGACCCTACCCAGGATGCCGAGTTTT |
| | | ESMAISEPIKGSRKPC | TAAAAAATCTGAATGAATTCCTGGATGCCGAGG |
| | | VNKEELALKKPMAK | GGCGCACCTGGGAGGATGTGGTCCGCCTGCTCC |
| | | CAWKGPREPPQDAR | AGCTCAACCACCCCACCCTGTCCCAGAACCAGC |
| | | AEAESPGGASESDQD | ATCAGCCCCCAGAGAACTGGGCAGAAGCTCTGG |
| | | GGHESPPKKKAVAW | GGGTGCTTCTGGGAGCAGTGGTGCAGATCATCT |
| | | VSAKNPAPMRKKKK | TCTGCATGGATGCCGAGATCCGCAGCCGGGAGG |
| | | NPERFDLGDHPY | AAGCCAGGGCCCAGGAGGCCGCTGAATTCGAG |
| | | (SEQ ID NO: 116) | GAGATGGCAGCCTGGGCTTTAGCAGCAGGGAG |
| | | | GAAGGTGAAGAAAGAACCGGGGCTTGCAGCAG |
| | | | AGGTGGGTTCTGCCTTAAAGGCAGAGACCCCCA |
| | | | ACAACTGGAATGCCACGGAAGACCAGCATGAG |
| | | | CCTACCAAACCTTTGGTTCGCAGGGCTGGAGCT |
| | | | AAGTCTCGCTCCAGGAGAAAGAAGCAGAAGAA |
| | | | GAACTCCAGGCAGGAAGCAGTGCCCTGGAAAA |
| | | | AACCCAAAGGCATCAATTCCAACAGCACAGCTA |
| | | | ACTTGGAGGATCCTGAGGTGGGTGATGCTGAAA |
| | | | GCATGGCGATCTCAGAGCCGATCAAGGGCAGC |
| | | | AGAAAGCCCTGTGTGAATAAGGAGGAGTTGGCT |
| | | | TTGAAGAAGCCCATGGCGAAATGTGCCTGGAAG |
| | | | GGTCCCAGAGAGCCACCTCAGGATGCCCGGGCA |
| | | | GAAGCCGAGAGCCCAGGAGGCGCCTCTGAGTC |
| | | | AGACCAAGATGGTGGCCATGAAAGCCCACCAA |
| | | | AGAAGAAGGCCGTGGCCTGGGTGTCTGCCAAG |
| | | | AACCCCGCTCCCATGAGGAAGAAGAAGAAGAA |
| | | | TCCAGAAAGGTTTGATCTGGGGGACCACCCATA |
| | | | CTGAGGAGTTGAAAGAACAAGGAAGAAGTACC |
| | | | AAGTCAACCAAGTTCTCTCTTGTCACTGAATAA |
| | | | GACTTTGGACTCTCTTAGGGCCCCTGTTGATAG |
| | | | AGATCTGGCCCTGAGGTAAACGATAGGTGAGGT |
| | | | CTTGGGTGGGAGGGAGTTGGGGAAGGGAGGTG |
| | | | GATCTCTATGCCCTTTTCTCTACCAGGCCTGCCG |
| | | | TTTCACCGCCTTCTCTACTCACCTTCTCTTCGGA |
| | | | GGCAGGAGAGTTGGTAAGAGGATTGGGAAAAT |
| | | | TCTAGAACATTCATTCCCCTTTATGCATGAGCAG |
| | | | GGTCACTGTTACACTCATCCATGTTCAGCTTTTC |
| | | | TCCCCCACGCCTTGCTCCCTCCTCGGAATGGTCA |
| | | | GCGACCTCTGCAGGCCCTGGCATTTGGAAGAGC |
| | | | TGGGTGGCCCTGCCATATTCTTCCTCCCGCCTTC |
| | | | CTCTCGTGTAACCAGCGGGGTAAGTTAAGCCAG |
| | | | GACCTTCGCTGCAAACCTGGTTTTATTGCTCCTT |
| | | | CAGTCTCCAGCTTCCATCCTCCAGTTATCTAGCC |
| | | | AGGAGGTCCCAAGAGTTAGTTTTAGGGAAAAA |
| | | | GAATGTCTGCCTAGACCTCAAAGTCTTTGAGTTT |
| | | | TAGAGTCTGTTAGTAAAAATGGCACTTTGATTC |
| | | | CCATTTGGGATGAGCCCTCTCAGGAATCCTGTG |
| | | | GGGAAGGGGGGTGATTATAGGGAAGGACGCAG |
| | | | GCATTCCTAGGTCCCCAGCTCTAATTCCATCCAT |
| | | | CTACCCAACTGTCACCATCTTTGCACCAAACTGT |
| | | | CACCATCTTTGCAGCAGAAGGTCACTACTCACA |
| | | | TTATAGTAAGAGGGGAAAAAAATCTTTTAAAAC |
| | | | TTGGCTGTTGGCCGGGCACGGTGGCTCACGCCT |
| | | | GTAATCCCAGCACTTTGGGAGGCTGAGGCAGGT |
| | | | CGATCACGAGGTCAGGCGTTTAAGACCAGCTTG |
| | | | ACCAACATGGTGAAACCCCATCTCTACAAAAAT |
| | | | TAGCTGGGCGTGGTGGCGCGCGCCTGTAATCCC |
| | | | AGCTACTCAGGAGGCTGAGGCAGATGAATTGCT |
| | | | TGAATCCAGGAGGCAGAGGTTGCAATGAGCCG |
| | | | AGATTGTGCCACGGCACTCCAGCCTGGGCAGCA |
| | | | GAGTGAGACTCTCTCTCAAAAAAACAAAACTTG |
| | | | GCTGTTAATGTCTGCCCTCTGAATTCAGACACA |
| | | | CTATATTAGACCAGACCACCATGTGTCATTGTG |
| | | | TGTGTGTGTGTGTGTGTTTGCGTGCGCATGTGTA |
| | | | GAGGAGAGAGCAGGGTCCCTGAGATAATGGTTT |
| | | | CCAAACTGTATCGTAGCCGTCCACAAAGATGTA |
| | | | ATCCAAATCTATTTTTCTCACGTGTTTAAAAAAC |
| | | | TGAAAAGTGACTACTCAGATATGTTGGAAGTCA |
| | | | CATCGAAGACTATCAGAATATTACCTTGTGTTC |
| | | | ATAGGTTAACTTGTTTTTGTACACGTCTTAGTTA |
| | | | TTTCAGGCATCTCTTTGCTTAAAATTGAGTTTCT |
| | | | TGTAAATGTGACTGATGAGCGAGGTTAGAAGTG |
| | | | GAAGAAATTCCTGTGCATGTTCTATAATCTGAC |
| | | | ACCCTGAAAGCAAGTTTCCTTTCGTCATTCACAT |
| | | | GCTCTTGTTCTGCCGTGACTGTTCAGGTGTATGG |
| | | | TAGTAAGTAAATGTATTAACATGGTGAACAGTA |
| | | | GTAATATTCTATCATAGAGTATTAGCCCTTGCA |
| | | | AGTTTTCAGGGCGTCTTTTCCGACTTCAGTTTTT |
| | | | GTGATAAAGAATGTGAACAGTTGTTAGATGTTC |
| | | | TCAGTGATTCAACTTTAAAACAAATTTCTCGTG |
| | | | ATGATTCATTTCAAAATCCTGAGTGAGTCTGAC |
| | | | TGAAAAATACGAGAGAAAAGAGAGTGGTTTCC |
| | | | GTTTGCAGCTACACAGCTGTGTGCATCGACGTT |
| | | | CTCCTGGGGTGTGTGCCAAGCGAAACCCAGGGG |
| | | | TGAATTGGATTCTTGAAGAGACCAAAGCCTGTA |
| | | | ACTGTCCAGCTTCTAATTTCAAAACGGGTCCATT |
| | | | AGGGCTTCGTTGTGTTAACAAGTTGACACCATG |
| | | | ACTAGTAAATGTAAACGTGTATGTATAAAATAA |
| | | | AGTTTAGCAAATTAA (SEQ ID NO: 117) |
| PNMA8B | NM_020709/ | MAMSLLQDWCRSLD | AGACGCGGCGCGAGCGCCAGGCAAGCTGCGGC |
| | NM_020709.3 | VDAHRALLVTGIPEG | TGCTACCTCCCACGCCTCTCCAGGTGCACTCGG |
| | (encoding | LEQADVEAVLQPTLL | CGCCGCCCCCCTGCACCTGGCTGCGGTGCCGAG |
| | polynucleotide) | PLGTFRLRHMKALM | TCACTCAGGCCTGTGTCAGGGAGAGAGGGAGG |
| | NP_065760/ | NEKAQAALVEFVED | GAGCTGTCCTGGAAAGCAGACACGTAAGCCCCC |
| | NP_065760.1 | VNHAAIPREIPGKDG | CGCGGATCCTCAGACAGCTCTGGAGAGGGGTCC |
| | (protein) | VWRVLWKDRAQDT | CGGGGGAAGGTCACTGCGTCCAGCCGGCCAGC |
| | | RVLRQMRRLLLDDG | AGGCAGCTAGAGCCCCCGAGCCCCGAGCCCCAC |
| | | PTQAAEAGTPGEAPT | TCCAGCCTTGCCACATTCACCGGAACCGGGACT |
| | | PPASETQAQDSGEVT | CTAAGCCCTGCAAGTGGCTTTCTAGGGTTGCAT |
| | | GQAGSLLGAARNPR | TGACACCGTGCGCTGCAGCCCACCCCTATCTCG |
| | | RGRRGRRNRTRRNR | GGCTCCCTGCTGCCCCAAGATCAGCGCCAAGGG |
| | | LTQKGKKRSRGGRPS | GGCTGCACCATGGCCATGAGCCTTTTGCAGGAC |
| | | APARSEAEDSSDESL | TGGTGCCGGAGCCTGGACGTGGACGCGCACAG |
| | | GIVIEEIDQGDLSGEE | GGCCCTGCTGGTCACCGGCATCCCGGAGGGCCT |
| | | DQSALYATLQAAAR | GGAGCAGGCAGACGTCGAAGCCGTCCTGCAGC |
| | | ELVRQWAPCNSEGEE | CGACCCTCCTGCCCCTGGGCACGTTCAGGTTGC |
| | | DGPREFLALVTVTDK | GACACATGAAGGCTTTGATGAACGAGAAGGCC |
| | | SKKEEAEKEPAGAES | CAGGCCGCCCTGGTGGAGTTTGTGGAGGACGTC |
| | | IRLNTKEDKNGVPDL | AATCACGCTGCCATTCCCAGGGAGATCCCAGGC |
| | | VALLAVRDTPDEEPV | AAGGATGGGGTCTGGAGGGTTCTGTGGAAGGA |
| | | DSDTSESDSQESGDQ | CCGTGCGCAGGACACGAGGGTCCTGAGGCAGA |
| | | ETEELDNPEFVAIVA | TGAGACGCCTGCTGCTGGATGACGGGCCCACGC |
| | | YTDPSDPWAREEML | AGGCCGCGGAGGCTGGGACCCCCGGGGAGGCA |
| | | KIASVIESLGWSDEK | CCCACCCCTCCCGCTTCGGAGACGCAGGCCCAG |
| | | DKRDPLRQVLSVMS | GATTCTGGGGAGGTAACAGGGCAGGCTGGCTCG |
| | | KDTNGTRVKVEEAG | CTTCTTGGGGCAGCCAGGAACCCAAGGAGGGG |
| | | REVDAVVLRKAGDD | CCGTCGGGGTCGCAGAAACAGAACCAGACGCA |
| | | GDLRECISTLAQPDLP | ACAGGTTGACCCAGAAGGGCAAGAAGAGAAGC |
| | | PQAKKAGRGLFGGW | CGAGGAGGACGGCCGTCTGCTCCCGCGAGGAGT |
| | | SEHREDEGGLLELVA | GAGGCCGAGGACTCTTCCGACGAGAGCCTGGGC |
| | | LLAAQDMAEVMKEE | ATCGTGATCGAGGAGATCGACCAGGGCGACCTG |
| | | KENAWEGGKYKYPK | AGCGGAGAAGAGGACCAGAGCGCGCTGTACGC |
| | | GKLGEVLALLAAREN | CACGCTGCAGGCCGCTGCCAGGGAGCTGGTTAG |
| | | MGSNEGSEEASDEQS | GCAGTGGGCGCCCTGCAACTCCGAGGGGGAAG |
| | | EEESEDTESEASEPED | AAGACGGTCCCCGCGAGTTCTTGGCTCTGGTCA |
| | | RASRKPRAKRARTAP | CCGTCACCGACAAATCGAAGAAAGAAGAGGCA |
| | | RGLTPAGAPPTASGA | GAGAAGGAGCCAGCTGGGGCCGAATCCATCCG |
| | | RKTRAGGRGRGRGV | CTTGAACACCAAAGAAGACAAAAATGGTGTCCC |
| | | TPEKKAGSRGSAQDD | CGACTTAGTGGCCCTGCTGGCTGTGAGAGACAC |
| | | AAGSRKKKGSAGAG | CCCGGACGAGGAGCCGGTGGACAGCGACACTT |
| | | AHARAGEAKGQAPT | CGGAGAGCGACTCGCAGGAAAGTGGGGACCAA |
| | | GSKAARGKKARRGR | GAAACAGAGGAGTTGGATAATCCTGAGTTCGTG |
| | | RLPPKCR (SEQ ID | GCCATTGTGGCCTATACCGACCCGTCGGACCCC |
| | | NO: 118) | TGGGCCCGGGAGGAGATGTTGAAAATCGCTTCT |
| | | | GTTATCGAGTCGCTGGGCTGGAGCGACGAGAAA |
| | | | GACAAGCGAGACCCCCTCCGACAGGTCTTGTCC |
| | | | GTCATGTCCAAGGACACTAACGGGACCCGCGTG |
| | | | AAGGTGGAAGAGGCGGGCCGCGAGGTGGACGC |
| | | | CGTGGTCCTGCGCAAGGCCGGGGATGACGGGG |
| | | | ACCTCCGGGAGTGCATTTCCACCTTGGCGCAGC |
| | | | CGGATCTCCCTCCCCAGGCGAAGAAGGCTGGGC |
| | | | GTGGCCTCTTCGGGGGCTGGAGCGAGCACCGTG |
| | | | AGGACGAAGGGGGTCTTCTGGAGCTGGTGGCGC |
| | | | TCCTGGCTGCCCAGGACATGGCGGAGGTGATGA |
| | | | AGGAGGAAAAAGAAAACGCCTGGGAAGGCGGG |
| | | | AAGTACAAATACCCCAAAGGCAAACTGGGGGA |
| | | | GGTATTGGCGCTCCTGGCCGCCCGGGAGAACAT |
| | | | GGGGTCCAACGAGGGGTCGGAGGAGGCTTCGG |
| | | | ACGAACAGTCCGAGGAGGAGTCGGAGGACACC |
| | | | GAGAGCGAGGCGTCGGAGCCGGAGGACAGGGC |
| | | | ATCCAGGAAGCCCCGGGCCAAGAGGGCGCGCA |
| | | | CGGCCCCCAGGGGCCTGACTCCGGCCGGCGCGC |
| | | | CTCCCACCGCTTCCGGGGCCCGCAAAACCCGCG |
| | | | CGGGCGGCCGAGGCCGAGGCCGGGGCGTCACT |
| | | | CCCGAGAAGAAAGCCGGGAGCCGGGGCTCGGC |
| | | | CCAGGACGACGCCGCAGGAAGCAGGAAGAAGA |
| | | | AGGGGAGCGCGGGCGCCGGGGCCCATGCCAGG |
| | | | GCAGGCGAGGCCAAGGGCCAGGCGCCCACTGG |
| | | | ATCCAAGGCCGCGCGCGGGAAGAAGGCCCGTC |
| | | | GGGGCCGGAGGCTGCCCCCTAAATGCCGCTAGT |
| | | | GGCCCCCCAAGAAGCCGCCCAGGCTGCGAGCA |
| | | | GGCCCCGCAGGGCACCCGCCCGCCTGTGGCCCC |
| | | | CGCCCTCCCCTCCCCTCTTCCTGTCCTCCGCAGA |
| | | | CGCAATCTCCTCGCTTCACAGCGCGCCCGGGCC |
| | | | GCGTTTTGCCAGCGTCACGTTCCCCTCTCGGGCC |
| | | | CTCGCAGGCCGGGGGCGCCAGCGATCCCGACG |
| | | | GAGGAAGCCCGGATGGGAGGAGGAAAGAGAAG |
| | | | TGGGCGCCCGAGGCAGCAGCGCAGGGCCGAGA |
| | | | TGGGGACGCGCCAAGTGGACCAGGATTGGGGG |
| | | | CCCGGGTTGCCCCCGGAGGGGGTGTGTGTGTGG |
| | | | ACGCCGGGCACCTGCAGAGGCGAGCAGGGCTC |
| | | | TTCGTGGCGCTCTCGGGGCCTGCGCCTGGCAGG |
| | | | TGCTGTAGGCCGCTGTCGCCCCTACCCCAGTCT |
| | | | GACTGGGCCCTGGGTCTGTGGTGGAGGCTCAGT |
| | | | CACCAGCCGCGCAGCGCGTGTCAGGGCGCAACT |
| | | | CTCAGCCAGGGGAGGCCCCAGCTCCCAGCCAGG |
| | | | GGAAGAGATGATTCCAGAAAGGAAAGTCTGAG |
| | | | AGATAGAAGGCGGTTGGGAAGGGGAGGAGGAG |
| | | | GAAAGGGGAGAGGAACGGTGGGAGAAGGGAA |
| | | | AGAGGAGGAGGAGGGGGAGGGGGGAGCAGAG |
| | | | GGAAGACACATGCCAGCCCTGCCTACTGGGGCG |
| | | | CCCCTGATAACAAAGGAACCAGCCCCAGGCCA |
| | | | AGGCCACCTGCCCCTGACCACAAGTTGAATTTG |
| | | | TCACTCAGACTGCAGTGTTTCCCAACATTCTAAT |
| | | | TATTTGCAGAGGTGTTCAATTTGGGGTAATTCA |
| | | | CTTAAAATCCAGTTTTGGTTCTTCTGGGCTGAGT |
| | | | GGGCCCTGGCCCCTCCCATAGGCTGTGGCTCCC |
| | | | CTGGGTGCCCCCTCTCCAGTGGAGCTGACCCAC |
| | | | CGCTCAGCGCTGGCCTTGCAGCCCTTACTAAAA |
| | | | GACTTGAAAGTCCCTGGGTTCACCCCCTGAGTG |
| | | | AATTAAAGGCCAGAGGGGCCCCGAAGGGCACT |
| | | | GTGAGGGACAGAGGCTCACCTGGGCAGTGCAG |
| | | | AAGCCGGCCGCGTGTCCCTCCTTACAGGGGATG |
| | | | AAATGACCTGGGGAGGAAACCCCAGCCCTGCCC |
| | | | TGGAGGTTCCAGAGTAGGCGGGCCGGTGCTGTG |
| | | | AGGCTTCACAACCTGCTGTCCCAAGCACGCTTG |
| | | | AGTTGTATGTGAGTCTGTGCCGTGCCGTGCCGT |
| | | | ATGCTTCAGCTCCTGCAACCCCGGCTGAGCTCG |
| | | | ATTTTTACCTAAATATCAGTCTCCACGGGACCCC |
| | | | ACCTTCATTCATGCCTTCTTGTCCCTGGGGCAAT |
| | | | GTGTGTGCTTCCTCGTCCCAATTTCCATTCCCTG |
| | | | GCAGTGAGGAGCCCATCGTGCCAGGGGGCCCTG |
| | | | CCCCACTTGTCCCTGGGAAGGAATAGGAGGGTT |
| | | | TGGGTGTGACCTCACAGTCCAGACCAGACTGTC |
| | | | CCAGTCCTATGTCAGGGACACCCAGATGTAGAA |
| | | | GCTGACTGAGACCTGCTGCAGGGCGTGGGTGCT |
| | | | CCCCTCTGCTTGGAGGCTGTCCCTGGACAGTGA |
| | | | CCCACCCACTGAGGACCAGGCTGGGTGTACCTT |
| | | | GAGCTGGGCACAGCAGCCTGTGGTGTTGCCTGT |
| | | | GGGTGGGGAGGGCCCCAGGTGTGCTTCTCCCGT |
| | | | AGCAGTCCTAGGCTTCTCTCCCTGTGCCCTGTGT |
| | | | CACCTGGATCCTCCAGTAAAGTGAAATTCAGCA |
| | | | CTGTACTCTCTCTGTGCTCTGGGCAGTGGGGCA |
| | | | GGCGGGGTGTGGGAGCGTGGGCCACAGATGTC |
| | | | CACGGTCTTGACTGTGGTTTGCCCAGAATACCT |
| | | | GGGAACTGTCCTGTCACTGGTTTACATACACTG |
| | | | TCCTTTGCTGCTTCGGGATCCCTGCCTGGCTCTC |
| | | | CCTACCCCCCAGCATCATCTCACCCCTTGCAGAT |
| | | | CTGAGCCAGCTTCCACTCCCACCCCTGATGCCTC |
| | | | CCCACTTCCAGCCTCAGCTCCGAAGCCCCTGGA |
| | | | CACCCATGGAGACCCCGCCCAGCCAATCCCCAC |
| | | | CCTAGCTTCCACCCAGATACACTCTGCCAGGCC |
| | | | ACAGCTGCAGGCACTCTCCCCCCAGCCTCCACC |
| | | | CCTCACCTGTGCCCTGGACCTCAGACTCAGCTTT |
| | | | CCATCCTACCTGAGTTTTCTGCCTCCCTCCATCC |
| | | | TGTGTCCCCCCACCATACATGGCTGCCAGAGAC |
| | | | GTCCTCTTAGAAGTCACACCTGGGGTCTGATTG |
| | | | CGTCCCTCGCCTCCCCAGATCCCCCAAGGTCTCC |
| | | | TTCCTGTGCCGTCATATCTGCAGTTCTTAGGACT |
| | | | GTCTAGACATGCTTTGTTCAACTAGGTAATCAC |
| | | | ACGGGGTAAATTGGATTTAAATGTAATTAAGAT |
| | | | TAAATAAAAATACACATGCATGTC (SEQ ID NO: |
| | | | 119) |
| PNMA8C | NM_001386793/ | MLFGVKDIALLEHGC | GACGTCACCGCGGGAGCAAGTGCAGCGGCCAC |
| | NM_001386793.1 | KALEVDSYKSLMILG | ATCCTCGTCCTAGTCCGGCGACAGGGCACTGAG |
| | (encoding | IPEDCNHEEFEEIIRLP | TGCAACCTCTGGGCCAGCGGTGAGGGACGCGCC |
| | polynucleotide) | LKPLGKFEVAGKAYL | TCCCTGCCTGCCAGGGCCGCGCCTACGACACTC |
| | NP_001373722/ | EEDKSKAAIIQLTEDI | TGTTGGCAGTTGTCCCAGGGAGATCAGCAGTCC |
| | NP_001373722.1 | NYAVVPREIKGKGG | CAGATCCGGAGAGACCGTTGGGGCCTGTGAGAC |
| | (protein) | VWRVVYMPRKQDIE | CTTCGGAGGCCACGCCAAGAAAGGGGAAGCAT |
| | | FLTKLNLFLQSEGRT | CATCTTTAGACCCTCCTTGGTGCAGCCTTGCCCA |
| | | VEDMARVLRQELCPP | GGACCCGGGAGCCACGAGTCAGGTGGCAAACA |
| | | ATGPRELPARKCSVP | CCGCCTTCTGCTTCTCCAGCACGCGTAAGTCCCT |
| | | GLGEKPEAGATVQM | GGGAATCTTCGAGCCCACTCAGAGGGAGCCACA |
| | | DVVPPLDSSEKESKA | GAAGCCCCGACGTTGCACAGCCCTGCAGGCAGG |
| | | GVGKRGKRKNKKNR | GGCTGGGGGCATCCTTCACTGAGCTGGACGAAG |
| | | RRHHASDKKL (SEQ | GGGTCCTGGGAGCGGCCTCCGGCCCCTGAGGCC |
| | | ID NO: 120) | TCACTCTCTCGTAGCCTTTGGGAAGGAATCGCG |
| | | | GCATCCTGGATAATTTCGTGGATTCCCGAGGCA |
| | | | GCTCAGGTGTCCACCAGCGGGCATACTGCTGCC |
| | | | TCAAAGAGTTGGGCAAGATGCTGTTCGGGGTGA |
| | | | AGGACATTGCACTGTTGGAGCACGGGTGCAAGG |
| | | | CCCTGGAGGTGGACAGTTACAAGTCCCTGATGA |
| | | | TCCTGGGGATCCCGGAGGACTGCAACCACGAGG |
| | | | AATTCGAAGAGATTATTCGGCTGCCCCTCAAAC |
| | | | CTCTAGGCAAGTTCGAAGTGGCTGGGAAGGCCT |
| | | | ATCTGGAAGAAGATAAATCCAAGGCGGCCATC |
| | | | ATTCAGCTGACGGAGGACATCAATTACGCCGTG |
| | | | GTCCCCAGGGAGATCAAGGGCAAGGGCGGTGT |
| | | | GTGGAGAGTGGTCTACATGCCCCGGAAGCAGG |
| | | | ACATTGAATTCCTGACCAAGCTGAACCTCTTTCT |
| | | | GCAGAGCGAGGGCAGGACGGTGGAGGATATGG |
| | | | CCCGGGTCCTGAGGCAGGAACTGTGTCCCCCAG |
| | | | CCACGGGCCCCAGAGAGCTGCCTGCAAGAAAG |
| | | | TGCTCTGTGCCTGGGCTGGGGGAGAAACCGGAG |
| | | | GCTGGGGCCACCGTCCAGATGGACGTGGTGCCA |
| | | | CCTCTGGACTCCTCCGAGAAGGAGAGCAAGGCT |
| | | | GGAGTTGGCAAGAGGGGCAAAAGGAAGAACAA |
| | | | GAAAAACCGCCGGCGGCATCACGCCTCAGACA |
| | | | AGAAGCTGTGAGGTGGTGGCATCGGGTGGGCGT |
| | | | GGGGAGTGTCTGAGGGGGTGTGGGTGATGACTG |
| | | | TGGGAGGGTAGGTACCACCTCACTACGTTTAAG |
| | | | GAGTGCGAGTGAGTGTGGGTAATGCAAGGGAG |
| | | | GGTGGGAATGACTCTAGTGAGTGGGTAACTTTG |
| | | | GAGTAAGAATGGGTATCATTTGGATAGCACAGG |
| | | | TAACATCTGACTAGTGCGAAAGAGGTGGATATC |
| | | | TTTATCTGAGTTAACTAACTTGTGGGTTACATCT |
| | | | GAGTGGGTGTGGTTAACGTCTGGGTGCCCGTGG |
| | | | CTCATTTATTCATCTTACAGATATTTATTGCATA |
| | | | CCTACTATGTTCCAGGAGCTGGGGATATGGAAG |
| | | | GGAGTAAACTCAATAAAAATCCCTGCCTTCATG |
| | | | GCACTCAGAGTAAGTGGGTAATATTTGAATTAC |
| | | | TTGGAGACATCAGAGTGAGCGTAGGTAGCATTT |
| | | | GAGTACATGTGGGTAACAGCTGAATGATCATAA |
| | | | AATCTGGTTGAGTGTGGATTGCTCATTCATCCAT |
| | | | GTATCCATGCAACCAACAAAATTTCCTGAGGAC |
| | | | CTACAGTGTGCCAGGCACTGGGGATACAGAAG |
| | | | GGGAATAAACAGACCAAAACCTTGCCCGTGACT |
| | | | CTCATAGTAGCTATTTGAGTAGGTGCGGGTAAC |
| | | | ATTTGGGTTACTTGAATACACGTTAGTGAGTGT |
| | | | GGGTAGCATTAGAGTGTGTGGGTGGCATCCGCG |
| | | | TATAATTGAAAGTAGTTGTGGGTAATATTTGAG |
| | | | AGTGTGCGGGTAACACCTG |
| | | | AATATGTGTATGGGTATCATTTGAGTAGATGAG |
| | | | AGTAATTTCTAAGCGAGGAAGGGTCTCAAAGAC |
| | | | CCACAAATATCTTCCGCTACTCGGGAGACCCCG |
| | | | CGGGACCAGCCGCGCAGGCGCAGTCAGAGCCA |
| | | | GAACCTAGCAGCGCGTCCGACCGTTGCTATGGA |
| | | | GACCACAGGCTGACCCCAGGCGCCTGCGCCCTT |
| | | | TGCAGAAAATGGAGGAGGTGGAAACGGTCCTG |
| | | | GATTCCAAGGTTTTGCAGATCTCTTACTGGAGTT |
| | | | GAAGAAAACTCACGCAGACCCAGGAGGAACGT |
| | | | GTCAGGGAGATCCCTACTCGGGAGAAGGGCCG |
| | | | CCATCCTCCTTCGGCTGTCACCCACGGCCTGATT |
| | | | CTGGGGACAGCCTCTCTTGCTGGGGGGAGGCGG |
| | | | GCGGCACGGACCCCTCTAGCTTTGCGCCCTCCT |
| | | | GGAAATCCTGTTATTGCAAAGTCTAGAGCCGTT |
| | | | TTCTGTGTTTCAAAAGCATGTACCTTGAATGTAC |
| | | | CTCTGGCACAAACTTGTTCGGAGTGGGCCACCC |
| | | | TGTTTTGCGCATGTCAAGCAGAAACCATGGCTG |
| | | | AGAGTGAGTTGCCTGCTCCCCACCTCTGAGGAA |
| | | | CTCACTGATCTGTAAGCGGCTCTTGAAAAGCTG |
| | | | TCGTCTTTCTCTACACGCTGAGTCTCTCTTACAG |
| | | | AAGTGGTCAGAAGCGGGACCCTTCTGCCCCGAT |
| | | | TGGCGCCTGGGGTGAGGGTGTGAGTCTCCCCAA |
| | | | GTTAAATTCCAGAAGCTCTGGCTTGGTGAGGAG |
| | | | GAGTGAACTCCACTCTCTAAAGAAGAGGCAAG |
| | | | GCTTCTGGGCAATGGTCCAAAAGGAGGTAAATC |
| | | | ATTAAGAGGCCATTTTCAAGAGTTACCAATGAA |
| | | | CGACGTTAACACTGAGGTGAAGAAGAATTTTAA |
| | | | AAATCAGGTAAAATCCAAATACATGTGAGTAGG |
| | | | CAGATCTTTTGCTCAGAGACAACGGACCTGTCA |
| | | | ATTTGCAAACTTGTTAGTTCAAGGATCTGCAAA |
| | | | GGTTTGACTGAATCTCTTTTCAAAAATGTAACTT |
| | | | ACACACAACACAAAGACTCAGCCTACCAAATTG |
| | | | TAATGTCAGAGACTCCTAACGTCTTTTTTTTTTG |
| | | | AGACAGAGTCTCGCTCTGTCACCAGGCTGGAGT |
| | | | GCAGAGGTGCAATCTCAGCTCACTGCAACCTCT |
| | | | GACTCCCTGGTCCAAGCGATTCTCCTGCCTCAG |
| | | | CCTCCCTAGTAGCTAGGATTACAGGCATGAACC |
| | | | ACCACGTCCAGCTAAATTTTGTATTTTTACTAGA |
| | | | GAAAGGGTTTCACCATGTTGGCCAGGATGGTCT |
| | | | CAATCTCCCGCCTTGGTCTCCCAAAGTGCTGGG |
| | | | ATTGCAGGTGTAAGCCACTGCGCCCAGCTTCTC |
| | | | TGTTGATTCTTTTTTTTTTTTTTTTTTTTTTTTTGA |
| | | | GGTGGAGTCTCCTTCTGTAGCCCAGGCTGGAGT |
| | | | GCAGTGGCACGATCTTGGCTCACTGCAACCTCT |
| | | | GCTTCCTGGGTTCAAGTGATTCTCCTGCCTCAGC |
| | | | CTCCTGAGTATCTGGGACTACAGGTGCGCACCA |
| | | | CCACGCCCAGCTAATTTTTGTATTTTTAGTAGAG |
| | | | ACAGGGTTTCACCGTATTAGCCAGGATGGTCTC |
| | | | AATCTCCTGACCTTGTGATCCGCCCACTTCGGCC |
| | | | TCCCAAAGTGTTGGGATTACAGGCGTGAGCCAC |
| | | | CACGCCCGGCCTGATTTTTCTATTCCTTGTAAAT |
| | | | GCCCAGCCTGGGCATACCCACTCGCAGAATATT |
| | | | GTACTACTGTGTTCGTTATTGCATCCCAGAGTCT |
| | | | GAGCTTGAATAAATGCTACGAAAGCCTCCAGCG |
| | | | TGTTTGTTGCATGCTGCTCTCCCATCAAGGCATA |
| | | | ATCGTTGTATAATAAGGGATAGTGTGACATTTC |
| | | | TTATCATATTGCAGCATGAAGACTGTATTTTGTG |
| | | | CTTATTGTGGATTTCAGGGGTATAACATTTATAT |
| | | | TTGTGATACACAACATGCGTGGTTGTTCAATGT |
| | | | TGCCCATGAAATCAAAATCCTACCACTTCTCTTT |
| | | | CAAGATTTTCTGGACCAAAGTGTATCCTGGGAC |
| | | | TCCAATGCAAGTTGTCATGCAAAACTGTCACCA |
| | | | TTTGAAGCTACTTAGCTGAGTGAAAACATCACT |
| | | | GGTAGGGCAAATCCCAAGCAAAAATTCACAAG |
| | | | TAAATTGAATGTGCCAGCTGTTGGAGGGCTGCA |
| | | | TCTGGCTTCTGTAAACCCCTGATTTCAAAAGGG |
| | | | GGTTCAGCTAAAACTCTTCATTGTTCTTTGTGAT |
| | | | TGAGATGCTCTTTTGTACAAATATGAACTCATA |
| | | | AAATAAATGTATGCTAATGAAA (SEQ ID NO: |
| | | | 121) |
| CCDC8 | NM_032040/ | MLQIGEDVDYLLI | GCAGAGCTCTAAGCGCGCGGGCTGGCAGGCTGC |
| | NM_032040.5 | PREVRLAGGVWR | GGCGCGTCAAGGTCAGCCTGGAGCTGGGTGGCG |
| | (encoding | VISKPATKEAEFR | GCCTGCCTGGGGGCGGGGGACCCTACTGGAGGC |
| | polynucleotide) | ERLTQFLEEEGRT | CCGGGCTGGGGCCTCCCAGCGCCTCGGCCATAT |
| | NP_114429/ | LEDVARIMEKSTP | TGAATAGCTTCGACTGGACCGTCTTTGTCTGCG |
| | NP_114429.2 | HPPQPPKKPKEPR | AAGTCCTGTCCCAAGTTCCAGCCGCGTCCCTGG |
| | (protein) | VRRRVQQMVTPP | GGCCTGGGGCAGGAAGAGTCGCTGGCAGCCCG |
| | | PRLVVGTYDSSN | CGCGCCCCAACTTGGAGCTGGGACACCACGTTT |
| | | ASDSEFSDFETSR | CCAGCTTGGAGTGGGCCTTGAGCCTTGGGACTG |
| | | DKSRQGPRRGKK | ACCTCGCCCCCGGCTCACGTAGGCATCCTGGAA |
| | | VRKMPVSYLGSK | ATTGATTCCCCCAAGTCCTTGGTGGGGGAGCCG |
| | | FLGSDLESEDDEE | GACTTGGTCAAGACTGTACTTGTTGCAGGCGAA |
| | | LVEAFLRRQEKQ | GAGATTGGAGGCGTTTGGCTCGTCCCTGGCTAG |
| | | PSAPPARRRVNLP | GGAGGTGAGACTCTCCGGTCAGCGTTGCTGGAA |
| | | VPMFEDNLGPQL | CTCCCCCCATCCAGTCCCTCCCTCAAGACTAAG |
| | | SKADRWREYVSQ | GGCTACAGTAGTTTGTTGGGGCTCATTGCCCCCT |
| | | VSWGKLKRRVK | CACCCCAGATATCACCCTGGAGATCTTAAAGAC |
| | | GWAPRAGPGVGE | TCTCGAGAAAAGCCACGTGGGGGGCTGGTTCCC |
| | | ARLASTAVESAG | CTGGGGCTTCCTGCCGTCCCCCGACTGCCTCATT |
| | | VSSAPEGTSPGDR | CTTTGGAGCGTCCCCGATGTCTGCAAAGATGTG |
| | | LGNAGDVCVPQA | GATTTGGACGTCCTCGTGGAAGCCCTAAAGCCC |
| | | SPRRWRPKINWA | GTGGGGACATTTAAGAAGATCGGCAAGGTGTTC |
| | | SFRRRRKEQTAPT | CGCAAGGAGGAGGACTCCACGGTGGGGATGCT |
| | | GQGADIEADQGG | GCAGATCGGGGAGGACGTCGACTATTTGCTCAT |
| | | EAADSQREEAIA | CCCCCGGGAGGTCAGGCTGGCTGGGGGCGTCTG |
| | | DQREGAAGNQR | GAGAGTCATCTCTAAGCCCGCCACCAAGGAAGC |
| | | AGAPADQGAEAA | AGAATTTCGGGAGCGGCTGACCCAGTTCCTGGA |
| | | DNQREEAADNQR | AGAAGAGGGCCGCACCCTGGAGGACGTGGCCC |
| | | AGAPAEEGAEAA | GCATCATGGAGAAGAGCACCCCGCACCCGCCCC |
| | | DNQREEAADNQR | AGCCCCCCAAAAAGCCCAAGGAGCCCCGAGTG |
| | | AEAPADQRSQGT | AGGAGGAGAGTGCAGCAGATGGTGACTCCTCC |
| | | DNHREEAADNQR | GCCCCGGCTGGTCGTGGGCACGTACGACAGCAG |
| | | AEAPADQGSEVT | CAACGCCAGCGACAGCGAGTTCAGCGACTTCGA |
| | | DNQREEAVHDQR | GACCTCCAGAGACAAGAGCCGCCAGGGCCCGC |
| | | ERAPAVQGADNQ | GGCGGGGCAAGAAGGTGCGCAAAATGCCCGTC |
| | | RAQARAGQRAEA | AGCTACCTGGGCAGCAAGTTCCTGGGAAGCGAC |
| | | AHNQRAGAPGIQ | CTGGAGAGTGAGGATGATGAGGAACTGGTCGA |
| | | EAEVSAAQGTTG | GGCCTTCCTCCGGCGACAGGAGAAGCAGCCCAG |
| | | TAPGARARKQVK | CGCGCCGCCTGCCCGCCGCCGCGTCAACCTGCC |
| | | TVRFQTPGRFSW | AGTGCCCATGTTTGAGGACAACCTGGGGCCTCA |
| | | FCKRRRAFWHTP | GCTGTCCAAAGCGGACAGGTGGCGGGAGTATGT |
| | | RLPTLPKRVPRAG | CAGCCAGGTGTCCTGGGGGAAGCTGAAGCGGA |
| | | EARNLRVLRAEA | GGGTGAAGGGTTGGGCGCCGAGGGCGGGCCCC |
| | | RAEAEQGEQEDQ | GGGGTGGGCGAGGCCCGGCTGGCCTCCACCGCA |
| | | L (SEQ ID NO: | GTGGAGAGCGCAGGGGTATCATCGGCGCCAGA |
| | | 122) | GGGCACCAGCCCGGGGGATCGCTTGGGAAACG |
| | | | CGGGAGATGTTTGTGTGCCCCAGGCTTCCCCTA |
| | | | GGCGATGGAGGCCCAAGATCAACTGGGCCTCCT |
| | | | TTCGGCGCCGCAGGAAGGAGCAGACAGCACCC |
| | | | ACAGGTCAGGGGGCAGACATCGAGGCTGATCA |
| | | | GGGGGGAGAGGCTGCAGATAGTCAAAGGGAAG |
| | | | AGGCCATAGCTGACCAGCGGGAAGGGGCTGCA |
| | | | GGTAATCAGAGGGCAGGGGCCCCAGCTGACCA |
| | | | GGGGGCAGAGGCTGCAGATAATCAGAGGGAAG |
| | | | AGGCTGCAGATAATCAGAGGGCAGGGGCCCCA |
| | | | GCTGAGGAGGGGGCAGAGGCTGCAGATAACCA |
| | | | GAGGGAAGAGGCTGCAGATAATCAGAGGGCAG |
| | | | AGGCCCCAGCTGACCAGAGGTCACAGGGCACA |
| | | | GATAACCACAGGGAAGAGGCTGCAGATAATCA |
| | | | GAGGGCGGAGGCCCCAGCTGACCAGGGGTCAG |
| | | | AGGTTACAGATAATCAAAGGGAAGAGGCCGTA |
| | | | CATGACCAGAGGGAAAGGGCCCCAGCTGTCCA |
| | | | GGGTGCAGATAATCAGAGGGCACAGGCCCGGG |
| | | | CTGGCCAGAGGGCAGAGGCTGCACATAATCAG |
| | | | AGGGCAGGGGCCCCAGGTATCCAGGAAGCTGA |
| | | | AGTCTCAGCTGCCCAAGGGACCACAGGAACAG |
| | | | CTCCAGGAGCCAGGGCCCGGAAACAGGTCAAG |
| | | | ACAGTGAGGTTCCAGACCCCTGGACGCTTTTCG |
| | | | TGGTTTTGCAAGCGCCGGAGAGCCTTCTGGCAC |
| | | | ACTCCCCGGTTGCCAACCCTGCCCAAGAGAGTC |
| | | | CCCAGGGCAGGAGAGGCCAGGAACCTCAGGGT |
| | | | GCTGAGGGCCGAGGCCAGAGCAGAAGCTGAGC |
| | | | AGGGAGAGCAAGAAGACCAGCTGTGAGGTGAG |
| | | | GGCTAGAGACAGCCCACGGGCCCTCCCTCCAAG |
| | | | TGTGGGAGGGAGAGATGCTCTGCCTCTGAACTT |
| | | | CAAAGTGGAGGTGGAGTGCTGGCCACGTCTCCA |
| | | | CCTAACAACCCTCTTTATTCTCTTGTTAAAGTTT |
| | | | TGTTCATGCTTTGATTTTTTTTTAAATTTTTTAGA |
| | | | GACAGGGTCTCACTCTGTTGCCCAGGCTGGAGT |
| | | | GCAGTGGCATGATCATAACTCACTGCAGCCTCA |
| | | | AACTTCTGGCCTCAAGTGATCCTCCTGCCTCGGC |
| | | | CTCCCAAAATGCTGGGATTACAGATGTGAGCCA |
| | | | CCACACACACCATCTGATTAAAAAAAAAAAATA |
| | | | CTGATTCCTTGTAGCAACCCAAAAAATGTAAAG |
| | | | AAAGAATTCGACACAATGGGGTTGCGTCACCTA |
| | | | TAGTTTTCATTTCCTCAAATGCAACTCAAGGTGC |
| | | | TCAGCTTGATAAAGGGAGAGAGGGAGGGCAGG |
| | | | GGAGAAACTGACTCCTTTTTCCATCTCTTTTTTG |
| | | | CCTGCACACTTAATGTGGACTGCCTCCTTGGACT |
| | | | TAAAAAGAGTTGAGCCAACATGCAAATCAAAA |
| | | | AACATTTATGAACTGATTTCTTAGCTTTGGACTT |
| | | | GACAACTGACGAATGCTCTGGGGGTGACCTGGC |
| | | | CCTTGCTGTCCGTTCCACTCCCATCCTTGATGAT |
| | | | GGAGACACCTGTGTTTATGTTGTCACTTCGCTTG |
| | | | CCATCCTCATGCCCCGGGCTCTGCTGGTTTGGA |
| | | | GGGGACAGGGCCCTTGGTTTGCACACCTCACCC |
| | | | GATCTTGGTTATAAGGTACAGTAAAAGCGTCTG |
| | | | AAAAATGCATTCAAAACACAGATTCTGGGGCCC |
| | | | TCGGCACCAGAGGTGTGGATTTGCTA (SEQ ID |
| | | | NO: 123) |
| ZCCHC12 | NM_173798/ | MASIIARVGNSRRLN | GGCGCTGCCTCGTCTCTGCTACCCCTGGTTGGGC |
| (PNMA7A) | NM_173798.4 | APLPPWAHSMLRSLG | GGCCCTGCGAAGCAGCTCCTTCGGGCAGCCCCG |
| | (encoding | RSLGPIMASMADRN | GGTCGCTTAGCGGCCAAGGAGGCTTCAGTTCTT |
| | polynucleotide) | MKLFSGRVVPAQGE | TGCCGCCTGCAAGGCGGAGACCAGAAGGCGGA |
| | NP_776159/ | ETFENWLTQVNGVLP | ATCCACAGCTGGCGACGCGGGAGCATCTGCTGT |
| | NP_776159.1 | DWNMSEEEKLKRLM | CCACCAGCGGAGCACAGGCCATCAAAGCCGCA |
| | (protein) | KTLRGPAREVMRVL | TCTGAACTTGAATTCTGTGCAGCTGATTGCAGA |
| | | QATNPNLSVADFLRA | GCTGGACCCGGATCTGCGACCCCCTGTGGACAG |
| | | MKLVFGESESSVTAH | AGGTTGACCGTACCCCGGAGAGGAGCTTTCTCA |
| | | GKFFNTLQAQGEKAS | CGGAGGGCACTGGTTGCAGAGGCTGGAAGTGA |
| | | LYVIRLEVQLQNAIQ | AATAAAGACGCGCTCTTGTTTCAGAGTTCGTCC |
| | | AGIIAEKDANRTRLQ | CCTGCTGAGATAGGAAGGCAGAGCCACCTCCTC |
| | | QLLLGGELSRDLRLR | TCCTCTCCCACCTGCAGATTAAGCTTTTCTAAAA |
| | | LKDFLRMYANEQER | AGCCTAGGCATCTTCTTATATTCAGATACCCTAT |
| | | LPNFLELIRMVREEE | CGTCGTCAGTCATGGCTAGCATCATTGCACGTG |
| | | DWDDAFIKRKRPKRS | TCGGTAACAGCCGGCGGCTGAATGCACCCTTGC |
| | | ESMVERAVSPVAFQG | CGCCTTGGGCCCATTCCATGCTGAGGTCCCTGG |
| | | SPPIVIGSADCNVIEID | GGAGAAGTCTCGGTCCTATAATGGCCAGCATGG |
| | | DTLDDSDEDVILVES | CAGACAGAAACATGAAGTTGTTCTCGGGGAGG |
| | | QDPPLPSWGAPPLRD | GTGGTGCCAGCCCAAGGGGAAGAAACCTTTGA |
| | | RARPQDEVLVIDSPH | AAACTGGCTGACCCAAGTCAATGGCGTCCTGCC |
| | | NSRAQFPSTSGGSGY | AGATTGGAATATGTCTGAGGAGGAAAAGCTCA |
| | | KNNGPGEMRRARKR | AGCGCTTGATGAAAACCCTTAGGGGCCCTGCCC |
| | | KHTIRCSYCGEEGHS | GCGAGGTCATGCGTGTGCTTCAGGCGACCAACC |
| | | KETCDNESDKAQVFE | CTAACCTAAGTGTGGCAGATTTCTTGCGAGCCA |
| | | NLIITLQELTHTEMER | TGAAATTGGTGTTTGGGGAGTCTGAAAGCAGTG |
| | | SRVAPGEYNDFSEPL | TGACTGCCCATGGTAAATTTTTTAACACCCTACA |
| | | (SEQ ID NO: 124) | AGCTCAAGGGGAGAAAGCCTCCCTTTATGTGAT |
| | | | CCGTTTAGAGGTGCAGCTCCAGAACGCTATTCA |
| | | | GGCAGGCATTATAGCTGAGAAAGATGCAAACC |
| | | | GGACTCGCTTGCAGCAGCTCCTTTTAGGCGGTG |
| | | | AGCTGAGTAGGGACCTCCGACTCAGACTTAAGG |
| | | | ATTTTCTCAGGATGTATGCAAATGAGCAGGAGC |
| | | | GGCTTCCCAACTTTCTGGAGTTAATCAGAATGG |
| | | | TAAGGGAGGAAGAGGATTGGGATGATGCTTTTA |
| | | | TTAAACGGAAGCGTCCAAAAAGGTCTGAGTCAA |
| | | | TGGTGGAGAGGGCAGTCAGCCCTGTGGCATTTC |
| | | | AGGGCTCCCCACCGATAGTGATCGGCAGTGCTG |
| | | | ACTGCAATGTGATAGAGATAGATGATACCCTCG |
| | | | ACGACTCCGATGAGGATGTGATCCTGGTGGAGT |
| | | | CTCAGGACCCTCCACTTCCATCCTGGGGTGCCC |
| | | | CTCCCCTCAGAGACAGGGCCAGACCTCAGGATG |
| | | | AAGTGCTGGTCATTGATTCCCCCCACAATTCCA |
| | | | GGGCTCAGTTTCCTTCCACCAGTGGTGGTTCTGG |
| | | | CTATAAGAATAACGGTCCTGGGGAGATGCGTAG |
| | | | AGCCAGGAAGCGAAAACACACAATCCGCTGTTC |
| | | | GTATTGTGGTGAGGAAGGCCACTCAAAAGAAA |
| | | | CCTGTGACAACGAGAGTGACAAGGCCCAGGTTT |
| | | | TTGAGAATTTGATCATCACTCTCCAGGAGCTGA |
| | | | CCCATACTGAGATGGAGAGGTCAAGAGTGGCCC |
| | | | CTGGCGAATACAATGACTTCTCTGAGCCACTGT |
| | | | AAGGGACCACCCCCAGGTTTCAGTGAACCCTTA |
| | | | CCTATATTCAGCATCCAGTAGTGGGAAAACTGG |
| | | | GGTGGGGGTGGGGGTGGGACTTCTAACTGCATG |
| | | | AATTAATCCACAAAGCGGCTATCTTTTGGGGTG |
| | | | GAGTAGAAAGGGTCTTGGATACCAGCACATTGG |
| | | | AGGGAGATAGCCTGACCTCTGTCCTTGCTCCTTC |
| | | | TCCCTGCAGCCTACGGGTCTGTTTTCTGTGTGTG |
| | | | CCCATTTCCTTGACAGCTTTATTCTTTGTGAAAG |
| | | | TGGTATAATTTATTGTTAAATATTTGAACAATAA |
| | | | AAAAGGTACAAAAAGTGAAGTACAAATTACCC |
| | | | AAATCTCTCCACCCTTATATAATCATTGTCAACC |
| | | | CTTTGATGAGTGATATTTCCCTATACCTATGTAC |
| | | | CCAGATAGATATATGCATAGATAAAAGTGATGA |
| | | | AATATAAGTGCTGTTCTATCTGTATTTTTTCACC |
| | | | AAACAATATATGTTGTGAGCTTCTATGTCAATA |
| | | | AATATATATATCAGCA (SEQ ID NO: 125) |
| ZCCHC12_ | NM_001312891/ | MASIIARVGNSRRLN | GGCGCTGCCTCGTCTCTGCTACCCCTGGTTGGGC |
| i2 | NM_001312891.2 | APLPPWAHSMLRSLG | GGCCCTGCGAAGCAGCTCCTTCGGGCAGCCCCG |
| (PNMA7A_ | (encoding | RSLGPIMASMADRN | GGTCGCTTAGCGGCCAAGGAGGCTTCAGTTCTT |
| i2) | polynucleotide) | MKLFSGRVVPAQGE | TGCCGCCTGCAAGGCGGAGACCAGAAGGCGGA |
| | NP_001299820/ | ETFENWLTQVNGVLP | ATCCACAGCTGGCGACGCGGGAGCATCTGCTGT |
| | NP_001299820.1 | DWNMSEEEKLKRLM | CCACCAGCGGAGCACAGGACCCGGATCTGCGA |
| | (protein) | KTLRGPAREVMRVL | CCCCCTGTGGACAGAGGTTGACCGTACCCCGGA |
| | | QATNPNLSVADFLRA | GAGGAGCTTTCTCACGGAGGGCACTGGTTGCAG |
| | | MKLVFGESESSVTAH | AGGCTGGAAGTGAAATAAAGACGCGCTCTTGTT |
| | | GKFFNTLQAQGEKAS | TCAGAGTTCGTCCCCTGCTGAGATAGGAAGGCA |
| | | LYVIRLEVQLQNAIQ | GAGCCACCTCCTCTCCTCTCCCACCTGCAGATTA |
| | | AGIIAEKDANRTRLQ | AGCTTTTCTAAAAAGCCTAGGCATCTTCTTATAT |
| | | QLLLGGELSRDLRLR | TCAGATACCCTATCGTCGTCAGTCATGGCTAGC |
| | | LKDFLRMYANEQER | ATCATTGCACGTGTCGGTAACAGCCGGCGGCTG |
| | | LPNFLELIRMVREEE | AATGCACCCTTGCCGCCTTGGGCCCATTCCATG |
| | | DWDDAFIKRKRPKRS | CTGAGGTCCCTGGGGAGAAGTCTCGGTCCTATA |
| | | ESMVERAVSPVAFQG | ATGGCCAGCATGGCAGACAGAAACATGAAGTT |
| | | SPPIVIGSADCNVIEID | GTTCTCGGGGAGGGTGGTGCCAGCCCAAGGGG |
| | | DTLDDSDEDVILVES | AAGAAACCTTTGAAAACTGGCTGACCCAAGTCA |
| | | QDPPLPSWGAPPLRD | ATGGCGTCCTGCCAGATTGGAATATGTCTGAGG |
| | | RARPQDEVLVIDSPH | AGGAAAAGCTCAAGCGCTTGATGAAAACCCTTA |
| | | NSRAQFPSTSGGSGY | GGGGCCCTGCCCGCGAGGTCATGCGTGTGCTTC |
| | | KNNGPGEMRRARKR | AGGCGACCAACCCTAACCTAAGTGTGGCAGATT |
| | | KHTIRCSYCGEEGHS | TCTTGCGAGCCATGAAATTGGTGTTTGGGGAGT |
| | | KETCDNESDKAQVFE | CTGAAAGCAGTGTGACTGCCCATGGTAAATTTT |
| | | NLIITLQELTHTEMER | TTAACACCCTACAAGCTCAAGGGGAGAAAGCCT |
| | | SRVAPGEYNDFSEPL | CCCTTTATGTGATCCGTTTAGAGGTGCAGCTCCA |
| | | (SEQ ID NO: 126) | GAACGCTATTCAGGCAGGCATTATAGCTGAGAA |
| | | | AGATGCAAACCGGACTCGCTTGCAGCAGCTCCT |
| | | | TTTAGGCGGTGAGCTGAGTAGGGACCTCCGACT |
| | | | CAGACTTAAGGATTTTCTCAGGATGTATGCAAA |
| | | | TGAGCAGGAGCGGCTTCCCAACTTTCTGGAGTT |
| | | | AATCAGAATGGTAAGGGAGGAAGAGGATTGGG |
| | | | ATGATGCTTTTATTAAACGGAAGCGTCCAAAAA |
| | | | GGTCTGAGTCAATGGTGGAGAGGGCAGTCAGCC |
| | | | CTGTGGCATTTCAGGGCTCCCCACCGATAGTGA |
| | | | TCGGCAGTGCTGACTGCAATGTGATAGAGATAG |
| | | | ATGATACCCTCGACGACTCCGATGAGGATGTGA |
| | | | TCCTGGTGGAGTCTCAGGACCCTCCACTTCCATC |
| | | | CTGGGGTGCCCCTCCCCTCAGAGACAGGGCCAG |
| | | | ACCTCAGGATGAAGTGCTGGTCATTGATTCCCC |
| | | | CCACAATTCCAGGGCTCAGTTTCCTTCCACCAGT |
| | | | GGTGGTTCTGGCTATAAGAATAACGGTCCTGGG |
| | | | GAGATGCGTAGAGCCAGGAAGCGAAAACACAC |
| | | | AATCCGCTGTTCGTATTGTGGTGAGGAAGGCCA |
| | | | CTCAAAAGAAACCTGTGACAACGAGAGTGACA |
| | | | AGGCCCAGGTTTTTGAGAATTTGATCATCACTCT |
| | | | CCAGGAGCTGACCCATACTGAGATGGAGAGGTC |
| | | | AAGAGTGGCCCCTGGCGAATACAATGACTTCTC |
| | | | TGAGCCACTGTAAGGGACCACCCCCAGGTTTCA |
| | | | GTGAACCCTTACCTATATTCAGCATCCAGTAGT |
| | | | GGGAAAACTGGGGTGGGGGGGGGGGGGACT |
| | | | TCTAACTGCATGAATTAATCCACAAAGCGGCTA |
| | | | TCTTTTGGGGTGGAGTAGAAAGGGTCTTGGATA |
| | | | CCAGCACATTGGAGGGAGATAGCCTGACCTCTG |
| | | | TCCTTGCTCCTTCTCCCTGCAGCCTACGGGTCTG |
| | | | TTTTCTGTGTGTGCCCATTTCCTTGACAGCTTTA |
| | | | TTCTTTGTGAAAGTGGTATAATTTATTGTTAAAT |
| | | | ATTTGAACAATAAAAAAGGTACAAAAAGTGAA |
| | | | GTACAAATTACCCAAATCTCTCCACCCTTATATA |
| | | | ATCATTGTCAACCCTTTGATGAGTGATATTTCCC |
| | | | TATACCTATGTACCCAGATAGATATATGCATAG |
| | | | ATAAAAGTGATGAAATATAAGTGCTGTTCTATC |
| | | | TGTATTTTTTCACCAAACAATATATGTTGTGAGC |
| | | | TTCTATGTCAATAAATATATATATCAGCA (SEQ |
| | | | ID NO: 127) |
| ZCCHC18 | NM_001143978/ | MASITACVGNSRQQN | GCTCTTTTGTCTCAGTCGCCAGAGACTGAAAGC |
| | NM_001143978.3 | APLPPWAHSMLRSLG | AACCGCGGCTGCCCGGAGGGCCGAACTGGAGG |
| | (encoding | RSLCPLVVKMAERN | GTGGGCGCAGCCTTGGCTGCCTTGGGAACCGTG |
| | polynucleotide) | MKLFSGRVVPAQGK | CTGCCTCGACTCGGCTACCCCTCGCTGGGCGGT |
| | NP_001137450/ | ETFENWLIQVNEVLP | CGTGCGCACTCGCTAGGTCGGGCAGCCCCGGGT |
| | NP_001137450.1 | DWSMSEEEKLKRLM | CGCTTAGCGGCCGAGGAGGCGGCAGAGATCCC |
| | (protein) | KTLRGPAREVMRLL | GTTCCCCTGCAGAGCTACGGGGACCGGAAGGCG |
| | | QAANPNLSVADFLRA | AAACAGAGCTGTGGACGCGGGAGCACATGCTG |
| | | MKLVFGESESSVTAH | CCCACCAGTGGAGCACAAGTAAAGAAAGAACG |
| | | GKFFNTLQAQGEKAS | CGCGATTGCCCGGCGCGGAGAGGGGTGGGGGA |
| | | LYVIRLEVQLQNAIQ | AGCCCCGCCAGGACTGGGCGGCGCCCGGCTTGG |
| | | AGILAEKDANQTRLQ | AGAATCAGTAACTGCGAGGCAGGGGATGGAGC |
| | | QLLLGAELNRDLRFR | ACTCCCTTGGCCTGGTTGCGCTGAGGAAGGAGT |
| | | LKHLLRMYANKQER | TGTGGTTCATCTGGATGCTGCCGACTGGATAGG |
| | | LPNFLELIKMIREEED | ATTTAGGATTTGCCTGGATTAAGGTCATCAAAG |
| | | WDDAFIKRKRPKRSE | CCGAATCCGAACTTGAATTCTGTGCGGCGGATT |
| | | PIMERAASPVAFQGA | GCAGAGCTGTCTTGAGGCCCTGAGGACTGGGAT |
| | | QPIAISSADCNCNVIEI | CTGCGACACCCAGTGGACAAAAGTCGGCCGTAC |
| | | DDTLDDSDEDVILVV | CCCAGAGGCTAATTTTCTCACGGCAGGCACCTG |
| | | SLYPSLTPTGAPPFRG | TTGCAGAGGCTGCACGTAAAATAAAGGCGAAC |
| | | RARPLDQVLVIDSPN | GCTAGTTTCCGAGCTCATGTAAGAATCTGAGAA |
| | | NSGAQSLSTSGGSGY | ATAACGGGGGAAACGTTGACAAGGGGGTGTGA |
| | | KNDGPGNIRRARKRK | TTGAGACCTGAGGCCATGAGGTTAATGGTTTCT |
| | | YTTRCSYCGEEGHSK | TATTATCATAAATCTGTGACACACAACCATTGA |
| | | ETCDNESNKAQVFEN | GTTCCGTTTTGAGGGCCCAGTATCAGCTCCCAA |
| | | LIITLQELTHTEERSK | CACTATGTCATCTAGAATTAGAAGCAACAGTTG |
| | | EVPGEHSDASEPQ | TCGCCCCTCCTGGAGTACAGGCAGGAGGGGATG |
| | | | GATGTGGCTCTGAAAAACTACCTACTAGCCCAG |
| | | | GGACACCTGCTAGCTCTGGAATGTTGTAATTGC |
| | | | CAGTCTCCTGCAGGGTATTGTTAAGGCACTGGG |
| | | | CAGATATAAAATGCACTGCAAGGCTATTCAGGA |
| | | | AGATAGAGAATGCTACTGCAGACTGCTTCCCCA |
| | | | CAGCCCACTATCTTATTAACCTTTTTTACTTTCC |
| | | | TTAGAATCCCTACTGAAATATAAGGCAGGCACA |
| | | | GCCCAGGAACGTTGCTTTGGAGAATCCTGCAGA |
| | | | TAAGGCTTTTCCAAAAAGCGCGAGCATCTTGTT |
| | | | GTATTCAGATACCCTATCGTCGTCAGTCATGGCT |
| | | | AGCATCACTGCGTGTGTGGGTAACAGCAGGCAG |
| | | | CAGAATGCACCTTTGCCGCCTTGGGCCCATTCC |
| | | | ATGTTGAGGTCTCTGGGGAGGAGTCTCTGTCCT |
| | | | TTAGTGGTCAAAATGGCAGAGAGAAACATGAA |
| | | | GTTGTTCTCAGGAAGAGTGGTGCCAGCCCAGGG |
| | | | GAAAGAAACCTTTGAAAACTGGCTGATCCAAGT |
| | | | CAATGAGGTCCTGCCAGATTGGAGTATGTCTGA |
| | | | GGAGGAAAAACTCAAGCGCTTGATGAAAACAC |
| | | | TTAGGGGCCCTGCCCGGGAGGTCATGCGTTTGC |
| | | | TTCAGGCGGCCAACCCCAACCTAAGTGTAGCAG |
| | | | ATTTCTTGCGGGCAATGAAATTGGTGTTTGGGG |
| | | | AGTCTGAAAGCAGTGTGACTGCCCATGGTAAAT |
| | | | TTTTTAACACCCTGCAGGCACAAGGGGAGAAAG |
| | | | CCTCCCTTTATGTGATCCGTTTAGAGGTGCAGCT |
| | | | CCAGAATGCTATTCAGGCAGGCATCCTAGCTGA |
| | | | GAAAGATGCAAACCAGACTCGCTTGCAACAGCT |
| | | | TCTTTTAGGCGCTGAGCTGAATAGGGACCTGCG |
| | | | CTTCAGGCTTAAGCATCTTCTCAGGATGTATGC |
| | | | AAATAAGCAGGAGCGGCTTCCCAATTTCCTGGA |
| | | | GTTAATCAAGATGATAAGGGAGGAAGAGGATT |
| | | | GGGATGATGCTTTTATTAAACGGAAGCGGCCGA |
| | | | AAAGGTCTGAGCCAATAATGGAGAGGGCAGCC |
| | | | AGCCCTGTGGCATTTCAGGGCGCCCAGCCAATA |
| | | | GCAATCAGCAGTGCTGACTGTAACTGCAACGTG |
| | | | ATAGAAATAGATGATACCCTTGATGACTCTGAT |
| | | | GAGGATGTGATCCTGGTGGTGTCTCTGTACCCTT |
| | | | CACTGACACCTACAGGTGCCCCTCCCTTCAGAG |
| | | | GAAGAGCCAGACCTCTGGATCAAGTGCTGGTTA |
| | | | TTGATTCCCCCAACAATTCTGGGGCTCAGTCTCT |
| | | | TTCTACCAGTGGTGGTTCTGGGTATAAGAATGA |
| | | | TGGTCCTGGGAATATTCGTAGAGCCAGGAAGCG |
| | | | AAAATACACAACCCGCTGCTCATATTGTGGGGA |
| | | | GGAGGGCCACTCAAAAGAAACCTGTGACAATG |
| | | | AGAGCAACAAGGCCCAGGTTTTTGAGAATCTGA |
| | | | TCATCACCCTGCAGGAGCTGACACATACAGAGG |
| | | | AGAGGTCAAAAGAGGTCCCTGGAGAACACAGT |
| | | | GATGCTTCTGAGCCACAGTAAGGATCTAGTCCA |
| | | | GCCCTAAATGAGTCCTTGACTGTATTCAGAGTC |
| | | | TGGTAATGGGAATAACAGGAGAGGGGGGTGGG |
| | | | TTTCTAACTGCATGAATTAATCCACAAAGCAGT |
| | | | TTTCCTTTGGGAAGGAGAAGAGGTCTTGCATAC |
| | | | CAGCACAGTGGATGGGGAATGGTGTGACCTCTG |
| | | | TCCCTGCTCTCTTCTCTGCTGGCTTAAGGGTCTA |
| | | | TTCTCCATGTGTCTATTTCTTTGATGACTTTATA |
| | | | CTTTTTATGAAAGGAGCATCTTTTCATTAAACCT |
| | | | TCAAAAATAAAGGAAGATACAAAAACTAAAGT |
| | | | ACAAATTACCTGAAACTCAGCACCCTTTTGTAA |
| | | | CCATTGTCAGCCCTTTGGTGAATGTCCTTCCAGA |
| | | | TATCTCCCTGTACCTGTGTACCCAGATAGATATA |
| | | | TGTATAGATAAGAGTGACCAAATATAAGTGCTG |
| | | | TTCTATACTGTGTATTTTTCACCAAATAATGTAT |
| | | | CTCGTGGACTTCTATGTCGATGAATATATATAA |
| | | | ATATCTAA (SEQ ID NO: 129) |

In certain example embodiments, the engineered PNMA polypeptide contains one or more modifications that modify the internal capsid surface of a capsid formed from the engineered PNMA polypeptide(s). In certain example embodiments, the engineered PNMA polypeptide contains one or more modifications that modify the outer capsid surface of a capsid formed from the engineered PNMA polypeptide(s). In certain example embodiments, the engineered PNMA polypeptide contains one or more modifications that modify the internal capsid surface of a capsid formed from the engineered PNMA polypeptide(s) and one or more modifications that modify the outer capsid surface of a capsid formed from the engineered PNMA polypeptide(s).

Exemplary modifications to the engineered PNMA polypeptides that result in internal or outer surface Capsid modifications are further detailed below.

### Internal Capsid Modifications

The engineered PNMA polypeptide contains one or more modifications that enhance binding or loading of the cargo. In certain example embodiments, the one or more modifications that enhance binding or loading of the cargo are or include addition of a peptide or polypeptide comprising charged residues; addition of a polynucleotide binding domain; addition of a polypeptide binding domain; or any combination thereof.

### Charged Polypeptides

The one or more modifications that enhance binding or loading of the cargo are or include addition of a peptide or polypeptide comprising charged residues such that after the modification the engineered PNMA capsid contains regions on the inner surface that are positively charged, negatively charged, or contains positively charged regions and negatively charged regions. Cargo that is oppositely charged as compared to charged region on the inner capsid surface can be bound or otherwise associated with or attracted to that charged region. Without being bound by theory this can enhance binding or capture of the cargo in the engineered PNMA capsid.

In some embodiments the peptide comprising charged residues is (a) inserted between any two consecutive amino acids in a loop domain of the PNMA; (b) an addition to the C- or N-terminus of the PNMA; (c) an addition to a C- or N-terminally truncated PNMA; or (a) combined with (b) or (c).

In some embodiments, the peptide comprising charged residues is inserted into a loop domain at amino acids 170-180, amino acids 256-263, or amino acids 302-305 of PNMA2, or a position corresponding thereto in another PNMA (e.g., PNMA1, PNMA3, PNMA3_i2, PNMA5, PNMA5_i2, PNMA5_i3, PNMA5_i4, PNMA6A, PNMA6E, PNMA6E_i2, PNMA6E_i3, PNMA6F, PNMA8A, PNMA8A_i2, PNMA8B, PNMA8B_i2, PNMA8C, CCDC8, ZCCHC12 (PNMA7A), ZCCHC12_i2 (PNMA7A_i2), ZCCHC12_i3 (PNMA7A_i3), ZCCHC18, or MOAP1 (PNMA4) or a functional domain thereof. Thus, in some embodiments, the engineered PNMA polypeptide is an engineered PNMA1, PNMA2, PNMA3, PNMA3_i2, PNMA5, PNMA5_i2, PNMA5_i3, PNMA5_i4, PNMA6A, PNMA6E, PNMA6E_i2, PNMA6E_i3, PNMA6F, PNMA8A, PNMA8A_i2, PNMA8B, PNMA8B_i2, PNMA8C, CCDC8, ZCCHC12 (PNMA7A), ZCCHC12_i2 (PNMA7A_i2), ZCCHC12_i3 (PNMA7A_i3), ZCCHC18, or MOAP1 (PNMA4).

In some embodiments, the peptide comprising charged residues is inserted between amino acids 175 and 176, 261 and 262, 303 and 304 of PNMA2, or at a position corresponding thereto of another PNMA (e.g., PNMA1, PNMA2, PNMA3, PNMA3_i2, PNMA5, PNMA5_i2, PNMA5_i3, PNMA5_i4, PNMA6A, PNMA6E, PNMA6E_i2, PNMA6E_i3, PNMA6F, PNMA8A, PNMA8A_i2, PNMA8B, PNMA8B_i2, PNMA8C, CCDC8, ZCCHC12 (PNMA7A), ZCCHC12_i2 (PNMA7A_i2), ZCCHC12_i3 (PNMA7A_i3), ZCCHC18, or MOAP1 (PNMA4) or a functional domain thereof. Thus, in some embodiments, the engineered PNMA polypeptide is an engineered PNMA1, PNMA2, PNMA3, PNMA3_i2, PNMA5, PNMA5_i2, PNMA5_i3, PNMA5_i4, PNMA6A, PNMA6E, PNMA6E_i2, PNMA6E_i3, PNMA6F, PNMA8A, PNMA8A_i2, PNMA8B, PNMA8B_i2, PNMA8C, CCDC8, ZCCHC12 (PNMA7A), ZCCHC12_i2 (PNMA7A_i2), ZCCHC12_i3 (PNMA7A_i3), ZCCHC18, or MOAP1 (PNMA4)).

In one embodiment, the wherein the size of the C-terminus truncation of the C-terminally truncated PNMA is 1 to 31 amino acids. In some embodiments, the size of the C-terminus truncation of the C-terminally truncated PNMA is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, to/or 31 amino acids.

In one embodiment, the peptide comprising charged residues is about 20 to about 150 amino acids in size. In some embodiments, the peptide comprising charged residues is about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or about 150 amino acids in size. In one embodiment, the peptide comprising charged residues ranges from 20 to 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112,113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 amino acids in size. In one embodiment, the percent of residues that are charged in the peptide comprising charged residues ranges from any non-zero number to 100 percent, such as 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 5100, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90%, 90.1%, 90.2%, 90.3%, 90.4%, 90.5%, 90.6%, 90.7%, 90.8%, 90.9%, 91%, 91.1%, 91.2%, 91.3%, 91.4%, 91.5%, 91.6%, 91.7%, 91.8%, 91.9%, 92%, 92.1%, 92.2%, 92.3%, 92.4%, 92.5%, 92.6%, 92.7%, 92.8%, 92.9%, 93%, 93.1%, 93.2%, 93.3%, 93.4%, 93.5%, 93.6%, 93.7%, 93.8%, 93.9%, 94%, 94.1%, 94.2%, 94.3%, 94.4%, 94.5%, 94.6%, 94.7%, 94.8%, 94.9%, 95%, 95.1%, 95.2%, 95.3%, 95.4%, 95.5%, 95.6%, 95.7%, 95.8%, 95.9%, 96%, 96.1%, 96.2%, 96.3%, 96.4%, 96.5%, 96.6%, 96.7%, 96.8%, 96.9%, 97%, 97.1%, 97.2%, 97.3% 97.4% 97.5% 97.6%, 97.7% 97.8%, 97.9% 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, to/or 100%.

In one embodiment, the peptide comprising charged residues comprises an arginine, lysine, and/or proline rich motif. In certain embodiments, the peptide comprising charged residues is rich in positively charged amino acids. In some embodiments, the peptide comprising charged amino acid residues is rich in arginine, lysine, proline, and any combination thereof.

In one embodiment, the peptide comprising charged residues comprises two or more RKK repeats (e.g., 2, 3, 4, 5, 6, 7, 8, 9, 10, 11, 12, 13, 14, 15 or more) or two or more RRLRRP (SEQ ID NO: 6) repeats (e.g., 2, 3, 4, 5, 6, 7, 8, 9, 10, 11, 12, 13, 14, 15 or more). In certain embodiments, the peptide comprising charged residues is RRKRRKRRKRRK (SEQ ID NO: 7). In one embodiments, the peptide comprising charged residues is RRLRRPRRLRRPRRPR (SEQ ID NO: 8).

### Polynucleotide Binding Domains

The one or more modifications that enhance binding or loading of the cargo may be, or include, addition of one or more polynucleotide binding domain(s).

In one embodiment, the polynucleotide binding domain is (a) inserted between any two consecutive amino acids in a loop domain of the PNMA; (b) inserted in place of at least a portion of a zinc finger region; (c) an extension of a C- or N-terminus of the PNMA; (d) an extension of a C- or N-terminally truncated PNMA; or (a) combined with (b); or (a) and (b) combined with (c) or (d); or (b) combined with (c) or (d).

In one embodiment, the polynucleotide binding domain is inserted between amino acids 256-263 or 302-305 of PNMA2 or at a position corresponding thereto in another PNMA, such as PNMA1, PNMA3, PNMA3_i2, PNMA5, PNMA5_i2, PNMA5_i3, PNMA5_i4, PNMA6A, PNMA6E, PNMA6E_i2, PNMA6E_i3, PNMA6F, PNMA8A, PNMA8A_i2, PNMA8B, PNMA8B_i2, PNMA8C, CCDC8, ZCCHC12 (PNMA7A), ZCCHC12_i2 (PNMA7A_i2), ZCCHC12_i3 (PNMA7A_i3), ZCCHC18, or MOAP1 (PNMA4).

In one embodiment, the polynucleotide binding domain is inserted between amino acids 261 and 262, or amino acid 303 and 304 of PNMA2 or an amino acid position corresponding thereto in another PNMA, such as PNMA1, PNMA3, PNMA3_i2, PNMA5, PNMA5_i2, PNMA5 i3, PNMA5_i4, PNMA6A, PNMA6E, PNMA6E_i2, PNMA6E_i3, PNMA6F, PNMA8A, PNMA8A_i2, PNMA8B, PNMA8B_i2, PNMA8C, CCDC8, ZCCHC12 (PNMA7A), ZCCHC12_i2 (PNMA7A_i2), ZCCHC12_i3 (PNMA7A_i3), ZCCHC18, or MOAP1 (PNMA4).

In one embodiment, the polynucleotide binding domain replaces amino acids 412 to 429 of PNMA3 or an amino acid position corresponding thereto in another PNMA such as PNMA1, PNMA2, PNMA3_i2, PNMA5, PNMA5_i2, PNMA5_i3, PNMA5_i4, PNMA6A, PNMA6E, PNMA6E_i2, PNMA6E_i3, PNMA6F, PNMA8A, PNMA8A_i2, PNMA8B, PNMA8B_i2, PNMA8C, CCDC8, ZCCHC12 (PNMA7A), ZCCHC12_i2 (PNMA7A_i2), ZCCHC12_i3 (PNMA7A_i3), ZCCHC18, or MOAP1 (PNMA4).

In one embodiment, the polynucleotide binding domain is inserted between M1 and P2 of the N-terminus of PNMA3 or a position corresponding thereto in another PNMA such as PNMA1, PNMA2, PNMA3_i2, PNMA5, PNMA5 i2, PNMA5_i3, PNMA5_i4, PNMA6A, PNMA6E, PNMA6E_i2, PNMA6E_i3, PNMA6F, PNMA8A, PNMA8A_i2, PNMA8B, PNMA8B_i2, PNMA8C, CCDC8, ZCCHC12 (PNMA7A), ZCCHC12_i2 (PNMA7A_i2), ZCCHC12_i3 (PNMA7A_i3), ZCCHC18, or MOAP1 (PNMA4).

In one embodiment, the polynucleotide binding domain comprises or consists of a PNMA RNA recognition motif. The polynucleotide binding domain may also be derived from RNA or DNA binding domains contained within other proteins. The DNA or RNA binding domain may be derived from a λN polypeptide, a P22N polypeptide, a MS2 polypeptide, an R17 polypeptide, a retroviral or lentiviral Rev polypeptide, polynucleotide binding domain of a nuclease, a Zinc Finger domain, a 14-3-3 polypeptide, a STAR-family polypeptide, a toll-like receptor polypeptide, and a CCMV N-terminal sequence, an arginine, lysine, and/or proline rich motif, or any combination thereof. The polynucleotide binding domain may comprise an RNA-binding motif (RRM), a DNA-binding AT-rich interative domain (ARID), bioRxiv 2021 doi.org/10.1101/2021.03.25.434787), a DNA binding domain from MarA or Rob, The polynucleotide binding domains may recognize specific sequences, local DNA or RNA structures (G-quadruplexes, 1-motifs, triplexes, cruciform, left-handed DNA/RNA form), or both. Bartas et al., Amino Acid Composition in Various Types of Nucleic Acid-Binding Proteins. Int J Mol Sci. 2021 Jan. 18; 22(2):92.

DNA and RNA binding domains may also be identified and/or designed using tools such as IDRBP-PPCT (Wang N, Zhang J, Liu B. IEEE/ACM Trans Comput Biol Bioinform. 2022 July-August; 19(4):2284-2293); Deep learning DNA-binding protein prediction. (Guan et al. (2021). DNA-Binding Protein Prediction Based on Deep Learning Feature Fusion. In: Huang, DS., Jo, KH., Li, J., Gribova, V., Premaratne, P. (eds) Intelligent Computing Theories and Application. ICIC 2021. Lecture Notes in Computer Scienceo, vol 12838. Springer, Cham), DeepMC-INABP (Cui et al. Computational and Structural Biotechnology Journal, 20:2020-20280 (2022).

Further methods for identifying and designing DNA and RNA binding proteins are described in, Mukherjee and Nitin, Advances in Protein Molecular and Structural Biology Methods. (2022) pages 163-180.

In some embodiments, the λN comprises or consists of MDAQTRRRERRAEKQAQWKAAN (SEQ ID NO: 1) or GNARTRRRERRAEKQAQWKAAN (SEQ ID NO: 2).

In some embodiments, the P22N comprises or consists of GNAKTRRHERRRKLAIERDTI (SEQ ID NO: 3).

In some embodiments, the Rev polypeptide comprises or consists of TRQARRNRRRRWRERQR (SEQ ID NO: 4).

In some embodiments, the zinc finger domain is a zinc finger domain from a PNMA. In some embodiments, the zinc finger domain is a PNMA3 zinc finger domain (e.g., PNMA3 residues 336-436).

In some embodiments, the polynucleotide binding domain comprises or consists of a CCMV N-terminal sequence (MSTVGTGKLTRAQRRAAARKNKRNTRVVQP, SEQ ID NO: 5).

Exemplary arginine, lysine, and/or proline rich motifs are described in greater detail elsewhere herein.

### Polypeptide Binding Domains

In some embodiments, the one or more modifications that enhance binding or loading of the cargo are or include addition of one or more polypeptide binding domain(s) (also referred to herein as protein binding domains). In some embodiments, the polypeptide binding domain(s) are capable of binding, such as specifically binding to a cargo polypeptide or peptide. In some embodiments, the cargo polypeptide or peptide contains a native sequence that will bind the protein binding domain of the engineered PNMA polypeptide and/or capsid. In other embodiments, the cargo can be modified to contain a domain that binds or otherwise interacts with the protein binding domain of the engineered PNMA polypeptide and/or capsid. Without being bound by theory, the protein binding domain of the engineered PNMA polypeptide and/or capsid binds or otherwise interacts with the cargo at the domain of or added to a cargo that will bind or otherwise interact with the protein binding domain of the engineered PNMA polypeptide and/or capsid to facilitate enhanced capture and/or loading of the cargo polypeptide or peptide. For example, and as demonstrated in the Working Examples herein, a dimerization domain can be added to both the cargo and the PNMA. Dimerization of the dimerization domains of the PNMA and cargo forms a complex between the PNMA and cargo thereby facilitating and/or enhancing capture and/or loading of the cargo into the capsid.

In some embodiments, a polypeptide binding domain is coupled to a cargo by directly fusing the polypeptide binding domain to the cargo. In some embodiments, a polypeptide binding domain is coupled to a cargo via a linker. In some embodiments, the linker is a peptide or polypeptide. In some embodiments, the linker is a Gly-Ser linker. One exemplary Gly-Ser linker is GSGGGS (SEQ ID NO: 9). Other exemplary linkers are described elsewhere herein and will be appreciated by one of ordinary skill in the art in view of the description herein. The polypeptide binding domain can be coupled to any suitable location on a cargo. In some embodiments, where the cargo is a peptide or polypeptide, the polypeptide binding domain is coupled to the C-terminus or N-terminus of the cargo peptide or polypeptide.

In some embodiments, a polypeptide binding domain is added to a C- or N-terminus of the PNMA. In some embodiments, a polypeptide binding domain is coupled to a PNMA by directly fusing the polypeptide binding domain to the PNMA. In some embodiments, a polypeptide binding domain is coupled to a PNMA via a linker. In some embodiments, the linker is a peptide or polypeptide. In some embodiments, the linker is a Gly-Ser linker. One exemplary Gly-Ser linker is GSGGGS (SEQ ID NO: 9). Other exemplary linkers are described elsewhere herein and will be appreciated by one of ordinary skill in the art in view of the description herein. The polypeptide binding domain can be coupled to any suitable location on a PNMA. In some embodiments, the polypeptide binding domain is coupled to the C-terminus or N-terminus of the PNMA.

In some embodiments, the protein binding domain (of a PNMA and/or cargo) is a protein-protein interaction domain, such as a receptor ligand, a ligand binding domain, antigenic polypeptide, an antibody antigen binding domain, or dimerization domain. In some embodiments, the protein binding domain is a leucine zipper.

The term antibody is as defined elsewhere herein. As used herein, “antigen” refers to a molecule or a portion of a molecule capable of being bound by an antibody, or by a T cell receptor (TCR) when presented by MHC molecules. At the molecular level, an antigen is characterised by its ability to be bound at the antigen-binding site of an antibody. The specific binding denotes that the antigen will be bound in a highly selective manner by its cognate antibody and not by the multitude of other antibodies which may be evoked by other antigens. An antigen is additionally capable of being recognised by the immune system. In some instances, an antigen is capable of eliciting a humoral immune response in a subject. In some instances, an antigen is capable of eliciting a cellular immune response in a subject, leading to the activation of B- and/or T-lymphocytes.

In some embodiments, the protein binding domain is a dimerization domain. In these embodiments, the cargo polypeptide can contain a dimerization domain capable of dimerizing with the dimerization domain of the engineered PNMA capsid. In some embodiments the protein binding domain modification of the engineered PNMA polypeptide and/or capsid is a leucine zipper. In some embodiments the dimerization domain on the cargo polypeptide is a leucine zipper. Other exemplary protein binding domains include PDZ domains (see e.g., Lee and Zhang. Cell Comm Signal. 8, 8 2010. https://doi.org/10.1186/1478-811X-8-8, which can dimerize with other PDZ domains or bind PDZ binding partners, type I deiodinase dimerization domain -DFL-YI-EAH-DGW- (see e.g., Leonard et al., J Biol. Chem. 2005. 28(12):11093-11100), LIM/double zinc finger motif (see e.g., Feuerstein et al., 1994. PNAS 91:10655-10659), WDR62 dimerization domain (see e.g., Cohen-Katsenelson et al., J. Biol. Chem. 2013. 288(10): 7294-7304), a CD28 YxxxxT dimerization motif (see e.g., Leddon et al., 2020. Front. Immunol. https://doi.org/10.3389/fimmu.2020.01519), von Willebrand factor cystine knot (CK) (CTCK) domain (see e.g., Zhou and Springer. Blood (2014) 123 (12): 1785-1793), a CX₃CX₂CX₃C (SEQ ID NO: 10) tetracysteine motif (see e.g., Schrimpf et al., Biochem. 2018 57 (26) 3658-3664)), a LIxxGVxxGVxxT (SEQ ID NO: 11) motif (see e.g., Lemmon et al., 1994, March; 1(3):157-63. doi: 10.1038/nsb0394-157), and/or the like.

### Outer Capsid Surface Modifications

In some embodiments, the engineered PNMA polypeptide contains one or more modifications that result in a modification to the outer surface of a capsid formed from the engineered PNMA polypeptide.

### Modifying Cell-Specificity

In some embodiments, the one or more modifications that result in a modification to the outer surface of a capsid formed from the engineered PNMA polypeptide modifies cell-specificity of the engineered PNMA capsid.

In some embodiments, the one or more modifications that modify cell-specificity comprise insertion of a cell surface binding peptide, cell penetrating peptide, monobody, nanobody, or antibody or fragment thereof, in the N-terminus of the PNMA. In some embodiments, the one or more modifications are inserted between amino acid residues P27-E31, G125 and S138, P196 and T198, D224 and S229, G319 and S323, or any combination thereof with reference to PNMA2 or PNMA3 or a position in another PNMA corresponding thereto, such as PNMA1, PNMA3_i2, PNMA5, PNMA5_i2, PNMA5_i3, PNMA5_i4, PNMA6A, PNMA6E, PNMA6E_i2, PNMA6E_i3, PNMA6F, PNMA8A, PNMA8A_i2, PNMA8B, PNMA8B_i2, PNMA8C, CCDC8, ZCCHC12 (PNMA7A), ZCCHC12_i2 (PNMA7A_i2), ZCCHC12_i3 (PNMA7A_i3), ZCCHC18, or MOAP1 (PNMA4). In some embodiments, the cell surface binding peptide is an integrin binding peptide (e.g., RGD and iRGD), a VEGFR-1 ligand, an EGF peptide, a human transferrin receptor binding peptide, a hepatocellular carcinoma targeting peptide. Other exemplary cell surface binding peptides include, without limitation, antibodies (including single domain antibodies) or fragments thereof (such as the antigen binding domain) (e.g., those that target CD52, VEGF, EGF receptor, CD33, CD20, CTLA-4, HER2, PSMA, among others), cell penetrating peptides (e.g., NGR, LAH4, and others which are described elsewhere herein), Angiopep-2 (complementary ligand for the low-density lipoprotein receptor-related protein), cell receptor binding peptide, peptides that bind and facilitate translocation across the blood-brain barrier (e.g., GLA, GYR, and MTfp see e.g., van Rooy et al., Pharm Res. 2010. 27(4):673-682 and Hudecz et al., PLoS ONE 16(4): e0249686), and/or the like. In some embodiments, one or more cell penetrating peptides or other molecules (e.g., cell surface binding peptide, cell penetrating peptide, monobody, nanobody, or antibody or fragment thereof) to be added to the outer surface of the capsid are introduced by mixing the engineered capsids with the one or more cell penetrating peptides or other molecule to be added to the engineered capsid. In some such embodiments, the modifications are added after engineered capsid formation and cargo packaging. In some embodiments, the cell penetrating peptides added via mixing is LHA4.

### Modifying Intracellular Delivery

In some embodiments, the one or more modifications that result in a modification to the outer surface of a capsid formed from the engineered PNMA polypeptide that enhances intracellular delivery. In some embodiments, the one or more modifications that enhance intracellular delivery enhance/increase cell entry of the capsid, enhance/increase endosomal escape of the capsid, or both. Without being bound by theory, internalization of the engineered PNMA capsids can be by endocytosis or related mechanism, which can result in the formation of intracellular endosomes containing the engineered PNMA capsids. When endosomes evolve into lysosomes this can result in the degradation of the engineered PNMA capsids and any cargo contained therein. Thus, improving endosomal escape of the engineered PNMA capsids can increase cargo delivery efficiency.

In some embodiments, the one or more modifications that enhance intracellular delivery are capable of enhancing cell entry, endosomal escape or both. In some embodiments, the one or more modifications that enhance intracellular delivery include or are endosomal escape polypeptides or peptides. Endosomal escape polypeptides or peptides are polypeptides or peptides that facilitate escape of the engineered PNMA capsid from the endosome. In some embodiments the endosomal escape polypeptides or peptides are selected from pVI, H5WYG, HIV tat, R5 and GALA peptide. In some embodiments, the endosomal escape polypeptide are poly histidine chains (see e.g., Moreira, C.; Oliveira, H.; Pires, L. R.; Simões, S.; Barbosa, M. A.; Pêgo, A. P. Acta Biomater. 2009, 5,2995-3006. doi:10.1016/j.actbio.2009.04.021). Tertiary amino groups in these polymers bring protons into the endosomes producing osmotic alterations that provoke endosomal rupture (proton sponge effect). The incorporation of peptides such as the GALA (SEQ ID NO: 12) peptide (WEAALAEALAEALAEHLAEALAEALEALAA (SEQ ID NO: 13)) capable to fuse with the endosomal/liposomal membrane.

### Polynucleotides, Vectors, and Delivery Vehicles

### Engineered PNMA Encoding Polynucleotides

Described in several exemplary embodiments herein are nucleic acid constructs and polynucleotides that encode one or more of the engineered PNMA proteins. In some embodiments, the nucleic acid construct or encoding polynucleotide includes one or more regulatory elements coupled to the polynucleotide(s) encoding the one or more engineered PNMA proteins. In some embodiments, the nucleic acid construct and/or encoding polynucleotide is DNA, RNA, DNA:RNA hybrid or other nucleic acid.

In certain example embodiments, the nucleic acid construct and/or encoding polynucleotide further comprises a polynucleotide encoding a reporter polypeptide, wherein the polynucleotide encoding the reporter polypeptide is operably coupled to the engineered PNMA proteins encoding polynucleotide, optionally via linker encoding polynucleotide or direct fusion, thereby encoding an engineered PNMA proteins operably coupled to a reporter protein. In certain example embodiments, the reporter polypeptide is an optically active polypeptide, optionally a fluorescent polypeptide. In certain example embodiments, the reporter polypeptide is configured to produce a signal or a loss of a signal, optionally an optical signal, upon expression. Reporter polypeptides and other configurations of the nucleic acid construct are described in greater detail elsewhere herein.

### Codon Optimization

As described elsewhere herein, the polynucleotide encoding one or more embodiments of the engineered PNMA proteins and other polypeptides of the present disclosure described herein can be codon optimized. In some embodiments, one or more polynucleotides contained in a vector (“vector polynucleotides”) described herein that are in addition to an optionally codon optimized polynucleotide encoding embodiments of the genetic modifying system described herein can be codon optimized. Vectors are described in greater detail elsewhere herein. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The engineered PNMA protein encoding polynucleotide or other polynucleotide of the present disclosure (such as a vector polynucleotide or cargo polynucleotide) can be codon optimized for expression in a specific cell-type, tissue type, organ type, and/or subject type, such as a mammalian cell, optionally a human cell. In some embodiments, a codon optimized sequence is a sequence optimized for expression in a eukaryote, e.g., bovines (i.e., being optimized for expression in a mammalian cell, optionally a human cell), or for another eukaryote, such as another animal (e.g., a bovine, equine, canine, feline, ovine, and/or the like). Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells (including embryonic stem cells, primordial germ cells, primordial germ cell like cells, pluripotent stem cells, totipotent stem cells, blastocysts, etc.) and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a mammal, including but not limited to human, or non-human eukaryote or animal or mammal as discussed herein, e.g., a bovine, ovine, canine, feline, equine, camelid, and/or the like.

### Delivery of Polynucleotides and Polypeptides

The engineered PNMA proteins and/or encoding polynucleotides can be delivered to a cell or cell population by any suitable method, technique, and/or system. In some embodiments, the PNMA capsids are the delivery vehicle.

### Physical Delivery

In some embodiments, the engineered PNMA proteins and/or encoding polynucleotides and/or nucleic acid constructs can be introduced to cells by physical delivery methods. Examples of physical methods include microinjection, electroporation, and hydrodynamic delivery. Both nucleic acids (DNA and/or RNA) and proteins may be delivered using such methods. For example, engineered PNMA peptide may be prepared in vitro, isolated, (optionally purified if needed) and allowed to self-assemble into a capsid, and introduced to cells by a physical delivery method or technique.

### Microinjection

Microinjection of the engineered PNMA proteins and/or encoding polynucleotides and/or nucleic acid constructs, or other delivery vehicles containing the same directly to cells can achieve high efficiency, e.g., above 90% or about 100%. In some embodiments, microinjection may be performed using a microscope and a needle (e.g., with 0.5-5.0 m in diameter) to pierce a cell membrane and deliver the cargo directly to a target site within the cell. Microinjection may be used for in vitro and ex vivo delivery.

Plasmids or other nucleic acid constructs containing the engineered PNMA proteins and/or encoding polynucleotides and/or nucleic acid constructs, coding sequences for Cas or other genetic modifying system effector proteins and/or any associated polynucleotides (e.g., guide RNAs, mRNAs, and/or guide RNAs), may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and Cas or other effector protein-encoding mRNA to the cytoplasm, e.g., facilitating translation and shuttling of Cas or other effector protein to the cell nucleus. The genetic modifying systems can be used to modify a cell, such as a genome of a cell, to contain the engineered PNMA proteins and/or encoding polynucleotides and/or nucleic acid construct.

Microinjection may be used to generate genetically modified animals or cells such as those described elsewhere herein, such as those containing and/or expressing the engineered PNMA proteins and/or encoding polynucleotides and/or nucleic acid constructs of the present disclosure. For example, gene modification systems or components thereof, including a PNMA encoding polynucleotide, may be injected into zygotes, blastomeres, blastocysts, embryonic stem cells, pluripotent stem cells, induced pluripotent stem cells, primordial germ cells, primordial germ cell like-cells, and/or the like to allow for gene modification, such as to express the engineered PNMA proteins and/or encoding polynucleotides of the present invention.

### Electroporation

In some embodiments, the engineered PNMA proteins and/or encoding polynucleotides and/or nucleic acid constructs, genetic modifying systems, and/or delivery vehicles containing the same may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer the engineered PNMA proteins and/or encoding polynucleotides and/or nucleic acid constructs, and/or delivery vehicles containing the same) into cells. Electroporation may be used for in vitro and ex vivo delivery.

Electroporation may also be used to deliver the engineered PNMA proteins and/or encoding polynucleotides and/or nucleic acid constructs, and/or delivery vehicles containing the same) to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the engineered PNMA proteins and/or encoding polynucleotides and/or nucleic acid constructs, genetic modifying systems, and/or delivery vehicles containing the same, e.g., for in vivo delivery. In some examples, hydrodynamic delivery may be performed by rapidly pushing a large volume (8-10% body weight) solution containing the gene modification system into the bloodstream of a subject (e.g., a bovine). As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins.

### Transfection

The engineered PNMA proteins and/or encoding polynucleotides and/or nucleic acid constructs, and/or delivery vehicles containing the same, genetic modifying system, and/or other polypeptides and/or polynucleotides described herein, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid. Nucleic acids and vectors and vector systems that can encode a genetic modifying system and/or components thereof are described in greater detail else wherein herein. Transfection has been used to deliver nucleic acid constructs to bovine cells. See e.g., Tajik et al., Iran J Vet Res. 2017 Spring; 18(2): 113-118; Jafarnejad et al., S African J Anim Sci, Vol. 48 No. 1 (2018) DOI: 10.4314/sajas.v48i1.13; Duarte et al., Anim Biotechnol. 2020 Dec. 30; 1-11. doi: 10.1080/10495398.2020.1862137; and Osorio Gene. 2017 Aug. 30; 626:200-208, which are incorporated by reference as if expressed in their entireties herein and can be adapted for use with the present disclosure.

### Transduction

The engineered PNMA proteins and/or encoding polynucleotides and/or nucleic acid constructs, and/or delivery vehicles containing the same, genetic modifying system, and/or other polypeptides and/or polynucleotides described herein, can be introduced to cells by transduction by a viral, pseudoviral, and/or virus like particle. Methods of packaging the engineered PNMA proteins and/or encoding polynucleotides and/or nucleic acid constructs, and/or delivery vehicles containing the same, genetic modifying system, and/or other polypeptides and/or polynucleotides described herein in viral particles can be accomplished using any suitable viral vector or vector systems. Such viral vector and vector systems are described in greater detail elsewhere herein. As used in this context herein “transduction” refers to the process by which foreign nucleic acids and/or proteins are introduced to a cell (prokaryote or eukaryote) by a viral, pseudoviral, and/or virus like particle. After packaging in a viral, pseudoviral, and/or virus like particle. the viral particles can be exposed to cells (e.g., in vitro, ex vivo, or in vivo) where the viral, pseudoviral, and/or virus like particle infects the cell and delivers the engineered PNMA proteins and/or encoding polynucleotides and/or nucleic acid constructs, and/or delivery vehicles containing the same, genetic modifying system, and/or other polypeptides and/or polynucleotides described herein to the cell via transduction. Viral, pseudoviral, and/or virus like particles can be optionally concentrated prior to exposure to target cells. In some embodiments, the virus titer of a composition containing viral and/or pseudoviral particles can be obtained and a specific titer be used to transduce cells. Viral vectors and systems and generation of viral (or pseudoviral, and/or virus like particle) delivery particles is described in greater detail elsewhere herein. In some embodiments, an engineered PNMA capsid, such as a cargo loaded engineered PNMA capsid, can be capable of transduction.

### Biolistics

The engineered PNMA proteins and/or encoding polynucleotides and/or nucleic acid constructs, and/or delivery vehicles containing the same, genetic modifying system, and/or other polypeptides and/or polynucleotides described herein can be introduced to cells using a biolistic method or technique. The term of art “biolistic”, as used herein refers to the delivery of nucleic acids to cells by high-speed particle bombardment. In some embodiments, the genetic modifying systems and/or components thereof can be attached, associated with, or otherwise coupled to particles, which than can be delivered to the cell via a gene-gun (see e.g., Liang et al. 2018. Nat. Protocol. 13:413-430; Svitashev et al. 2016. Nat. Comm. 7:13274; Ortega-Escalante et al., 2019. Plant. J. 97:661-672). In some embodiments, the particles can be gold, tungsten, palladium, rhodium, platinum, or iridium particles.

### Implantable Devices

In some embodiments, the delivery system can include an implantable device that incorporates or is coated with a genetic modifying system and/or components thereof described herein. Various implantable devices are described in the art, and include any device, graft, or other composition that can be implanted into a subject, such as a human or other non-human animal.

### Delivery Vehicles

Polynucleotides and/or polypeptides of the present disclosure, such as the engineered PNMA proteins and/or encoding polynucleotides and/or nucleic acid constructs, and/or delivery vehicles containing the same, genetic modifying system, and/or other polypeptides and/or polynucleotides described herein, can be delivered (e.g., to a target cell to be modified) via one or more delivery vehicles. The delivery vehicles can deliver a cargo, such as a polynucleotide or polypeptide of the present disclosure (such as the engineered PNMA proteins and/or encoding polynucleotides and/or nucleic acid constructs, and/or delivery vehicles containing the same, genetic modifying system, and/or other polypeptides and/or polynucleotides described herein), into cells, tissues, organs, or organisms (e.g., animals or plants). In some embodiments, delivery vehicles are used to deliver a cargo, such as a polynucleotide or polypeptide of the present disclosure to a target human or other non-human animal cell. The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses (e.g., virus particles, pseudoviral particles, or virus like particles), non-viral vehicles (e.g., exosomes, liposomes, etc.), and other delivery reagents described herein and those appreciated by one of ordinary skill in the art in view of the present disclosure. It will be appreciated that the engineered PNMA proteins can self-assemble into capsids capable of packaging/capturing a cargo. As such, the engineered PNMA capsids can also be delivery vehicles in the context of polynucleotide and/or polypeptide delivery, including to deliver an engineered PNMA polypeptide or encoding polynucleotide. Engineered PNMA protein-based delivery systems methods of their use are described in greater detail elsewhere herein.

The delivery vehicles described herein can have a greatest dimension or greatest average dimension (e.g., diameter or greatest average diameter) of less than 100 microns (μm). In some embodiments, the delivery vehicles have a greatest dimension or greatest average dimension of less than 10 μm. In some embodiments, the delivery vehicles may have a greatest dimension or greatest average dimension of less than 2000 nanometers (nm). In some embodiments, the delivery vehicles may have a greatest dimension or greatest average dimension of less than 1000 nanometers (nm). In some embodiments, the delivery vehicles may have a greatest dimension or greatest average dimension (e.g., diameter or average diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension or greatest average dimension ranging between 25 nm and 200 nm.

### Particles

In some embodiments, the delivery vehicles may be or comprise particles. For example, the delivery vehicle may be or comprise nanoparticles (e.g., particles with a greatest dimension or greatest average dimension (e.g., diameter or greatest average diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

Nanoparticles may also be used to deliver the compositions and systems to cells, as described in US20130185823, WO2008042156, and WO2015089419. In general, a “nanoparticle” refers to any particle having a diameter of less than 1000 nm. In certain embodiments, nanoparticles of the invention have a greatest dimension or greatest average dimension (e.g., diameter or average diameter) of 500 nm or less. In other embodiments, nanoparticles of the invention have a greatest dimension or greatest average dimension ranging between 25 nm and 200 nm. In other embodiments, nanoparticles of the invention have a greatest dimension or greatest average dimension of 100 nm or less. In other embodiments, nanoparticles of the invention have a greatest dimension or greatest average dimensions ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured and are within the scope of the present invention. Nanoparticles with one half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (e.g., one or more components of a genetic modifying system (e.g., a CRISPR-Cas system or component(s) thereof) and can include additional carriers and/or excipients) to provide particles of an optimal size for delivery for any in vitro, ex vivo and/or in vivo application of the present disclosure. In some embodiments, particle dimension (e.g., diameter) characterization is based on measurements using dynamic laser scattering (DLS). See also e.g., U.S. Pat. Nos. 8,709,843; 6,007,845; 5,855,913; 5,985,309; 5,543,158; and Dahlman et al. Nature Nanotechnology (2014), doi:10.1038/nnano.2014.84, describes particles, methods of making and using them, and measurements thereof, which can be adapted for use with the present disclosure.

### Vectors and Vector Systems

In some embodiments the delivery vehicle is a vector or vector system or particle, such as a virus or viral like particle, produced from such a vector or vector system. As such, also provided herein are vectors that can contain one or more of the engineered PNMA protein encoding polynucleotides described herein. In certain embodiments, the vector can contain one or more polynucleotides encoding one or more elements of a genetic modifying system described herein. The vectors can be useful in producing bacterial, fungal, yeast, plant cells, animal cells, and transgenic animals that can express one or more components of the genetic modifying system described herein, and as such, contain a genetic modification, such as one or more of the engineered PNMA protein encoding polynucleotides described herein or be rendered capable of producing particles (e.g., viral or viral like particles) that can be used to deliver a genetic modifying system and/or a one or more of the engineered PNMA protein encoding polynucleotides described herein described herein to a cell, such as a human or non-human animal cell.

Within the scope of this disclosure are vectors containing one or more of the polynucleotide sequences described herein, such as those relevant to introducing one or more of the engineered PNMA protein encoding polynucleotide. One or more of the polynucleotides that are part of a genetic modifying system can be included in a vector or vector system. The vectors and/or vector systems can be used, for example, to express one or more of the polynucleotides in a cell, such as a producer cell, to produce a genetic modifying system containing virus particles described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In some embodiments, the vector can be a bicistronic vector. In some embodiments, a bicistronic vector can be used for one or more elements of the genetic modifying system described herein. In some embodiments, expression of elements of the genetic modifying system described herein can be driven by the CBh promoter or other ubiquitous promoter. Where the element of the genetic modifying system is an RNA, its expression can be driven by a Pol III promoter, such as a U6 promoter. In some embodiments, the two are combined.

### Cell-Based Vector Amplification and Expression

Vectors may be introduced and propagated in a prokaryotic cell or eukaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In some embodiments, a prokaryote is used to amplify copies of a vector and express one or more nucleic acids, such as to provide a source of one or more proteins for delivery to a host cell or host organism.

Vectors can be designed for expression of one or more elements of the genetic modifying system described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, and mammalian cells. In some embodiments, the suitable host cell is a eukaryotic cell. In some embodiments the host cell is a cell to be modified by a genetic modifying system. In some embodiments the host cell is a producer cell capable of producing particles (e.g., virus particles, virus like particles, exosomes, and/or the like) that can be used to deliver a genetic modifying system or component thereof to a cell.

In some embodiments, the suitable host cell is a suitable bacterial cell. Suitable bacterial cells include but are not limited to bacterial cells from the bacteria of the species Escherichia coli. Many suitable strains of E. coli are known in the art for expression of vectors. These include, but are not limited to Pir1, Stb12, Stb13, Stb14, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990). In some embodiments, the suitable host cell is a bovine cell, including but not limited to, bovine embryonic stem cells, bovine induced pluripotent stem cells, bovine blastocyst cells, bovine spermatogonia stem cells, bovine oogonial cells, bovine primordial germ cells, bovine primordial germ cell like cells, bovine totipotent cells, or other bovine cell described elsewhere herein.

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif), and picZ (InVitrogen Corp, San Diego, Calif). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. In some embodiments, the suitable host cell is an insect cell. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements is provided elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET lid (Studier et al., GENE EXPRESSION TECHNOLOGY: METHODS IN ENZYMOLOGY 185, Academic Press, San Diego, Calif. (1990) 60-89).

In some embodiments, one or more vectors driving expression of one or more of the engineered PNMA protein encoding polynucleotides are introduced into a host cell such that expression of the one or more of the engineered PNMA protein encoding polynucleotides occurs within the host cell.

In some embodiments, one or more vectors driving expression of one or more elements of a genetic modifying system described herein are introduced into a host cell such that expression of the elements of the delivery system described herein direct formation of a genetic modifying system complex (e.g., a CRISPR-Cas complex) at one or more target sites at on a target polynucleotide, such as in a target cell or target cell genome. For example, a CRISPR-Cas effector protein described herein and a nucleic acid component (e.g., a guide polynucleotide) can each be operably linked to separate regulatory elements on separate vectors. RNA(s) of different elements of a genetic modifying (e.g., CRISPR-Cas) system can be delivered to an animal, plant, microorganism or cell thereof to produce an animal (e.g., a mammal, such as a bovine)), that constitutively, inducibly, or conditionally expresses different elements of the genetic modifying (e.g., CRISPR-Cas) system described herein that incorporates one or more elements of the genetic modifying system (e.g., a CRISPR-Cas system) described herein or contains one or more cells that incorporates and/or expresses one or more elements of the genetic modifying (e.g., CRISPR-Cas) system described herein.

### Cell-Free Vector and Polynucleotide Expression

In some embodiments, the engineered PNMA protein encoding polynucleotide or other polynucleotide described herein can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg²⁺, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Vector Features

The vectors can include additional features that can confer one or more functionalities to the vector, the polynucleotide to be delivered, a virus or other particle (e.g., viral like particle or exosome) produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In certain embodiments, the polynucleotides and/or vectors thereof described herein (such as the engineered PNMA protein encoding polynucleotides described herein) can include one or more regulatory elements that can be operatively linked to the polynucleotide. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization or export signals). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). Exemplary promoters also include bovine U6 (bU6) and bovine 7SK (b7SK), and other bovine PolII promoters (see e.g., Lambeth et al., Anim Genet. 2006 August; 37(4):369-72), bovine papillomavirus-1 promoters (BPV-1) (Linz and Baker. J Virol. 1988 August; 62(8):2537-43. doi: 10.1128/JVI.62.8.2537-2543.1988), the bovine SIX1 gene promoter (see e.g., Wei et al. Scientific Reports volume 7, Article number: 12599 (2017)), bovine growth hormone promoter (see e.g., Jiang et al., Nuc Acid Prot Syn Mol Gen. 1999. 274(12): 7893-7900), bovine pyruvate carboxylase (see e.g., Hazelton et al. J. Dairy Sci. 91:91-99), a bidirectional promoter (see e.g., Meersserman et al. DNA Research, Volume 24, Issue 3, June 2017, Pages 221-233), a bovine Akt3 promoter (see e.g., Farmanullah et al. Journal of Genetic Engineering and Biotechnology (2021) 19:164), bovine alpha-lactalbumin promoter (see e.g., FEBS Lett. 1991 Jun. 17; 284(1):19-22), bovine beta-casein promoter (see e.g., Cerdan et al., Mol Reprod Dev. 1998 March; 49(3):236-45), any combination thereof.

In some embodiments, the regulatory sequence can be a regulatory sequence described in U.S. Pat. No. 7,776,321, U.S. Pat. Pub. No. 2011/0027239, or International Patent Publication No. WO 2011/028929, the contents of which are incorporated by reference herein in their entireties. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. As used herein, “regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g., INS, IRS2, Pdxl, Alx3, Ppy), cardiac specific promoters (e.g. Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g., FLG, K14, TGM3), immune cell specific promoters, (e.g., ITGAM, CD43 promoter, CD14 promoter, CD45 promoter, CD68 promoter), urogenital cell specific promoters (e.g., Pbsn, Upk2, Sbp, Ferl14), endothelial cell specific promoters (e.g., ENG), pluripotent and embryonic germ layer cell specific promoters (e.g., Oct4, NANOG, Synthetic Oct4, T brachyury, NES, SOX17, FOXA2, MIR122), and muscle cell specific promoter (e.g. myostatin, Desmin). Other tissue and/or cell specific promoters are generally known in the art and are within the scope of this disclosure.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. The components of a light inducible system may include one or more elements of the CRISPR-Cas system described herein, a light-responsive cytochrome heterodimer (e.g., from Arabidopsis thaliana), and a transcriptional activation/repression domain. In some embodiments, the vector can include one or more of the inducible DNA binding proteins provided in International Patent Publication No. WO 2014/018423 and U.S. Patent Publication Nos., 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-11-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters that are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments where multiple elements are to be expressed from the same vector or within the same vector system, different promoters or regulatory elements can be used for each element to be expressed to avoid or limit loss of expression due to competition between promoters and/or other regulatory elements.

In some embodiments, the polynucleotide, vector or system thereof can include one or more elements capable of translocating and/or expressing a polynucleotide to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., genome.unmc.edu/LocSigDB/and Negi et al., 2015. Database. 2015: bav003; doi: 10.1093/database/bav003), nuclear export signals (e.g., LXXXLXXLXL (SEQ ID NO: 14) and others described elsewhere herein), endoplasmic reticulum localization/retention signals (e.g., KDEL (SEQ ID NO: 15), KDXX, KKXX, KXX, and others described elsewhere herein; and see e.g., Liu et al. 2007 Mol. Biol. Cell. 18(3):1073-1082 and Gorleku et al., 2011. J. Biol. Chem. 286:39573-39584), mitochondria targeting signals (see e.g., Chin, R. M., et al, 2018, Cell Reports. 22:2818-2826, particularly at FIG. 2; Doyle et al. 2013. PLoS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430), and peroxisome targeting signals (e.g., (S/A/C)-(K/R/H)-(L/A), SLK, (R/K)-(L/V/I)-XXXXX-(H/Q)-(L/A/F)). Suitable protein targeting motifs can also be designed or identified using any suitable database or prediction tool, including but not limited to Minimotif Miner (minimotifminer.org, mitominer.mrc-mbu.cam.ac.uk/release-4.0/embodiment.do?name=Protein %20MTS), LocDB (see above), PTSs predictor, TargetP-2.0 www.cbs.dtu.dk/services/TargetP/), ChloroP (www.cbs.dtu.dk/services/ChloroP/); NetNES (www.cbs.dtu.dk/services/NetNES/), Predotar (urgi.versailles.inra.fr/predotar/), and SignalP (www.cbs.dtu.dk/services/SignalP/).

### Selectable Markers and Tags

One or more of the polynucleotides described herein, such as those of or encoding the engineered PNMA protein(s) can be operably linked, fused to, or otherwise modified to include a polynucleotide that encodes or is a selectable marker or tag, which can be a polynucleotide or polypeptide. In some embodiments, the polypeptide encoding a polypeptide selectable marker is incorporated in the genetic modifying system polynucleotide or other polynucleotide of the present disclosure such that the selectable marker polypeptide, when translated, is inserted between two amino acids between the N- and C-terminus of the genetic modifying system polypeptide (or other polypeptide of the present disclosure) or at the N- and/or C-terminus of the genetic modifying system polypeptide (or other polypeptide of the present disclosure). In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the genetic modifying system (or other polynucleotide) described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the genetic modifying system (or other polypeptide) described herein via suitable linker, such as a glycine or glycine serine linkers as short as GS or GG up to (GGGGG)₃ (SEQ ID NO: 16) or (GGGGS)₃ (SEQ ID NO: 17). Other suitable linkers are described elsewhere herein.

### Targeting Moieties

The vector or vector system (or other polynucleotide, such as the engineered PNMA protein encoding polynucleotide) can include one or more polynucleotides that are or encode one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the genetic modifying system polynucleotide(s) and/or products expressed therefrom include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated genetic modifying system polynucleotide(s) to specific cells, tissues, organs, etc. In some embodiments, the targeting moieties can target integrins on cell surfaces. Optionally, the binding affinity of the targeting moiety is in the range of 1 nM to 1 μM.

Exemplary targeting moieties that can be included are described elsewhere herein. See description related to “Targeted Delivery” and/or “Responsive Delivery” herein.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vectors described herein. nAAV vectors are discussed elsewhere herein.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. When multiple different guide polynucleotides are used, a single expression construct may be used to target nucleic acid-targeting activity to multiple different, corresponding target sequences within a cell. For example, a single vector may comprise about or more than about 1, 2, 3, 4, 5, 6, 7, 8, 9, 10, 15, 20, or more guide polynucleotides. In some embodiments, about or more than about 1, 2, 3, 4, 5, 6, 7, 8, 9, 10, or more such guide-polynucleotide-containing vectors may be provided, and optionally delivered to a cell.

Delivery vehicles, vectors, particles, nanoparticles, formulations and components thereof for expression of one or more elements of a genetic modifying system or other polynucleotides described herein are as used in the foregoing documents, such as International Patent Publication No. WO 2014/093622 and are discussed in greater detail herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as a genetic modifying system polynucleotide of the present invention, into a virus particle and producing said virus particle when used alone or with one or more other viral vectors (such as in a viral vector system). Viral vectors and systems thereof can be used for producing viral particles for delivery of and/or expression of one or more components of the genetic modifying system described herein. The viral vector can be part of a viral vector system involving multiple vectors. In some embodiments, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produce therefrom are described elsewhere herein. In some embodiments, the viral vectors are configured to produce replication incompetent viral particles for improved safety of these systems.

In certain embodiments, the virus structural component, which can be encoded by one or more polynucleotides in a viral vector or vector system, comprises one or more capsid proteins including an entire capsid. In certain embodiments, such as wherein a viral capsid comprises multiple copies of different proteins, the delivery system can provide one or more of the same protein or a mixture of such proteins. For example, AAV comprises 3 capsid proteins, VP1, VP2, and VP3, thus delivery systems of the invention can comprise one or more of VP1, and/or one or more of VP2, and/or one or more of VP3. Accordingly, the present invention is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Thus, a virus of within the family Adenoviridae is contemplated as within the invention with discussion herein as to adenovirus applicable to other family members. Target-specific AAV capsid variants can be used or selected. Non-limiting examples include capsid variants selected to bind to chronic myelogenous leukemia cells, human CD34 PBPC cells, breast cancer cells, cells of lung, heart, dermal fibroblasts, melanoma cells, stem cell, glioblastoma cells, coronary artery endothelial cells and keratinocytes. See, e.g., Buning et al, 2015, Current Opinion in Pharmacology 24, 94-104. From teachings herein and knowledge in the art as to modifications of adenovirus (see, e.g., U.S. Pat. Nos. 9,410,129, 7,344,872, 7,256,036, 6,911,199, 6,740,525; Matthews, “Capsid-Incorporation of Antigens into Adenovirus Capsid Proteins for a Vaccine Approach,” Mol Pharm, 8(1): 3-11 (2011)), as well as regarding modifications of AAV, the skilled person can readily obtain a modified adenovirus that has a large payload protein or a CRISPR-protein, despite that heretofore it was not expected that such a large protein could be provided on an adenovirus. And as to the viruses related to adenovirus mentioned herein, as well as to the viruses related to AAV mentioned elsewhere herein, the teachings herein as to modifying adenovirus and AAV, respectively, can be applied to those viruses without undue experimentation from this disclosure and the knowledge in the art.

In some embodiments, the viral vector is configured such that when the cargo is packaged the cargo(s) (e.g., one or more components of a genetic modifying system, including but not limited to a Cas effector), the engineered PNMA protein encoding polynucleotide, is external to the capsid or virus particle. In the sense that it is not inside the capsid (enveloped or encompassed with the capsid) but is externally exposed so that it can contact the target genomic DNA. In some embodiments, the viral vector is configured such that all the cargo(s) (e.g., the engineered PNMA protein encoding polynucleotide, or other polynucleotides and/or polypeptides) are contained within (or are internal to) the viral capsid after packaging.

### Split Viral Vector Systems

When the viral vector or vector system (be it a retroviral (e.g., AAV) or lentiviral vector) is designed so as to position the cargo(s) (e.g., one or more of the engineered PNMA protein encoding polynucleotide(s) and/or genetic modifying system) at the internal surface of the capsid once formed, the cargo(s) will fill most or all of internal volume of the capsid. In other embodiments, the genetic modifying effector (e.g., Cas) (or other exogenous gene or protein e.g., the engineered PNMA protein and/or encoding polynucleotide) may be modified or divided so as to occupy a less of the viral capsid internal volume. Accordingly, in certain embodiments, the genetic modifying system or component thereof or other exogenous gene or protein can be divided in two portions, which can be packaged in separate viral or viral like particles. In certain embodiments, by splitting the genetic modifying system or component thereof in two (or more) portions, space is made available to link one or more heterologous domains to one or both genetic modifying system component or other protein portions (e.g., the engineered PNMA protein). Such systems can be referred to as “split vector systems”. This split protein approach is also described elsewhere herein. When the concept is applied to a vector system, it thus describes putting pieces of the split proteins on different vectors thus reducing the payload of any one vector. This approach can facilitate delivery of systems where the total system size is close to or exceeds the packaging capacity of the vector. This is independent of any regulation of the genetic modifying system (e.g., a CRISPR-Cas) system that can be achieved with a split system or split protein design.

Split CRISPR proteins or other exogenous proteins whose encoding polynucleotides can be incorporated into the viral or other vectors described herein are set forth elsewhere herein and in documents incorporated herein by reference in further detail herein. In certain embodiments, each part of a split protein is attached to a member of a specific binding pair, and when bound with each other, the members of the specific binding pair maintain the parts of the spit protein in proximity. In certain embodiments, each part of a split protein is associated with an inducible binding pair. An inducible binding pair is one which is capable of being switched “on” or “off” by a protein or small molecule that binds to both members of the inducible binding pair. In general, according to the invention, some proteins may preferably split between domains, leaving domains intact. Where the cargo is a Cas protein, non-limiting examples of such Cas proteins include, without limitation, Cas protein, and orthologues. Non-limiting examples of split points include, with reference to SpCas9: a split position between 202A/203S; a split position between 255F/256D; a split position between 310E/311I; a split position between 534R/535K; a split position between 572E/573C; a split position between 713S/714G; a split position between 1003L/104E; a split position between 1054G/1055E; a split position between 1114N/1115S; a split position between 1152K/1153S; a split position between 1245K/1246G; or a split between 1098 and 1099. Corresponding positions in other Cas proteins can be appreciated in view of these positions made with reference to SpCas9.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the delivery of a cargo (e.g., a genetic modifying systems or other exogenous polynucleotide) can include, but are not limited to, those vectors based upon murine leukemia virus (MuLV), gibbon ape leukemia virus (GaLV), Simian immunodeficiency virus (SIV), human immunodeficiency virus (HIV), equine infections anemia (EIA), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); WO1994026877). Other exemplary retroviral vectors are described elsewhere herein.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Exemplary lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In some embodiments, an HIV-based lentiviral vector system can be used. In some embodiments, a FIV-based lentiviral vector system can be used.

In some embodiments, the lentiviral vector is an EIAV-based lentiviral vector or vector system. See e.g., Balagaan, J Gene Med 2006; 8: 275-285; Binley et al., HUMAN GENE THERAPY 23:980-991 (September 2012)), which can be modified for use with the present disclosure.

In some embodiments, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In some embodiments, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In some embodiments, the second-generation vector lacks one or more accessory virulence factors (e.g., vif, vprm, vpu, nef, and combinations thereof). Unlike the first-generation lentiviral vectors, no single second generation lentiviral vector includes all features necessary to express and package a polynucleotide into a virus particle. In some embodiments, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In some embodiments, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included upstream of the LTRs), and they can include one or more deletions in the 3′LTR to create self-inactivating (SIN) vectors having disrupted promoter/enhancer activity of the LTR. In some embodiments, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g. gag, pol, and rev) and upstream regulatory sequences (e.g. promoter(s)) to drive expression of the features present on the packaging vector, and (iii) an “envelope vector” that contains one or more envelope protein genes and upstream promoters. In certain embodiments, the third-generation lentiviral vector system can include at least two packaging vectors, with the gag-pol being present on a different vector than the rev gene.

In some embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used/and or adapted to deliver a genetic modifying system or exogenous polynucleotide of the present disclosure.

In some embodiments, the pseudotype and infectivity or tropism of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In some embodiments, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD 114) (see e.g., Hanawa et al. Molec. Ther. 2002 5 (3) 242-251), modified Sindbis virus envelope proteins (see e.g., Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124: 1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In some embodiments, the tropism of the resulting lentiviral particle can be tuned by incorporating cell targeting peptides into a lentiviral vector such that the cell targeting peptides are expressed on the surface of the resulting lentiviral particle. In some embodiments, a lentiviral vector can contain an envelope protein that is fused to a cell targeting protein (see e.g., Buchholz et al. 2015. Trends Biotechnol. 33:777-790; Bender et al. 2016. PLoS Pathog. 12 (e1005461); and Friedrich et al. 2013. Mol. Ther. 2013. 21: 849-859).

In some embodiments, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233. In these embodiments, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In some embodiments, a covalent-bond-forming protein-peptide pair can be incorporated into one or more of the lentiviral vectors described herein to conjugate a cell targeting peptide to the virus particle (see e.g., Kasaraneni et al. 2018. Sci. Reports (8) No. 10990). In some embodiments, a lentiviral vector can include an N-terminal PDZ domain of InaD protein (PDZ1) and its pentapeptide ligand (TEFCA (SEQ ID NO: 18)) from NorpA, which can conjugate the cell targeting peptide to the virus particle via a covalent bond (e.g., a disulfide bond). In some embodiments, the PDZ1 protein can be fused to an envelope protein, which can optionally be binding deficient and/or fusion competent virus envelope protein and included in a lentiviral vector. In some embodiments, the TEFCA (SEQ ID NO: 18) can be fused to a cell targeting peptide and the TEFCA-CPT fusion construct can be incorporated into the same or a different lentiviral vector as the PDZ1-envenlope protein construct. During virus production, specific interaction between the PDZ1 and TEFCA (SEQ ID NO: 18) facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Various exemplary lentiviral vectors, such as those used in the treatment of Parkinson's disease, ocular diseases, delivery to the brain, are described in e.g., US Patent Publication No. 20120295960, 20060281180, 20090007284, US20110117189; US20090017543; US20070054961, US20100317109, US20110293571; US20110293571, US20040013648, US20070025970, US20090111106, and U.S. Pat. Nos. 7,259,015, 7,303,910 and 7,351,585. Any of these systems can be used or adapted to deliver a genetic modifying system polynucleotide or other exogenous polynucleotide of the present disclosure.

In some embodiments, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (Ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

In another embodiment, the viral vector is a Cocal vesiculovirus envelope pseudotyped retroviral or lentiviral vector particles are contemplated (see, e.g., US Patent Publication No. 20120164118). Cocal virus is in the Vesiculovirus genus and is a causative agent of vesicular stomatitis in mammals, and as such vectors based on this virus can be used to deliver cells to a wide variety of animals, including insects, cattle, and horses (see e.g., Jonkers et al., Am. J. Vet. Res. 25:236-242 (1964) and Travassos da Rosa et al., Am. J. Tropical Med. & Hygiene 33:999-1006 (1984)). In some embodiments, Cocal vesiculovirus envelope pseudotyped retroviral vector particles may include for example, lentiviral, alpharetroviral, betaretroviral, gammaretroviral, deltaretroviral, and epsilonretroviral vector particles that may comprise retroviral Gag, Pol, and/or one or more accessory protein(s) and a Cocal vesiculovirus envelope protein. In certain embodiments of these embodiments, the Gag, Pol, and accessory proteins are lentiviral and/or gammaretroviral. In some embodiments, a retroviral vector can contain encoding polypeptides for one or more Cocal vesiculovirus envelope proteins such that the resulting viral or pseudoviral particles are Cocal vesiculovirus envelope pseudotyped.

### Adenoviral Vectors, Helper-Dependent Adenoviral Vectors, and Hybrid Adenoviral Vectors

In some embodiments, the vector can be an adenoviral vector. In some embodiments, the adenoviral vector can include elements such that the virus particle produced using the vector or system thereof can be any suitable serotype, such as serotype 2, 5, 8, 9, and others. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In some embodiments the vector can be a helper-dependent adenoviral vector or system thereof. These are also referred to in the art as “gutless” or “gutted” vectors and are a modified generation of adenoviral vectors (see e.g., Thrasher et al. 2006. Nature. 443:E5-7). In certain embodiments of the helper-dependent adenoviral vector system one vector (the helper) can contain all the viral genes required for replication but contains a conditional gene defect in the packaging domain. The second vector of the system can contain only the ends of the viral genome, one or more exogenous polynucleotides, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the CRISPR-Cas system polynucleotides described herein. In some embodiments, the polynucleotide to be delivered via the viral particle produced from a helper-dependent adenoviral vector or system thereof can be up to about 37 kb. Thus, in some embodiments, an adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use to deliver a polynucleotide or system of the present invention. In some embodiments, a hybrid-adenoviral vector can include one or more features of a retrovirus and/or an adeno-associated virus. In some embodiments, the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the delivery system of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the delivery system of the present invention.

### Adeno Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments the AAV can integrate into a specific site on chromosome 19 of a human cell with no observable side effects. In some embodiments, the capacity of the AAV vector, system thereof, and/or AAV particles can be up to about 4.7 kb. The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In some embodiments, the AAV capsid can contain 60 capsid proteins. In some embodiments, the ratio of VP1:VP2:VP3 in a capsid can be about 1:1:10.

In some embodiments, the AAV vector or system thereof can include one or more adenovirus helper factors or polynucleotides that can encode one or more adenovirus helper factors. Such adenovirus helper factors can include, but are not limited, ElA, E1B, E2A, E4ORF6, and VA RNAs. In some embodiments, a producing host cell line expresses one or more of the adenovirus helper factors.

The AAV vector or system thereof can be configured to produce AAV particles having a specific serotype.

AAV particles, packaging polynucleotides encoding compositions of the present disclosure may comprise or be derived from any natural or recombinant AAV serotype. According to the present disclosure, the AAV particles may utilize or be based on a serotype selected from any of the following serotypes, and variants thereof including, but not limited to, AAV1, AAV10, AAV106.1/hu.37, AAV11, AAV114.3/hu.40, AAV12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/rl 1.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAVA3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LKO1, AAV-LK02, AAVLK03, AAV-LK03, AAV-LK04, AAV-LK05, AAV-LK06, AAV-LK07, AAV-LK08, AAV-LKO9, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, BNP61 AAV, BNP62 AAV, BNP63 AAV, bovine AAV, caprine AAV, Japanese AAV 10, true type AAV (ttAAV), UPENN AAV 10, AAV-LK16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and/or AAV SM 10-8.

In some embodiments s, the AAV serotype may be, or have, a mutation in the AAV9 sequence as described by N Pulicherla et al. (Molecular Therapy 19(6):1070-1078 (2011)), such as but not limited to, AAV9.9, AAV9.11, AAV9.13, AAV9.16, AAV9.24, AAV9.45, AAV9.47, AAV9.61, AAV9.68, AAV9.84.

In some embodiments, the AAV serotype may be, or have, a sequence as described in U.S. Pat. No. 6,156,303, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the serotype may be AAVDJ or a variant thereof, such as AAVDJ8 (or AAV-DJ8), as described by Grimm et al. (Journal of Virology 82(12): 5887-5911 (2008). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772 may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In some embodiments, the AAV serotype may be, or have, a sequence as described in International Publication No. WO2015121501, such as, but not limited to, true type AAV (ttAAV) (SEQ ID NO: 2 of WO2015121501), “UPenn AAV10” (SEQ ID NO: 8 of WO2015/121501), “Japanese AAV10” (SEQ ID NO: 9 of WO2015/121501), or variants thereof.

According to the present disclosure, AAV capsid serotype selection or use may be from a variety of species. In one example, the AAV may be an avian AAV (AAAV). The AAAV serotype may be, or have, a sequence as described in U.S. Pat. No. 9,238,800, such as, but not limited to, AAAV (SEQ ID NO: 1, 2, 4, 6, 8, 10, 12, and 14 of U.S. Pat. No. 9,238,800), or variants thereof.

In one example, the AAV may be a bovine AAV (BAAV). The BAAV serotype may be, or have, a sequence as described in U.S. Pat. No. 9,193,769, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, such as, but not limited to, BAAV (SEQ ID NO: 5 and 6 of U.S. Pat. No. 7,427,396), or variants thereof.

In one example, the AAV may be a caprine AAV. The caprine AAV serotype may be, or have, a sequence as described in U.S. Pat. No. 7,427,396, such as, but not limited to, caprine AAV (SEQ ID NO: 3 of U.S. Pat. No. 7,427,396), or variants thereof.

In other examples the AAV may be engineered as a hybrid AAV from two or more parental serotypes. In one example, the AAV may be AAV2G9 which comprises sequences from AAV2 and AAV9. The AAV2G9 AAV serotype may be, or have, a sequence as described in United States Patent Publication No. US2016/0017005.

In one example, the AAV may be a serotype generated by the AAV9 capsid library with mutations in amino acids 390-627 (VP1 numbering) as described by Pulicherla et al. (Molecular Therapy 19(6):1070-1078 (2011). The serotype and corresponding nucleotide and amino acid substitutions may be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T5821), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K5281), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In one example, the AAV may be a serotype including at least one AAV capsid CD8+ T-cell epitope. As a non-limiting example, the serotype may be AAV1, AAV2 or AAV8.

In one example, the AAV may be a variant, such as PHP.A or PHP.B as described in Deverman. 2016. Nature Biotechnology. 34(2): 204-209.

AAV vector serotypes can be matched to target cell types. For example, the following exemplary cell types can be transduced by the indicated AAV serotypes among others.

In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5 or any combination thereof. One can select the AAV of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the second plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5.

A tabulation of certain AAV serotypes as to these cells can be found in Grimm, D. et al, J. Virol. 82: 5887-5911 (2008).

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the genetic modifying system polynucleotide(s)).

In some embodiments, the AAV vectors are produced in in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, an AAV vector or vector system can contain or consists essentially of one or more polynucleotides encoding one or more components of a genetic modifying system or other exogenous polynucleotide to be delivered to a cell. Specific cassette configuration for delivery of a genetic modifying system and/or other exogenous polynucleotide(s) will be appreciated by one of ordinary skill in the art in view of the description herein.

In some embodiments, one or more components of a delivery system of the present invention or other polypeptides and/or polynucleotides (e.g., an engineered PNMA protein encoding polynucleotide) are associated with Adeno Associated Virus (AAV), (e.g., an AAV comprising a polypeptide of the present disclosure as a fusion, with or without a linker, to or with an AAV capsid protein such as VP1, VP2, and/or VP3. More in particular, modifying the knowledge in the art, e.g., Rybniker et al., “Incorporation of Antigens into Viral Capsids Augments Immunogenicity of Adeno-Associated Virus Vector-Based Vaccines,” J Virol. December 2012; 86(24): 13800-13804, Lux K, et al. 2005. Green fluorescent protein-tagged adeno-associated virus particles allow the study of cytosolic and nuclear trafficking. J. Virol. 79:11776-11787, Munch R C, et al. 2012. “Displaying high-affinity ligands on adeno-associated viral vectors enables tumor cell-specific and safe gene transfer.” Mol. Ther. [Epub ahead of print.] doi:10.1038/mt.2012.186 and Warrington K H, Jr, et al. 2004. Adeno-associated virus type 2 VP2 capsid protein is nonessential and can tolerate large peptide insertions at its N terminus. J. Virol. 78:6595-6609, each incorporated herein by reference, one can obtain a modified AAV capsid as described herein. It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3). One can modify the cap gene to have expressed at a desired location a non-capsid protein advantageously a large payload protein, such as a Cas protein or other exogenous polypeptide. Likewise, these can be fusions, with the protein, e.g., large payload protein such as a Cas protein fused in a manner analogous to prior art fusions. See, e.g., US Patent Publication 20090215879; Nance et al., “Perspective on Adeno-Associated Virus Capsid Modification for Duchenne Muscular Dystrophy Gene Therapy,” Hum Gene Ther. 26(12):786-800 (2015) and documents cited therein, incorporated herein by reference. The skilled person, from this disclosure and the knowledge in the art can make and use modified AAV or AAV capsid as with other aspects of the present disclosure, and through this description herein one knows now that large payload proteins can be fused to the AAV capsid. Accordingly, the approaches described herein are also applicable to a virus in the genus Dependoparvovirus or in the family Parvoviridae, for instance, AAV, or a virus of Amdoparvovirus, e.g., Carnivore amdoparvovirus 1, a virus of Aveparvovirus, e.g., Galliform aveparvovirus 1, a virus of Bocaparvovirus, e.g., Ungulate bocaparvovirus 1, a virus of Copiparvovirus, e.g., Ungulate copiparvovirus 1, a virus of Dependoparvovirus, e.g., Adeno-associated dependoparvovirus A, a virus of Erythroparvovirus, e.g., Primate erythroparvovirus 1, a virus of Protoparvovirus, e.g., Rodent protoparvovirus 1, a virus of Tetraparvovirus, e.g., Primate tetraparvovirus 1.

In some embodiments, a polypeptide of the present disclosure is external to the capsid or virus particle, such as an AAV capsid. Although this approach is discussed in the context of AAVs, such an approach is applicable to other viral systems or viral like particle systems where capsids are formed. In these embodiments, the cargo polypeptide is not inside the capsid (enveloped or encompassed with the capsid) but is externally exposed so that it can contact the target genomic or other target DNA or RNA). In some embodiments, the cargo polypeptide is associated with the AAV VP2 domain by way of a fusion protein. In some embodiments, the association may be considered to be a modification of the VP2 domain. In some embodiments, the AAV VP2 domain may be associated (or tethered) to a cargo polypeptide (such as an engineered PNMA polypeptide or other polypeptide of the present disclosure) via a connector protein, for example using a system such as the streptavidin-biotin system. Also provided herein are polynucleotides encoding a cargo polypeptide (e.g., an engineered PNMA polypeptide or other polypeptide of the present disclosure) and associated AAV VP2 domain. In some preferred embodiments, the cargo polypeptide is fused or tethered (e.g., via linker) to the VP2 domain so that, a non-naturally occurring modified AAV having a VP2-cargo polypeptide fusion or otherwise modified capsid protein is formed. In some embodiments, where the cargo is tethered via a linker, the cargo can be distanced from the remainder of the AAV (or other viral or viral like particle). The fusion or tether can be at the N-terminus, C-terminus, or both of the capsid polypeptide. In some embodiments, an NLS and/or a linker (such as a GlySer linker) or other tether is positioned between the C-terminal end of the cargo and the N-terminal end of the capsid domain. In some embodiments, an NLS and/or a linker (such as a GlySer linker) or other tether is positioned between the N-terminal end of the cargo and the C-terminal end of the capsid domain. In some embodiments, the capsid polypeptide that is modified with a cargo polypeptide is truncated or contains a loss of one or more internal amino acids with the N- and C-terminal amino acids (e.g., the first (or last) 2-10 amino acids of the capsid domain intact. In these embodiments, the cargo polypeptide can be inserted between the intact N- and/or C-terminal amino acids via a fusion (e.g., an in-frame fusion) or linker or other tether (such as a streptavidin/biotin system or other adaptor molecule such as MS2). In some embodiments where a linker is used, the linker can be a branched linker, which can allow for more distance between the cargo polypeptide and capsid. A cargo polypeptide can be incorporated into other capsid domains of the AAV (e.g., VP1 and/or VP3) in a similar fashion as described with respect to VP2. Likewise, similar approaches (e.g., fusion or tethered) can be used to modified non-AAV capsids of other viral and viral-like delivery systems described herein.

### Herpes Simplex Viral Vectors

In some embodiments, the vector is a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g., 2009. Trobridge. Exp. Opin. Biol. Ther. 9:1427-1436, whose techniques and vectors described therein can be modified and adapted for use in the CRISPR-Cas system of the present invention. In some embodiments where an HSV vector or system thereof is utilized, the host cell can be a complementing cell. In some embodiments, HSV vector or system thereof can be capable of producing virus particles capable of delivering a polynucleotide cargo ofup to 150 kb. Thus, in some embodiments the cargo polynucleotide(s) included in the HSV-based viral vector or system thereof can sum from about 0.001 to about 150 kb. HSV-based vectors and systems thereof have been successfully used in several contexts including various models of neurologic disorders. See e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use with the present disclosure.

### Poxvirus Vectors

In some embodiments, the vector can be a poxvirus vector or system thereof. In some embodiments, the poxvirus vector can result in cytoplasmic expression of one or more cargo polynucleotides of the present disclosure (e.g., an engineered PNMA protein encoding polynucleotide). In some embodiments the capacity of a poxvirus vector or system thereof can be about 25 kb or more. In some embodiments, a poxvirus vector or system thereof can include one or more cargo polynucleotides described herein.

Virus Particle Production from Viral Vectors

### Retroviral Production

In some embodiments, one or more viral vectors and/or system thereof can be delivered to a suitable cell line for production of virus particles containing the polynucleotide of the present disclosure to a host cell, such as for producing engineered PNMA proteins and/or engineered capsids (see also, section “Engineered Cells and Animal Bioreactors”). Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g., pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., a genetic modifying system polynucleotide or other polynucleotide of the present disclosure), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ or more particles/mL.

Lentiviruses may be prepared from any lentiviral vector or vector system described herein. In one example embodiment, after cloning a polynucleotide to be delivered into a suitable lentiviral vector (which contains a lentiviral transfer plasmid backbone), HEK293FT at low passage (p=5) can be seeded in a T-75 flask to 50% confluence the day before transfection in DMEM with 10% fetal bovine serum and without antibiotics. After 20 hours, the media can be changed to OptiMEM (serum-free) media and transfection of the lentiviral vectors can done 4 hours later. Cells can be transfected with 10 μg of lentiviral transfer plasmid (pCasES10) and the appropriate packaging plasmids (e.g., 5 μg of pMD2.G (VSV-g pseudotype), and 7.5 ug of psPAX2 (gag/pol/rev/tat)). Transfection can be carried out in 4 mL OptiMEM with a cationic lipid delivery agent (50 uL Lipofectamine 2000 and 100 ul Plus reagent). After 6 hours, the media can be changed to antibiotic-free DMEM with 10% fetal bovine serum. These methods can use serum during cell culture, but serum-free methods are preferred.

Following transfection and allowing the producing cells (also referred to as packaging cells) to package and produce virus particles with packaged cargo, the lentiviral particles can be purified. In an exemplary embodiment, virus-containing supernatants can be harvested after 48 hours. Collected virus-containing supernatants can first be cleared of debris and filtered through a 0.45 um low protein binding (PVDF) filter. They can then be spun in an ultracentrifuge for 2 hours at 24,000 rpm. The resulting virus-containing pellets can be resuspended in 50 ul of DMEM overnight at 4 degrees C. They can be then aliquoted and used immediately or immediately frozen at −80 degrees C. for storage.

See also Merten et al., 2016. “Production of lentiviral vectors.” Mol. Ther. 3: 10617 for additional methods and techniques for lentiviral vector and particle production, which can be adapted for use with the present disclosure.

### AAV Particle Production

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

In general, there are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the cargo polynucleotide to be packaged and delivered by the resulting AAV particle (e.g., the genetic modifying system polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a cargo polynucleotide (e.g., the engineered PNMA protein encoding polynucleotides)) between 2 ITRs; (2) a vector that carries the AAV Rep-Cap encoding polynucleotides; and (helper polynucleotides). One of skill in the art will appreciate various methods and variations thereof that are both helper and -helper free and as well as the different advantages of each system. See also Kimur et al., 2019. Sci. Rep. 6:13601; Shin et al., Meth. Mol Biol. 2012. 798:267-284; Negrini et al., 2020. Curr. Prot. Neurosci. 93:e103; Dobrowsky et al., 2021. Curr. Op. Biomed. Eng. 20:100353 for additional methods and techniques for AAV vector and particle production, which can be adapted for use with the present disclosure.

### Non-Viral Vectors

In some embodiments, the vector is a non-viral vector or vector system. The term of art “non-viral vector” and as used herein in this context refers to molecules and/or compositions that are vectors but that are not based on one or more component of a virus or virus genome (excluding any nucleotide to be delivered and/or expressed by the non-viral vector) that can be capable of incorporating cargo polynucleotide(s) and delivering said cargo polynucleotide(s) (e.g., an engineered PNMA protein encoding polynucleotide) to a cell and/or expressing the polynucleotide in the cell. It will be appreciated that this does not exclude vectors containing a polynucleotide designed to target a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors can include, without limitation, naked polynucleotides and polynucleotide (non-viral) based vector and vector systems.

### Naked Polynucleotides

In some embodiments, one or more polynucleotides of the present disclosure described elsewhere herein (e.g., an engineered PNMA protein encoding polynucleotide) are included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the cargo polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the cargo polynucleotide(s). In some embodiments, the naked polynucleotide can contain other nucleic acids and/or polynucleotides in addition to the cargo polynucleotide(s). The naked polynucleotides can include one or more elements of a transposon system. Transposons and system thereof are described in greater detail elsewhere herein.

### Non-Viral Polynucleotide Vectors

In some embodiments, one or more of the polynucleotides of the present disclosure herein (e.g., an engineered PNMA protein encoding polynucleotide) are included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g. minicircles, minivectors, miniknots), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In certain embodiments, the S/MAR sequence is located downstream of an actively transcribed polynucleotide (e.g., one or more cargo polynucleotides) included in the non-viral polynucleotide vector. In some embodiments, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the cargo polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the cargo polynucleotide(s) of the present invention) and integrate it into one or more positions in the host cell's genome. In some embodiments the transposon vector or system thereof can be configured as a gene trap. In some embodiments, the TIRs can be configured to flank a strong splice acceptor site followed by a reporter and/or other gene (e.g., one or more of the cargo polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include without limitation Sleeping Beauty transposon system (Tcl/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tcl/mariner superfamily) (see e.g., Miskey et al. 2003 Nucleic Acid Res. 31(23):6873-6881) and variants thereof.

### Non-Vector Delivery Vehicles

The delivery vehicles may comprise non-vector vehicles. In general, methods and vehicles capable of delivering nucleic acids and/or proteins may be used for delivering the systems compositions herein. Examples of non-vector vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, other inorganic nanoparticles, and self-assembling viral like particles (such as the engineered PNMA capsids of the present invention).

### Lipid Particles

The delivery vehicles can include or be composed of lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples, LNPs may be used for delivering DNA molecules (e.g., those comprising coding sequences of a polypeptide of the present disclosure (e.g., a PNMA encoding polynucleotide)) and/or RNA molecules (e.g., mRNA of encoding a polypeptide of a present disclosure (e.g., a PNMA encoding polynucleotide) and/or other RNA cargos such as gRNAs). In certain cases, LNPs may be use for delivering RNP complexes of e.g., Cas/gRNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol) 2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

In some embodiments, an LNP delivery vehicle can be used to deliver a virus particle containing cargo polypeptides or polynucleotides. In some embodiments, the virus particle(s) can be adsorbed to the lipid particle, such as through electrostatic interactions, and/or can be attached to the liposomes via a linker.

In some embodiments, the LNP contains a nucleic acid, wherein the charge ratio of nucleic acid backbone phosphates to cationic lipid nitrogen atoms is about 1:1.5-7 or about 1:4.

In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxyethylglucose (HEG) based polymers, polyhydroxyethyl starch (polyHES) and polypropylene. In some embodiments, the PEG, HEG, polyHES, and a polypropylene weight between about 500 to 10,000 Da or between about 2000 to 5000 Da. In some embodiments, the shielding compound is PEG2000 or PEG5000.

In some embodiments, the LNP can include one or more helper lipids. In some embodiments, the helper lipid can be a phosphor lipid or a steroid. In some embodiments, the helper lipid is between about 20 mol % to 80 mol % of the total lipid content of the composition. In some embodiments, the helper lipid component is between about 35 mol % to 65 mol % of the total lipid content of the LNP. In some embodiments, the LNP includes lipids at 50 mol % and the helper lipid at 50 mol % of the total lipid content of the LNP.

Other non-limiting, exemplary LNP delivery vehicles are described in U.S. Patent Publication Nos. US 20160174546, US 20140301951, US 20150105538, US 20150250725, Wang et al., J. Control Release, 2017 Jan. 31. pii: 50168-3659(17) 30038-X. doi: 10.1016/j.jconrel.2017.01.037; Altmoǧlu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthc Mater., 3(9):1398-403, September 2014; and Wang et al., Agnew Chem Int Ed Engl., 53(11):2893-8, Mar. 10, 2014; James E. Dahlman and Carmen Barnes et al. Nature Nanotechnology (2014) published online 11 May 2014, doi:10.1038/nnano.2014.84; Coelho et al., N Engl J Med 2013; 369:819-29; Aleku et al., Cancer Res., 68(23): 9788-98 (Dec. 1, 2008), Strumberg et al., Int. J. Clin. Pharmacol. Ther., 50(1): 76-8 (January 2012), Schultheis et al., J. Clin. Oncol., 32(36): 4141-48 (Dec. 20, 2014), and Fehring et al., Mol. Ther., 22(4): 811-20 (Apr. 22, 2014); Novobrantseva, Molecular Therapy-Nucleic Acids (2012) 1, e4; doi:10.1038/mtna.2011.3; WO2012135025; US 20140348900; US 20140328759; US 20140308304; WO 2005/105152; WO 2006/069782; WO 2007/121947; US 2015/082080; US 20120251618; 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316.

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In some embodiments, a liposome delivery vehicle can be used to deliver a virus particle or other particle, such as an engineered PNMA capsid, containing polypeptide(s) and/or polynucleotide(s). In some embodiments, the virus particle(s) can be adsorbed to the liposome, such as through electrostatic interactions, and/or can be attached to the liposomes via a linker.

In some embodiments, the liposome can be a Trojan Horse liposome (also known in the art as Molecular Trojan Horses), see e.g., cshprotocols.cshlp.org/content/2010/4/pdb.prot5407.long, the teachings of which can be applied and/or adapted to generated and/or deliver the genetic modifying systems and/or other cargo polypeptides or polynucleotides described herein.

Other non-limiting, exemplary liposomes can be those as set forth in Wang et al., ACS Synthetic Biology, 1, 403-07 (2012); Wang et al., PNAS, 113(11) 2868-2873 (2016); Spuch and Navarro, Journal of Drug Delivery, vol. 2011, Article ID 469679, 12 pages, 2011. doi:10.1155/2011/469679; WO 2008/042973; U.S. Pat. No. 8,071,082; WO 2014/186366; 20160257951; US 20160129120; US 20160244761; US 20120251618; WO 2013/093648; Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Nucleic-Acid-Lipid Particles (SNALPs)

In some embodiments, the lipid particles contain or are composed entirely of stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol) 2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMAo).

Other non-limiting, exemplary SNALPs that can be used to deliver the polypeptides and/or polynucleotides of the present disclosure described herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375: 1896-905; Judge, J. Clin. Invest. 119:661-673 (2009); and Semple et al., Nature Niotechnology, Volume 28 Number 2 Feb. 2010, pp. 172-177.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

In some embodiments, the delivery vehicle can be or include a lipidoid, such as any of those set forth in, for example, US 20110293703.

In some embodiments, the delivery vehicle can be or include an amino lipid, such as any of those set forth in, for example, Jayaraman, Angew. Chem. Int. Ed. 2012, 51, 8529-8533.

In some embodiments, the delivery vehicle can be or include a lipid envelope, such as any of those set forth in, for example, Korman et al., 2011. Nat. Biotech. 29:154-157.

### Lipoplexes Polyplexes

In some embodiments, the delivery vehicles contain or be composed entirely of lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2 [symbol] (e.g., forming DNA/Ca²⁺ microcomplexes), polyethenimine (PEI) (e.g., branched PEI), and poly(L-lysine) (PLL).

### Sugar-Based Particles

In some embodiments, the delivery vehicle can be a sugar-based particle. In some embodiments, the sugar-based particles can be or include GalNAc, such as any of those described in WO2014118272; US 20020150626; Nair, J K et al., 2014, Journal of the American Chemical Society 136 (49), 16958-16961; Ostergaard et al., Bioconjugate Chem., 2015, 26 (8), pp 1451-1455.

### Cell Penetrating Peptides

In some embodiments, the delivery vehicles contain or are composed entirely of cell penetrating peptides (CPPs). CPPs are short peptides that facilitate cellular uptake of various molecular cargo (e.g., from nanosized particles to small chemical molecules and large fragments of DNA or RNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs. Other exemplary CPPs include NGR and LAH4.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles contain or are composed entirely of DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequences to be partially complementary to the gRNA within the Cas:gRNA ribonucleoprotein complex. A DNA nanoclew may be coated, e.g., coated with PEI to induce endosomal escape.

### Metal Nanoparticles

In some embodiments, the delivery vehicles contain or are composed entirely of metal nanoparticles. In some embodiments, the delivery vehicles contain or are composed entirely of gold nanoparticles (also referred to AuNPs or colloidal gold). Gold nanoparticles may form complex with cargos, e.g., polynucleotides and/or polypeptides, and/or complexes thereof of the present disclosure. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal nanoparticles include, without limitation, tungsten, palladium, rhodium, platinum, and iridium particles. Other non-limiting, exemplary metal nanoparticles suitable for delivery vehicles are described in US 20100129793.

iTOP

In some embodiments, the delivery vehicles contain or are composed entirely of iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles contain or are composed entirely of polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids (siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data, doi:10.13140/RG.2.2.23912.16642. Other exemplary and non-limiting polymeric particles suitable for delivery vehicles are described in US 20170079916, US 20160367686, US 20110212179, US 20130302401, 6,007,845, 5,855,913, 5,985,309, 5,543,158, WO2012135025, US 20130252281, US 20130245107, US 20130244279; US 20050019923, 20080267903.

### Streptolysin O (SLO)

The delivery vehicles can contain or be composed entirely of streptolysin 0 (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles can contain or be composed entirely of multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles can contain or be composed entirely of lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos, e.g., polypeptides and/or polynucleotides of the present disclosure. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles can contain or be composed entirely of inorganic nanoparticles. Examples of inorganic nanoparticles include carbon nanotubes (CNTs) (e.g., as described in Bates K and Kostarelos K. (2013). Adv Drug Deliv Rev 65:2023-33), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles can contain or be composed entirely of exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome forms a complex (e.g., by binding directly or indirectly) to one or more components of the cargo (e.g., polynucleotides and/or polypeptides of the present disclosure). In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/dObm00427h.

Other non-limiting, exemplary exosomes include any of those set forth in Alvarez-Erviti et al. 2011, Nat Biotechnol 29: 341; El-Andaloussi et al. (Nature Protocols 7:2112-2126(2012); and Wahlgren et al. (Nucleic Acids Research, 2012, Vol. 40, No. 17 e130).

### Spherical Nucleic Acids (SNAs)

Spherical nucleic acids (SNA) are three-dimensional arrangements of nucleic acids, with densely packed and radially arranged oligonucleotides on a central nanoparticle core. In its simplest form the SNA is composed of oligonucleotides and a core. In some embodiments, the delivery vehicle can contain or be composed entirely of SNAs. SNAs are three dimensional nanostructures that can be composed of densely functionalized and highly oriented nucleic acids that can be covalently attached to the surface of spherical nanoparticle cores. The core may be a hollow core which is produced by a 3-dimensional arrangement of molecules which form the outer boundary of the core. For instance, the molecules may be in the form of a lipid layer or bilayer which has a hollow center. In other embodiments, the molecules may be in the form of lipids, such as amphipathic lipids, i.e., sterols which are linked to an end the oligonucleotide. Sterols such as cholesterol linked to an end of an oligonucleotide may associate with one another and form the outer edge of a hollow core with the oligonucleotides radiating outward from the core. The core may also be a solid or semi-solid core.

The oligonucleotides to be delivered can be associated with the core of an SNP. An oligonucleotide that is associated with the core may be covalently linked to the core or non-covalently linked to the core, i.e., potentially through hydrophobic interactions. For instance, when a sterol forms the outer edge of the core an oligonucleotide may be covalently linked to the sterol directly or indirectly. When a lipid layer forms the core, the oligonucleotide may be covalently linked to the lipid or may be non-covalently linked to the lipids e.g., by interactions with the oligonucleotide or a molecule such as a cholesterol attached to the oligonucleotide directly or indirectly through a linker.

A spherical nucleic acid (SNA) can be functionalized in order to attach a polynucleotide. Alternatively or additionally, the polynucleotide can be functionalized. One mechanism for functionalization is the alkanethiol method, whereby oligonucleotides are functionalized with alkanethiols at their 3′ or 5′ termini prior to attachment to gold nanoparticles or nanoparticles comprising other metals, semiconductors or magnetic materials. Such methods are described, for example Whitesides, Proceedings of the Robert A. Welch Foundation 39th Conference on Chemical Research Nanophase Chemistry, Houston, Tex., pages 109-121 (1995), and Mucic et al. Chem. Commun. 555-557 (1996). Oligonucleotides can also be attached to nanoparticles using other functional groups such as phosophorothioate groups, as described in and incorporated by reference from U.S. Pat. No. 5,472,881, or substituted alkylsiloxanes, as described in and incorporated by reference from Burwell, Chemical Technology, 4, 370-377 (1974) and Matteucci and Caruthers, J. Am. Chem. Soc., 103, 3185-3191 (1981). In some instances, oligonucleotides are attached to nanoparticles by terminating the polynucleotide with a 5′ or 3′ thionucleoside. In other instances, an aging process is used to attach oligonucleotides to nanoparticles as described in and incorporated by reference from U.S. Pat. Nos. 6,361,944, 6,506,569, 6,767,702 and 6,750,016 and PCT Publication Nos. WO 1998/004740, WO 2001/000876, WO 2001/051665 and WO 2001/073123. In some embodiments, the core is a metal core. In some embodiments, the core is an inorganic metal core. In some embodiments, the core is a gold core.

In some instances, the oligonucleotide is attached or inserted in the SNA. A spacer sequence can be included between the attachment site and the oligonucleotide. In some embodiments, a spacer sequence comprises or consists of an oligonucleotide, a peptide, a polymer or an oligoethylene glycol. In a preferred embodiment, the spacer is oligoethylene glycol and more preferably, hexaethyleneglycol.

Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., U.S. Pat. App. Pub. US20210002640 and US20200188521.

### Self-Assembling Nanoparticles

In some embodiments, the delivery vehicle contains or is composed entirely of a self-assembling nanoparticle. The self-assembling nanoparticles can contain one or more polymers. The self-assembling nanoparticles can be PEGylated. Self-assembling nanoparticles are known in the art. Non-limiting, exemplary self-assembling nanoparticles can any as set forth in Schiffelers et al., Nucleic Acids Research, 2004, Vol. 32, No. 19, Bartlett et al. (PNAS, Sep. 25, 2007, vol. 104, no. 39; Davis et al., Nature, Vol 464, 15 Apr. 2010.

### Supercharged Proteins

In some embodiments, the delivery vehicle contains or is composed entirely of supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112.

### Targeted Delivery

In some embodiments, the delivery vehicle is configured for targeted delivery of cargo(s) (e.g., (e.g., the engineered PNMA polypeptides and/or encoding polynucleotides, and/or other polypeptides and/or polynucleotides, vectors, and/or the like of the present disclosure)) to a specific cell, tissue, organ, or system. In such embodiments, the delivery vehicle can include one or more targeting moieties that can direct targeted delivery of the cargo(s). In an embodiment, the delivery vehicle comprises a targeting moiety, such as on its surface. Exemplary targeting moieties include, without limitation, small molecule, polypeptide, and/or polynucleotide ligands for cell surface molecules, antibodies, affibodies, aptamers, or any combination thereof. In some embodiments, a targeted delivery vehicle can be generated by coupling, conjugating, attaching, or otherwise associating a targeting moiety with a delivery vehicle described elsewhere herein. In some embodiments, multiple targeting moieties with different targets are coupled to a delivery vehicle. In some embodiments a multivalent approach can be employed. Multivalent presentation of targeting moieties (e.g., antibodies) can also increase the uptake and signaling properties of targeting moiety fragments. In some embodiments, targeted delivery can be to one cell type or to multiple cell types. Methods of coupling conjugating, attaching, or otherwise associating a targeting moiety with a delivery vehicle are generally known in the art.

In some embodiments, the targeting moiety is an aptamer. Aptamers are ssDNA or RNA oligonucleotides that impart high affinity and specific recognition of the target molecules by electrostatic interactions, hydrogen bonding and hydrophobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets.

Targeted delivery includes intracellular delivery. Delivery vehicles that utilize the endocytic pathway are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH can be included in the delivery vehicle. Such lipids or peptides can include amines, which are protonated at an acidic pH and cause endosomal swelling and rupture by a buffer effect, pore-forming protein listeriolysin 0, histidine-rich peptides have the ability to fuse with the endosomal membrane, resulting in pore formation, and can buffer the proton pump causing membrane lysis, and/or unsaturated dioleoylphosphatidylethanolamine (DOPE) that readily adopt an inverted hexagonal shape at a low pH and causes fusion of liposomes to the endosomal membrane. Inclusion of such molecules can result in an efficient endosomal release and/or may provide an endosomal escape mechanism to increase cargo delivery by the vehicle.

In some embodiments, the delivery vehicle is or includes modified CPP(s) that can facilitate intracellular delivery via macropinocytosis followed by endosomal escape. CPPs are described in greater detail elsewhere herein.

In some embodiments, targeted delivery is organelle-specific targeted delivery. A delivery vehicle can be surface-functionalized with a targeting moiety that can direct organelle specific delivery, such as a nuclear localization sequence, ribosomal entry sequence, mitochondria specific moiety, and/or the like. The invention further comprehends a lipid entity of the invention targeting the nucleus, e.g., via a DNA-intercalating moiety.

In some embodiments, the targeted delivery is multifunctional targeted delivery that can be accomplished by attaching more than one targeting moiety to the surface of the delivery vehicle. In some embodiments, such an enhances accumulation in a desired site and/or promotes organelle-specific delivery and/or target a particular type of cell and/or respond to the local environmental stimuli such as temperature (e.g., elevated), pH (e.g., acidic or basic), respond to targeted or localized externally applied stimuli such as a magnetic field, light, energy, heat or ultrasound (e.g., responsive delivery, which is described in greater detail elsewhere herein) and/or promote intracellular delivery of the cargo.

Exemplary targeting moieties are generally known in the art, and include without limitation, those described in e.g., in e.g., Deshpande et al, “Current trends in the use of liposomes for tumor targeting,” Nanomedicine (Lond). 8(9), doi:10.2217/nnm.13.118 (2013), International Patent Publication No. WO 2016/027264, Lorenzer et al, “Going beyond the liver: Progress and challenges of targeted delivery of siRNA therapeutics,” Journal of Controlled Release, 203: 1-15 (2015); Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8):1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao J et al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), Zhao et al., 2020. Cell 181:151-167, particularly at tables 1-5; Liu et al., Front. Bioeng. Biotechnol. 2021. 9:701504. doi: 10.3389/fbioe.2021.701504; US20210379192 (describes exemplary skeletal muscle cell targeting moieties), Snow-Lisy et al., Drug. Deliv. Transl. Res. 1:351(2011); US20060263336 (describes exemplary progenitor cell targeting moieties) each of which and the documents cited therein are hereby incorporated herein by reference.

Other exemplary targeting moieties are described elsewhere herein, such as epitope tags, reporter and selectable markers, and/or the like which can be configured for and/or operate in some embodiments as targeting moieties.

### Responsive Delivery

In some embodiments, the delivery vehicle can allow for responsive delivery of the cargo(s) (e.g., the engineered PNMA polypeptides and/or encoding polynucleotides, and/or other polypeptides and/or polynucleotides, vectors and/or the like of the present disclosure). Responsive delivery, as used in this context herein, refers to delivery of cargo(s) by the delivery vehicle in response to an external stimuli. Examples of suitable stimuli include, without limitation, an energy (light, heat, cold, and the like), a chemical stimuli (e.g., chemical composition, etc.), and a biologic or physiologic stimuli (e.g., environmental pH, osmolarity, salinity, biologic molecule, etc.). In some embodiments, a targeting moiety is responsive to an external stimuli and facilitate responsive delivery. In other embodiments, responsiveness is determined by a non-targeting moiety component of the delivery vehicle.

In some embodiments, the responsive delivery is stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)).

In some embodiments, the responsive delivery is temperature-triggered delivery. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes.

In some embodiments, the responsive delivery is redox-triggered delivery. The difference in redox potential between normal and inflamed or tumor tissues, and between the intra- and extra-cellular environments has been exploited for delivery, e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment.

Enzymes can also be used as a trigger to release payload. Enzymes, including MMPs (e.g., MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln (SEQ ID NO: 19)) can be incorporated into a linker, and can have antibody targeting, e.g., antibody 2C5.

In some embodiments, the responsive delivery is light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or γ-Fe2O3, e.g., those that are less than 10 nm in size. Targeted delivery can be then by exposure to a magnetic field.

Responsive delivery to the testis has been described. See e.g., He et al., 2015. Oncol. Rep. 34(5)-2318 (describes ultrasound microbubble-mediated delivery to the testis); Li et al., Curr. Drug. Deliv. 2020 17(5):438-446 (describes heat stress and pulsed unfocused ultrasound delivery into testicular seminiferous tubules), which can be adapted for use with the present disclosure to provide responsive delivery to the testis or testicular cell.

### Cells and Organisms

Described herein are engineered cells that are engineered to express and/or optionally secrete one or more of the engineered PNMA polypeptides, engineered PNMA capsids (including cargo loaded capsids) and/or encoding polynucleotides described herein. In some embodiments, engineered cells can be administered as part of a therapy to a subject in need thereof. In some embodiments, the engineered cells are used to produce the engineered PNMA polypeptides and/or encoding polynucleotides described herein for subsequent harvesting, engineered capsid formation and/or loading, and administration. In some embodiments, the engineered cells are part of an in vitro production system for producing the engineered PNMA polypeptides, engineered PNMA capsids (including cargo loaded capsids) and/or encoding polynucleotides described herein. In some embodiments, engineered non-human animals or plants are generated that express he engineered PNMA polypeptides, engineered PNMA capsids (including cargo loaded capsids) and/or encoding polynucleotides described herein, such as in a biological fluid or harvestable plant part, are used as bioreactors for the production of the engineered PNMA polypeptides, engineered PNMA capsids (including cargo loaded capsids) and/or encoding polynucleotides described herein.

In general, an engineered PNMA protein encoding polynucleotide is expressed, such as via a vector or vector system, or is otherwise integrated into the genome of the cell, such that the engineered PNMA proteins are expressed in the cell, engineered PNMA capsids are formed, and/or are optionally secreted. Methods and techniques of modifying animal and plant cells are generally known in the art and can be used to engineer the cells to express and optionally secrete the engineered PNMA proteins and/or capsids of the present disclosure.

### Cells for Cell-Based Therapies

In some embodiments, engineered cells can be administered as part of a therapy to a subject in need thereof. In some embodiments, the cells are allogeneic to a subject in which they are delivered. In some embodiments, the cells are autologous to a subject in which they are delivered. In some embodiments, the cells are engineered ex vivo to express and/or secrete one or more of the engineered PNMA proteins and/or capsids (loaded with a cargo or not) of the present disclosure prior to optional cargo loading/capture and/or administration to a subject in need thereof. In some embodiments, the cells can be administered to a subject in need thereof and provide production of the engineered PNMA proteins and/or capsids of the present disclosure within the subject in need thereof.

The use of autologous cells can minimize graft-versus-host disease (GVHID) issues. In some embodiments, allogenic cells are modified to reduce alloreactivity and prevent graft-versus-host disease. This approach can have the advantage as being able to provide “off the shelf” solutions and cells for cell-based therapies and delivery of the engineered PNMA polypeptides and/or capsids and provide a scalable alternative to autologous approaches. Platforms for manufacturing allogenic and autologous cells (including modified cells) are generally known in the art. See e.g., Abbasalizadeh Expert Opin Biol Ther. 2017 October; 17(10):1201-1219; Pigeau et al., Front Med (Lausanne). 2018 Aug. 22; 5:233; Abraham et al., Adv Biochem Eng Biotechnol. 2018; 165:323-350. Exemplary modifications that can be introduced allogenic cells to reduce complications to alloreactivity are known in the art. See e.g., Perez et al., Front Immunol. 2020 Nov. 11; 11:583716. doi: 10.3389.

In some embodiments, the engineered cells for delivery of the engineered PNMA polypeptides and/or capsids can be engineered to include a transgenic safety switch, in the form of a transgene that renders the cells vulnerable to exposure to a specific signal. For example, in some embodiments, the cells can be engineered with a thymidine kinase (TK) gene that, in response to a nucleoside prodrug (e.g., ganciclovir or acyclovir), causes cell death (see e.g., Greco, et al., Improving the safety of cell therapy with the TK-suicide gene. Front. Pharmacol. 2015; 6: 95). Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In the instance of allogenic cells, the allogenic cells can be engineered to be resistant to immunosuppressive agents (e.g., calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite), which are often given to subjects receiving an allogenic therapy. In some embodiments, immunosuppressive resistance is conferred to the allogeneic cells by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

The engineered cells can be any suitable cell type. The engineered cells can be cultured and expanded as desired prior to delivery to the subject. Collection, isolation, selection, culture, expansion, and storage methods for the engineered cells are generally known in the art.

### Engineered Cells and Animal Bioreactors

Described in several exemplary embodiments herein are engineered cells and non-human animal organisms that can be used to produce the engineered PNMA proteins, engineered PNMA capsids, including cargo loaded PNMA capsids. In some embodiments, the engineered cells can be used in the context of a cell-based therapy in which the cells are engineered to express the engineered PNMA capsids and/or cargo loaded engineered PNMA capsids and can be delivered to a subject in need thereof. In some embodiments, the engineered cells are used in an in vitro production system for generating the engineered PNMA proteins and/or engineered PNMA capsids (including cargo loaded capsids).

In general, an engineered PNMA polypeptide encoding polynucleotide, and optionally a cargo, is expressed, such as via a vector or vector system, or is otherwise integrated into the genome of the cell, such that the engineered PNMA polypeptide is produced in the cell where it can be harvested (such as for use in vitro capsid formation) or optionally self-assemble into a capsid before or after being optionally secreted by a cell.

Plants and non-human animal organisms can also be generated with engineered cells to produce the engineered PNMA polypeptide, engineered PNMA capsids (including cargo loaded engineered PNMA capsids. Such compositions can be harvested and subsequently delivered to a subject in need thereof.

### Engineered Cells and Organisms for Engineered PNMA Protein and Capsid Production

In some embodiments, the cells can be engineered to express and produce the engineered PNMA polypeptides and/or engineered PNMA capsids (including cargo loaded capsids) for subsequent harvesting and delivery to a subject in need thereof. Various cell and organism-based bioreactors for recombinant protein production are generally known in the art and can be employed for production of the engineered PNMA polypeptides and/or engineered PNMA capsids (including cargo loaded capsids) of the present invention. Exemplary cells and organism are discussed below.

### Plants

In some embodiments, the engineered organism is a plant or algae. Plants have been engineered for the production of therapeutics, such as therapeutic proteins. Plant-based biofactories are an established production system with the benefits of cost-effectiveness, high scalability, rapid production, enabling post-translational modification, and being devoid of harmful pathogens contamination. See e.g., Sethi et al., Mol Biotechnol. 2021 December; 63(12):1125-1137; Yusibov et al., Annu Rev Plant Biol. 2016 Apr. 29; 67:669-701; Huang and McDonald. Biotechnol Adv. 2012 March-April; 30(2):398-409; Yang et al., Biomolecules. 2021 Jan. 13; 11(1):93; Vitale et al., Mol Interv. 2005 August; 5(4):216-25. doi: 10.1124/mi.5.4.5; Singh et al., Curr Mol Biol Rep. 2017; 3(4):306-316; Decker et al., Curr Opin Plant Biol. 2004 April; 7(2):166-70; Fischer et al., Curr Opin Plant Biol. 2004 April; 7(2):152-8; Yemets et al., Cell Biol Int. 2014 September; 38(9):989-1002; Daniell et al., Trends Plant Sci. 2009 December; 14(12):669-79, which are incorporated herein by reference and can be adapted for use with the present disclosure.

The engineered plants (and plant cells) include, but are not limited to, monocotyledonous and dicotyledonous plants (and plant cells), such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Engineered plant cells and tissues include, without limitation, roots, stems, leaves, flowers, and reproductive structures, undifferentiated meristematic cells, parenchyma, collenchyma, sclerenchyma, xylem, phloem, epidermis, and germplasm. In some embodiments, the dicotyledonous plants belong to the order Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, or Asterales. In some embodiments, the monocotyledonous plants (or plant cells) belong to the order Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or belonging to Gymnospermae, e.g those belonging to the orders Pinales, Ginkgoales, Cycadales, Araucariales, Cupressales and Gnetales.

In some embodiments, the engineered plant is a dicot, monocot, or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, or Pseudotsuga.

In some embodiments, the engineered cells are “algae” or “algae cells”; including for example algae selected from several eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). The term “algae” includes for example algae selected from: Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

Also encompassed herein are gametes, seeds, germplasm, embryos, either zygotic or somatic, progeny or hybrids of plants comprising the recombinant SCNB1 peptides and/or encoding polynucleotides, which are produced by traditional breeding methods.

### Bacterial Cells

In some embodiments, the engineered cells are bacterial cells. Suitable bacterial cell systems for recombinant protein production are generally known in the art See e.g., Baeshen et al., Microb Cell Fact. 2014 Oct. 2; 13:141, Mergulhão et al., Biotechnol Adv. 2005 May; 23(3):177-202. doi: 10.1016/j.biotechadv.2004.11.003; Sørensen and Mortensen. J Biotechnol. 2005 Jan. 26; 115(2):113-28; Porowinska et al., Postepy Hig Med Dosw (Online). 2013 Mar. 1; 67:119-29, which are incorporated herein by reference and can be adapted for use with the present disclosure.

### Fungal and Insect Cells

In some embodiments, the engineered cells are fungal or insect cells. Suitable fungal and insect cell systems for recombinant protein production are generally known in the art. See e.g., Mattanovich et al., Methods Mol Biol. 2012; 824:329-58, Ahmad et al., et al., Appl Microbiol Biotechnol. 2014 June; 98(12):5301-17; Vandermies and Fickers. Microorganisms. 2019 Jan. 30; 7(2):40; Ferrer-Miralles et al., Methods Mol Biol. 2015; 1258:1-24; Yang and Zhang. Biotechnol Adv. 2018 January-February; 36(1):182-195; Karbalaei et al., J Cell Physiol. 2020 September; 235(9):5867-5881; Baghban et al., Mol Biotechnol. 2019 May; 61(5):365-384; Manfraõ-Netto et al., Front Bioeng Biotechnol. 2019 May 1; 7:94; Baeshen et al., Microb Cell Fact. 2014 Oct. 2; 13:141, Chambers et al., Curr Protoc Protein Sci. 2018 Feb. 21; 91:5.4.1-5.4.6; Irons et al., Curr Protoc Protein Sci. 2018 Feb. 21; 91:5.5.1-5.5.22, which are incorporated herein by reference and can be adapted for use with the present disclosure.

### Animal Cells and Non-Human Animals

In some embodiments, the engineered cells are animal cells, optionally mammalian cells. Suitable mammalian cells and systems for recombinant protein expression and production are generally known in the art. See e.g., O'Flaherty R. Biotechnol Adv. 2020 Nov. 1; 43:107552; Wurm. Nat Biotechnol. 2004 November; 22(11):1393-8; Li et al., MAbs. 2010 September-October; 2(5):466-79; Dyson et al., Adv Exp Med Biol. 2016; 896:217-24; Panavas et al., Methods Mol Biol. 2009; 497:303-17; Chevallier et al., Biotechnol Bioeng. 2020 April; 117(4):1172-1186; Dumont et al., Crit Rev Biotechnol. 2016 December; 36(6):1110-1122; Hacker and Balasubramanian. Curr Opin Struct Biol. 2016 June; 38:129-36; Bielser et al., Biotechnol Adv. 2018 July-August; 36(4):1328-1340; Lalonde and Durocher et al., J Biotechnol. 2017 Jun. 10; 251:128-140, which are incorporated herein by reference and can be adapted for use with the present disclosure.

In some embodiments, the engineered organism bioreactor is a non-human animal, such as a swine, bovine, ovine, caprine, camelid, equine, avian and/or the like. In some embodiments, a non-human animal bioreactor is used to produce the engineered PNMA polypeptides and/or engineered PNMA capsids (including cargo loaded capsids). See e.g., Houdebine et al., Transgenic Res. 2000; 9 (4-5):305-20; Demain and Vaishnav. Biotechnol Adv. 2009 May-June; 27(3):297-306; Woodfint et al., Mol Biotechnol. 2018 December; 60(12):975-983; Lubon et al., Transfus Med Rev. 1996 April; 10(2):131-43; Redwan el-R M. J Immunoassay Immunochem. 2009; 30(3):262-90; Lubon, H. Biotechnol Annu Rev. 1998; 4:1-54; Bertolini et al., Transgenic Res. 2016 June; 25(3):329-43; Monzani et al., Bioengineered. 2016 April; 7(3):123-31; Ivarie et al., ends Biotechnol. 2006 March; 24(3):99-101; Lillico et al., Drug Discov Today. 2005 Feb. 1; 10(3):191-6; Dyck et al., Trends Biotechnol. 2003 September; 21(9):394-9; Sid and Schusser et al 2018. Front. Genet. Doi.org/10.3389/fgene.2018.00456; Scott et al. 2010. ILAR J. 51(4):353-361; Yum et al., 2016. Scientific Reports. 6:27185; Tait-Burkard et al. 2018. Genome Biology. 19:2014; Kalds et al., 2019. Front. Genet. Doi.org//10.3389/fgene.2019.00750; Hryhorowicz et al., Genes (Basel). 2020 Jun. 19; 11(6):670; Monzani et al., Bioengineered. 2016 April; 7(3):123-31, which are incorporated herein by reference and can be adapted for use with the present disclosure.

### Engineered Cells for Cell-Based Therapies

In some embodiments, engineered cells can be administered as part of a therapy to a subject in need thereof. In some embodiments, the cells are allogeneic to a subject in which they are delivered. In some embodiments, the cells are autologous to a subject in which they are delivered. In some embodiments, the cells are engineered ex vivo to express and/or secrete one or more of the engineered PNMA polypeptides and/or engineered PNMA capsids (including cargo loaded capsids) described herein prior to or after administration to a subject in need thereof. The cells can provide production of engineered PNMA polypeptides and/or engineered PNMA capsids (including cargo loaded capsids) described herein within the subject in need thereof. In some embodiments, the cargo is a cargo native to the cell in which the PNMA capsid is produced.

The use of autologous cells for a cell-based therapy described herein can minimize graft-versus-host disease (GVHD) issues. In some embodiments, allogenic cells are modified to reduce alloreactivity and prevent graft-versus-host disease. This approach can have the advantage as being able to provide “off the shelf” solutions and cells for cell-based therapies and delivery of the SCN1B mimetic peptides and provide a scalable alternative to autologous approaches. Platforms for manufacturing allogenic and autologous cells (including modified cells) are generally known in the art. See e.g., Abbasalizadeh Expert Opin Biol Ther. 2017 October; 17(10):1201-1219; Pigeau et al., Front Med (Lausanne). 2018 Aug. 22; 5:233; Abraham et al., Adv Biochem Eng Biotechnol. 2018; 165:323-350. Exemplary modifications that can be introduced allogenic cells to reduce complications to alloreactivity are known in the art. See e.g., Perez et al., Front Immunol. 2020 Nov. 11; 11:583716. doi: 10.3389.

In some embodiments, the engineered cells for delivery of engineered PNMA polypeptides and/or engineered PNMA capsids (including cargo loaded capsids) are engineered to include a transgenic safety switch, in the form of a transgene that renders the cells vulnerable to exposure to a specific signal. For example, in some embodiments, the cells can be engineered with a thymidine kinase (TK) gene that, in response to a nucleoside prodrug (e.g., ganciclovir or acyclovir), causes cell death (see e.g., Greco, et al., Improving the safety of cell therapy with the TK-suicide gene. Front. Pharmacol. 2015; 6: 95). Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In the instance of allogenic cells, the allogenic cells can be engineered to be resistant to immunosuppressive agents (e.g., calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite), which are often given to subjects receiving an allogenic therapy. In some embodiments, immunosuppressive resistance is conferred to the allogeneic cells by inactivating the target of the immunosuppressive agent in the cells. As non-limiting examples, targets for an immunosuppressive agent can be a receptor for an immunosuppressive agent such as: CD52, glucocorticoid receptor (GR), a FKBP family gene member and a cyclophilin family gene member.

The engineered cells can be any suitable cell type. The engineered cells can be cultured and expanded as desired prior to delivery to the subject. Collection, isolation, selection, culture, expansion, and storage methods for the engineered cells are generally known in the art.

### Engineered Pnma Capsid Delivery Systems

Described in certain example embodiments herein are engineered delivery systems that include a capsid comprising the engineered PNMA polypeptide of the present invention described in greater detail elsewhere herein and a cargo captured by, or packaged within, the capsid.

In some embodiments, the capsid comprises only one type of engineered PNMA polypeptide. In other embodiments, the capsid comprises two or more (e.g., 2, 3, 4, 5, 6, 7, 8, 9, 10 or more) types of engineered PNMA polypeptides. The term “type of engineered PNMA polypeptide” is used to distinguish engineered PNMA polypeptides that differ in at least one aspect but are engineered PNMA polypeptides of the present invention. In some embodiments, the capsid is formed of one or more types of engineered PNMA polypeptides of the present invention and one or more other PNMA polypeptides, such as wild-type or non-engineered PNMA polypeptides.

Cargo capturing and/or packaging can occur in vivo (e.g., within a producer or host cell). For example, the engineered PNMA (or other) polypeptides capable of forming a capsid can be expressed within in a cell (e.g., from a vector system or other expression system) along with the cargo to be captured or packaged. The cargo can be natively expressed in the cell or can be expressed from an exogenous construct introduced stably or transiently into the cell. Upon formation of the capsid within the cell, the cargo is captured and/or packaged. In some embodiments, the cargo is a cargo native to the cell in which the capsid is produced. In some embodiments the cargo is exogenous to the cell in which the capsid is produced. Exemplary expression systems and bioreactors are described in greater detail elsewhere herein.

Cargo capturing and/or packaging can occur in vitro. In some embodiments, the engineered PNMA peptides can be generated and harvested by any suitable in vitro or in vivo system. The engineered PNMA polypeptides and cargo can be combined in vitro where, upon self-assembly of the engineered PNMA polypeptides into engineered PNMA capsids, the cargo is captured and/or packaged within the engineered PNMA capsid.

The engineered capsids can contain one or more types of cargos. Exemplary cargos are further described below.

### Exemplary Cargos

The engineered PNMA capsids described herein may be used and further comprise a one or more cargo molecules for delivery. Representative cargo molecules may include, but are not limited to, nucleic acids, polynucleotides, proteins, polypeptides, polynucleotide/polypeptide complexes, small molecules, sugars, or a combination thereof. Cargos that can be delivered in accordance with the systems and methods described herein include, but are not necessarily limited to, biologically active agents, including, but not limited to, therapeutic agents, imaging agents, and monitoring agents. A cargo may be an exogenous material (a molecule or composition not native to a cell in which it is produced or, in the context of an in vitro production, a molecule or composition not native to the engineered PNMA capsid) or an endogenous material, such as a molecule native to a cell in which it is produced. One or more or two or more different cargoes may be delivered by the delivery particles described herein.

As is also described in greater detail elsewhere herein, in some embodiments, the cargos, optionally peptide and polypeptide cargos, are coupled to (e.g., fused directly to or indirectly via a linker) to a dimerization or other functional domain. In some embodiments, the dimerization domain or other functional domain can dimerize or otherwise interact with a corresponding dimerization domain or other functional domain on the PNMA. In some embodiments, such dimerization or interaction increases or enhances capture or encapsulation of the cargo within a PNAM capsid. In some embodiments, the dimerization domain is a leucine zipper. In some embodiments, the linker is a Gly-Ser linker, optionally GSGGGS (SEQ ID NO: 9). In one exemplary embodiment and as described elsewhere herein the cargo is an SpCas9 or functional fragment thereof, which is fused at the C- or N-terminus via a Gly-Ser linker, optionally GSGGGS (SEQ ID NO: 9), to a dimerization domain, optionally a leucine zipper. Other suitable linkers, including Gly-Ser and other linkers appropriate for linking cargos to functional domains are described elsewhere herein will be appreciated in view of the description herein. Exemplary dimerization and functional domains that can be coupled to the cargos, optionally peptide and polypeptide cargos, are also described in greater detail elsewhere herein and will be appreciated in view of the description herein.

### Polynucleotides

In some embodiments, the cargo is a cargo polynucleotide. As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “deoxyribonucleic acid (DNA)” and “ribonucleic acid (RNA)” can generally refer to any polyribonucleotide or polydeoxribonucleotide, which may be unmodified RNA or DNA or modified RNA or DNA. RNA can be in the form of non-coding RNA, including but not limited to, tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA), or coding mRNA (messenger RNA).

In some embodiments, the cargo polynucleotide is DNA. In some embodiments, the cargo polynucleotide is RNA. In some embodiments, the cargo polynucleotide is a polynucleotide (a DNA or an RNA) that encodes an RNA and/or a polypeptide. As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

### Polynucleotide Modifications

In some embodiments, the cargo polynucleotides include one or more modifications capable of modifying the e.g., functionality, packaging ability, stability, degradation localization, increase expression lifetime, resistance to degradation, or any combination thereof, of the at least one or more cargo polynucleotides. Modifications can be sequence modifications (e.g., mutations), chemical modifications, or other modifications, such as complexing to a lipid, polymer, etc. In some embodiments, the cargo polynucleotide is modified to protect it against degradation, by e.g., nucleases or otherwise prevent its degradation.

In some embodiments, one or more polynucleotides in the engineered polynucleotide are modified. In some embodiments, the engineered polynucleotide includes one or more non-naturally occurring nucleotides, which can be the result of modifying a naturally occurring nucleotide. In some embodiments, the modification is selected independently for each polynucleotide modified. In some embodiments, the modification(s) increase or decrease the stability of the polynucleotide, reduce the immunogenicity of the polynucleotide, increase or decrease the rate of transcription and/or translation, or any combination thereof. Non-naturally occurring nucleotides and/or nucleotide analogs may be modified at the ribose, phosphate, and/or base moiety.

Suitable modifications include, without limitation, methylpseudouridine, a phosphorothioate linkage, a locked nucleic acid (LNA) nucleotides comprising a methylene bridge between the 2′ and 4′ carbons of the ribose ring, or bridged nucleic acids (BNA), 2′-O-methyl analogs, 2′-deoxy analogs, or 2′-fluoro analogs, 2-aminopurine, 5-bromo-uridine, pseudouridine, (Ψ), N1-methylpseudouridine (me1Ψ), 5-methoxyuridine(5moU), inosine, 7-methylguanosine, inosine, 7-methylguanosine. Examples of RNA, including but not limited to gudide RNA, chemical modifications include, without limitation, incorporation of 2′-O-methyl (M), 2′-O-methyl 3′phosphorothioate (MS), S-constrained ethyl(cEt), or 2′-O-methyl 3′thioPACE (MSP) at one or more terminal nucleotides.

In some embodiments, the polynucleotide (DNA and/or RNA) is modified with a 5′- and/or 3′-cap structure. In some embodiments, the 5′ cap structure is cap0, cap1, ARCA, inosine, N1-methyl-guanosine, 2′-fluoro-guanosine, 7-deaza-guanosine, 8-oxo-guanosine, 2-amino-guanosine, LNA-guanosine, or 2-azido-guanosine. In some embodiments, the 5′terminal cap is 7mG(5′)ppp(5′)N1mpNp, m7GpppG cap, N7-methylguanine. In some embodiments, the 3′terminal cap is a 3′-O-methyl-m7GpppG, 2′Fluoro bases, inverted dT and dTTs, phosphorylation of the 3′ end nucleotide, a C3 spacer. Exemplary 5′- and/or 3′ that protect against degradation are described in e.g., Gagliardi and Dziembowski. Philosophical transactions of the Royal Society B. 2018. 313 (1762). https://doi.org/10.1098/rstb.2018.0160; Boo and Kim. Experimental & Molecular Medicine volume 52, pages 400-408 (2020); and Adachai et al., 2021. Biomedicines 2021, 9, 550. https://doi.org/10.3390/biomedicines9050550.

In some embodiments, the 5′-UTR comprises a Kozak sequence.

In some embodiments, the polynucleotide can be modified with a tailing sequence may range from absent to 500 nucleotides in length (e.g., at least 60, 70, 80, 90, 120, 140, 160, 180, 200, 250, 300, 350, 400, 450, or 500 nucleotides). In some embodiments, the tailing region is or includes a polyA tail. Where the tailing region is a polyA tail, the length may be determined in units of or as a function of polyA Binding Protein binding. In this embodiment, the polyA tail is long enough to bind at least 4 monomers of PolyA Binding Protein. Poly A Binding Protein monomers bind to stretches of approximately 38 nucleotides. As such, it has been observed that polyA tails of about 80 nucleotides and 160 nucleotides are functional. In some embodiments, the poly-A tail is at least 160 nucleotides in length.

In some embodiments, about 10%, 15%, 20%, 24%, 30%, 35%, 40%, 45%, 50%, 55%, 60%, 65%, 70%, 75%, 80%, 85%, 90%, 95%, to/or about 100% of the uracils in of a polynucleotide of the present invention have a chemical modification, In some embodiments, about 10%, 15%, 20%, 24%, 30%, 35%, 40%, 45%, 50%, 55%, 60%, 65%, 70%, 75%, 80%, 85%, 90%, 95%, to/or about 100% of the uracils of a polynucleotide of the present invention have a N1-methyl pseudouridine in the 5-position of the uracil.

In some embodiments, the polynucleotide, optionally an RNA (e.g., an mRNA) includes a stabilization element. In some embodiments, the stabilization element is a histone stem-loop. In some embodiments, the stabilization element is a nucleic acid sequence having increased GC content relative to wild type sequence.

In some embodiments, a polynucleotide of the present invention includes a sequence encoding a self-cleaving peptide. The self-cleaving peptide may be, but is not limited to, a 2A peptide. In one embodiment, this sequence may be used to separate the coding regions of two or more polypeptides.

In some embodiments, the polynucleotides (e.g., mRNAs) are linear. In yet another embodiment, the polynucleotides of the present invention that are circular are known as “circular polynucleotides” or “circP.” As used herein, “circular polynucleotides” or “circP” means a single stranded circular polynucleotide which acts substantially like, and has the properties of, an R A. The term “circular” is also meant to encompass any secondary or tertiary configuration of the circP.

Other RNA modifications, such as mRNA modifications, that can be incorporated into a polynucleotide of the present invention include, but are not limited to, any one or more of those described e.g., U.S. Pat. Nos. 8,278,036, 8,691,966, 8,748,089, 9,750,824, 10,232,055, 10,703,789, 10,702,600, 10,577,403, 10,442,756, 10,266,485, 10,064,959, 9,868,692, 10,064,959, 10,272,150; U.S. Publications, US20130197068, US20170043037, US20130261172, US20200030460, US20150038558, US20190274968, US20180303925, US20200276300; International Patent Application Publication Nos. WO/2018/081638A1, WO/2016/176330A1, which are incorporated herein by reference and can be adapted for use with the present invention.

### Signaling and Localization Molecules

In some embodiments, the cargo polynucleotide includes a signaling and/or localization molecule (e.g., a polynucleotide that is a signaling or localization molecule or a polynucleotide that encodes a signaling or localization peptide or polypeptide).

In some embodiments, the signaling or localization molecule directs a function (e.g., secretion, folding, etc.) and/or trafficking to a particular location within a cell (e.g., nucleus, Golgi, lysosome, peroxisome, cytoplasm, membrane, chloroplast, vacuole, mitochondria, etc.). In some embodiments, the signaling or localization molecule(s) is/are positioned at the 3′ and/or 5′ end of a polynucleotide of the present invention, such as a cargo polynucleotide. In some embodiments, the signaling or localization molecule(s) is/are located at one or more positions between the 3′ and 5′ end of a polynucleotide of the present invention. In some embodiments, the signaling or localization molecule(s) are located at the 3′ and/or 5′ end of a polynucleotide of the present invention and at one or more positions between the 3′ and 5′ end of a polynucleotide of the present invention. In some embodiments, the signaling and/or localization molecule(s) is/are incorporated in a polynucleotide, such as a cargo polynucleotide, such that it is at the C-terminus, N-terminus, or one or more positions between the C-terminus and N-terminus of a polypeptide encoded by the polynucleotide.

In some embodiments, a polynucleotide of the present invention includes a polynucleotide sequence that is or encodes one or more signal peptides, leucine rich repeat (LRR) sequences, nuclear localization signals, a Type IX secretion system (T9SS) substrate, secretion signal peptide, an amino acid sequence capable of directing clearance from a cell or organism, an Fc receptor directing binding to a dendritic cell, and/or directing antigen processing, an F-box domain or polypeptide, a subcellular localization sequence, a TOM70, TOM20, or TOM22 binding polypeptide, a stromal import sequence, a thylakoid targeting sequence, a peroxisome targeting signal 1 sequence, a peroxisome targeting signal 2 sequence, an endoplasmic reticulum signaling sequence.

Exemplary nuclear localization molecules are described in e.g., Lu et al., Cell Communication and Signaling. 2021. 19(60): 1-10 (particularly at Table 1 therein), which can be adapted for use with the present invention. Exemplary signal peptides are described in e.g., Owji et al., European J Cell Biol. 2018. 97(6):422-441, which can be adapted for use with the present invention. Exemplary peroxisome targeting sequences are described in e.g., Baerends et al., 2000. FEMS Microbiol Rev. 24(3): 291-301, which can be adapted for use with the present invention. Exemplary endoplasmic reticulum signaling molecules are described in e.g., Walter et al., J Cell Biol. 1981. 91 (2 Pt. 1):545-50 doi:10.1083/jcb.91.2.545, which can be adapted for use with the present invention. Exemplary lysosomal and endosomal signaling molecules are described in e.g., Bonifacino and Traub. 2003. Ann. Rev. Biochem. 72:395-447, which can be adapted for use with the present invention. Exemplary endoplasmic reticulum signaling sequences are described in e.g., J Cell Biol. 1996 Jul. 2; 134(2): 269-278, which can be adapted for use with the present invention. Exemplary Golgi signaling sequences are described in e.g., Gleeson et al., 1994. Glycoconjugat J. 11:381-394, which can be adapted for use with the present invention.

### Interference RNAs

In certain example embodiments, the one or more cargo polynucleotides are or encode one or more interference RNAs. Interference RNAs are RNA molecules capable of suppressing gene expressions. Example types of interference RNAs include small interfering RNA (siRNA), microRNA (miRNA), and short hairpin RNA (shRNA).

In certain example embodiments, the interference RNA may be a siRNAs. Small interfering RNA (siRNA) molecules are capable of inhibiting target gene expression by interfering RNA. siRNAs may be chemically synthesized, or may be obtained by in vitro transcription, or may be synthesized in vivo in a target cell. siRNAs may comprise double-stranded RNA from 15 to 40 nucleotides in length and can contain a protuberant region 3′ and/or 5′ from 1 to 6 nucleotides in length. Length of protuberant region is independent from total length of siRNA molecule. siRNAs may act by post-transcriptional degradation or silencing of target messenger. In some cases, the exogenous polynucleotides encode shRNAs. In shRNAs, the antiparallel strands that form siRNA are connected by a loop or hairpin region.

The interference RNA (e.g., siRNA) may suppress expression of genes to promote long term survival and functionality of cells after transplanted to a subject. In some examples, the interference RNAs suppress genes in TGFβ pathway, e.g., TGFβ, TGFβ receptors, and SMAD proteins. In some examples, the interference RNAs suppress genes in colony-stimulating factor 1 (CSF1) pathway, e.g., CSF1 and CSF1 receptors. In certain embodiments, the one or more interference RNAs suppress genes in both the CSF1 pathway and the TGFβ pathway. TGFβ pathway genes may comprise one or more of ACVR1, ACVR1C, ACVR2A, ACVR2B, ACVRL1, AMH, AMIHR2, BMP2, BMP4, BMP5, BMP6, BMP7, BMP8A, BMP8B, BMPR1A, BMPR1B, BMPR2, CDKN2B, CHRD, COMP, CREBBP, CUL1, DCN, E2F4, E2F5, EP300, FST, GDF5, GDF6, GDF7, ID1, ID2, ID3, ID4, IFNG, INHBA, INHBB, INHBC, INHBE, LEFTY1, LEFTY2, LOC728622, LTBP1, MAPK1, MAPK3, MYC, NODAL, NOG, PITX2, PPP2CA, PPP2CB, PPP2R1A, PPP2R1B, RBL1, RBL2, RBX1, RHOA, ROCK1, ROCK2, RPS6KB1, RPS6KB2, SKP1, SMAD1, SMAD2, SMAD3, SMAD4, SMAD5, SMAD6, SMAD7, SMAD9, SMURF1, SMURF2, SP1, TFDP1, TGFB1, TGFB2, TGFB3, TGFBR1, TGFBR2, THBS1, THBS2, THBS3, THBS4, TNF, ZFYVE16, and/or ZFYVE9.

In some embodiments, the cargo polynucleotide is an RNAi molecule, antisense molecule, and/or a gene silencing oligonucleotide or a polynucleotide that encodes an RNAi molecule, antisense molecule, and/or gene silencing oligonucleotide.

As used herein, “gene silencing oligonucleotide” refers to any oligonucleotide that can alone or with other gene silencing oligonucleotides utilize a cell's endogenous mechanisms, molecules, proteins, enzymes, and/or other cell machinery or exogenous molecule, agent, protein, enzyme, and/or polynucleotide to cause a global or specific reduction or elimination in gene expression, RNA level(s), RNA translation, RNA transcription, that can lead to a reduction or effective loss of a protein expression and/or function of a non-coding RNA as compared to wild-type or a suitable control. This is synonymous with the phrase “gene knockdown” Reduction in gene expression, RNA level(s), RNA translation, RNA transcription, and/or protein expression can range from about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, to 1% or less reduction. “Gene silencing oligonucleotides” include, but are not limited to, any antisense oligonucleotide, ribozyme, any oligonucleotide (single or double stranded) used to stimulate the RNA interference (RNAi) pathway in a cell (collectively RNAi oligonucleotides), small interfering RNA (siRNA), microRNA, and short-hairpin RNA (shRNA). Commercially available programs and tools are available to design the nucleotide sequence of gene silencing oligonucleotides for a desired gene, based on the gene sequence and other information available to one of ordinary skill in the art.

### Therapeutic Polynucleotides

In some embodiments, the cargo molecule is a therapeutic polynucleotide. Therapeutic polynucleotides are those that provide a therapeutic effect when delivered to a recipient cell. The polynucleotide can be a toxic polynucleotide (a polynucleotide that when transcribed or translated results in the death of the cell) or polynucleotide that encodes a lytic peptide or protein. In embodiments, delivery vesicles having a toxic polynucleotide as a cargo molecule can act as an antimicrobial or antibiotic. This is discussed in greater detail elsewhere herein. In some embodiments, the cargo molecule can be exogenous to the producer cell and/or a first cell. In some embodiments, the cargo molecule can be endogenous to the producer cell and/or a first cell. In some embodiments, the cargo molecule can be exogenous to the recipient cell and/or a second cell. In some embodiments, the cargo molecule can be endogenous to the recipient cell and/or second cell.

As described herein the cargo polynucleotide can be any polynucleotide endogenous or exogenous to the eukaryotic cell. For example, the cargo polynucleotide can be a polynucleotide residing in the nucleus of the eukaryotic cell. The cargo polynucleotide can be a sequence coding a gene product (e.g., a protein) or a non-coding sequence (e.g., a regulatory polynucleotide).

In some embodiments, the cargo polynucleotide is a DNA or RNA (e.g., a mRNA) vaccine.

### Aptamers

In certain example embodiments, the cargo polynucleotide is an aptamer. In certain embodiments, the one or more agents is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be or capable of being linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 3′ of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH2), 2′-fluoro (2′-F), and/or 2′-O-methyl (2′-OMe) substituents. Modifications of aptamers may also include modifications at exocyclic amines, substitution of 4-thiouridine, substitution of 5-bromo or 5-iodo-uracil; backbone modifications, phosphonothioate or allyl phosphate modifications, methylations, and unusual base-pairing combinations such as the isobases isocytidine and isoguanosine. Modifications can also include 3′ and 5′ modifications such as capping. As used herein, the term phosphonothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, 0-allyl, S-alkyl, S-allyl, or halo of synthesis of 2-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include but are not limited to those described in Rohloff et al., “Nucleic Acid Ligands with Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colorado). In certain embodiments, the present invention may utilize any aptamer containing any modification as described herein.

In certain other example embodiments, the polynucleotide may be a ribozyme or other enzymatically active polynucleotide.

### Biologically Active Agents

In some embodiments, the cargo is a biologically active agent. Biologically active agents include any molecule that induces, directly or indirectly, an effect in a cell. Biologically active agents may be a protein, a nucleic acid, a small molecule, a carbohydrate, a lipid or any combination thereof. Biologically active agents can be therapeutic agents. Exemplary Therapeutic agents include, without limitation, chemotherapeutic agents, anti-oncogenic agents, anti-angiogenic agents, tumor suppressor agents, anti-microbial agents, enzyme replacement agents, gene expression modulating agents and expression constructs comprising a nucleic acid encoding a therapeutic protein or nucleic acid, and vaccines. Therapeutic agents may be peptides, proteins (including enzymes, antibodies and peptidic hormones), ligands of cytoskeleton, nucleic acid, small molecules, non-peptidic hormones and the like. To increase affinity for the nucleus, agents may be conjugated to a nuclear localization sequence. Nucleic acids that may be delivered by the method of the invention include synthetic and natural nucleic acid material, including DNA, RNA, transposon DNA, antisense nucleic acids, dsRNA, siRNAs, transcription RNA, messenger RNA, ribosomal RNA, small nucleolar RNA, microRNA, ribozymes, plasmids, expression constructs, etc.

### Imaging Agents

Imaging agents include contrast agents, such as ferrofluid-based MRI contrast agents and gadolinium agents for PET scans, fluorescein isothiocyanate and 6-TAMARA. Monitoring agents include reporter probes, biosensors, green fluorescent protein and the like. Reporter probes include photo-emitting compounds, such as phosphors, radioactive moieties and fluorescent moieties, such as rare earth chelates (e.g., europium chelates), Texas Red, rhodamine, fluorescein, FITC, fluo-3, 5 hexadecanoyl fluorescein, Cy2, fluor X, Cy3, Cy3.5, Cy5, Cy5.5, Cy7, dansyl, phycocrytherin, phycocyanin, spectrum orange, spectrum green, and/or derivatives of any one or more of the above. Biosensors are molecules that detect and transmit information regarding a physiological change or process, for instance, by detecting the presence or change in the presence of a chemical. The information obtained by the biosensor typically activates a signal that is detected with a transducer. The transducer typically converts the biological response into an electrical signal. Examples of biosensors include enzymes, antibodies, DNA, receptors and regulator proteins used as recognition elements, which can be used either in whole cells or isolated and used independently (D'Souza, 2001, Biosensors and Bioelectronics 16:337-353).

As used herein the term “altered expression” may particularly denote altered production of the recited gene products by a cell. As used herein, the term “gene product(s)” includes RNA transcribed from a gene (e.g., mRNA), or a polypeptide encoded by a gene or translated from RNA.

Also, “altered expression” as intended herein may encompass modulating the activity of one or more endogenous gene products. Accordingly, “altered expression”, “altering expression”, “modulating expression”, or “detecting expression” or similar may be used interchangeably with respectively “altered expression or activity”, “altering expression or activity”, “modulating expression or activity”, or “detecting expression or activity” or similar terms. As used herein, “modulating” or “to modulate” generally means either reducing or inhibiting the activity of a target or antigen, or alternatively increasing the activity of the target or antigen, as measured using a suitable in vitro, cellular or in vivo assay. In particular, “modulating” or “to modulate” can mean either reducing or inhibiting the (relevant or intended) activity of, or alternatively increasing the (relevant or intended) biological activity of the target or antigen, as measured using a suitable in vitro, cellular or in vivo assay (which will usually depend on the target or antigen involved), by at least 5%, at least 10%, at least 25%, at least 50%, at least 60%, at least 70%, at least 80%, or 90% or more, compared to activity of the target or antigen in the same assay under the same conditions but without the presence of the inhibitor/antagonist agents or activator/agonist agents described herein.

As will be clear to the skilled person, “modulating” can also involve effecting a change (which can either be an increase or a decrease) in affinity, avidity, specificity and/or selectivity of a target or antigen, for one or more of its targets compared to the same conditions but without the presence of a modulating agent. Again, this can be determined in any suitable manner and/or using any suitable assay known per se, depending on the target. In particular, an action as an inhibitor/antagonist or activator/agonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the inhibitor/antagonist agent or activator/agonist agent. Modulating can also involve activating the target or antigen or the mechanism or pathway in which it is involved.

### Polynucleotide Modifying Systems

In some embodiments, the cargo is a polynucleotide modifying system or component(s) thereof. In some embodiments the polynucleotide modifying system is a gene modifying system. In some embodiments, the gene modifying system is or is composed of a gene (or genetic) modulating agent. In some embodiments, the genetic modulating agent may comprise one or more polypeptide and/or polynucleotide components of a polynucleotide modification system (e.g., a gene editing system) and/or polynucleotides encoding thereof.

In some embodiments, the gene editing system may be an RNA-guided system or other programmable nuclease system. In some embodiments, the gene editing system is an IscB system. In some embodiments, the gene editing system may be a CRISPR-Cas system. In some embodiments, the polynucleotide modifying system is a recombinase, zinc finger nuclease, or TALEN. Other suitable polynucleotide modifying systems and/or components thereof that can be included as cargos will be appreciated by one of ordinary skill in the art in view of the description herein.

### CRISPR-Cas Systems

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

### Class 1 Systems

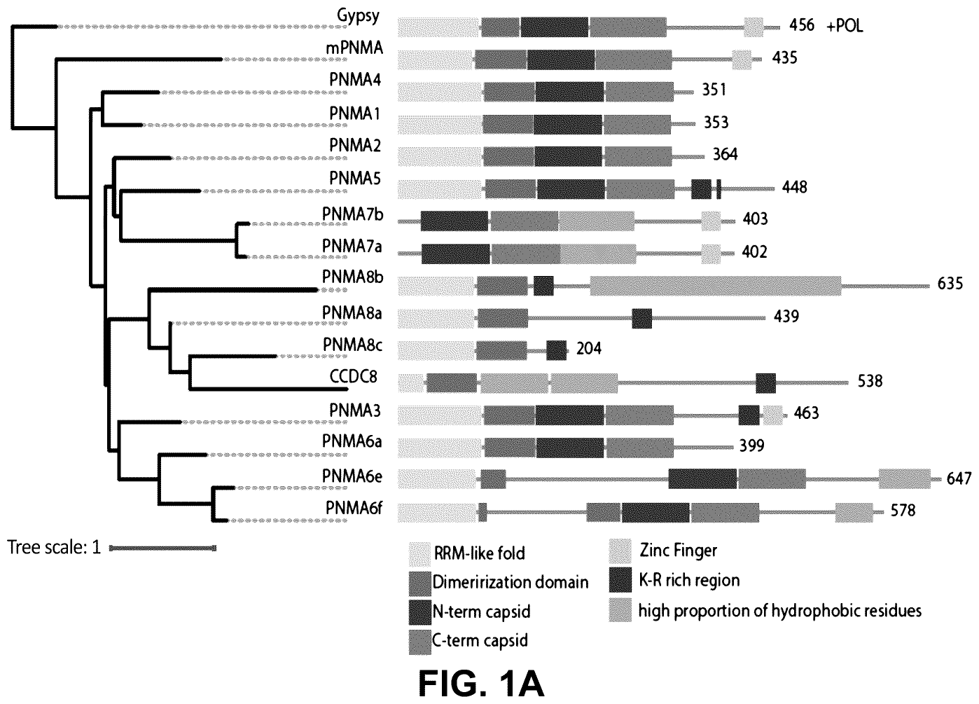

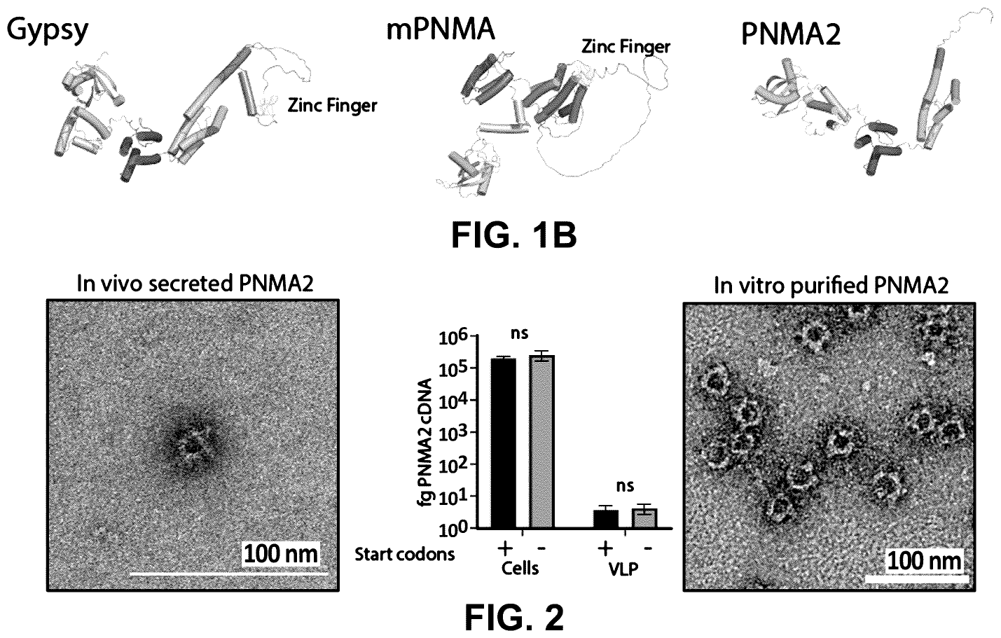

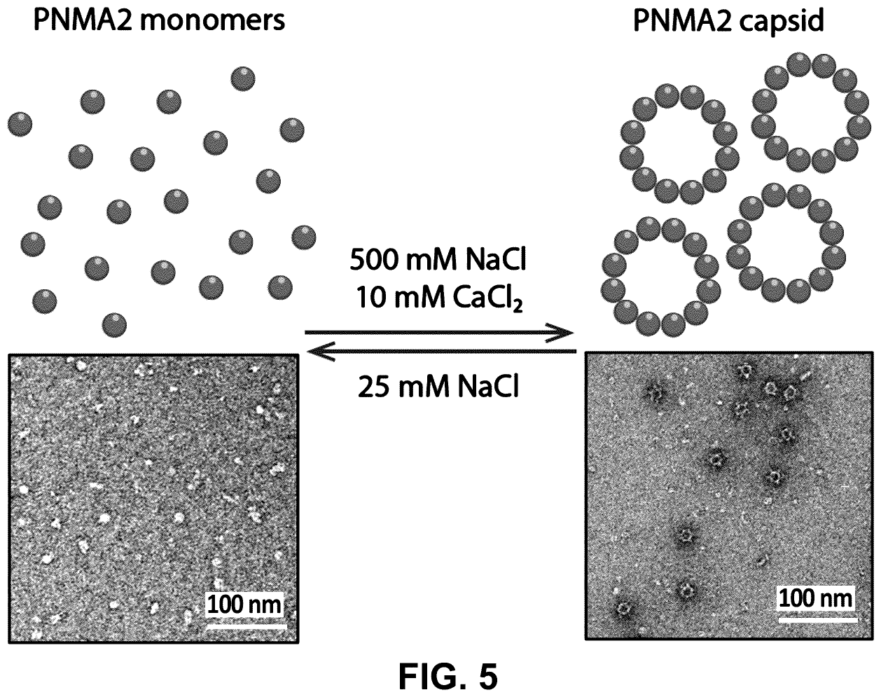

The methods, systems, and tools provided herein may be designed for use with Class 1 CRISPR proteins. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020), incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g., Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade in particular Class 1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

### Class 2 Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1 (V-U3), V-F2, V-F3, V-G, V-H, V-I, V-K (V-U5), V-U1, V-U2, and V-U4. Class 2, Type VI systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9. In some embodiments, the Cas9 is an SpCas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), Cas14, and/or CasΦ.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Guide Molecules

The CRISPR-Cas or Cas-Based system described herein can, in some embodiments, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4) 702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 2 (from Gleditzsch et al. 2019) below shows several Cas polypeptides and the PAM sequence they recognize.

TABLE 2
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including LbCpf1 and AsCpf1) | TTTV |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155 (Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples ofNLSs include an NLS sequence derived from: the NLS of the SV40 virus large T-antigen, having the amino acid sequence PKKKRKV (SEQ ID NO: 20) or PKKKRKVEAS (SEQ ID NO: 21); the NLS from nucleoplasmin (e.g., the nucleoplasmin bipartite NLS with the sequence KRPAATKKAGQAKKKK (SEQ ID NO: 22)); the c-myc NLS having the amino acid sequence PAAKRVKLD (SEQ ID NO: 23) or RQRRNELKRSP (SEQ ID NO: 24); the hRNPA1 M9 NLS having the sequence NQSSNFGPMKGGNFGGRSSGPYGGGGQYFAKPRNQGGY (SEQ ID NO: 25); the sequence RMRIZFKNKGKDTAELRRRRVEVSVELRKAKKDEQILKRRNV (SEQ ID NO: 26) of the IBB domain from importin-alpha; the sequences VSRKRPRP (SEQ ID NO: 27) and PPKKARED (SEQ ID NO: 28) of the myoma T protein; the sequence PQPKKKPL (SEQ ID NO: 29) of human p53; the sequence SALIKKKKKMAP (SEQ ID NO: 30) of mouse c-abl IV; the sequences DRLRR (SEQ ID NO: 31) and PKQKKRK (SEQ ID NO: 32) of the influenza virus NS1; the sequence RKLKKKIKKL (SEQ ID NO: 33) of the Hepatitis virus delta antigen; the sequence REKKKFLKRR (SEQ ID NO: 34) of the mouse Mxl protein; the sequence KRKGDEVDGVDEVAKKKSKK (SEQ ID NO: 35) of the human poly(ADP-ribose) polymerase; and the sequence RKCLQAGMNLEARKTKK (SEQ ID NO: 36) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g., aptamers) for adapter proteins, which may be linked to or fused to a nucleotide deaminase or catalytic domain thereof. When such a guide forms a CRISPR complex (e.g., CRISPR-Cas protein binding to guide and target), the adapter proteins bind and the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

It will be appreciated that NLS and NES described herein with respect to Cas proteins can be used with other cargos, in particularly, gene modifying agents herein, and other proteins that can benefit from translocation in or out of a nuclease of a cell, such as a target cell.

### Donor Templates

In some embodiments, the composition for engineering cells comprises a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, or 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, or 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

In certain, embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149), which can be adapted for use with the present invention.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and International Patent Publication WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein is attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild-type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a CG base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In one example embodiment, the cargo may be prime editing systems or one or more polynucleotides encoding one or more components thereof. Prime editing systems comprise a programable nuclease (e.g., Cas), most often a nickase, linked to a reverse transcriptase domain and a guide molecule (prime editing guide pegRNA), which comprises a target-specific spacer, a primer binding site, and RT template. See e.g., Anzalone et al. 2019. Nature. 576: 149-157; and International Patent Application Publication No. WO2022150790A2. In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

Prime editing systems can also be used in tandem such that, the two pegRNAs template the synthesis of complementary DNA flaps on opposing strands of genomic DNA, which replace the endogenous DNA sequence between the PE-induced nick sites. See, e.g., WO 2021/138469; Anzalone A V, Gao X D, Podracky C J, et al. Programmable deletion, replacement, integration and inversion of large DNA sequences with twin prime editing. Nat Biotechnol. 2022; 40(5):731-740. Thus, use of two pegRNAs allows for larger insertions or deletions because of the two overlapping 3′ flaps created by the two nicked sites. In one example embodiment, the system can be used to insert or replace a sequence into one or more target genes. In example embodiments, the insertion or replacement results in an inactive target gene or less active form of the target gene. In one example embodiment, the system is used to replace all or a portion of the entire target gene. In one example embodiment, the system is used to replace all or a portion of an enhancer controlling the target gene expression.

### Recombinase-Mediated Modifications

Prime editing and twinPE systems can also be further combined with site-specific recombinases, such as integrases, to facilitate even larger insertions, substitutions and deletions. See e.g., WO 2021/138469; Anzalone A V, Gao X D, Podracky C J, et al. Programmable deletion, replacement, integration and inversion of large DNA sequences with twin prime editing. Nat Biotechnol. 2022; 40(5):731-740; Yarnall et al., Nat Biotechnol (2022). doi.org/10.1038/s41587-022-01527-4, which is incorporated by reference as if expressed in its entirety herein. The prime editing system is used to insert a recombinase recognition site at the desire site of modification and an integrase facilitates the insertion of a donor sequence from a donor template. “Uni-directional recombinases” or “integrases” refer to recombinase enzymes whose recognition sites are destroyed after the recombination has taken place. The term “integrase” refers to a type of recombinase. In other words, the sequence recognized by the recombinase is changed into one that is not recognized by the recombinase upon recombination. As a result, once a sequence is subjected to recombination by the uni-directional recombinase, the continued presence of the recombinase cannot reverse the previous recombination event.

Typically, two different sites are involved (in regards to recombination termed “complementary sites”), one present in the target nucleic acid (e.g., a chromosome or episome of a eukaryote) and another on the nucleic acid that is to be integrated at the target recombination site. The terms “attB” and “attP,” which refer to attachment (or recombination) sites originally from a bacterial target (attachment site of bacteria) and a phage donor (attachment site of phage), respectively, are used herein although recombination sites for particular enzymes may have different names. The two attachment sites can share as little sequence identity as a few base pairs. The recombination sites typically include left and right arms separated by a core or spacer region. Thus, an attB recombination site consists of BOB′, where B and B′ are the left and right arms, respectively, and O is the core region. Similarly, attP is POP′, where P and P′ are the arms and O is again the core region. Upon recombination between the attB and attP sites, and concomitant integration of a nucleic acid at the target, the recombination sites that flank the integrated DNA are referred to as “attL” and “aatR.” The attL and attR sites, using the terminology above, thus consist of BOP‘ and POB’, respectively. In some representations herein, the “O” is omitted and attB and attP, for example, are designated as BB‘ and PP’, respectively.

In example embodiments, the recombinase of the present invention is a serine integrase. In example embodiments, serine integrases specifically recombine when recognizing the two attachment sites specific for the integrase. In example embodiments, the heterologous sites are referred to as attP and attB, however, these terms refer to the specific sequences recognized by the specific integrase and do not refer to a single consensus sequence. Serine integrases mediate site-specific recombination between short recognition sites located in phage genomes and bacterial chromosomes, respectively, the attachment site of phage (attP) and attachment site of bacteria (attB) (i.e., the target sites of the integrase), to form the hybrid attachment sites attL and attR. Unlike Cre and Flp recombinases that catalyze reversible site-specific recombination reactions, serine integrases are unidirectional and catalyze only attP and attB recombination without RDF or Xis accessory proteins. Thus, in the absence of any accessory factors integrase is unidirectional. In addition, DNA substrates identified by serine integrases (attP and attB) are relatively short (30-50 bp) and have a minimal length of approximately 34-40 base pairs (bp) (Groth A C et al., Proc. Natl. Acad. Sci. USA 97, 5995-6000 (2000)). The compatibility of distinct DNA topological structures is also quite different from recognition of DNA by Hin recombinase or Tn3 resolvase. Serine integrases recognize DNA substrates specifically, not at random, but can facilitate recombination at sequences with partial identity with wild-type recombination sites, termed pseudo attachment sites (either pseudo attP or pseudo attB). A “pseudo-recombination site” is a DNA sequence recognized by a recombinase enzyme such that the recognition site differs in one or more base pairs from the wild-type recombinase recognition sequence and/or is present as an endogenous sequence in a genome that differs from the genome where the wild-type recognition sequence for the recombinase resides. “Pseudo attP site” or “pseudo attB site” refer to pseudo sites that are similar to wild-type phage or bacterial attachment site sequences, respectively, for phage integrase enzymes. “Pseudo att site” is a more general term that can refer to either a pseudo attP site or a pseudo attB site. Specific attB and attP sequences for use in the present invention include all wildtype sequences as well as pseudo attB and attP sequences.

Recombination sites used in the present methods include those recognized by unidirectional, site-directed recombinases (e.g., integrases). Non-limiting examples of serine integrases and recombination sites applicable to the present invention include C31 integrase, Bxb1, ΦBT1 integrase, A118, TP901-1, and R4 and the corresponding recombination sites for each (see, e.g., Groth, A. C. and Calos, M. P. (2004) J. Mol. Biol. 335, 667-678; Lei, et al., FEBS Lett. 2018 April; 592(8):1389-1399; Singh, et al., Attachment Site Selection and Identity in Bxb1 Serine Integrase-Mediated Site-Specific Recombination, PLoS Genet. 2013 May; 9(5):e1003490; and Gupta, et al., Nucleic Acids Res. 2007 May; 35(10): 3407-3419). Additional serine recombinases and recombination sites may be any of those disclosed in US 20180346934A1 and US 2010/0190178. In certain embodiments, a functional domain of the serine integrase is used.

In one example embodiment, the system can be used to insert or replace a sequence into one or more target genes. In example embodiments, the insertion or replacement results in an inactive target gene or less active form of the target gene. In one example embodiment, the system is used to replace all or a portion of the entire target gene. In one example embodiment, the system is used to replace all or a portion of an enhancer controlling the target gene expression.

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In one embodiment, the cargo may be a CAST system and/or one or more polynucleotides encoding one or more components thereof. CAST systems are able to insert DNA sequences at a target site in a DNA molecule without relying on host cell repair machinery. CAST systems can be Class 1 or Class 2 CAST systems. For example, a Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference. Suitable hybrid systems have also been described such as those described in Tou et al. bioRxiv 2022.01.07.475005, doi.org/10.1101/2022.01.07.475005, which is incorporated herein by reference. Additional CAST systems are disclosed, e.g., in WO 2020/131862 to Zhang et al and WO/2021/257997 to Zhang et al. (CAST-12k).; WO 2021/087394 to Zhang et al. and WO/2022/147321 to Zhang et al. (CAST-1b); WO 2022/076820 to Zhang et al. (CAST-1f); WO/2022/150651 to Zhang et al. (minimal Tn7-like CAST systems), all of which are incorporated herein by reference.

The CAST system may comprise a Cas linked to a transposase subunit to achieve RNA-guided DNA-transposition, optionally linked to a guide molecule. In some embodiments, the Cas may be catalytically inactive (e.g., Type I, IV, or Type V systems). Transposases suitable for the CAST system may be of any variety generally derived Tn7-like transposons (e.g. non-limiting examples, including TnsA, TnsB, TnsC, or TniQ). Guide molecules can guide the catalytically inactive Cas and a Tn7 or Tn7-like subunit to a target site to direct insertion of a donor at the target site.

CAST systems may require combinatorial transposases for efficient deposition. For example, TnsA is an endonuclease that cleaves the 5′-ends of the transposon and interacts with TnsB, TnsC, and DNA. TnsB is a recombinase capable of cleaving the 3′-end of the transposon. In some CAST systems with these components, the interaction between TnsA and TnsB achieves catalysis. In another instance, TnsC can direct TnsA and TnsB to the insertion site. In another instance, TniQ and DNA can be recognized by TnsC and enable the Cas complex to achieve insertion at the site.

In one example embodiment, the CAST system can be used to insert or replace a sequence into one or more target genes. In example embodiments, the insertion or replacement results in an inactive target gene or less active form of the target gene. In one example embodiment, a CAST system is used to replace all or a portion of an enhancer controlling the target gene expression. In an example embodiment, the enhancer controls the expression of one or more target genes or transcription factors selected from Tables 1 or 2.

CAST systems may be used to introduce one or more modifications (insertions, deletions, substitutions) that modify expression of the one or more genes of Tables 1A, 1B, 2A, and 2B, or a combination thereof. The modifications may be made in a non-coding region that controls expression of the one or more target genes, in a coding region encoding a gene expression product (e.g., a polypeptide), or both. Example modifications are described in further detail below.

### Non-LTR Systems

In one embodiment, the cargo may comprise a Non-LTR Retrotransposon system and/or one or more polynucleotide encoding to either decrease expression of one or more target genes or target transcription factors from Tables 1A and/or 1B or increase expression of one or more target genes or transcription factors from Tables 2A and/or 2B, or a combination thereof.

The a Non-LTR retrotransposon system may comprise one or more components of a retrotransposon, e.g., a non-LTR retrotransposon. Native or wild-type non-LTR retrotransposons encode the protein machinery necessary for their self-mobilization. The non-LTR retrotransposon element comprises a DNA element integrated into a host genome. The DNA element may encode one or two open reading frames (ORFs). For example, the R2 element of Bombyx mori encodes a single ORF containing reverse transcriptase (RT) activity and a restriction enzyme-like (REL) domain. Li elements encode two ORFs, ORF1 and ORF2. ORF1 contains a leucine zipper domain involved in protein-protein interactions and a C-terminal nucleic acid binding domain. ORF2 has a N-terminal apurinic/apyrimidinic endonuclease (APE), a central RT domain, and a C-terminal cysteine histidine rich domain. An example replicative cycle of a non-LTR retrotransposon may comprise transcription of the full-length retrotransposon element to generate an mRNA active element (retrotransposon RNA). The active element mRNA is translated to generate the encoded retrotransposon proteins or polypeptides. A ribonucleoprotein complex comprising the active element and retrotransposon protein or polypeptide is formed and this RNP facilitates integration of the active element into the genome. In an example embodiment, the RNA-transposase complex nicks the genome and the 3′ end of the nicked DNA serves as a primer to allow the reverse transcription of the transposon RNA into cDNA. The transposase proteins may then integrate the cDNA into the genome.

Elements of these systems may be engineered to work within the context of the invention. For example, a non-LTR retrotransposon polypeptide may be fused to a programmable nuclease. The binding elements that allow a non-LTR retrotransposon polypeptide to bind to the native retrotransposon DNA element, may be engineered into a donor construct to facilitate entry of a donor polynucleotide sequence into a target polypeptide.

In certain embodiments, the protein component of the non-LTR retrotransposon may be connected to or otherwise engineered to form a complex with a programmable nuclease, e.g., a Cas polypeptide. The retrotransposon RNA may be engineered to encode a donor polynucleotide sequence. Thus, in certain example embodiments, the Cas polypeptide, via formation of a CRISPR-Cas complex with a guide sequence, directs the retrotransposon complex (i.e., the retrotransposon polypeptide(s) and retrotransposon RNA to a target sequence in a target polynucleotide, where the retrotransposon RNP complex facilitates integration of the donor polynucleotide sequence into the target polynucleotide. Accordingly, the one or more non-LTR retrotransposon components may comprise retrotransposon polypeptides, or function domains thereof, that facilitate binding of the retrotransposon RNA, reverse transcription of the retrotransposon RNA into cDNA, and/or integration of the donor polynucleotide into the target polynucleotide, as well as retrotransposon RNA elements modified to encode the donor polynucleotide sequence. Example non-LTR retrotransposon systems are disclosed in WO 2021/102042, WO 2022/173830, which are incorporated herein by reference.

Examples of non-LTR retrotransposons may include those described in Christensen S M et al., RNA from the 5′ end of the R2 retrotransposon controls R2 protein binding to and cleavage of its DNA target site, Proc Natl Acad Sci USA. 2006 Nov. 21; 103(47):17602-7; Eickbush T H et al, Integration, Regulation, and Long-Term Stability of R2 Retrotransposons, Microbiol Spectr. 2015 April; 3(2):MDNA3-0011-2014. doi: 10.1128/microbiolspec.MDNA3-0011-2014; Han J S, Non-long terminal repeat (non-LTR) retrotransposons: mechanisms, recent developments, and unanswered questions, Mob DNA. 2010 May 12; 1(1):15. doi: 10.1186/1759-8753-1-15; MalikHS et al., The age and evolution of non-LTR retrotransposable elements, Mol Biol Evol. 1999 June; 16(6):793-805, which are incorporated by reference herein in their entireties.

Examples of the non-LTR retrotransposon polypeptides also include R2 from Clonorchis sinensis, or Zonotrichia albicollis. Example non-LTR retrotransposon polypeptides and binding components (5′ and 3′ UTRs) that may be used in the context of the invention are listed in Table 1 along with codon optimized variants of the non-LTR retrotransposons for expression in eukaryotic cells.

A non-LTR retrotransposon may comprise multiple retrotransposon polypeptides or polynucleotides encoding same. In some embodiments, the retrotransposon polypeptides may form a complex. For example, a non-LTR retrotransposon is a dimer, e.g., comprising two retrotransposon polypeptides forming a dimer. The dimer subunits may be connected or form a tandem fusion. A Cas protein or polypeptide may be associate with (e.g., connected to) one or more subunits of such complex. In some examples, the non-LTR retrotransposon is a dimer of two retrotransposon polypeptides; one of the retrotransposon polypeptides comprises nuclease or nickase activity and is connected with a Cas protein or polypeptide.

The retrotransposon polypeptides may be enzymes or variants thereof. In some examples, a retrotransposon polypeptide may be a reverse transcriptase, a nuclease, a nickase, a transposase, nucleic acid polymerase, ligase, or a combination thereof. In one example, a retrotransposon polypeptide is a reverse transcriptase. In another example, a retrotransposon polypeptide is a nuclease. In another example, a retrotransposon polypeptide is nickase. In a particular example, a non-LTR retrotransposon comprises a first retrotransposon polypeptide and a second retrotransposon polypeptide, wherein the second retrotransposon polypeptide comprises nuclease or nickase activity. In certain cases, a retrotransposon polypeptide may comprise an inactive enzyme. For example, a retrotransposon polypeptide may comprise a nuclease domain that is inactivated. Such inactivated domain may serve as a nucleic acid binding domain.

The retrotransposon polypeptides may comprise one or more modifications to, for example, enhance specificity or efficiency of donor polynucleotide recognition, target-primed template recognition (TPTR), and/or reduce or eliminate homing function. The retrotransposon polypeptides may also comprise one or more truncations or excisions to remove domains or regions of wild-type protein to arrive at a minimal polypeptide that retain donor polynucleotide recognition and TPTR. In some example embodiments, the native endonuclease activity may be mutated to eliminate endonuclease activity.

In certain example embodiments, the modifications or truncations of the non-LTR retrotransposon peptide may be in a zinc finger region, a Myb region, a basic region, a reverse transcriptase domain, a cysteine-histidine rich motif, or an endonuclease domain.

A non-LTR retrotransposon may comprise polynucleotide encoding one or more retrotransposon RNA molecules. The polynucleotide may comprise one or more regulatory elements. The regulatory elements may be promoters. The regulatory elements and promoters on the polynucleotides include those described throughout this application. For example, the polynucleotide may comprise a po12 promoter, a po13 promoter, or a T7 promoter.

In some cases, the polynucleotide encodes a retrotransposon RNA with at least a portion of its sequence complementary to a target sequence. For example, the 3′ end of the retrotransposon RNA may be complementary to a target sequence. The RNA may be complementary to a portion of a nicked target sequence. In some embodiments, a retrotransposon RNA may comprise one or more donor polynucleotides. In certain cases, a retrotransposon RNA may encode one or more donor polynucleotides.

A retrotransposon RNA may be capable of binding to a retrotransposon polypeptide. Such retrotransposon RNA may comprise one or more elements for binding to the retrotransposon polypeptide. Examples of binding elements include hairpin structures, pseudoknots (e.g., a nucleic acid secondary structure containing at least two stem-loop structures in which half of one stem is intercalated between the two halves of another stem), stem loops, and bulges (e.g., unpaired stretches of nucleotides located within one strand of a nucleic acid duplex). In certain examples, the retrotransposon RNA comprises one or more hairpin structures. In some examples, the retrotransposon RNA comprises one or more pseudoknots. In certain examples, a retrotransposon RNA comprises a sequence encoding a donor polynucleotide and one or more binding elements for forming a complex with the retrotransposon polypeptide. The binding elements may be located on the 5′ end, the 3′ end, or a location in between.

In some embodiments, a retrotransposon RNA comprises a region capable of hybridizing with an overhang of a target polynucleotide at the target site. The overhang may be a stretch of single-stranded DNA. The overhang may function as a primer for reverse transcription of at least a portion of the retrotransposon RNA to a cDNA. In some cases, a region of the cDNA may be capable of hybridizing a second overhang of the target polynucleotide. The second overhang may function as a primer for the synthesis of a second strand to generate a double-stranded cDNA. The cDNA may comprise a donor polynucleotide sequence. The two overhangs may be from different strands of the target polynucleotide.

### Donor Constructs

The systems may comprise one or more donor constructs comprising one or more donor polynucleotide sequences for insertion into a target polynucleotide. The donor construct comprises one or more binding elements. Examples of binding elements include hairpin structures, pseudoknots (e.g., a nucleic acid secondary structure containing at least two stem-loop structures in which half of one stem is intercalated between the two halves of another stem), stem loops, and bulges (e.g., unpaired stretches of nucleotides located within one strand of a nucleic acid duplex). In certain examples, the retrotransposon RNA comprises one or more hairpin structures. In some examples, the retrotransposon RNA comprises one or more pseudoknots. In certain examples, a retrotransposon RNA comprises a sequence encoding a donor polynucleotide and one or more binding elements for interacting to the retrotransposon polypeptide.

In certain example embodiments, the donor construct comprises a 5′ binding element and a 3′ binding element with a donor polynucleotide sequence located between the 5′ and 3′ prime binding element.

A donor polynucleotide may be any type of polynucleotides, including, but not limited to, a gene, a gene fragment, a non-coding polynucleotide, a regulatory polynucleotide, a synthetic polynucleotide, etc.

A target polynucleotide may comprise a protospacer adjacent motif (PAM) sequence. An example of the PAM sequence is AT.

The donor construct may further comprise one or more processing element. The processing element is an element that may be added to ensure accurate processing and incorporation of the donor polynucleotide sequence by the fusion proteins disclosed herein. Example processing elements include, but are not limited to, LRNA processing elements (e.g. GGCTCGTTGGGAGGTCCCGGGTTGAAATCCCGGACGAGCCCG (SEQ ID NO: 135)), human 28s processing elements (e.g. TAGCCAAATGCCTCGTCATCTAATTAGTGACGCGCATGAATGGATGAACGAGATT CCCACTGTCCCTACCTACTATCCAGCGAAACCACAGCCAAGGGAA (SEQ ID NO: 136)), and natural retrotransposon processing elements such as R2 processing elements from Bombyx mori (e.g. tagccaaatgcctcgtcatctaattagtgacgcgcatgaatggattaacgagattcccactgtccctatctactatctagcgaaaccacag ccaagggaacgggcttgggagaatcagcggggaa (SEQ ID NO: 137)).

The donor construct may comprise one or more homology sequence. A homology sequence is a sequence that shares or complete or partial homology with a target sequence at the site the targeted site of insertion. The homology sequence may be located on the 5′ end, ′3 end, or on both the 5′ and 3′ end of the donor construct. In certain example embodiments, the homology sequence is only located on the 5′ end of the donor construct. In certain example embodiments, the homology sequence is located only on the 3′ end of the donor construct. In certain example embodiments, the location of the homology sequence may depend on whether the site-specific nuclease is being directed to create a nick or cut 5′ or 3′ of the targeted insertion site, e.g. a 5′ homology sequence on the donor construct may be used when the site specific nuclease creates a nick or cut 5′ of the targeted insertion site and a 3′ homology sequence may be used when the site-specific nuclease is configured to create a nick or cut 3′ of the targeted insertion site. In certain example embodiments, the homology sequence is included on both the 5′ and 3′ ends of the donor construct regardless of whether the site-specific nuclease creates a nick or cut 5′ or 3′ of the targeted insertion site. In certain example embodiments the donor construct may comprise in a 5′ to 3′, a binding element, and the donor sequence In certain example embodiments the donor construct may comprise in a 5′ to 3′ direction a homology sequence, a binding element, and the donor sequence. In certain example embodiments the donor construct may comprise in a 5′ to 3′ direction a homology sequence, a first binding element, the donor sequence, and second binding element. In certain example embodiments, the donor construct may comprise in a 5′ to 3′ direction a first homology sequence, a first binding element, the donor sequence, and a second homology sequence. In certain example embodiments the donor construct may comprise, in a 5′ to 3′ direction, a first homology sequence, a first binding element, the donor sequence, a second binding element, and a second homology sequence. In certain example embodiments, the donor construct may comprise, in a 5′ to 3′ direction, the donor sequence and a binding element. In certain example embodiments, the donor construct may comprise, in a 5′ to 3′ direction, the donor sequence, a binding element, and a homology sequence. A processing element may be further incorporated 3′ of the donor sequence in any of the above donor construct configurations.

The homology sequence may have at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 175, 200 bases of homology to the target DNA. In certain example embodiments, the homology sequence may have between 8, 9, 10, 11, 12, 13, 14, 15, 16, 17, 18, 19, 20, 21, 22, 23, 24, or 25 base pairs of homology to the target sequence. In embodiments, with a homology sequence on both the 5′ and 3′ end of the donor construct, the size of the homology may be the same or different on each end. In some examples, the homology sequence comprises from 1 to 30, from 4 to 10, or from 10 to 25 nucleotides. For example, the homology sequence comprises from 4 to 10 nucleotides. For example, the homology sequence comprises from 10 to 25 nucleotides. For example, the homology sequence comprises 1 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides.

The donor polynucleotides may be inserted to the upstream or downstream of the PAM sequence of a target polynucleotide. For example, the donor polynucleotide may be inserted at a position between 10 bases and 200 bases, e.g., between 20 bases and 150 bases, between 30 bases and 100 bases, between 45 bases and 70 bases, between 45 bases and 60 bases, between 55 bases and 70 bases, between 49 bases and 56 bases or between 60 bases and 66 bases, from a PAM sequence on the target polynucleotide. In some cases, the insertion is at a position upstream of the PAM sequence. In some cases, the insertion is at a position downstream of the PAM sequence. In some cases, the insertion is at a position from 49 to 56 bases or base pairs downstream from a PAM sequence. In some cases, the insertion is at a position from 60 to 66 bases or base pairs downstream from a PAM sequence.

In a strand of a polynucleotide, anything towards the 5′ end of a reference point is “upstream” of that point, and anything towards the 3′ end of a reference point is “downstream” of that point. A location upstream of a PAM sequence refers to a location at the 5′ side of the PAM sequence on the PAM-containing strand of the target sequence. A location downstream of a PAM sequence refers to a location at the 3′ side of the PAM sequence on the PAM-containing strand of the target sequence.

The compositions and systems herein may be used to insert a donor polynucleotide with desired orientation. For example, appropriate homology sequence may be selected to control the orientation of insertion on the 5′ or 3′ strand of the target sequence.

The donor polynucleotide comprises a homology sequence of a region of the target sequence. The homology sequence may share at least 50%, at least 60%, at least 70%, at least 80%, at least 90%, at least 95%, or 100% sequence identity with the region of the target sequence. In an example, the homology sequence shares 100% sequence identity with the region of the target sequence.

In some embodiments, the donor polynucleotide may be inserted to the strand on the target sequence that contains the PAM (e.g., the PAM sequence of the site-specific nuclease such as Cas). In such cases, the donor polynucleotide may comprise a homology sequence of a region on the PAM containing strand of the target sequence. Such region may comprise the PAM sequence. The region may be at the 3′ side of the cleavage site of the site-specific nuclease. In some examples, the homology sequence may comprise from 4 to 10, or from 10 to 25 nucleotides in length. An example of such homology sequence may be of the “h1” region shown in FIG. 36.

In some embodiments, the donor polynucleotide may be inserted to the strand on the target sequence that binds to the guide, e.g., the strand that contains a guide-binding sequence. In such cases, the donor polynucleotide may comprise a homology sequence of a region that comprises at least a portion of the guide-binding sequence. In some cases, the region may comprise the entire guide-binding sequence. Such region may further comprise a sequence at the 3′ side of the guide-binding sequence. For example, the region may comprise from 5 to 15 nucleotides, e.g., 5, 6, 7, 8, 9, 10, 11, 12, 13, 14, 15 nucleotides from the 3′ side of the guide-binding sequence. In some cases, the region may be adjacent to the R-loop of the guide. For example, in the cases where the guide forms a RNA-DNA duplex with the guide-binding sequence, the region comprises a sequence at the 3′ side from the RNA-DNA duplex, e.g., from 5 to from 5 to 15 nucleotides, e.g., 5, 6, 7, 8, 9, 10, 11, 12, 13, 14, 15 nucleotides from the 3′ side from the RNA-DNA duplex. An example of such homology sequence may be of the “h2” region shown in FIG. 36.

In some examples, the homology sequence is of a region on the target sequence at 3′ side of a PAM-containing strand. In certain examples, the homology sequence is of a region on the target sequence 10 nucleotides from 3′ side of a RNA-DNA duplex formed by a guide molecule and a target sequence. For example, the guide molecule forms an RNA-DNA duplex with the target sequence, and the homology sequence is of a region on the target sequence 5 to 15 nucleotides from 3′ side of the RNA-DNA duplex. In some embodiments, the donor polynucleotide is inserted to a region on the target sequence that is 3′ side of a PAM-containing strand. In some cases, the donor polynucleotide is inserted to a region on the target sequence that is 3′ side of a sequence complementary to the guide molecule.

The donor polynucleotide may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor polynucleotide alters a stop codon in the target polynucleotide. For example, the donor polynucleotide may correct a premature stop codon. The correction may be achieved by deleting the stop codon or introduces one or more mutations to the stop codon. In other example embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g., sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of the corresponding wild-type gene. In certain example embodiments, these defective genes may be associated with one or more disease phenotypes. In certain example embodiments, the defective gene or gene fragment is not replaced but the systems described herein are used to insert donor polynucleotides that encode gene or gene fragments that compensate for or override defective gene expression such that cell phenotypes associated with defective gene expression are eliminated or changed to a different or desired cellular phenotype.

In certain embodiments, the donor may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain cases, the donor polynucleotide manipulates a splicing site on the target polynucleotide. In some examples, the donor polynucleotide disrupts a splicing site. The disruption may be achieved by inserting the polynucleotide to a splicing site and/or introducing one or more mutations to the splicing site. In certain examples, the donor polynucleotide may restore a splicing site. For example, the polynucleotide may comprise a splicing site sequence.

The donor polynucleotide to be inserted may has a size from 5 bases to 50 kb in length, e.g., from 50 to 40 kb, from 100 and 30 kb, from 100 bases to 300 bases, from 200 bases to 400 bases, from 300 bases to 500 bases, from 400 bases to 600 bases, from 500 bases to 700 bases, from 600 bases to 800 bases, from 700 bases to 900 bases, from 800 bases to 1000 bases, from 900 bases to from 1100 bases, from 1000 bases to 1200 bases, from 1100 bases to 1300 bases, from 1200 bases to 1400 bases, from 1300 bases to 1500 bases, from 1400 bases to 1600 bases, from 1500 bases to 1700 bases, from 600 bases to 1800 bases, from 1700 bases to 1900 bases, from 1800 bases to 2000 bases, from 1900 bases to 2100 bases, from 2000 bases to 2200 bases, from 2100 bases to 2300 bases, from 2200 bases to 2400 bases, from 2300 bases to 2500 bases, from 2400 bases to 2600 bases, from 2500 bases to 2700 bases, from 2600 bases to 2800 bases, from 2700 bases to 2900 bases, from 2800 bases to 3000 bases, from 2900 bases to 3100 bases, from 3000 bases to 3200 bases, from 3100 bases to 3300 bases, from 3200 bases to 3400 bases, from 3300 bases to 3500 bases, from 3400 bases to 3600 bases, from 3500 bases to 3700 bases, from 3600 bases to 3800 bases, from 3700 bases to 3900 bases, from 3800 bases to 4000 bases, from 3900 bases to 4100 bases, from 4000 bases to 4200 bases, from 4100 bases to 4300 bases, from 4200 bases to 4400 bases, from 4300 bases to 4500 bases, from 4400 bases to 4600 bases, from 4500 bases to 4700 bases, from 4600 bases to 4800 bases, from 4700 bases to 4900 bases, or from 4800 bases to 5000 bases in length.

### OMEGA System

In one example embodiment, the cargo may be oligonucleotides encoding one or more component of a transposon-encoded RNA-guided nuclease system, referred to herein as OMEGA (obligate mobile element-guided activity). See, e.g., Altae-Tran H, Kannan S, Demircioglu F E, et al. The widespread IS200/IS605 transposon family encodes diverse programmable RNA-guided endonucleases. Science. 2021; 374(6563):57-65. OMEGA systems include, but are not limited to IscB, IsrB, TnpB systems.

In some embodiments, the nucleic acid-guided nucleases herein may be an IscB protein (see, e.g., International patent application publication No. WO2022087494A1; and Altae-Tran H, et al. 2021). An IscB protein may comprise an X domain and a Y domain as described herein. In some examples, the IscB proteins may form a complex with one or more guide molecules. In some cases, the IscB proteins may form a complex with one or more hRNA molecules which serve as a scaffold molecule and comprise guide sequences. In some examples, the IscB proteins are CRISPR-associated proteins, e.g., the loci of the nucleases are associated with an CRISPR array. In some examples, the IscB proteins are not CRISPR-associated. In some examples, the IscB protein may be homolog or ortholog of IscB proteins described in Kapitonov V V et al., ISC, a Novel Group of Bacterial and Archaeal DNA Transposons That Encode Cas9 Homologs, J Bacteriol. 2015 Dec. 28; 198(5):797-807. doi: 10.1128/JB.00783-15, which is incorporated by reference herein in its entirety.

In some embodiments, the nucleic acid-guided nucleases herein may be an IsrB (Insertion sequence RuvC-like OrfB) protein (see, e.g., International patent application publication No. WO2022087494A1; and Altae-Tran H, et al. 2021). IsrB refers to a group of shorter, ˜350 aa IscB homologs that are also encoded in IS200/605 superfamily transposons. These proteins contain a PLMP domain and split RuvC but lack the HNH domain.

In some embodiments, the nucleic acid-guided nucleases herein may be a TnpB protein (see, e.g., International patent application publication No. WO2022159892A1; and Altae-Tran H, et al. 2021). TnpB is a putative endonuclease distantly related to IscB and thought to be the ancestor of Cas12, the type V CRISPR effector. The TnpB system comprises a TnpB polypeptide and a nucleic acid component capable of forming a complex with the TnpB polypeptide and directing the complex to a target polynucleotide. The TnpB systems and TnpB/nucleic acid component complexes may also be referred to herein as OMEGA (Obligate Mobile Element Guided Activity) systems or complexes, or Q systems or complexes for short. TnpB systems are a distinct type of Q system, which further include IscB, IsrB, and IshB systems. The nucleic acid component of Q systems is structurally distinct from other RNA-guided nucleases, such as CRISPR-Cas systems, and may also be referred to as a oRNA. In certain example embodiments, the TnpB systems are RNA-predominate, that is the nucleic acid component makes a larger contribution to the overall size of the TnpB complex relative to other RNA-guided nuclease systems such as CRISPR-Cas. Also, given the more minimal structural features of TnpB relative other known programmable nucleases such as CRISPR-Cas, the polynucleotide binding pocket is open and more accessible, which can facilitate greater access to and ability to manipulate, modify, edit, remove, or delete nucleotides at a target region on the bound polynucleotide.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or ₃₄ or ₃₅, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or ₃₄ or ₃₅)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 45)
M D P I R S R T P S P A R E L L S G P Q P D G V Q P T A D R G V S P P A G G P L D G
L P A R R T M S R T R L P S P P A P S P A F S A D S F S D L L R Q F D P S L F N T S L
F D S L P P F G A H H T E A A T G E W D E V Q S G L R A A D A P P P T M R V A V T
A A R P P R A K P A P R R R A A Q P S D A S P A A Q V D L R T L G Y S Q Q Q Q E K
I K P K V R S T V A Q H H E A L V G H G F T H A H I V A L S Q H P A A L G T V A V
K Y Q D M I A A L P E A T H E A I V G V G K Q W S G A R A L E A L L T V A G E L R
G P P L Q L D T G Q L L K I A K R G G V T A V E A V H A W R N A L T G A P L N

An exemplary amino acid sequence of a C-terminal capping region is:

(SEQ ID NO: 46)
R P A L E S I V A Q L S R P D P A L A A L T N D H L V A L A C L G G R P A L D A V K
K G L P H A P A L I K R T N R R I P E R T S H R V A D H A Q V V R V L G F F Q C H S
H P A Q A F D D A M T Q F G M S R H G L L Q L F R R V G V T E L E A R S G T L P P
A S Q R W D R I L Q A S G M K R A K P S P T S T Q T P D Q A S L H A F A D S L E R
D L D A P S P M H E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full-length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

### Zinc Finger Nucleases

Zinc Finger proteins can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### RNAi

In certain embodiments, the genetic modifying agent is an RNAi molecule (e.g., shRNA). RANi can reduce the transcription and/or translation of a target RNA molecule, by e.g., blocking RNA transcription and/or translation and/or reducing the amount of target RNA molecules by inducing their degradation.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Polypeptides

In certain example embodiments, the cargo is one or more polypeptides. The polypeptide may be a full-length protein or a functional fragment or functional domain thereof, that is a fragment or domain that maintains the desired functionality of the full-length protein. As used within this section “protein” is meant to refer to full-length proteins and functional fragments and domains thereof. A wide array of polypeptides may be delivered using the engineered PNMA capsids described herein, including but not limited to, secretory proteins, immunomodulatory proteins, anti-fibrotic proteins, proteins that promote tissue regeneration and/or transplant survival functions, hormones, anti-microbial proteins, anti-fibrillating polypeptides, and antibodies. The one or more polypeptides may also comprise combinations of the aforementioned example classes of polypeptides. It will be appreciated that any of the polypeptides described herein can also be delivered via the engineered PNMA capsids described herein via delivery of the corresponding encoding polynucleotide. As previously described,

### Secretory Proteins

In certain example embodiments, the one or more polypeptides may comprise one or more secretory proteins. A secretory is a protein that is actively transported out of the cell, for example, the protein, whether it be endocrine or exocrine, is secreted by a cell. Secretory pathways have been shown conserved from yeast to mammals, and both conventional and unconventional protein secretion pathways have been demonstrated in plants. Chung et al., “An Overview of Protein Secretion in Plant Cells,” MIMB, 1662:19-32, Sep. 1, 2017. Accordingly, identification of secretory proteins in which one or more polynucleotides may be inserted can be identified for particular cells and applications. In embodiments, one of skill in the art can identify secretory proteins based on the presence of a signal peptide, which consists of a short hydrophobic N-terminal sequence.

In embodiments, the protein is secreted by the secretory pathway. In embodiments, the proteins are exocrine secretion proteins or peptides, comprising enzymes in the digestive tract. In embodiments the protein is endocrine secretion protein or peptide, for example, insulin and other hormones released into the blood stream. In other embodiments, the protein is involved in signaling between or within cells via secreted signaling molecules, for example, paracrine, autocrine, endocrine or neuroendocrine. In embodiments, the secretory protein is selected from the group of cytokines, kinases, hormones and growth factors that bind to receptors on the surface of target cells.

As described, secretory proteins include hormones, enzymes, toxins, and antimicrobial peptides. Examples of secretory proteins include serine proteases (e.g., pepsins, trypsin, chymotrypsin, elastase and plasminogen activators), amylases, lipases, nucleases (e.g. deoxyribonucleases and ribonucleases), peptidases enzyme inhibitors such as serpins (e.g., al-antitrypsin and plasminogen activator inhibitors), cell attachment proteins such as collagen, fibronectin and laminin, hormones and growth factors such as insulin, growth hormone, prolactin platelet-derived growth factor, epidermal growth factor, fibroblast growth factors, interleukins, interferons, apolipoproteins, and carrier proteins such as transferrin and albumins. In some examples, the secretory protein is insulin or a fragment thereof. In one example, the secretory protein is a precursor of insulin or a fragment thereof. In certain examples, the secretory protein is c-peptide. In a preferred embodiment, the one or more polynucleotides is inserted in the middle of the c-peptide. In some embodiments, the secretory protein is GLP-1, glucagon, betatrophin, pancreatic amylase, pancreatic lipase, carboxypeptidase, secretin, CCK, a PPAR (e.g., PPAR-alpha, PPAR-gamma, PPAR-delta or a precursor thereof (e.g., preprotein or preproprotein). In aspects, the secretory protein is fibronectin, a clotting factor protein (e.g., Factor VII, VIII, IX, etc.), α2-macroglobulin, al-antitrypsin, antithrombin III, protein S, protein C, plasminogen, α2-antiplasmin, complement components (e.g., complement component C1-9), albumin, ceruloplasmin, transcortin, haptoglobin, hemopexin, IGF binding protein, retinol binding protein, transferrin, vitamin-D binding protein, transthyretin, IGF-1, thrombopoietin, hepcidin, angiotensinogen, or a precursor protein thereof. In aspects, the secretory protein is pepsinogen, gastric lipase, sucrase, gastrin, lactase, maltase, peptidase, or a precursor thereof. In aspects, the secretory protein is renin, erythropoietin, angiotensin, adrenocorticotropic hormone (ACTH), amylin, atrial natriuretic peptide (ANP), calcitonin, ghrelin, growth hormone (GH), leptin, melanocyte-stimulating hormone (MSH), oxytocin, prolactin, follicle-stimulating hormone (FSH), thyroid stimulating hormone (TSH), thyrotropin-releasing hormone (TRH), vasopressin, vasoactive intestinal peptide, or a precursor thereof.

### Immunomodulatory Polypeptides

In certain example embodiments, the one or more polypeptides may comprise one or more immunomodulatory protein. In certain embodiments, the present invention provides for modulating immune states. The immune state can be modulated by modulating T cell function or dysfunction. In particular embodiments, the immune state is modulated by expression and secretion of IL-10 and/or other cytokines as described elsewhere herein. In certain embodiments, T cells can affect the overall immune state, such as other immune cells in proximity.

In some embodiments, the immunomodulatory protein is an immunosuppressive protein or an immunostimulant protein. In some embodiments, the cargo is polynucleotide(s) that encode one or more immunomodulatory proteins. The term “immunosuppressive” means that immune response in an organism is reduced or depressed. An immunosuppressive protein may suppress, reduce, or mask the immune system or degree of response of the subject being treated. For example, an immunosuppressive protein may suppress cytokine production, downregulate or suppress self-antigen expression, or mask the MHC antigens. The term “immunostimulant” means that immune response in an organism is increased or activated.

As used herein, the term “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”) and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In some cases, the immunosuppressive proteins may exert pleiotropic functions. In some cases, the immunomodulatory proteins may maintain proper regulatory T cells versus effector T cells (Treg/Teff) balance. For examples, the immunomodulatory proteins may expand and/or activate the Tregs and blocks the actions of Teffs, thus providing immunoregulation without global immunosuppression. Target genes associated with immune suppression include, for example, checkpoint inhibitors such PD1, Tim3, Lag3, TIGIT, CTLA-4, and combinations thereof.

The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thaβ, CD4⁺, CD8⁺, effector Th, memory Th, regulatory Th, CD4⁺/CD8⁺ thymocytes, CD4−/CD8− thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

“T cell response” refers more specifically to an immune response in which T cells directly or indirectly mediate or otherwise contribute to an immune response in a subject. T cell-mediated response may be associated with cell mediated effects, cytokine mediated effects, and even effects associated with B cells if the B cells are stimulated, for example, by cytokines secreted by T cells. By means of an example but without limitation, effector functions of MHC class I restricted Cytotoxic T lymphocytes (CTLs), may include cytokine and/or cytolytic capabilities, such as lysis of target cells presenting an antigen peptide recognized by the T cell receptor (naturally-occurring TCR or genetically engineered TCR, e.g., chimeric antigen receptor, CAR), secretion of cytokines, preferably IFN gamma, TNF alpha and/or or more immunostimulatory cytokines, such as IL-2, and/or antigen peptide-induced secretion of cytotoxic effector molecules, such as granzymes, perforins or granulysin. By means of example but without limitation, for MHC class II restricted T helper (Th) cells, effector functions may be antigen peptide-induced secretion of cytokines, preferably, IFN gamma, TNF alpha, IL-4, IL5, IL-10, and/or IL-2. By means of example but without limitation, for T regulatory (Treg) cells, effector functions may be antigen peptide-induced secretion of cytokines, preferably, IL-10, IL-35, and/or TGF-beta. B cell response refers more specifically to an immune response in which B cells directly or indirectly mediate or otherwise contribute to an immune response in a subject. Effector functions of B cells may include in particular production and secretion of antigen-specific antibodies by B cells (e.g., polyclonal B cell response to a plurality of the epitopes of an antigen (antigen-specific antibody response)), antigen presentation, and/or cytokine secretion.

During persistent immune activation, such as during uncontrolled tumor growth or chronic infections, subpopulations of immune cells, particularly of CD8+ or CD4+ T cells, become compromised to different extents with respect to their cytokine and/or cytolytic capabilities. Such immune cells, particularly CD8+ or CD4+ T cells, are commonly referred to as “dysfunctional” or as “functionally exhausted” or “exhausted”. As used herein, the term “dysfunctional” or “functional exhaustion” refer to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation (e.g., in response to a cytokine, such as IFN-gamma) or cell division, entrance into the cell cycle, cytokine production, cytotoxicity, migration and trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is dysfunctional is a CD8+ T cell that expresses the CD8+ cell surface marker. Such CD8+ cells normally proliferate and produce cell killing enzymes, e.g., they can release the cytotoxins perforin, granzymes, and granulysin. However, exhausted/dysfunctional T cells do not respond adequately to TCR stimulation, and display poor effector function, sustained expression of inhibitory receptors and a transcriptional state distinct from that of functional effector or memory T cells. Dysfunction/exhaustion of T cells thus prevents optimal control of infection and tumors. Exhausted/dysfunctional immune cells, such as T cells, such as CD8+ T cells, may produce reduced amounts of IFN-gamma, TNF-alpha and/or one or more immunostimulatory cytokines, such as IL-2, compared to functional immune cells. Exhausted/dysfunctional immune cells, such as T cells, such as CD8+ T cells, may further produce (increased amounts of) one or more immunosuppressive transcription factors or cytokines, such as IL-10 and/or Foxp3, compared to functional immune cells, thereby contributing to local immunosuppression. Dysfunctional CD8+ T cells can be both protective and detrimental against disease control. As used herein, a “dysfunctional immune state” refers to an overall suppressive immune state in a subject or microenvironment of the subject (e.g., tumor microenvironment). For example, increased IL-10 production leads to suppression of other immune cells in a population of immune cells. In some embodiments, the cargo mitigates or prevents T cell exhaustion.

CD8+ T cell function is associated with their cytokine profiles. It has been reported that effector CD8+ T cells with the ability to simultaneously produce multiple cytokines (polyfunctional CD8+ T cells) are associated with protective immunity in patients with controlled chronic viral infections as well as cancer patients responsive to immune therapy (Spranger et al., 2014, J. Immunother. Cancer, vol. 2, 3). In the presence of persistent antigen CD8+ T cells were found to have lost cytolytic activity completely over time (Moskophidis et al., 1993, Nature, vol. 362, 758-761). It was subsequently found that dysfunctional T cells can differentially produce IL-2, TNFa and IFNg in a hierarchical order (Wherry et al., 2003, J. Virol., vol. 77, 4911-4927). Decoupled dysfunctional and activated Cell states have also been described (see, e.g., Singer, et al. (2016). A Distinct Gene Module for Dysfunction Uncoupled from Activation in Tumor-Infiltrating T Cells. Cell 166, 1500-1511 e1509; WO/2017/075478; and WO/2018/049025).

In some embodiments, the cargo(s) modulate T cell balance. The invention provides T cell modulating agents that modulate T cell balance. For example, in some embodiments, the invention provides T cell modulating agents and methods of using these T cell modulating agents to regulate, influence or otherwise impact the level of and/or balance between T cell types, e.g., between Th17 and other T cell types, for example, Th1-like cells. For example, in some embodiments, the invention provides T cell modulating agents and methods of using these T cell modulating agents to regulate, influence or otherwise impact the level of and/or balance between Th17 activity and inflammatory potential. As used herein, terms such as “Th17 cell” and/or “Th17 phenotype” and all grammatical variations thereof refer to a differentiated T helper cell that expresses one or more cytokines selected from the group the consisting of interleukin 17A (IL-17A), interleukin 17F (IL-17F), and interleukin 17A/F heterodimer (IL17-AF). As used herein, terms such as “Th1 cell” and/or “Th1 phenotype” and all grammatical variations thereof refer to a differentiated T helper cell that expresses interferon gamma (IFNγ). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3.

In some examples, immunomodulatory proteins are immunosuppressive cytokines. In general, cytokines are small proteins and include interleukins, lymphokines and cell signal molecules, such as tumor necrosis factor and the interferons, which regulate inflammation, hematopoiesis, and response to infections. Examples of immunosuppressive cytokines include interleukin 10 (IL-10), TGF-β, IL-Rα, IL-18Rα, IL-2, IL-3, IL-4, IL-5, IL-6, IL-7, IL-8, IL-9, IL-11, IL-12, IL-13, IL-14, IL-15, IL-16, IL-17, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, PGE2, SCF, G-CSF, CSF-1R, M-CSF, GM-CSF, IFN-α, IFN-β, IFN-γ, IFN-λ, bFGF, CCL2, CXCL1, CXCL8, CXCL12, CX3CL1, CXCR4, TNF-α and VEGF. Examples of immunosuppressive proteins may further include FOXP3, AHR, TRP53, IKZF3, IRF4, IRF1, and SMAD3. In one example, the immunosuppressive protein is IL-10. In one example, the immunosuppressive protein is IL-6. In one example, the immunosuppressive protein is IL-2.

### Anti-Fibrotic Proteins

In certain example embodiments, the one or more cargo polypeptides may comprise an anti-fibrotic protein. Examples of anti-fibrotic proteins include any protein that reduces or inhibits the production of extracellular matrix components, fibronectin, proteoglycan, collagen, elastin, TGIFs, and SMAD7. In embodiments, the anti-fibrotic protein is a peroxisome proliferator-activated receptor (PPAR) or may include one or more PPARs. In some embodiments, the protein is PPARα, PPARγ is a dual PPARα/γ. Derosa et al., “The role of various peroxisome proliferator-activated receptors and their ligands in clinical practice” Jan. 18, 2017 J. Cell. Phys. 223:1 153-161.

Proteins that Promote Tissue Regeneration and/or Transplant Survivale Functions

In certain example embodiments, the one or more cargo polypeptides may comprise a proteins that proteins that promote tissue regeneration and/or transplant survival functions. In some cases, such proteins may induce and/or up-regulate the expression of genes for pancreatic β cell regeneration. In some cases, the proteins that promote transplant survival and functions include the products of genes for pancreatic β cell regeneration. Such genes may include proislet peptides that are proteins or peptides derived from such proteins that stimulate islet cell neogenesis. Examples of genes for pancreatic β cell regeneration include Reg1, Reg2, Reg3, Reg4, human proislet peptide, parathyroid hormone-related peptide (1-36), glucagon-like peptide-1 (GLP-1), extendin-4, prolactin, Hgf, Igf-1, Gip-1, adipsin, resistin, leptin, IL-6, IL-10, Pdxl, Ptfal, Mafa, Pax6, Pax4, Nkx6.1, Nkx2.2, PDGF, vglycin, placental lactogens (somatomammotropins, e.g., CSH1, CHS2), isoforms thereof, homologs thereof, and orthologs thereof. In certain embodiments, the protein promoting pancreatic B cell regeneration is a cytokine, myokine, and/or adipokine.

### Hormones

In certain embodiments, the one or more cargo polypeptides may comprise one or more hormones. The term “hormone” refers to polypeptide hormones, which are generally secreted by glandular organs with ducts. Hormones include proteins from natural sources or from recombinant cell culture and biologically active equivalents of the native sequence hormone, including synthetically produced small-molecule entities and pharmaceutically acceptable derivatives and salts thereof. Included among the hormones are, for example, growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); prolactin, placental lactogen, mouse gonadotropin-associated peptide, inhibin; activin; mullerian-inhibiting substance; and thrombopoietin, growth hormone (GH), adrenocorticotropic hormone (ACTH), dehydroepiandrosterone (DHEA), cortisol, epinephrine, thyroid hormone, estrogen, progesterone, placental lactogens (somatomammotropins, e.g., CSH1, CHS2), testosterone. and neuroendocrine hormones. In certain examples, the hormone is secreted from pancreas, e.g., insulin, glucagon, somatostatin, pancreatic polypeptide and ghrelin. In some examples, the hormone is insulin.

Hormones herein may also include growth factors, e.g., fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, and glucocorticoids. In a particular embodiment, the hormone is insulin or incretins such as exenatide, GLP-1.

### Neurohormones

In embodiments, the cargo is a neurohormone, which is a hormone produced and released by neuroendocrine cells. In some embodiments, the neurohormone is a polypeptide. Example neurohormones include Thyrotropin-releasing hormone, Corticotropin-releasing hormone, Histamine, Growth hormone-releasing hormone, Somatostatin, Gonadotropin-releasing hormone, Serotonin, Dopamine, Neurotensin, Oxytocin, Vasopressin, Epinephrine, and Norepinephrine.

### Anti-Microbial Proteins

In some embodiments, the one or more polypeptides may comprise one or more anti-microbial proteins. In embodiments where the cell is mammalian cell, human host defense antimicrobial peptides and proteins (AMPs) play a critical role in warding off invading microbial pathogens. In certain embodiments, the anti-microbial is α-defensin HD-6, HNP-1 and β-defensin hBD-3, lysozyme, cathelcidin LL-37, C-type lectin RegIIIalpha, for example. See, e.g., Wang, “Human Antimicrobial Peptide and Proteins” Pharma, May 2014, 7(5): 545-594, incorporated herein by reference.

### Anti-Fibrillating Proteins

In certain example embodiments, the one or more polypeptides may comprise one or more anti-fibrillating polypeptides. The anti-fibrillating polypeptide can be the secreted polypeptide. In some embodiments, the anti-fibrillating polypeptide is co-expressed with one or more other polynucleotides and/or polypeptides described elsewhere herein. The anti-fibrillating agent can be secreted and act to inhibit the fibrillation and/or aggregation of endogenous proteins and/or exogenous proteins that it may be co-expressed with. In some embodiments, the anti-fibrillating agent is P4 (VITYF (SEQ ID. NO: 47)), P5 (VVVVV (SEQ ID. NO: 48)), KR7 (KPWWPRR (SEQ ID. NO: 49)), NK9 (NIVNVSLVK (SEQ ID. NO: 50)), iAb5p (Leu-Pro-Phe-Phe-Asp (SEQ ID. NO: 51)), KLVF (SEQ ID. NO: 52) and derivatives thereof, indolicidin, carnosine, a hexapeptide as set forth in Wang et al. 2014. ACS Chem Neurosci. 5:972-981, alpha sheet peptides having alternating D-amino acids and L-amino acids as set forth in Hopping et al. 2014. Elife 3:e01681, D-(PGKLVYA (SEQ ID. NO: 53)), RI-OR2-TAT, cyclo(17, 21)-(Lys17, Asp21)A_(1-28), SEN304, SEN1576, D3, R8-Aβ(25-35), human yD-crystallin (HGD), poly-lysine, heparin, poly-Asp, polyGl, poly-L-lysine, poly-L-glutamic acid, LVEALYL (SEQ ID. NO: 54), RGFFYT (SEQ ID. NO: 55), a peptide set forth or as designed/generated by the method set forth in U.S. Pat. No. 8,754,034, and combinations thereof. In aspects, the anti-fibrillating agent is a D-peptide. In aspects, the anti-fibrillating agent is an L-peptide. In aspects, the anti-fibrillating agent is a retro-inverso modified peptide. Retro-inverso modified peptides are derived from peptides by substituting the L-amino acids for their D-counterparts and reversing the sequence to mimic the original peptide since they retain the same spatial positioning of the side chains and 3D structure. In aspects, the retro-inverso modified peptide is derived from a natural or synthetic Aβ peptide. In some embodiments, the polynucleotide encodes a fibrillation resistant protein. In some embodiments, the fibrillation resistant protein is a modified insulin, see e.g., U.S. Pat. No. 8,343,914.

### Antibodies

In some embodiments, the one or more cargo polypeptides may be or comprise one or more antibodies. The term “antibody” is used interchangeably with the term “immunoglobulin” throughout the specification herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic′ treatment of an intact or complete antibody or antibody chain. Fragments can also be obtained by recombinant means. Exemplary fragments include Fab, Fab′, F(ab′)2, Fabc, Fd, dAb, V_{HH} and scF “and/or Fv fragments.” As used herein, a preparation of antibody protein “having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g., the IgG1, IgG2, IgG3, and IgG4 subclasses of IgG obtained from any source (e.g., humans and non-human primates, and in rodents, lagomorphs, caprines, bovines, equines, ovines, etc.).

The term “Ig class” or “immunoglobulin class”, as used herein”, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, 1gM antibodies exist in pentameric f-rm, and IgA antibodies exist in monomeric, dimeric or multimeric form.

The term “IgG subclass” refers to the four subclasses of immunoglobulin class IgG—IgG1, IgG2, IgG3, and IgG4 that have been identified in humans and higher mammals by the heavy chains of the immunoglobulins, V1—γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 or 4 peptide loops) stabilized, for example, by R pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant domains” of an antibody light chain are referred to interchangeably as “light chain constant regions”, “light chain constant domains”, “CL regions” or “CL domains” The constant domains of an antibody heavy chain are referred to interchangeably as “heavy chain constant region”, “heavy chain constant domains”, “CH regions” or “CH domains”. The “variable domains” of an antibody light chain are referred to interchangeably as “light chain variable regions”, “light chain variable domains”, “VL” regions or “VL” domains.” The variable domains of an “antibody heavy” chain are referred to interchangeably as “heavy chain constant regions”, “heavy chain constant domains”, “VH” regions or “VH” domains.

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (58 residues) and robust, disulphide-crosslinked serine protease inhibitor, typically of human origin (e.g., LACI-D1), which can be engineered for different protease spec^{if}icities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins-harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin″peptides (Kolmar″ Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant cross reactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly cross-react with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity, but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding′fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which ′ is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii)′isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/4028 1; U.S. Pat. No. 5,811,097; Deng et al., Blood 92 (6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-321 4 (1998); Yoon et al., J. Immunol. 160(7):3170-3179 (1998); Pr at et al., J. Cell. Sci. III (Pt2):237-247 (1998); Pitard et al., J. Immunol. Methods 205 (2):177-190 (1997); Liautard et al., Cytokine 9(4):233-241 (1997); Carlson et al., J. Biol. Che m. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### Nanobodies and Monobodies

In some embodiments, the one or more cargo polypeptides are nanobodies. As used throughout the specification herein, “nanobody(sies)” refers to engineered antigen binding V_{H}H fragments, typically of an antibody, such as an IgG. They are also referred to in the art a single domain antibodies (sdAb). Methods of designing, engineering, and producing nanobodies for specific targets and uses is generally known in the art. See e.g., S. Muyldermans. Annu Rev Anim Biosci. 2021 Feb. 16; 9:401-421; S. Muyldermans. Annu Rev Biochem. 2013; 82:775-97; and S. Muyldermans. FEBS J. 2021 April; 288(7):2084-2102.

In some embodiments, the one or more cargo polypeptides are monobodies. As used throughout the specification herein, “monobody(ies)” refers to engineered or synthetic binding proteins constructed using a fibronectin type III domain or variant thereof (see e.g., Koide et al. 1998, J. Mol. Biol. 284(4) 1141-1151, Koide et al. 2012. Meth. Enzymol. 503:135-156; and Koide et al., 2012. J. Mol. Biol. 415(2): 393-405). Methods of designing, engineering, and producing monobodies for specific targets and uses is generally known in the art. See e.g., Sha et al., Protein Sci. 2017 May; 26(5):910-924; Annu Rev Pharmacol Toxicol. 2020 Jan. 6; 60:391-415; and Hantschel et al., Curr Opin Struct Biol. 2020 February; 60:167-17.

### Protease Cleavage Sites

The one or more cargo polypeptides, as exemplified above, may comprise one or more protease cleavage sites, i.e., amino acid sequences that can be recognized and cleaved by a protease. The protease cleavage sites may be used for generating desired gene products (e.g., intact gene products without any tags or portion of other proteins). The protease cleavage site may be one end or both ends of the protein. Examples of protease cleavage sites that can be used herein include an enterokinase cleavage site, a thrombin cleavage site, a Factor Xa cleavage site, a human rhinovirus 3C protease cleavage site, a tobacco etch virus (TEV) protease cleavage site, a dipeptidyl aminopeptidase cleavage site and a small ubiquitin-like modifier (SUMO)/ubiquitin-like protein-1 (ULP-1) protease cleavage site. In certain examples, the protease cleavage site comprises Lys-Arg.

### Small Molecules

In some embodiments, the cargo is or includes one or more small molecule compounds. In some embodiments, the small molecule compound(s) can be indirectly linked or directly attached to a polynucleotide or polypeptide that can bind a polynucleotide and/or polypeptide that can be included in the engineered PNMA capsid. In some embodiments, the engineered PNMA capsid can include a small molecule binding protein (e.g., a receptor) for the small molecule. Small molecules can include biological molecules and chemical molecules and others.

Exemplary small molecule cargos include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, radiation sensitizers, chemotherapeutics.

Exemplary hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g., arachidonic acid, lipoxins, and prostaglandins), and steroid hormones (e.g., estradiol, testosterone, tetrahydro testosterone, cortisol).

Exemplary immunomodulators include, but are not limited to, prednisone, azathioprine, 6-MP, cyclosporine, tacrolimus, methotrexate, interleukins (e.g., IL-2, IL-7, and IL-12), cytokines (e.g., interferons (e.g., IFN-a, IFN-β, IFN-ε, IFN-K, IFN-ω, and IFN-γ), granulocyte colony-stimulating factor, and imiquimod), chemokines (e.g., CCL3, CCL26 and CXCL7), cytosine phosphate-guanosine, oligodeoxynucleotides, glucans, antibodies, and aptamers).

Exemplary antipyretics include, but are not limited to, non-steroidal anti-inflammants (e.g., ibuprofen, naproxen, ketoprofen, and nimesulide), aspirin and related salicylates (e.g., choline salicylate, magnesium salicylate, and sodium salicylate), paracetamol/acetaminophen, metamizole, nabumetone, phenazone, and quinine.

Exemplary anxiolytics include, but are not limited to, benzodiazepines (e.g., alprazolam, bromazepam, chlordiazepoxide, clonazepam, clorazepate, diazepam, flurazepam, lorazepam, oxazepam, temazepam, triazolam, and tofisopam), serotenergic antidepressants (e.g., selective serotonin reuptake inhibitors, tricyclic antidepressants, and monoamine oxidase inhibitors), mebicar, afobazole, selank, bromantane, emoxypine, azapirones, barbiturates, hydroxyzine, pregabalin, validol, and beta blockers.

Exemplary antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Exemplary analgesics include, but are not limited to, paracetamol/acetaminophen, nonsteroidal anti-inflammants (e.g., ibuprofen, naproxen, ketoprofen, and nimesulide), COX-2 inhibitors (e.g., rofecoxib, celecoxib, and etoricoxib), opioids (e.g., morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

Exemplary antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene. Suitable anti-inflammatories include, but are not limited to, prednisone, non-steroidal anti-inflammants (e.g., ibuprofen, naproxen, ketoprofen, and nimesulide), COX-2 inhibitors (e.g., rofecoxib, celecoxib, and etoricoxib), and immune selective anti-inflammatory derivatives (e.g., submandibular gland peptide-T and its derivatives).

Exemplary anti-histamines include, but are not limited to, H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, rafitidine, and roxatidine), tritoqualine, catechin, cromoglicate, nedocromil, and p2-adrenergic agonists.

Exemplary anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, abendazole, thiabendazole, oxamniquine), antifungals (e.g., azole antifungals (e.g., itraconazole, fluconazole, posaconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g., aminosalicylates (e.g., aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g., vancomycin, dalbavancin, oritavancin, and telvancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxaxillin, dicloxacillin, and nafcillin), quinolones (e.g., lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfasoxazole), tetracyclines (e.g., doxycycline, demeclocycline, minocycline, doxycycline/salicyclic acid, doxycycline/omega-3 polyunsaturated fatty acids, and tetracycline), and urinary anti-infectives (e.g., nitrofurantoin, methenamine, fosfomycin, cinoxacin, nalidixic acid, trimethoprim, and methylene blue).

Exemplary chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, arsenic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

Suitable radiation sensitizers include, but are not limited to, 5-fluorouracil, platinum analogs (e.g., cisplatin, carboplatin, and oxaliplatin), gemcitabine, DNA topoisomerase I-targeting drugs (e.g., camptothecin derivatives (e.g., topotecan and irinotecan)), epidermal growth factor receptor blockade family agents (e.g., cetuximab, gefitinib), farnesyltransferase inhibitors (e.g., L-778-123), COX-2 inhibitors (e.g., rofecoxib, celecoxib, and etoricoxib), bFGF and VEGF targeting agents (e.g., bevazucimab and thalidomide), NBTXR3, Nimoral, trans sodium crocetinate, NVX-108, and combinations thereof. See also e.g., Kvols, L. K., J Nucl Med 2005; 46:187S-190S.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof, such as a PNMA protein, PNMA capsid (e.g., a cargo loaded PNMA capsid), and/or the like of the present invention (which are also referred to as the primary active agent or ingredient elsewhere herein) described in greater detail elsewhere herein and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, one or more of the PNMA proteins, PNMA capsids (e.g., a cargo loaded PNMA capsid), and/or the like of the present invention.

In some embodiments, the active ingredient, such as a cargo, is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents (e.g., in addition to a cargo loaded PNMA capsid), including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the one or more therapeutic effects are reducing or altering an arrythmia.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, M, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments where a cell or cell population is present in the pharmaceutical formulation (e.g., as a primary and/or or secondary active agent), the effective amount of cells can be any amount ranging from about 1 or 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL or any numerical value or subrange within any of these ranges.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be about 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In some embodiments, the amount or effective amount of the one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Kits

Any of the compounds, compositions, formulations, particles, cells, and/or the like, such as the PNMA proteins, capsids, and/or cargos described herein, or any combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, and/or the like, such as the PNMA proteins, capsids, and/or cargos described herein, or any combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, cells, and/or the like, such as the PNMA proteins, capsids, and/or cargos described herein, or any combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, and/or the like, such as the PNMA proteins, capsids, and/or cargos described herein or any combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, and cells, and/or the like, such as the PNMA proteins, capsids, and/or cargos described herein, or a combination thereof to a subject in need thereof.

### Methods of Cargo Delivery

Described in several example embodiments herein are methods of delivering one or more cargos to one or more cells using the engineered PNMA capsids of the present invention. In some embodiments, the method comprises delivering the engineered delivery system comprising a PNMA capsid containing a cargo of the present invention or a pharmaceutical formulation thereof to a cell or population of cells in vitro or in vivo. In some embodiments, the cells are human cells. In some embodiments, the cells are non-human animal cells.

In some embodiments, the method comprises delivering a cell or cells capable of producing the engineered delivery system comprising a PNMA capsid containing a cargo of the present invention to a subject in need thereof. Exemplary cell based therapies comprising a cell or cells capable of producing the engineered delivery system comprising a PNMA capsid containing a cargo of the present invention are described in greater detail elsewhere herein. In some embodiments, the cells are autologous. In some embodiments, the cells are allogenic.

In some embodiments, the number of particles (e.g., capsids) delivered can be about 1-1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles. In some embodiments, the amount of particles (e.g., capsids) delivered can be about 1×10¹ particles) per pL, nL, μL, mL, or L to 1×10²⁰/particles per pL, nL, pL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L.

In some embodiments where a cell or cell population capable of producing the engineered PNMA capsids are administered, amount of cells delivered to a subject can be any amount ranging from about 1 or 2 cells to 1×10¹ cells/mL, 1×10²⁰ cells/mL or more, such as about 1×10¹ cells/mL, 1×10² cells/mL, 1×10³ cells/mL, 1×10⁴ cells/mL, 1×10⁵ cells/mL, 1×10⁶ cells/mL, 1×10⁷ cells/mL, 1×10⁸ cells/mL, 1×10⁹ cells/mL, 1×10¹⁰ cells/mL, 1×10¹¹ cells/mL, 1×10¹² cells/mL, 1×10¹³ cells/mL, 1×10¹⁴ cells/mL, 1×10¹⁵ cells/mL, 1×10¹⁶ cells/mL, 1×10¹⁷ cells/mL, 1×10¹⁸ cells/mL, 1×10¹⁹ cells/mL, to/or about 1×10²⁰ cells/mL or any numerical value or subrange within any of these ranges.

Administration can be by any suitable route. Exemplary administration routes include, without limitation, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

The engineered delivery system comprising a PNMA capsid containing a cargo of the present invention are administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the p engineered delivery system comprising a PNMA capsid containing a cargo of the present invention are administered continuously over a period of time ranging from minutes to hours to days. Devices and dosages forms are known in the art and described herein that are effective to provide continuous administration of engineered delivery system comprising a PNMA capsid containing a cargo of the present invention. In some embodiments, the first one or a few initial amount(s) administered can be a higher dose than subsequent doses. This is typically referred to in the art as a loading dose or doses and a maintenance dose, respectively. In some embodiments, the engineered delivery system comprising a PNMA capsid containing a cargo of the present invention are administered such that the doses over time are tapered (increased or decreased) overtime so as to wean a subject gradually off of an engineered delivery system comprising a PNMA capsid containing a cargo of the present invention or gradually introduce a subject to the engineered delivery system comprising a PNMA capsid containing a cargo of the present invention.

The engineered delivery system comprising a PNMA capsid containing a cargo of the present invention can be delivered as a co-therapy. Where co-therapies or engineered delivery system comprising a PNMA capsid containing a cargo of the present invention are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

The combination treatment can include the engineered delivery system comprising a PNMA capsid containing a cargo of the present invention and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Methods of Loading Cargo Molecules in Delivery Vesicle Systems

In some embodiments, the cargo can be loaded into an engineered delivery vesicle of the present invention in vivo. Generally, in vivo loading or packaging involves expressing an engineered delivery system of the present invention in a cell that contains cargo to be loaded into the engineered delivery vesicle that is generated by expression of the engineered delivery system. When the engineered delivery system is expressed and the engineered delivery particle is formed, cargo present in the cell can be packaged into the engineered delivery vesicles. The loaded engineered delivery vesicles can be harvested, isolated, and/or purified from the cells and/or culture supernatant (if the loaded engineered delivery vesicles are secreted by the cells) by any suitable method which will be appreciated by one of ordinary skill in the art in view of the description herein. The cargo can be endogenous or exogenous to the cell used for loading/particle production.

The cargo, which is of a size sufficiently small to be enclosed in the delivery vesicle, e.g., nucleic acids and/or polypeptides, can be introduced to cells by transduction by a viral or pseudoviral particle. Methods of packaging the cargos in viral particles can be accomplished using any suitable vector systems. Such vector systems are described in greater detail elsewhere herein. As used in this context herein “transduction” refers to the process by which foreign nucleic acids and/or proteins are introduced to a cell (prokaryote or eukaryote) by a viral or pseudo viral particle. Cargo-loaded delivery vesicles of the present invention can be exposed to cells (e.g., in vitro, ex vivo, or in vivo) where the delivery vesicles deliver the cargo to the target cell, for example, by transduction. Delivery vesicles can be optionally concentrated prior to exposure to target cells.

One approach for packaging cargo inside vesicles involves the use of one or more “bioreactors” which produce and subsequently secrete one or more cargo-carrying vesicles. Bioreactors may comprise cells, microorganisms, or acellular systems. A bioreactor cell is generated by administering to a cell one or more polynucleotides encoding one or more (e.g., endogenous) LTR retroelement polypeptides for forming a delivery vesicle and one or more capture moieties for packaging a cargo within the delivery vesicle. Accordingly, the bioreactor may be capable of producing cargo-carrying vesicles that not only deliver the biologically active RNA molecule(s) to the extracellular matrix, but also to specific cells and tissues. Cells suitable for being bioreactor cells for producing engineered delivery system polynucleotides, polypeptides, and/or engineered delivery vesicles (loaded with a cargo(s) or not) are described elsewhere herein.

In some embodiments, the cargo can be loaded or packaged into the engineered delivery vesicle in vitro or acellularly. Generally, in these embodiments, PNMA, e.g., PNMA2 or engineered PNMA2, monomers are incubated with cargos under conditions sufficient to promote capsid formation and packaging of the cargos within the capsids (see e.g., FIG. 5, which demonstrates as an example, in vitro packaging of RNA cargo by PNMA2 monomers and subsequent capsid formation).

In some embodiments, in vitro capsid formation from PNMA monomers can be controlled via the salt type and/or concentration of the in vitro environment, (e.g., solution). In some embodiments, a mixture of NaCl and CaCl₂ drives formation of capsids. In some embodiments, a solution containing at about 100-600 or more mM NaCl and about 5 to about 100 mM CaCl₂ can promote formation of PNMA, e.g., PNAM2, capsids and loading of cargo when present. In some embodiments, the a solution containing about 100 to/or 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600 mM of NaCl and about 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mM CaCl₂ can promote formation of the PNMA capsids and loading of cargo when present. In some embodiments, the in vitro solution for generating and/or loading the PNMA capsids contains about 500 mM NaCl and about 10 mM CaCl₂.

In some embodiments, an in vitro solution for disassembling the PNMA capsids contains about 5 to about 50 mM NaCl (e.g., 5 to/or 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nM NaCl). In some embodiments, an in vitro solution for disassembling the PNMA capsids contains about 25 mM NaCl. In some embodiments, an in vitro solution for disassembling PNMA capsids does not contain CaCl₂. In some embodiments, an in vitro solution for disassembling PNMA capsids contains about 5 to about 5 mM NaCl and does not contain CaCl₂. In some embodiments, an in vitro solution for disassembling PNMA capsids contains about 25 mM NaCl and does not contain CaCl₂. In some embodiments, an in vitro solution for disassembling PNMA capsids contains about 1 to about 10 M urea. In some embodiments, an in vitro solution for disassembling PNMA capsids contains about 1.0 M, to/or about 1.1M, 1.2M, 1.3M, 1.4M, 1.5M, 1.6M, 1.7M, 1.8M, 1.9M, 2M, 2.1M, 2.2M, 2.3M, 2.4M, 2.5M, 2.6M, 2.7M, 2.8M, 2.9M, 3M, 3.1M, 3.2M, 3.3M, 3.4M, 3.5M, 3.6M, 3.7M, 3.8M, 3.9M, 4M, 4.1M, 4.2M, 4.3M, 4.4M, 4.5M, 4.6M, 4.7M, 4.8M, 4.9M, 5M, 5.1M, 5.2M, 5.3M, 5.4M, 5.5M, 5.6M, 5.7M, 5.8M, 5.9M, 6M, 6.1M, 6.2M, 6.3M, 6.4M, 6.5M, 6.6M, 6.7M, 6.8M, 6.9M, 7M, 7.1M, 7.2M, 7.3M, 7.4M, 7.5M, 7.6M, 7.7M, 7.8M, 7.9M, 8M, 8.1M, 8.2M, 8.3M, 8.4M, 8.5M, 8.6M, 8.7M, 8.8M, 8.9M, 9M, 9.1M, 9.2M, 9.3M, 9.4M, 9.5M, 9.6M, 9.7M, 9.8M, 9.9M, 10 M urea.

In certain example embodiments a method of in vitro packaging a cargo in a capsid comprising one or more engineered paraneoplastic Ma proteins (PNMA) described herein includes combining a cargo and a plurality of engineered PNMA protein monomers of the present disclosure in an assembly solution comprising an amount of a salt and an amount of calcium chloride thereby promoting assembly of the capsid and packaging of the cargo in the capsid. In some embodiments, the one or more PNMA is PNMA2 and/or PMNA3.

In certain example embodiments, the amount of salt in the assembly solution is about 100 mM to about 600 mM, wherein the amount of calcium chloride in the assembly solution is about 5 to about 100 mM, or both. In some embodiments, the amount of salt in the assembly solution is about 500 mM, wherein the amount of calcium chloride in the assembly solution is about 10 mM, or both.

In certain example embodiments, the method further comprises generating the plurality of engineered PNMA monomers prior to combining, wherein generating the plurality of engineered PNMA monomers comprises disassembling one or more capsids comprising a plurality of engineered PNMAs by exposing the capsid comprising one or more engineered PNMAs to a disassembly solution thereby generating the plurality of PNMA monomers.

In certain example embodiments, the disassembly solution comprises an amount of a salt or an amount of urea effective to disassembly the one or more capsids comprising a plurality of engineered PNMAs. In certain example embodiments, the disassembly solution comprises an amount of salt or an amount of urea effective to promote disassembly of the one or more capsids. In certain example embodiments, the disassembly solution comprises about 5 mM to about 50 mM salt or about 6 M urea. In certain example embodiments, the disassembly solution does not contain calcium chloride.

Further embodiments are illustrated in the following Examples which are given for illustrative purposes only and are not intended to limit the scope of the invention.

### EXAMPLES

### Example 1—Engineered PNMAs

Engineered PNMA proteins can be generated that include elements to facilitate purification of an engineered PNMA protein. Exemplary PNMA purification constructs are shown in Table 4, where “PNMA” in Table 4 represents PNMA proteins with one or more or all of the modifications described below.

TABLE 4
PNMA Purification Constructs

| | Protein Sequence (From N-terminus to C-terminus) |
|---|---|
| N-terminal Tag | 6XHis-MBP-bdSUMO-PNMA |
| C-terminal Tag-1 | PNMA-Mxe GyrA Intein-MBP-6XHis |
| C-terminal Tag-2 | PNMA-Mxe GyrA Intein-2XMBP-6XHis |

### Inner Capsid Surface Modifications

The engineered PNMA proteins can be generated that include one or more modifications that modify the inner surface of a capsid formed from the engineered PNMA proteins. The modifications are described below. It will be appreciated that any combination of the modifications below can be introduced into the engineered PNMA protein. Further, inner capsid surface modifications can be coupled with outer capsid surface modifications later described.

### Charge Modifications

The modifications of the PNMA can change the charge of the inner surface of the capsid formed from the PNMA protein. This can allow for capture/packaging of negatively charged cargos (e.g., when the PNMA is modified such that the inner surface of a capsid formed from the modified PNMA contains positively charged modifications) and/or positively charged cargos (e.g., when the PNMA is modified such that the inner surface of a capsid formed from the modified PNMA contains negatively charged modifications). PNMA2 was modified at the loop corresponding to amino acid residues 170-180, at the loop corresponding to amino acid residues 256-263, and/or at the loop corresponding to amino acid residues 302-305 of the PNMA2, modified at the C-terminus with an extension or a truncation and an extension, or a combination thereof. Modifications of the loop(s) included insertion of positively charged residues (e.g., lysine and/or arginine). The C-terminal extensions included addition of an extension sequence rich in arginine, leucine, and/or proline. PNMA2 C-terminal truncations included truncations of PNMA2 to amino acid 348E or 3405. Table 5 below provides a summary of the modifications that can be made to the PNMA2. The inner capsid surface charge modifications can be made alone or in any permissible combination. The inner capsid surface charge modification(s) can be introduced with any one or more of the other outer or inner capsid surface modifications detailed in this Example and elsewhere in the present disclosure.

TABLE 5
PNMA2 Charge modifications (inner capsid surface)
Modification

| | |
|---|---|
| Loop 170-180 Insertion | Insertion of RRKRRKRRKRRK (SEQ ID NO: 7) |
| | between 175A and 176P |
| Loop 256-263 Insertion | Insertion of RRKRRKRRKRRK (SEQ ID NO: 7) |
| | between 261E and 262G |
| Loop 302-205 Insertion | Insertion of RRKRRKRRKRRK (SEQ ID NO: 7) |
| | between 303T and 304L |
| C-terminus extension | Extent the C-terminus with RRLRRPRRLRRPRRPR |
| | (SEQ ID NO: 8) |
| Truncation and C-terminal | PNMA2 truncation to 348E then extended with |
| extension-1 | RRLRRPRRLRRPRRPR (SEQ ID NO: 8) |
| Truncation and C-terminal | PNMA2 truncation to 340S then extended with |
| extension-2 | RRLRRPRRLRRPRRPR (SEQ ID NO: 8) |

### RNA Binding Peptide Modifications

The modifications can introduce RNA binding domains into the PNMA and inner capsid surface of a capsid formed from the engineered PNMA proteins. This can allow for capture/packaging of RNA cargos within the capsid. PNMA2 was modified with an RNA binding peptide at the loop corresponding to amino acids 256-263, at the loop corresponding to 302-305, at C-terminus, or any combination thereof. PNMA3 was modified at the N Terminus, at the Zinc finger region, and/or C-terminus with an RNA binding peptide. Table 6 below sets forth exemplary modifications of the PNMA2 with various RNA binding peptides. Table 7 below sets forth exemplary modifications of the PNMA3 with various RNA binding peptides. Other modifications included replacing the λN in the modifications of Table 6 or Table 7 with any one of the following sequences: λN with enhanced binding: GNARTRRRERRAEKQAQWKAAN (SEQ ID NO: 2); P22N: GNAKTRRHERRRKLAIERDTI (SEQ ID NO: 3); or HIV-1 Rev: TRQARRNRRRRWRERQR (SEQ ID NO: 4), CCMV N-terminal sequence (MSTVGTGKLTRAQRRAAARKNKRNTRVVQP (SEQ ID NO: 5)), PNMA3 residues 336-463 (when the PNMA being modified was PNMA2 or other non-PNMA3 PNMA).

PNMA2(1-340)-CCMV demonstrated RNA packaging properties. PNMA2(1-340)-CCMV contained PNMA2 amino acids 1-340 followed by (fused to) the CCMV N-terminal sequence.

PNMA2(1-340)-PNMA3(336-463) demonstrated RNA packaging properties. PNMA2(1-340)-PNMA3(336-463) is PNMA2 residue 1 to residue 340 followed by (fused to) PNMA3 residues 336 to residue 463.

PNMA2(1-333)-AS-PNMA3(336-463) demonstrated RNA packaging properties. PNMA2(1-333)-AS-PNMA3(336-463) is PNMA2 residue 1 to 333 followed by (fused to) an Alanine and Serine (AS), which was followed by (fused to) PNMA3 residues 336-463.

The RNA binding modification can be introduced with any one or more of the other outer or inner capsid surface modifications detailed in this Example and elsewhere in the present disclosure.

TABLE 6
PNMA2 RNA binding peptide modification (inner capsid surface)
Modification

| | |
|---|---|
| Loop 256-263 Insertion | Insertion of GSGGGSG-λN-GSGGGSG-between 261E |
| | and 262G (SEQ ID NO: 56-61) |
| Loop 302-205 Insertion | Insertion of GS-λN-GS between 303T and 304L (SEQ ID |
| | NO: 62-67) |
| C-terminus extension | Extent the C-terminus with GS-λN (SEQ ID NO: 68-73) |
| Truncation and C-terminal | PNMA2 truncation to 340S then extended with GS-λN |
| extension | (SEQ ID NO: 68-73) |

λN peptide: MDAQTRRRERRAEKQAQWKAAN (SEQ ID NO: 1)
λN peptide can be replaced with the following λN sequence
λN with enhanced binding = GNARTRRRERRAEKQAQWKAAN (SEQ ID NO: 2)
P22N = GNAKTRRHERRRKLAIERDTI (SEQ ID NO: 3)
HIV-1 Rev = TRQARRNRRRRWRERQR (SEQ ID NO: 4)
CCMV N-terminal = MSTVGTGKLTRAQRRAAARKNKRNTRVVQP (SEQ ID NO: 5)
PNMA3 Zinc Finger = PNMA3 (336-463)

TABLE 7
PNMA3 RNA binding peptide modification (inner capsid surface)
Modification

| | |
|---|---|
| N-terminus extension | Insertion of GS-λN-GS between M1 and P2 (SEQ ID NO: |
| | 62-65) |
| Zinc finger region substitution | Substitution of 412-TFCYSCGEDGHIRVQCIN-429 with |
| | GS-λN-GS (SEQ ID NO: 62-65) |
| C-terminus extension | Extend the C-terminus with GS-λN (SEQ ID NO: 68-71) |

λN peptide: MDAQTRRRERRAEKQAQWKAAN (SEQ ID NO: 1)
λN peptide can be replaced with the following sequence
λN with enhanced binding = GNARTRRRERRAEKQAQWKAAN (SEQ ID NO: 2)
P22N = GNAKTRRHERRRKLAIERDTI (SEQ ID NO: 3)
HIV-1 Rev = TRQARRNRRRRWRERQR (SEQ ID NO: 4)

### Protein-Protein Interaction Domain Modifications

The engineered PNMA can be modified to include a protein interaction peptide such that the inner surface of a capsid formed from the engineered PNMA includes one or more protein interaction peptides. This can allow for capture/packaging of protein cargos within the capsid. PNMA2 was modified at the C-terminus by extension with a protein-protein interaction peptide, such as a Cas9, or a dimerization domain (e.g., a leucine zipper domain). Table 8 below shows the exemplary C-terminus modifications. In Table 8, the underlined GSGGS (SEQ ID NO: 74) in each exemplary modification is a Gly-Ser linker, which is fused to a leucine zipper (bold), which is a dimerization domain. “PNMA2 C-terminus extension” in Table 8 refers to a fusion protein containing a [non-truncated PNMA2]-[Gly-Ser linker]-Leucine Zipper Domain]. In other words, “PNMA2 C-terminus extension” refers to an engineered protein in which a leucine zipper domain was fused to a non-truncated C-terminus of a PNMA2 via a Gly-Ser linker thereby extending the C-terminus of the PNMA2 with the leucine zipper domain and linker. “SpCas9 C-terminus extension” in Table 8 refers to a fusion protein containing a [full length SpCas9]-[Gly-Ser linker]-Leucine Zipper Domain]. In other words, “SpCas9 C-terminus extension” refers to an engineered protein in which a leucine zipper domain was fused to a non-truncated C-terminus of a PNMA2 via a Gly-Ser linker thereby extending the C-terminus of the PNMA2 with the leucine zipper domain and linker. Without being bound by theory, dimerization of the PNMA2 C-terminus extension engineered protein and the SpCas9 C-terminus extension engineered protein at the leucine zipper domains complexes the PNMA2 and SpCas9. Without being bound by theory, this configuration via a dimerization domain can enhance capture and/or encapsidation of the cargo SpCas9 in the PNMA capsid. The protein-protein interaction domain modification can be introduced with any one or more of the other outer or inner capsid surface modifications detailed in this Example and elsewhere in the present disclosure.

TABLE 8
PNMA2 protein interaction peptide modification (inner capsid surface)
Modification

| | |
|---|---|
| PNMA2 C-terminus | Extension of C-terminus with |
| extension | GSGGGSLEIRAAFLRQRNTALRTEVAELEQEVQRLENE |
| | VSQYETRYGPLGGGK (SEQ ID NO: 75) |
| SpCas9 C-terminus | Extension of C-terminus with |
| extension | GSGGGSLEIEAAFLERENTALETRVAELRQRVQRLRNR |
| | VSQYRTRYGPLGGGK (SEQ ID NO: 76) |

### Outer Capsid Surface Modifications

The PNMA protein can be modified such that the outer surface of a capsid formed from the modified PNMA can include one or more modifications, such as to provide cell targeting or other functionalities such as to enhance endosomal escape.

### Cell Targeting Modifications

The PNMA protein can be modified to include cell surface binding peptide(s) or other cell targeting peptide(s) (e.g., receptor ligands or other peptides that facilitate cell targeting). The PNMA2 was modified at the N-terminus with a cell surface binding peptide as detailed in Table 9. The cell targeting modification can be introduced with any one or more of the other outer or inner capsid surface modifications detailed in this Example and elsewhere in the present disclosure.

TABLE 9
PNMA cell surface binding peptide modification (outer capsid surface)
Modification

| | |
|---|---|
| PNMA2 | Insertion of integrin binding peptide (RGD containing peptide): |
| insertion of | CYGGRGDTP (SEQ ID NO: 77) |
| peptide between | Insertion of VEGFR-1 ligand peptide F56: |
| M1 and A2 | WHSDMEWWYLLG (SEQ ID NO: 78) |
| | Insertion of human EGF peptide: |
| | NSDSECPLSHDGYCLHDGVCMYIEALDKYACNCVVGYIGERCQYRDLKWWELR |
| | (SEQ ID NO: 79) |
| | Insertion of human transferrin receptor binding peptide: |
| | GSREGCASRCMKYNDELEKCEARMMSMSNTEEDCEQELEDLLYCLDHCHSQ |
| | (SEQ ID NO: 80) |
| | Insertion of human hepatocellular carcinoma targeting peptide SP94: |
| | SFSIIHTPILPL (SEQ ID NO: 81) |

### Endosomal Escape Modifications

The PNMA protein can be modified to include peptide(s) that facilitate endosomal escape of a capsid formed from the PNMA protein(s) such that the peptides are present on the outer surface of the capsid formed from the modified PNMA protein(s). PNMA2 was modified with a peptide to facilitate endosomal escape of a capsid formed therefrom. The peptides shown in Table 10 were inserted between M1 and A2 of PNMA2.

TABLE 10
PNMA endosomal escape peptide modification (outer capsid surface)
Modification

| | |
|---|---|
| pVI peptide | AFSWGSLWSGIKNFGSTVKN (SEQ ID NO: 82) |
| H5WYG | GLFHAIAHFIHGGWHGLIHGWYG (SEQ ID NO: 83) |
| HIV tat peptide | YGRKKRRQRRR (SEQ ID NO: 84) |
| R5 peptide | RRRRR (SEQ ID NO: 85) |

### Example 2—RNA Delivery Using In Vitro Purified Icosahedral Capsids Formed by PNMA2

This Example at least demonstrates development of a platform for RNA delivery using a self-assembling engineered PNMA capable of selective packaging and cell entry.

PNMA family members are derived from retrotransposons and contain GAG domains (FIGS. 1A-1B). PNMA2 was identified as being able to form capsids in vivo and in vitro. It was observed that PNMA2 did not package an RNA genome (FIG. 2).

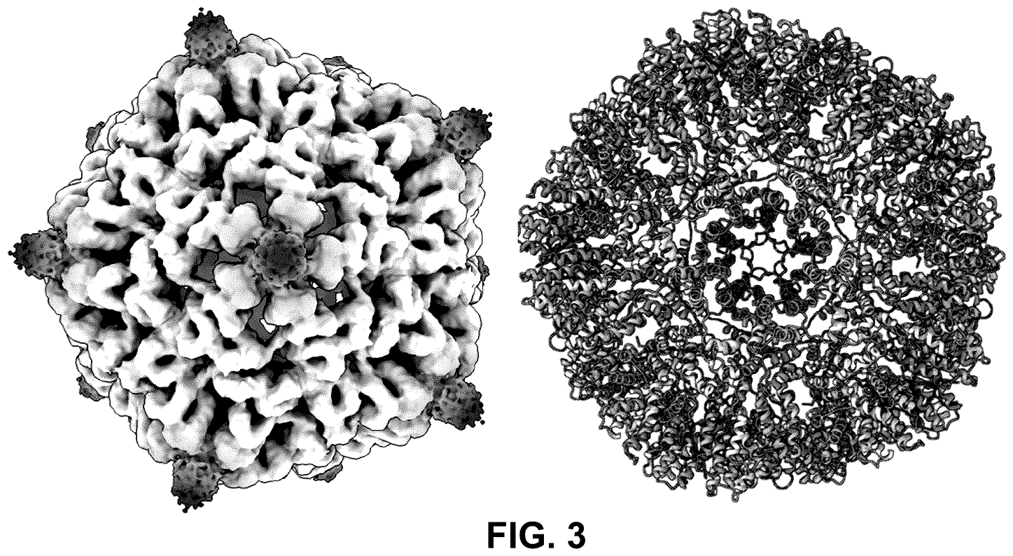

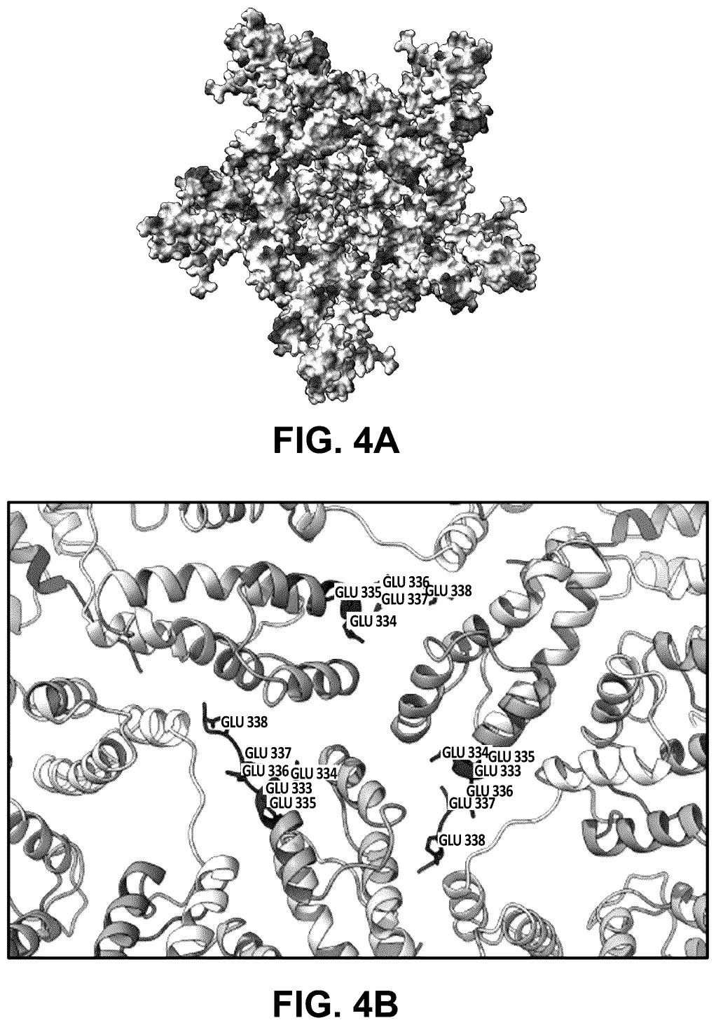

Characterization of PNMA2 capsids revealed that they form icosahedral capsids (FIG. 3). Further, it was observed that the inner surface of PNMA2 capsids were negatively charged with poly E stretches that extend into the capsid (FIG. 4A-4B).

PNMA2 capsids were formed in vitro from PNAM2 monomers and assembly was dependent on ionic strength (FIG. 5).

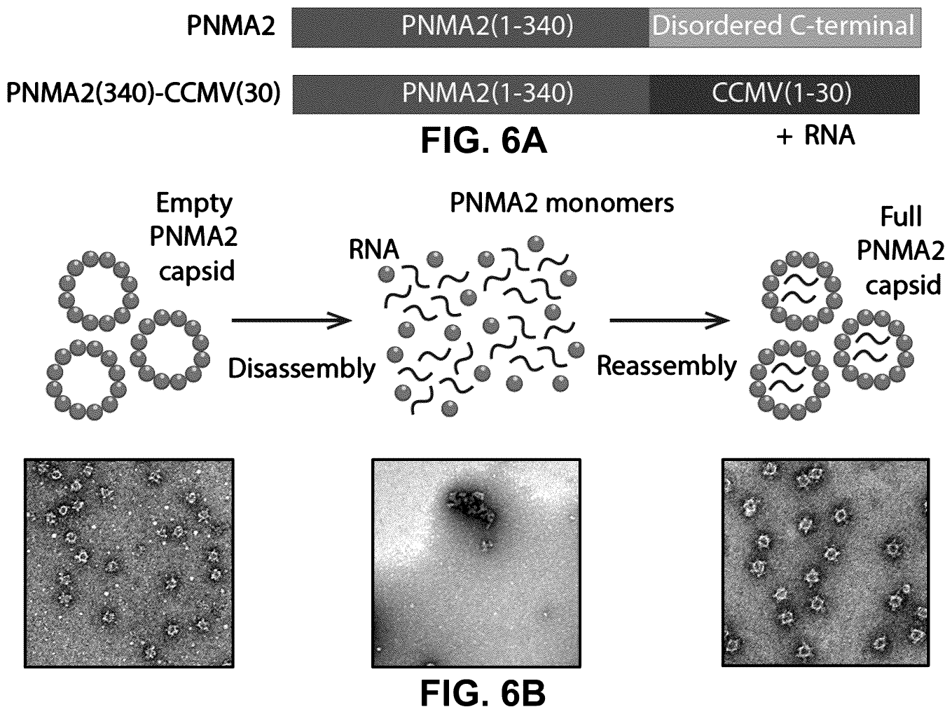

The PNMA2 was engineered by adding a RNA packaging peptide to the C-terminus of residues 1-340 of the human PNMA2. (FIG. 6A). As shown in FIG. 6A, the disordered C-terminal region of the human PNMA2 was replaced with 30 amino acid CCMV. CCMV is the cowpea chlorotic mottel virus N-terminal peptide that is responsible for packaging RNA. FIG. 6B shows packaging of RNA by PNMA2 monomers from the construct of FIG. 6A.

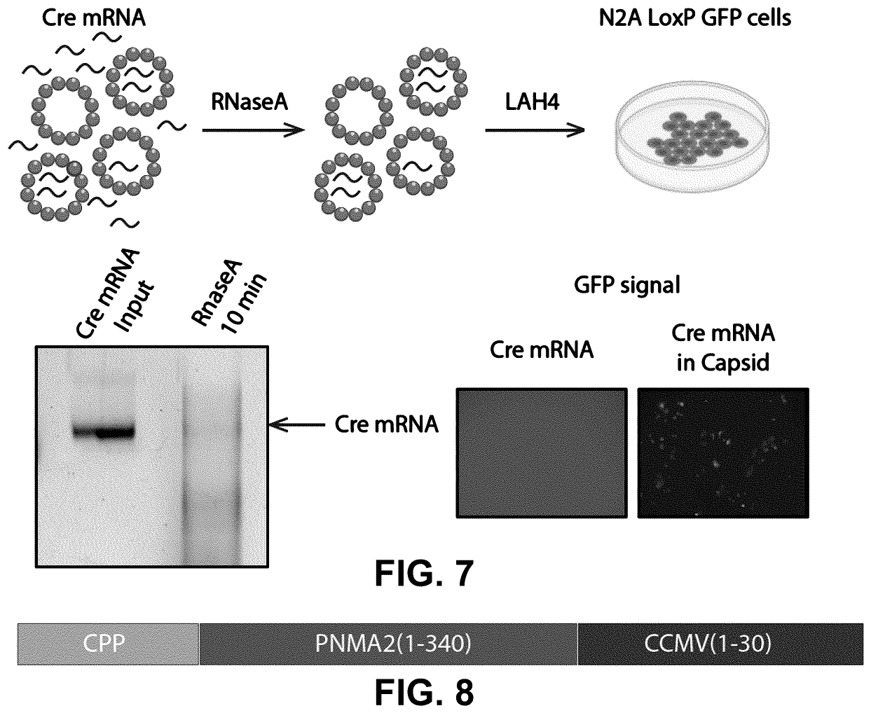

A functional reporter assay was developed that utilized Cre mRNA that was packaged by capsids followed by RNAseA treatment to remove unpackaged mRNA. These were delivered in the presence of LAH4 to N2A LoxP GFP cells. GFP signal was then measured in the cells. Where Cre RNA was packaged and delivered to the cell, the cells would express the GFP, which was measured microscopically. See e.g., FIG. 7.

Further constructs were designed that incorporate a cell penetrating peptide coupled to the N-terminus of PNMA2 or PNMA2-CCMV. This can increase cell entry of the capsid into cells. See e.g., FIG. 8.

### Example 3—Human Paraneoplastic Antigen Ma2 (PNMA2) Forms Icosahedral Capsids that can be Engineered for Gene Delivery

Broad application of genetic medicine can benefit from the development of new delivery approaches that are capable of tissue-specific targeting. As described at least in this Example human PNMA2 protein can be recombinantly purified and engineered to package mRNA for delivery into mammalian cells. This protein-based PNMA2 capsid provides a basis for rational engineering of enhanced delivery efficiency and cell-type targeting. Continued exploration of endogenous capsid-forming proteins is a promising avenue for engineering new therapeutic gene transfer systems.

RNA-based therapeutics, including mRNA-based vaccines, have the potential to be deployed in a wide range of disease contexts. To achieve this potential, however, a suite of delivery vehicles that can efficiently package and safely deliver therapeutic RNA cargoes to specific tissues is needed. Several delivery modalities have already been developed, including non-viral approaches such as lipid nanoparticles (LNPs), which have been successfully developed for oligonucleotide and mRNA therapeutics, and viral vectors such as adeno-associated virus (AAV) (1). However, the broad applicability of these approaches is limited due to a combination of factors including packaging constraints, immunogenicity, difficulty in achieving tissue-specific targeting, and costly large scale manufacturability. Thus, there continues to be a need for new delivery approaches.

The human genome contains many endogenous GAG-like genes, which resemble retroviral structural proteins (2). Previous work showed that the Arc and PEG10 GAG-like proteins have the ability to form capsid structures that can package their cognate mRNAs (3-6). Extending this natural ability, PEG10 was recently engineered to programmably package and deliver an exogenous cargo mRNA into human cells, demonstrating the potential of these endogenous retrotransposon-derived proteins as a new nucleic acid delivery modality (6). To further explore the potential of endogenous GAG proteins for therapeutic RNA delivery, Applicant sought to systematically characterize the paraneoplastic Ma antigen (PNMA) protein family (7), some of which have been shown to form capsid structures (6, 8).

Amongst these family members, Applicant found that PNMA2 is robustly secreted as an icosahedral capsid from mammalian cells and can self-assemble in vitro from recombinant proteins. Applicant used cryo-electron microscopy (cryo-EM) to resolve the structure of the PNMA2 capsid and structure-guided engineering to modify the PNMA2 protein capsid to package mRNA. Applicant demonstrates at least in this Example that these engineered PNMA2 capsids can functionally deliver mRNA into recipient cells, a promising approach for therapeutic mRNA delivery. In addition to PNMA2, Applicant found that other PNMA family members are capable of forming virus-like capsids, suggesting they may also be suitable for delivery and, without being bound by theory, raises the possibility that these proteins are involved in intercellular communication.

### Domain Architectures and Origins of the PNMA Family of Domesticated Retroelements

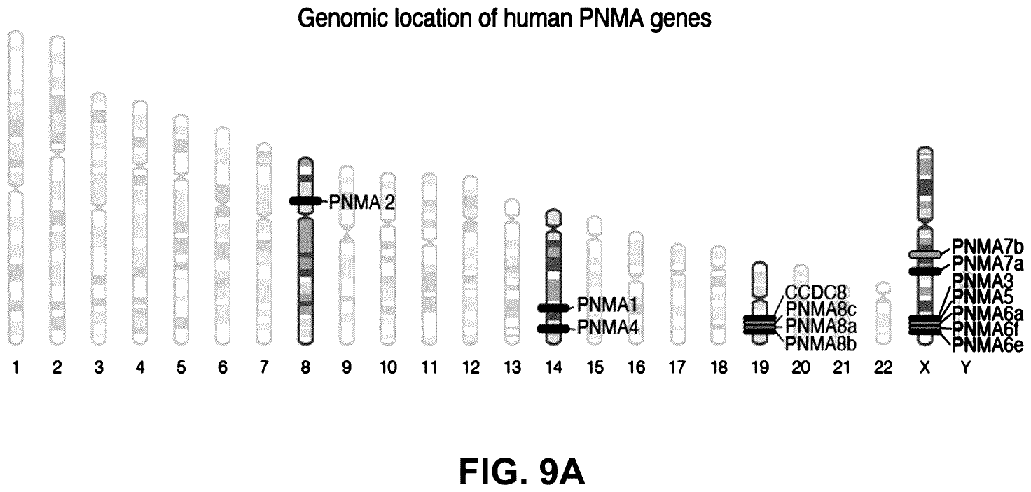

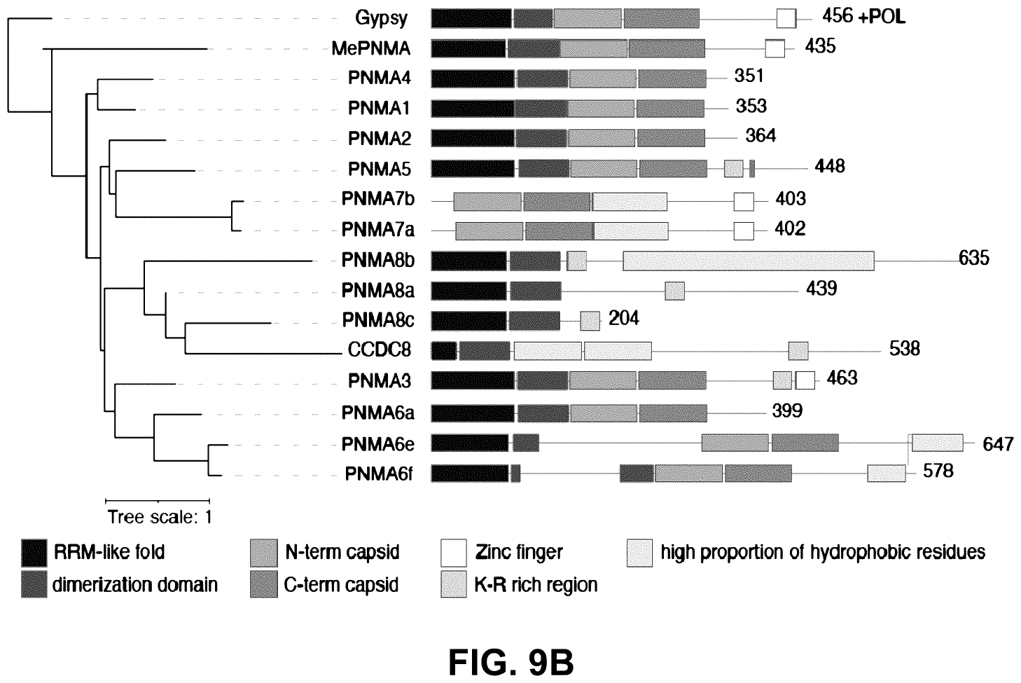

The PNMA family likely emerged from the domestication of a Ty3/Gypsy retrotransposon by the loss of the polymerase (POL) region (7, 9). Multiple duplications of the ancestral PNMA occurred in Eutherians giving rise to a large family of PNMAs in some mammalian species, including humans. In the human genome, PNMAs are spread across 4 chromosomes, with some clustered together—PNMA8a/b/c and CCDC8 share the same locus in chromosome 19, and PNMA3/5/6a/6e/6f share the same locus in chromosome X (FIG. 9A).

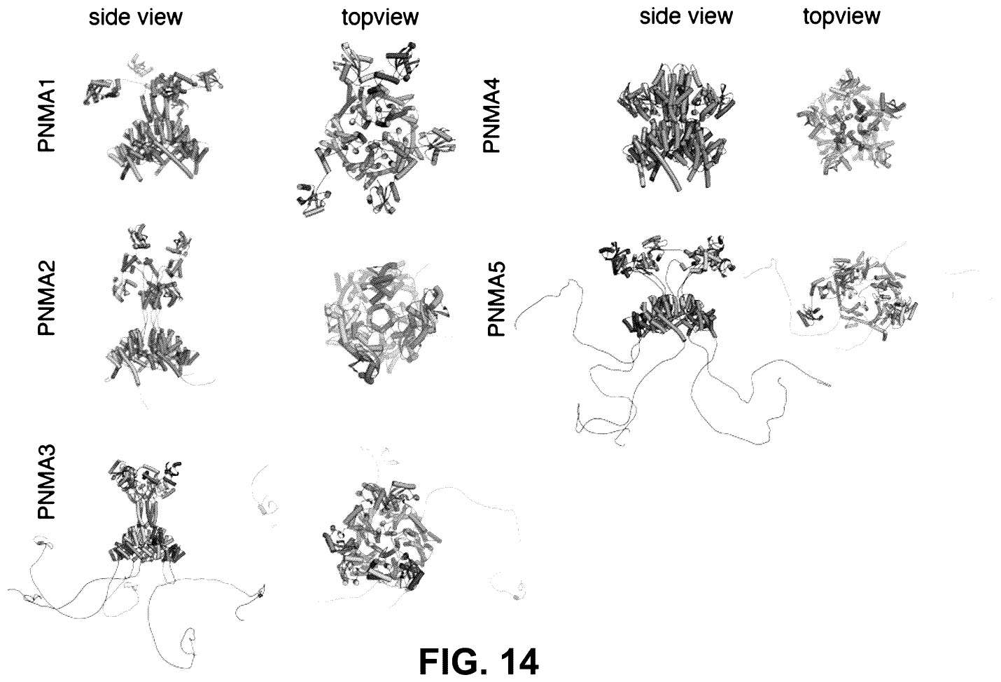

Applicant used structural modeling to compare the domain architectures of 14 human PNMA proteins (1, 2, 3, 4, 5, 6a, 6e, 6f, 7a, 7b, 8a, 8b, 8c, and CCDC8), as well as the marsupial PNMA from the tammar wallaby, Macropus eugenii (MePNMA), which is a close relative to Gypsy (see Methods) (FIG. 9B). Most of the human PNMAs contain the capsid domain, except PNMA8a/b/c and CCDC8, and AlphaFold-based oligomeric prediction suggests that PNMAs 1, 2, 3, 4, and 5 may form multimers (FIG. 14). The RRM domain is also highly conserved, with only PNMA7a and 7b lacking it. The zinc finger domain, which may be involved in interaction with nucleic acids (10), is found in only some PNMAs. Of the proteins that lack the zinc finger, some alternatively contain a K-R rich domain, which could similarly function to interact with nucleic acids.

### PNMA2 is Secreted by Human Cells as a Non-Enveloped Capsid

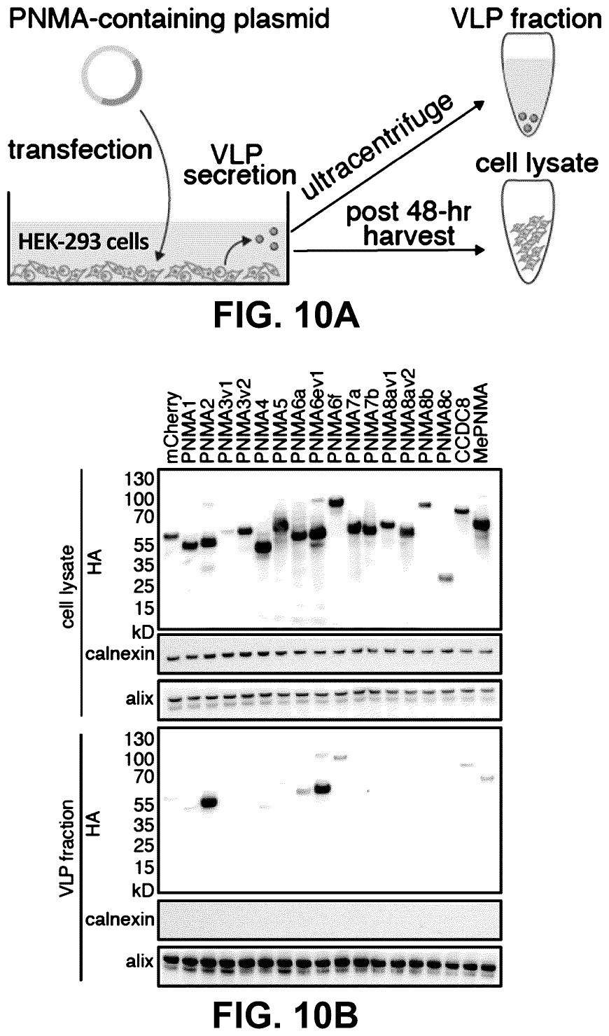

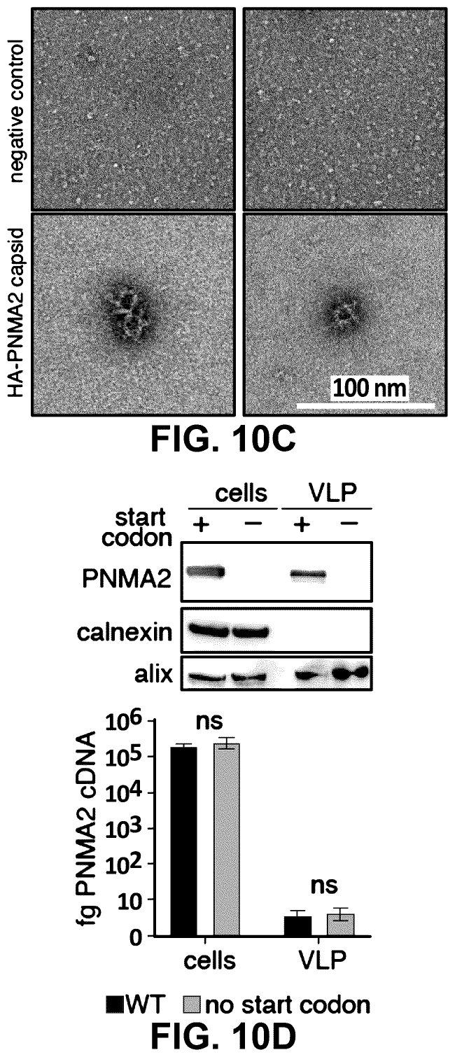

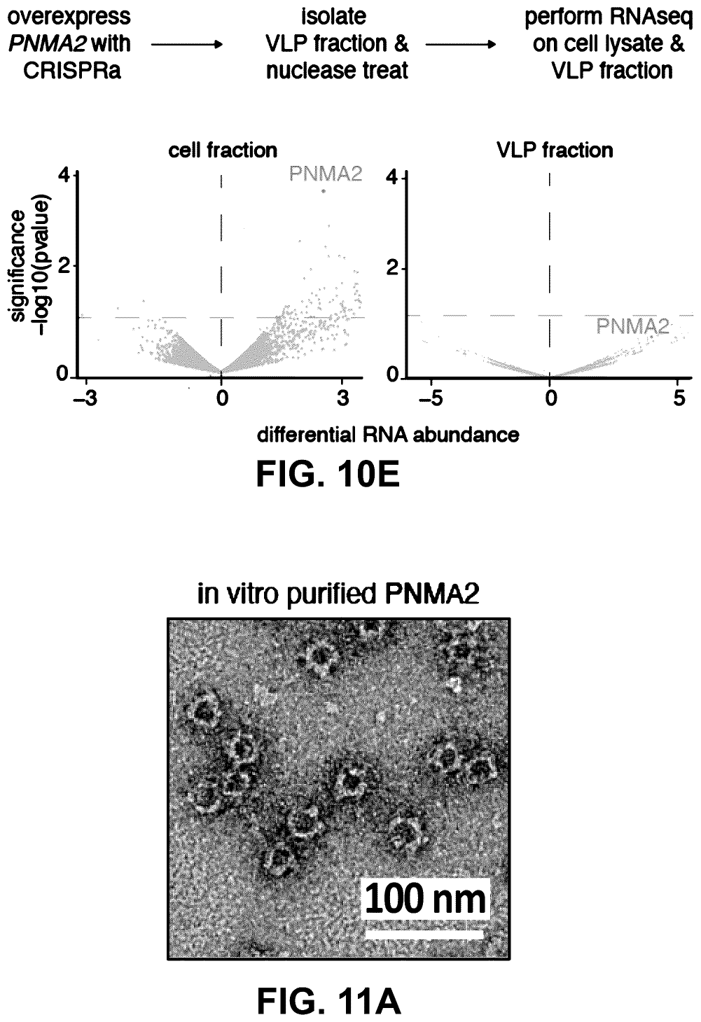

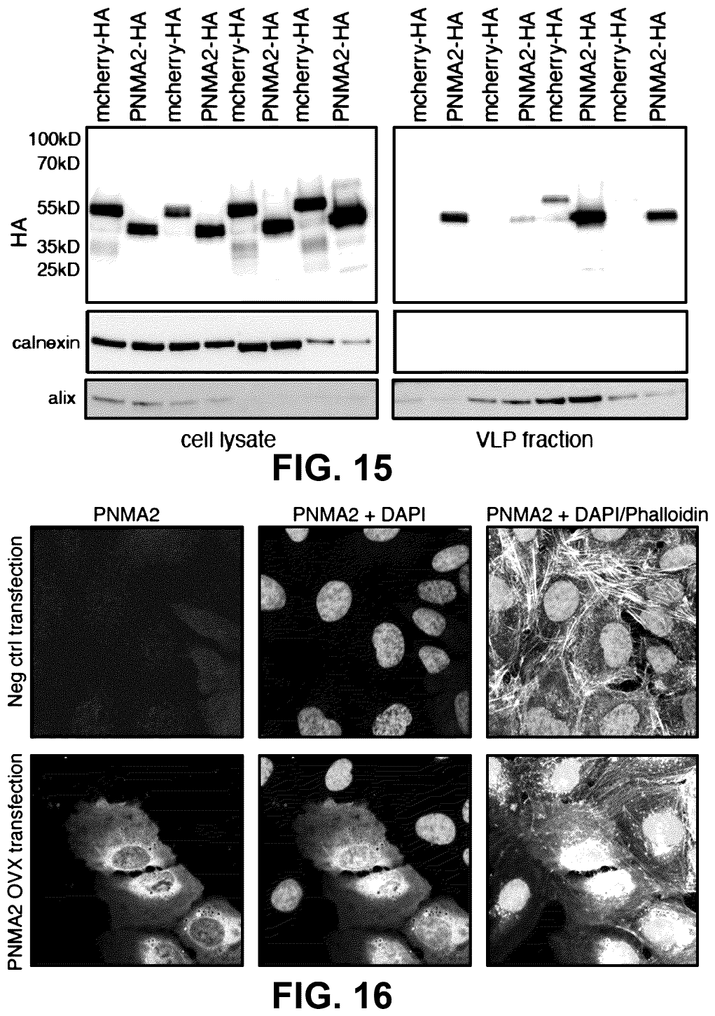

Given that Arc and PEG10 are secreted from mammalian cells, Applicant tested PNMA secretion by transfecting each PNMA into HEK293FT cells (FIG. 10A-10B). Although all PNMAs expressed robustly, only a subset of PNMAs were secreted into the virus-like particle (VLP) fraction, with PNMA2 demonstrating especially robust secretion across multiple cell lines (FIG. 10B and FIG. 15). Applicant observed that PNMA2 expression in cells is localized to the cytosol (FIG. 16), and PNMA2 is secreted by cells as non-enveloped capsid-like structures (FIG. 10C). This is consistent with similar findings in mouse cells (8).

Applicant investigated whether PNMA2 capsids package their own mRNA by comparing the levels of PNMA2 mRNA in cellular and VLP fractions from HEK293FT cells overexpressing either PNMA2 or a start codon deficient version (FIG. 10D). Applicant found no significant difference in PNMA2 mRNA levels between the VLP fraction with PNMA2 capsid and the deficient version, indicating that PNMA2 does not package its own mRNA. Applicant also sequenced mRNA from the VLP fractions of U20S cells transfected with either a PNMA2 CRISPRa cassette or a non-targeting control (6), and found no significant increase in transcript abundance in the VLP fraction for any mRNA species, suggesting that PNMA2 capsids do not specifically package PNMA2 mRNA or any other cellular mRNA (FIG. 10E).

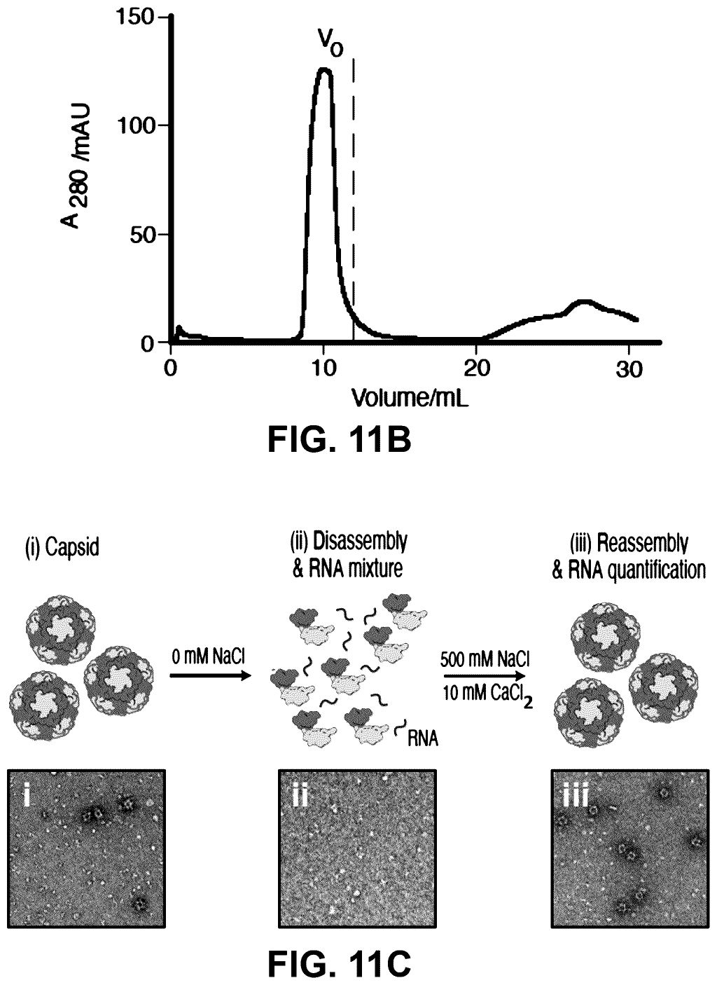

Although Applicant's results indicate PNMA2 does not package mRNA in vivo, Applicant sought to determine if purified PNMA2 could assemble around an mRNA in vitro. Applicant first tested whether PNMA2 capsids can self-assemble in vitro using recombinant proteins, finding that PNMA2 purified from E. coli readily assembles into capsid structures similar to those secreted from mammalian cells (compare FIG. 10C with FIG. 11A-11B). Applicant hypothesized that orchestrating PNMA2 disassembly and reassembly around RNA might facilitate RNA packaging (FIG. 11C). To test this, Applicant introduced Cre mRNA into the assembly reaction and then assayed if it was resistant to nuclease degradation. However, self-assembled PNMA2 did not protect Cre mRNA from nuclease degradation, despite the ability to control its disassembly and reassembly with salt concentration (FIG. 11C).

PNMA2 Forms an Icosahedral Capsid Structure with a Negatively Charged Lumen

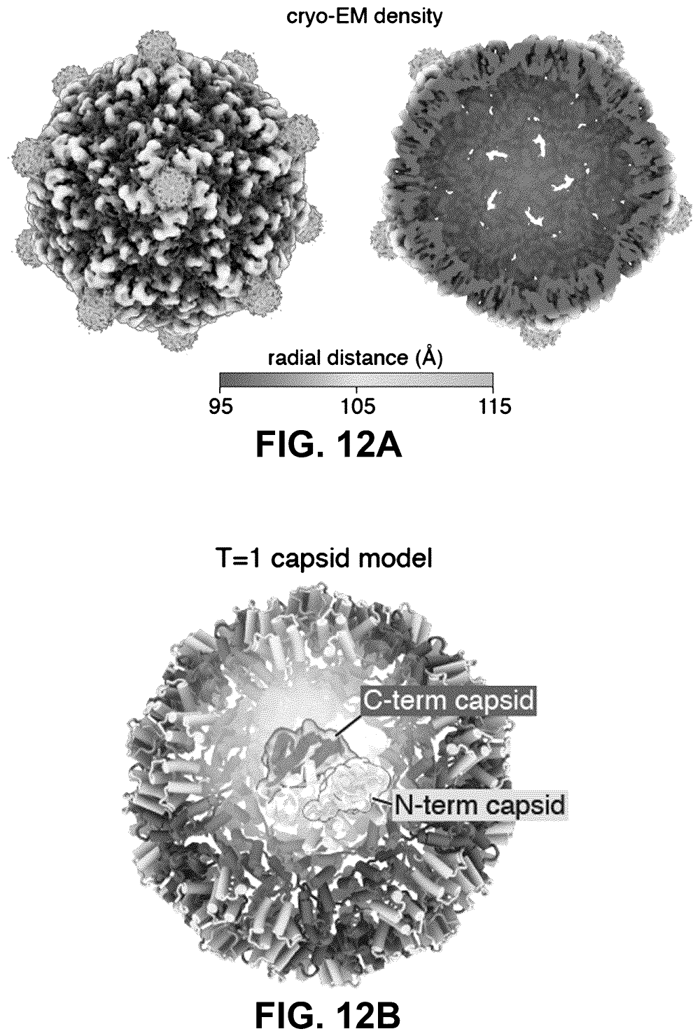

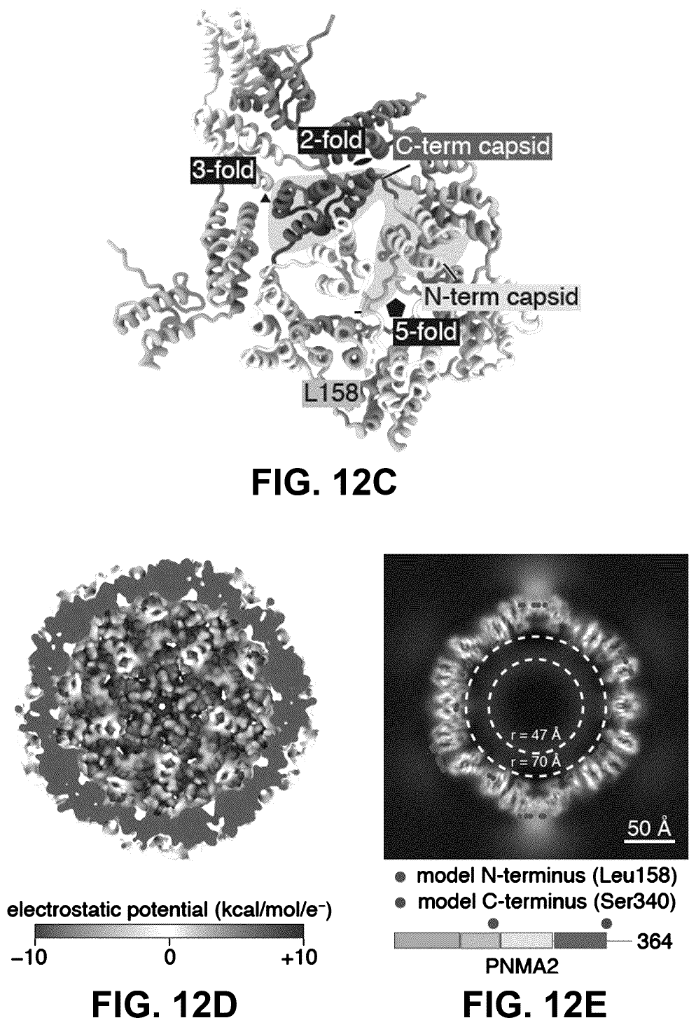

To guide Applicant's engineering efforts to package RNA within PNMA2, Applicant used cryo-EM to resolve the structure of recombinant human PNMA2 expressed in E. coli at 3.1 Å resolution (FIG. 12A). Sixty identical copies of the PNMA2 monomer assemble to form a capsid with icosahedral symmetry and a triangulation number T=1 (FIG. 12B). The capsid has a mean diameter of 210 Å and encloses a volume of 1,400,000 cubic Ångstrom—approximately 60% of the volume of AAV-2 (11). Cryo-EM density was only resolvable for residues 158-340 of PNMA2, corresponding to the N- and C-terminal capsid domains, which fold into α-helical domains similar to other GAG proteins. The interfaces at the 2- and 3-fold symmetry axes are composed of the C-terminal capsid domains, while the 5-fold symmetry axis is composed of the N-terminal capsid domains (FIG. 12C). The first ordered residue of PNMA2, Leu158, is found at the 5-fold axis on the exterior side of the capsid, suggesting that diffuse cryo-EM density forming “spikes” on the 5-fold axis (FIG. 12A) is attributable to the N-terminal dimerization and RRM-like domains of PNMA2. The interior of the capsid is rich in acidic residues, including the last resolvable residues which form a poly-Glu tract (333-EEEEEEAS-340 (SEQ ID NO: 130)). The interior of the capsid is therefore predicted to have a negative charge (FIG. 12D). Applicant did not resolve the final 24 residues of PNMA2 (ten of which are also acidic), but a cloud of cryo-EM density is visible inside the capsid and is likely accounted for by these residues (FIG. 12E).

Engineering PNMA2 for Functional mRNA Delivery

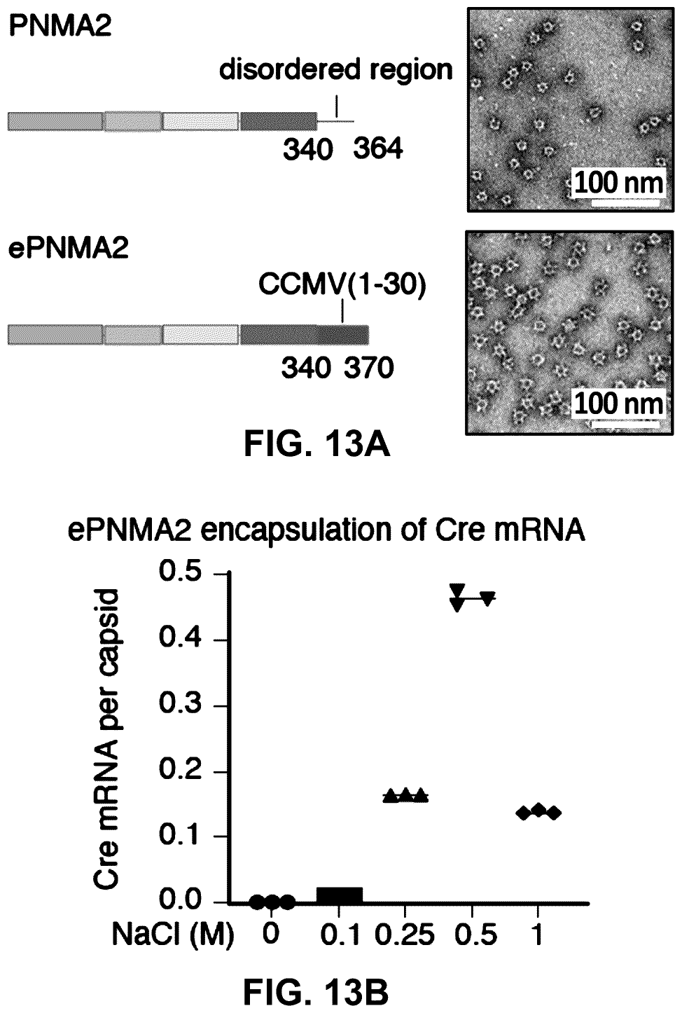

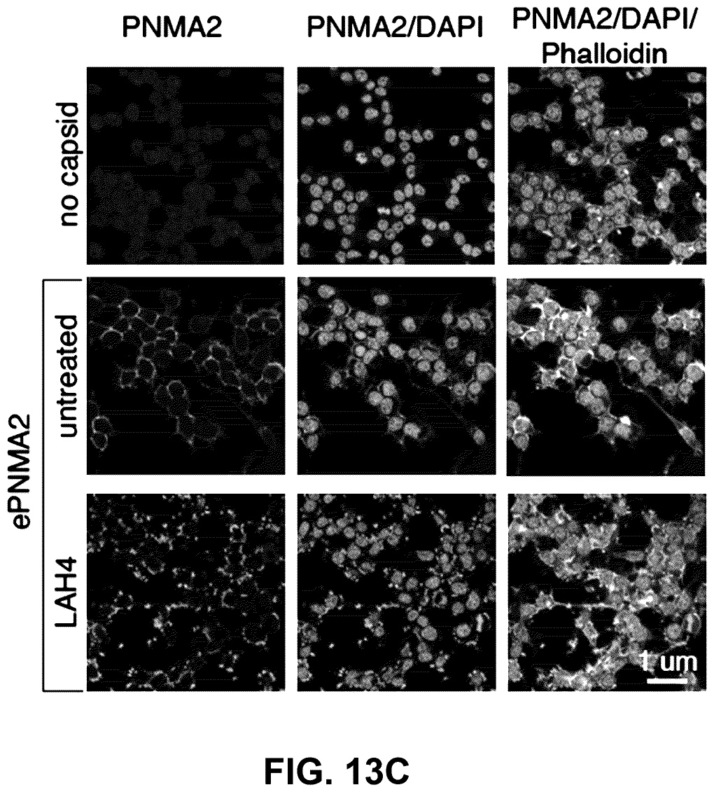

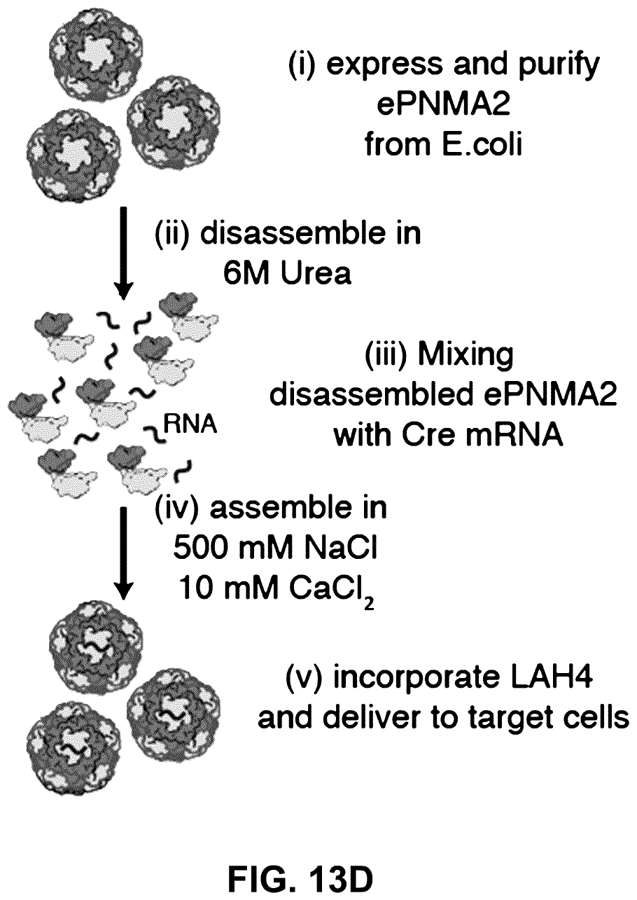

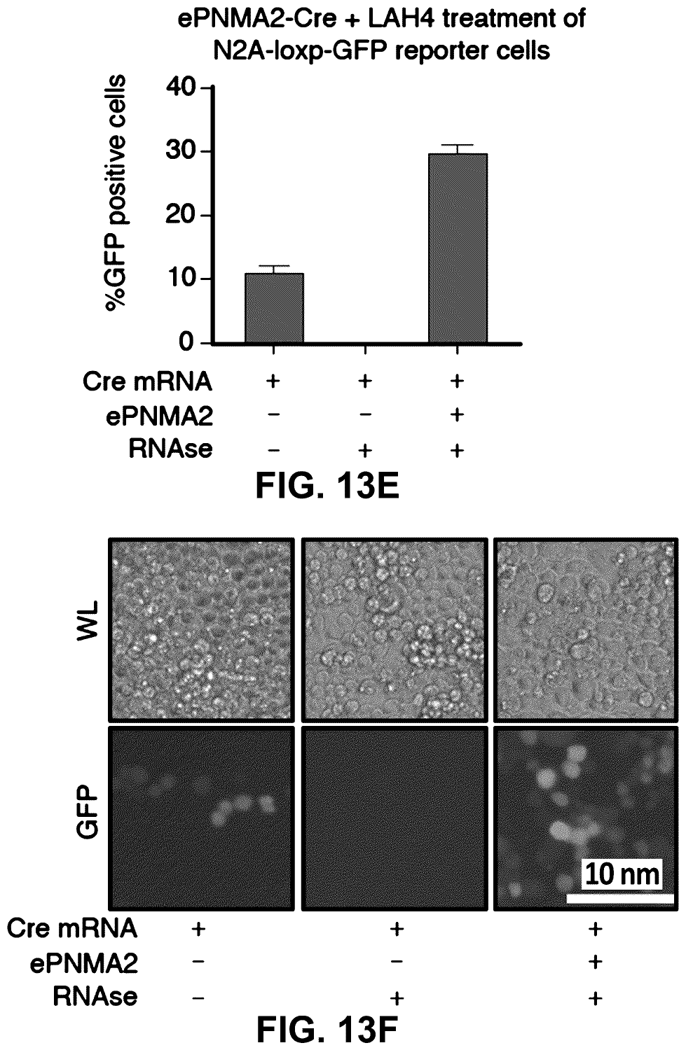

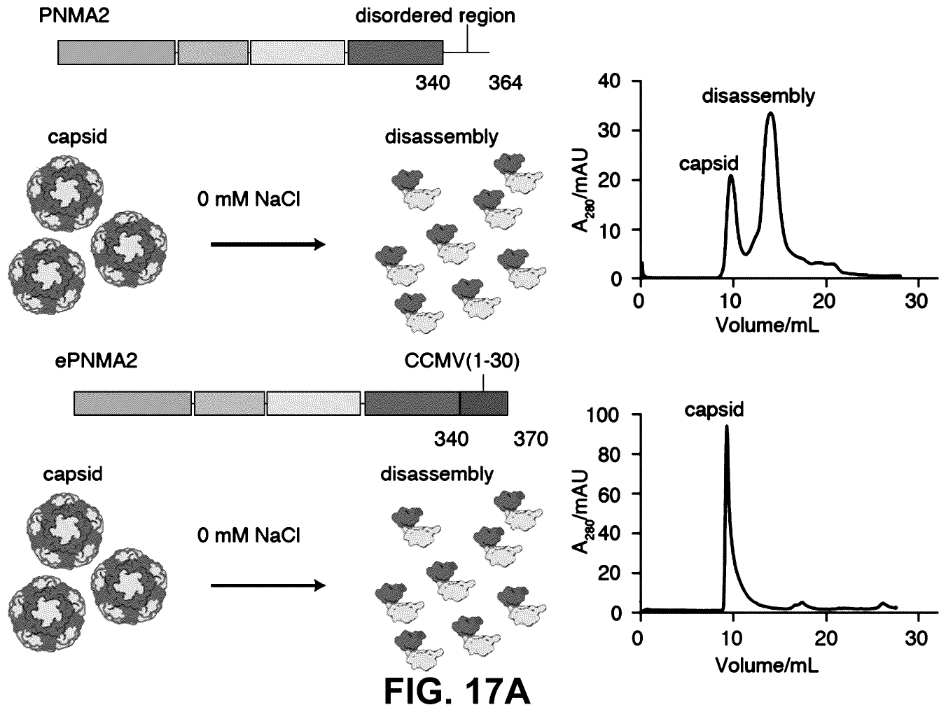

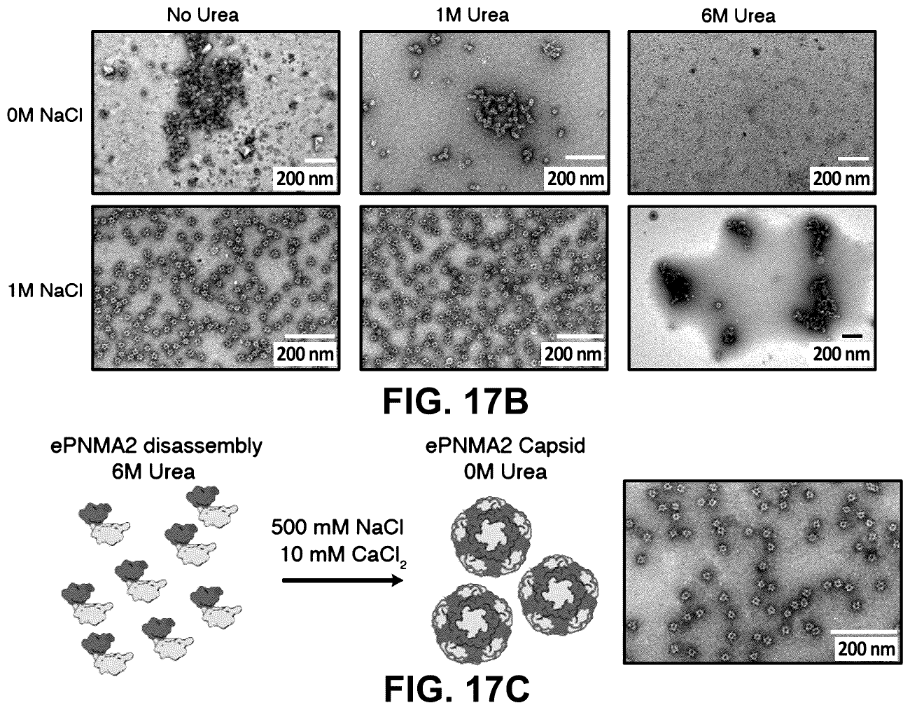

To enhance mRNA packaging efficiency, Applicant used structure-guided engineering to modify the capsid lumen by replacing the C-terminal disordered region with an RNA-binding motif, cowpea chlorotic mottle virus N-terminal 30 residues (CCMV1-30), which is known to efficiently bind single stranded RNA without obvious sequence preference (12, 13) (FIG. 13A). Applicant purified the resulting PNMA2(340)-CCMV(30) (referred to as engineered PNMA2 (ePNMA2)) from E. coli and confirmed capsid formation similar to wild-type PNMA2 (FIG. 13A). In contrast to wild-type PNMA2 capsids, ePNMA2 capsids were more stable at low ionic strengths and required 6 M urea for disassembly (FIG. 17A-17C). Applicant tested various conditions for reassembly in the presence of cargo RNA and found that 500 mM NaCl and 10 mM CaCl₂) led to the most efficient packaging (about 0.5 genomes per capsid) and protection of cargo RNA from nuclease degradation (FIG. 13B and FIG. 17C).

Applicant examined whether ePNMA2 capsids (which are non-enveloped) can enter cells via endocytosis. Confocal microscopy of Neuro2A cells 6 hours after treatment showed ePNMA2 capsids at the cell periphery (FIG. 13C). Given previous data showing that the amphipathic peptide LAH4 can aid proteins in both cellular entry and endosomal escape (14), Applicant assessed whether treating ePNMA2 with LAH4 before addition to cells could enhance entry beyond the cell periphery. Applicant found that LAH4 treatment of ePNMA2 before addition to cells increased the cytosolic localization of ePNMA2 (FIG. 13C).

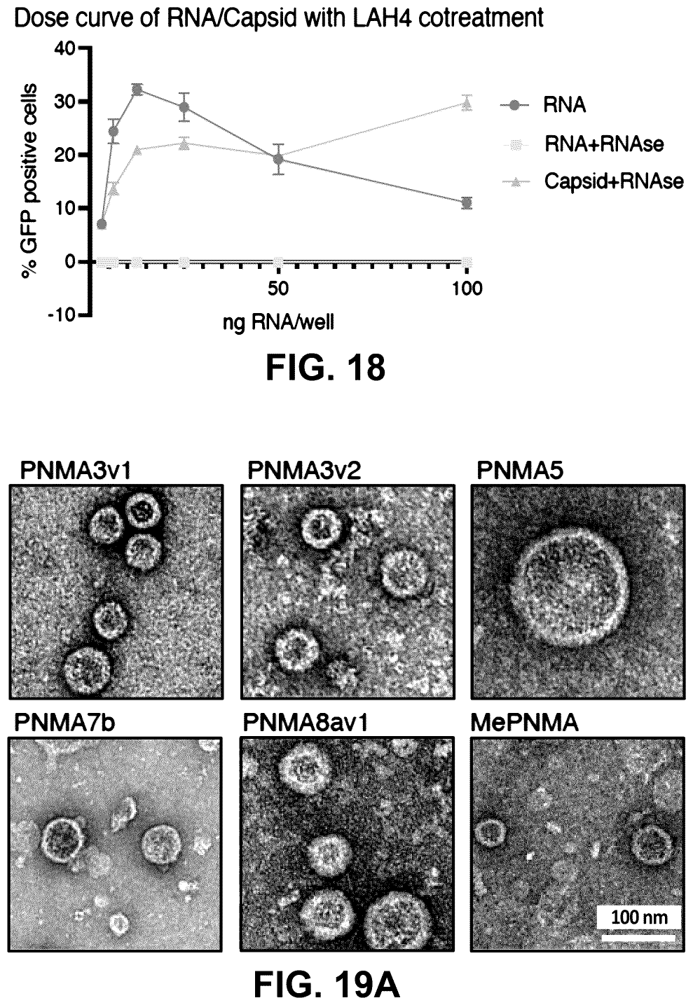

Finally, Applicant tested if LAH4-treated ePNMA2 capsids could deliver a Cre RNA cargo to Neuro2A-loxP-GFP reporter cells (FIG. 13D). Applicant RNase treated ePNMA2(Cre) to degrade unpackaged mRNA and prepared equivalent naked Cre mRNA with and without RNase treatment, all of which were combined with LAH4 peptide before being added to Neuro2A-loxP-GFP reporter cells (FIG. 13E-13F and FIG. 18). RNase treatment completely degraded mRNA in the absence of ePNMA2, as confirmed by the absence of GFP expression (FIG. 13E-13F and FIG. 18). At the highest dose (100 ng RNA per 2.5e4 cells), ePNMA2(Cre) delivery produced higher levels of GFP reporter expression relative to an equivalent amount of unpackaged RNA that had not been RNase digested (FIG. 13E-13F). Even a low dose (3.125 ng RNA per 2.5e4 cells) of ePNMA2(Cre) was sufficient to induce GFP expression in roughly 7% of Neuro2A-loxP-GFP reporter cells (FIG. 18). These data suggest that ePNMA2 can protect a functional RNA cargo from nuclease degradation, a key characteristic for nucleic acid delivery vehicles due to abundant nuclease activity in the extracellular milieu (15, 16). These results demonstrate the potential of ePNMA2 as a gene transfer tool in human cell lines.

### DISCUSSION

In the current study, Applicant demonstrated that human PNMA2 is robustly secreted from cells as an icosahedral, non-enveloped capsid. Applicant showed that although PNMA2 does not package RNA in human cells, an engineered variant with an RNA-binding domain grafted on to the C-terminus enables in vitro packaging of a cargo RNA. Combining these self-assembled, packaged ePNMA2 capsids with the cell-penetrating peptide LAH4 led to efficient functional delivery of mRNA.

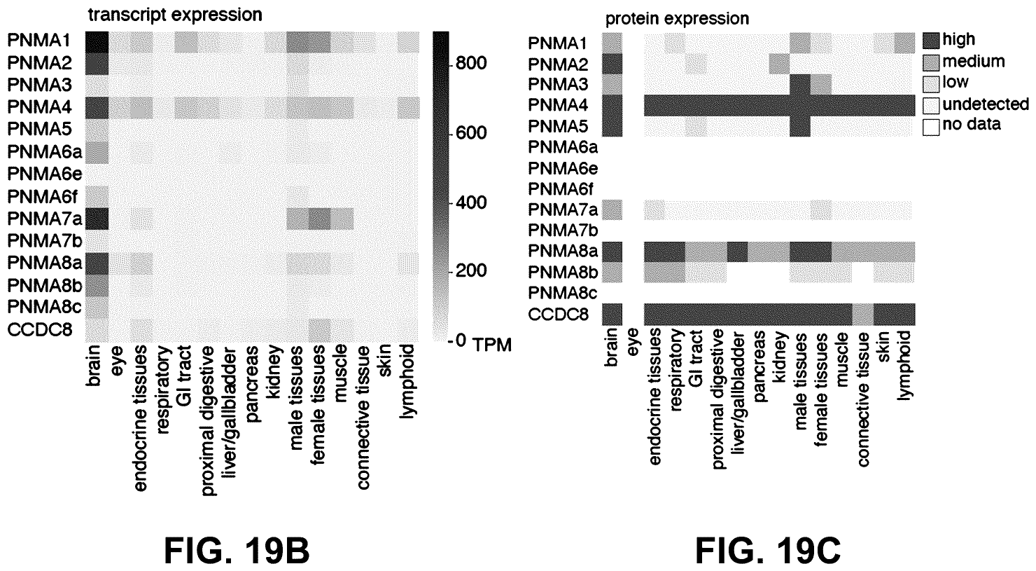

Applicant's demonstration of an all protein, in vitro produced delivery vehicle offers a starting point for further bioengineering. For example, increasing positive charges in the ePNMA2 capsid lumen could allow packaging of larger RNA cargoes or enhance RNA packaging efficiency. Further engineering of the ePNMA2 capsid surface residues may allow robust cell entry without LAH4, or targeted cell-type or tissue tropism. Engineering strategies applied to AAVs, which bear a similar T=1 icosahedral capsid structure, could be used to genetically modify the ePNMA2 capsid surface with integrin binding motifs or nanobodies and thus modulate ePNMA2 tropism (17, 18). The tropism and immunogenicity of these vectors in vivo merits further investigation. Finally, Applicant's work with PNMA2 may be extended to other PNMA family members, some of which also form capsids (FIG. 19A). This, together with the fact that the expression of many PNMA family members is highest in the central nervous system (FIG. 19B-19C), raises the possibility that these proteins may be harnessed for delivery of genetic cargoes to the brain, a long-standing goal in the delivery field.

### Materials and Methods

### Determination and Comparison of Domain Architecture of the PNMA Family

A structural model was built for each member of the PNMA family using AlphaFold2 (19) under the colabfold framework (20) using default parameters. Models with plddt >=70 were selected for analysis, and additional AlphaFold2 cycles were performed until plddt was greater than 70. Structures were analyzed and compared usingPyMOL (The PyMOL Molecular Graphic System Version 1.2, Schrodinger, LLC) to annotate protein domain architecture. Hydrophobic Cluster Analysis was used to compare local structure and patterns across all PNMAs (21, 22). The RRM-like domain was identified from structural mining using the Dali server (23, 24). Domain architectures were compared across all human PNMAs, the marsupial PNMA (NCBI accession number: BAK55632.1), and the turtle Gypsy (NCBI accession number: XP_048704523), which was the closest non-PNMA relative Applicant identified from a preliminary phylogenetic analysis from homologs of PNMAs. A final tree was built using PhyML (25) on the MPI Bioinformatics Toolkit website (26) with LG model and 200 replicates. The final tree was visualized with the interactive tree of life (itol) webserver (27) (FIG. 9B).

### Prediction and Analysis Ofcapsomer Assemblies

Pentamer assembly of PNMA2 was predicted using Alphafold2 multimer (28) under the colabfold framework using 40 cycles and 5 replicas. All replicas formed a capsomer in which the capsid domain forms a ring pentamer, and the N-terminal region forms dimers leading to two dimers and one monomer in the pentamer assembly. The interaction region was evaluated and analyzed using PyMOL software.

### Plasmid Cloning

PNMA open reading frames (ORFs) were human codon optimized and ordered as gblocks from Twist. These gblocks were then cloned into an E. coli expression backbone (Addgene #104129) with an N-terminal Maltose Binding Protein (MBP) tag and bdSUMO for purification via Gibson Assembly. Gblocks were also cloned into a CMV promoter driven mammalian expression backbone (Addgene #11153) with the WPRE and SV40 polyadenylation signal (Addgene #83281) via Gibson Assembly. CRISPRa guide RNAs and a non-targeting control were cloned into the PB-Unisam CRISPRa backbone (Addgene #99866).

A plasmid encoding the PNMA2 transcript driven by the CMV promoter was generated by PCR of the human PNMA2 exons from HeLa genomic DNA (New England Biolabs N4006S). PNMA2 sequence specific primers were designed using PrimerBlast (NCBI), and PCR fragments were joined via Gibson Assembly. For in vitro transcription, a plasmid was generated with Cre RNA downstream of T7 promoter. One hundred A's were inserted at the 3′ end of the Cre RNA to serve as a poly A sequence. The PsiI digestion site was inserted downstream of the poly A sequence.

### In Vitro Production and Purification of PNMA Proteins

A plasmid encoding PNMA2 with an N-terminal MBP tag was transformed into Rosetta 2 (DE3) pLyse S cells. A single colony was inoculated in Terrific Broth (TB) media overnight at 37° C. with 100 ug/ML. ampicillin and 25 μg/mL chloramphenicol. When optical density 600 (OD600) reached 0.6, the culture was cooled to 4° C. for 30 minutes. IPTG was added to a final concentration of 0.5 mM, and the culture was incubated at 21° C. for 20 hours. Bacteria were centrifuged at 4000 rpm for 15 minutes, media supernatant was decanted, and the bacterial pellet was then resuspended in a lysis buffer containing 50 mM Tris pH 8, 250 mM NaCl and 0.5 mM TCEP. Lysis was achieved with two passes through the LM20 Microfluidizer system at 27,000 p.s.i. The lysis was cleared with centrifugation at 9000 rpm for 30 minutes. The lysis was incubated with 2 mL amylose beads for 2 hours at 4° C. The amylose beads were washed, and the bound PNMA2 was cleaved overnight with lysis buffer with 1.5% NP-40 and lug/mL bdSENP1. The elution was collected and used for SEC analysis using an AKTA pure system with Superdex200 increase 10/300 GL column with an isocratic run using lysis buffer at 0.4 mL/min.

### Negative Staining and Transmission Electron Microscopy

For sample preparation of TEM imaging grids, 5 ul of sample at a protein concentration of approximately 0.3 mg/ml was loaded onto glow-discharged, carbon-coated 300-mesh copper grids (Electron Microscopy Sciences #Q3100CR2-2 nm). Sample was adhered to the grid for one minute at room temperature and stained in five sequential droplets for a total of one minute in freshly filtered 2% uranyl formate. Following the staining procedure, excess uranyl formate was carefully blotted off with Whatman filter paper (Cytiva, #1001-032). The grid was dried at room temperature for 1 minute before placement into a grid holder. All TEM images were acquired using the FEI Tecnai (G2 Spirit TWIN) 120 kV multipurpose TEM at the MIT MRL facility. The grid was mounted on a JEOL single tilt holder equipped in the TEM column and cooled down with liquid nitrogen. The microscope was operated at 200 kV and with a magnification in the range of 10,000-60,000×, and all images were recorded on a Gatan 2k×2k UltraScan CCD camera.

### Cell Culture

U20S cells (ATCC HTB-96) were maintained in McCoy's 5A (Modified) Medium supplemented with 10% fetal bovine serum and 100 U/mL penicillin-streptomycin.

HEK293FT (Thermo Fisher R700-07), HeLa (ATCC CCL-2), U87 (ATCC HTB-14) and Neuro2A (ATCC CCL-131) cells were maintained in Dulbecco's Modified Eagle Medium supplemented with 10% fetal bovine serum and 100 U/mL penicillin-streptomycin. U20S, HeLa, U87 and Neuro2A cells were transfected with Lipofectamine 3000 (ThermoFisher, L3000001) at 80% confluence, and media was changed 4 hours post transfection to reduce toxicity. HEK293FT were transfected at 70% confluence with PEI HCl MAX (Polysciences 24765-1).

Neuro2A-loxP-GFP stable reporter cells were generated by subcloning the loxP-GFP cassette from RV-Cag-Dio-GFP (Addgene #87662) into a lentiviral transfer plasmid encoding a Blasticidin resistance gene for stable integration. To produce virus, HEK293FT cells were seeded at 1e7 cells per 15-cm dish. 16 hours later, cells were co-transfected with 5 ug psPAX2 (Addgene #12260), 4.7 ug pMD2.G (Addgene #12259), and 7.7 ug of the Cre reporter plasmid using PEI HCl MAX (Polysciences 24765-1), and media was changed 4 hours post transfection. Forty-eight hours later, viral supernatant was harvested, spun at 2000 g for 10 mins to remove cell debris, filtered through a 0.45-μm filter, and stored at −80° C. degrees. N2a reporter cell lines were created by lentiviral transduction with 8 μg/mL polybrene(TR1003G). Media was changed one day later, and cells were selected for two weeks starting on day 3 with 10 μg/mL Blasticidin-HCl (Thermo Fisher Scientific A1113903). Single clones of Blasticidin-resistant cells were isolated by serial dilution, expanded, and then screened for successful reporter expression by transfection of a Cre encoding plasmid.

Isolation of VLPs from Human Cells

Forty-eight hours post transfection, media supernatant for sucrose cushion purification was filtered through a 0.45-μm filter, added to conical ultracentrifuge tubes (Beckman Coulter 358126), and underlaid with 4 mL of 20% sucrose in 1×PBS. Tubes were then spun at 120,000 g for 2 hours in a Beckman Coulter SW28 rotor, after which supernatant was decanted and the pellet was resuspended in 100 μL of 1×PBS.

### Western Blot Analysis

For cellular lysate, cells were washed in 1×PBS and lysed in RIPA buffer (ThermoFisher 89901) with Halt protease inhibitor (ThermoFisher 87786) for 30 minutes at 4° C. Lysate was then spun at 20,000 g for 10 minutes at 4° C. to pellet insoluble protein. Cellular lysate supernatant and resuspended VLP were combined with 1× Bolt LDS Sample Buffer (Life Technologies B0007) and 100 mM DTT and boiled at 95° C. for 10 minutes. Samples were loaded into Bolt 4-12% Bis-Tris Plus gels (ThermoFisher) and run at 200V for 30 minutes, before being transferred onto PVDF membrane with the iBlot2 system (ThermoFisher). Membranes were blocked in 5% milk in 1×TBS Tween 20 (TBST) Buffer, and incubated at 4° C. overnight with primary antibody in 2% milk in 1×TBST. Following three 1×TBST washes, samples were incubated with secondary antibody at 1:20,000 for 1 hour and then imaged.

### Immunofluorescence and Confocal Microscopy

Cells were seeded at 5e4 cells/well on Poly-D-Lysine/Laminin coated glass coverslips (VWR 354087). The following day, cells were washed with 1×PBS, fixed in 4% PFA in PBS for 30 minutes, permeabilized in 0.1% Triton X100 for 30 minutes, and then blocked in 1% BSA for 30 minutes. Cells were stained with rabbit anti-PNMA2 primary antibody (ThermoFisher PA5-81995) diluted 1:200 in 1% BSA for one hour, washed and then stained with AlexaFluor488 conjugated secondary antibody at 1:1000 for 1 hour in the dark. Cells were then stained with Alexa-Fluor 647 Phalloidin (Cell Signaling Technologies 8940S) and DAPI at 0.01 mg/mL for 5 minutes, washed three times with 1×PBS, and mounted in Diamond ProLong mounting media on glass slides. Mounted specimens were imaged on a Leica (Cat #here) with the 63× oil objective.

### HA Immunoprecipitation of HEK Secreted HA-PNMA2

ePNMA2 VLPs were isolated from the supernatant of HEK293FT cells transfected with PNMA2 with an HA-tag at the N-terminus as described previously. Following ultracentrifugation, HA-tag pulldown was performed using the HA tagged protein purification kit from MBL (#3320) using the manufacturer's instructions. Successful isolation was confirmed by Coommassie staining and protein capsids were imaged using TEM as described previously.

### RNA Isolation and RT-gPCR

Cells or VLPs were resuspended in Trizol (ThermoFisher 15596026), vortexed, and incubated at room temperature for 5 minutes. Total RNA was then prepared via phenol chloroform extraction. DNA contaminants were removed using the Ambion Turbo DNA-free kit (Thermo Fisher AM1907), after which DNAsed RNA was reverse transcribed using random hexamer priming and the SmartScribe Reverse Transcriptase Kit (Takara Bio 639537). cDNA was then input into qPCR reactions with Fast Sybr Green Master Mix (Life Technologies 4385612) and signal was quantified with the BioRad CFX Opus system. Reading from 5′ to 3′, PNMA2 primers were CCCAGCTTCCTTGAGCTAAT (SEQ ID NO: 131) and GTTCCTCTGGCTCTTCGATAC (SEQ ID NO: 132), and Cre recombinase primers were CGATGCAACGAGTGATGAG (SEQ ID NO: 133) and GCAAACGGACTGAAGCAT (SEQ ID NO: 134).

### RNAseq of Cells and VLPs

U2OS cells were seeded at 6e6 cells per plate in 15-cm tissue culture dishes and transfected the following day with CRISPRa cassettes containing non-targeting guides or guides targeted against the transcriptional start site of PNMA2 with Lipofectamine 3000 (ThermoFisher L3000001) per the manufacturer's protocol. Forty-eight hours post transfection, media was harvested and centrifuged on a 20% sucrose cushion as described above. VLP pellets were resuspended in 1×PBS with 2 mM MgCl₂ and 250 units of Benzonase (Sigma-Aldrich E1014), incubated at 37° C. for 1 hour to degrade non-encapsidated genomes, and then resuspended in Trizol (ThermoFisher 15596026). Cells were washed in 1×PBS, after which 6e5 cells were resuspended in lysis buffer and subject to western blot as described above to confirm CRISPRa efficacy, while another aliquot of 6e5 cells were separately resuspended in Trizol for mRNA isolation. Following DNAse treatment, RNA concentrations were normalized, and RNAseq libraries were prepared with the NEBNext Ultra II Directional RNA Library Prep Kit (New England Biolabs E7765S) per the manufacturer's directions. RNAseq libraries were quantified and normalized with the KAPA library quantification kit (Roche 07960204001) and loaded onto an Illumina NextSeq 550 with 50 cycles for read 1 and 25 cycles for read 2. Raw reads were trimmed using Trimmomatic (29) and quality control was performed using fastqc (30) to eliminate low quality reads and adaptors. Resulting reads were mapped to a reference of the human genome (GRCh38) using STAR (31), and full read alignments were converted to indexed BAM files with SAMtools (32). A counts table was generated using htseq (33) and used to perform differential gene expression analysis using DESeq2 (34) in R.

### Cryo-Electron Microscopy

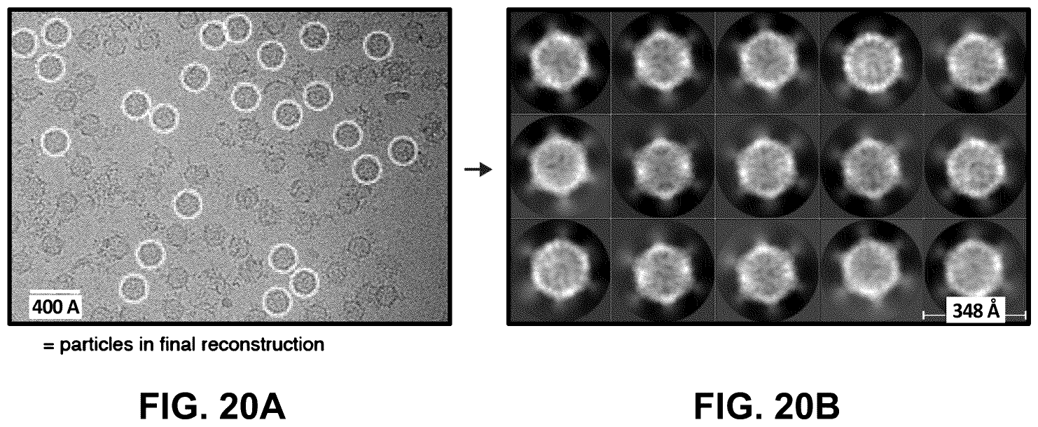

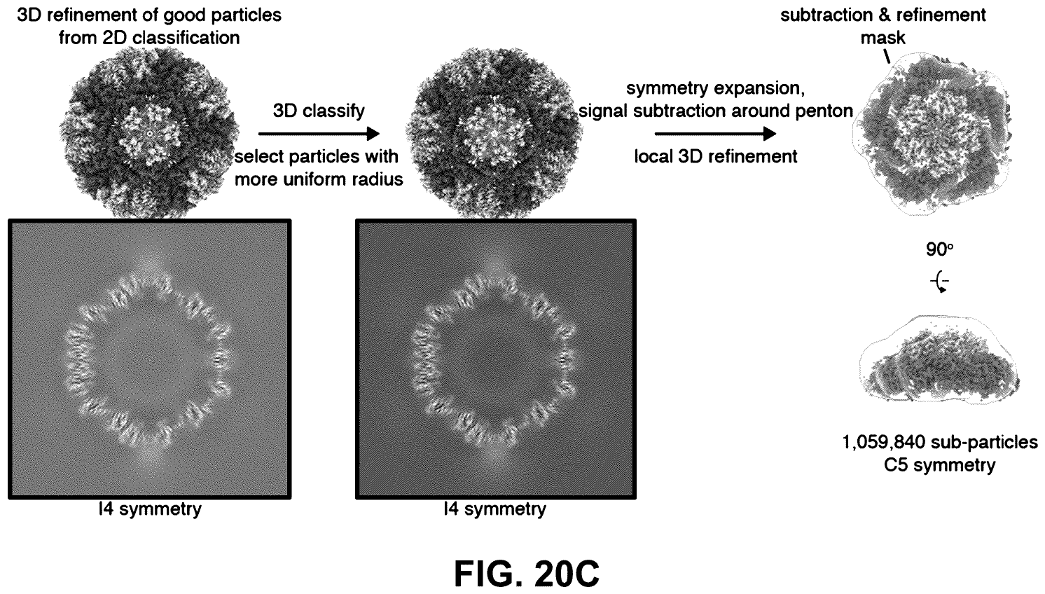

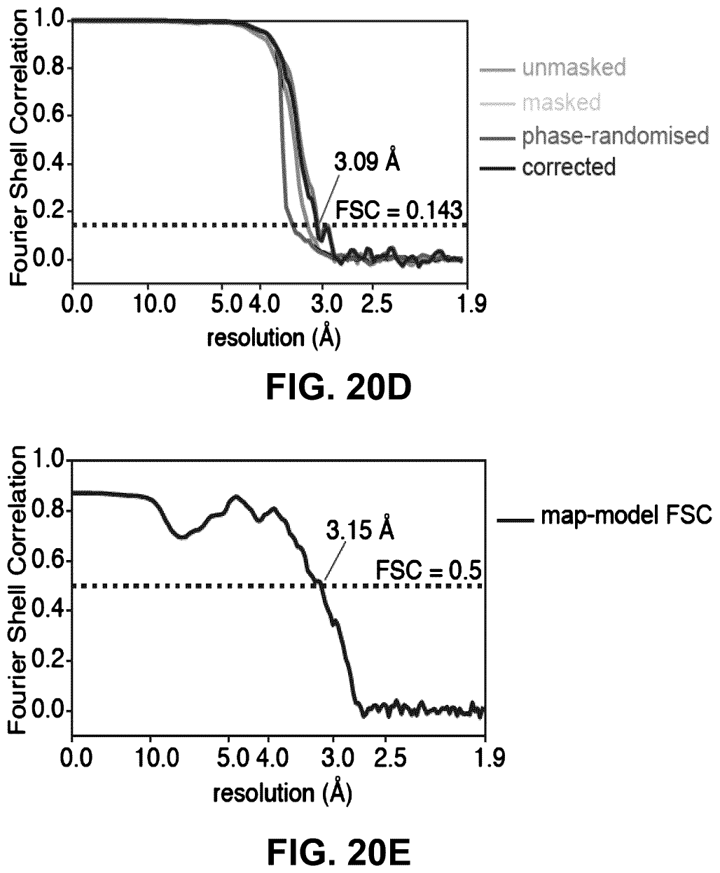

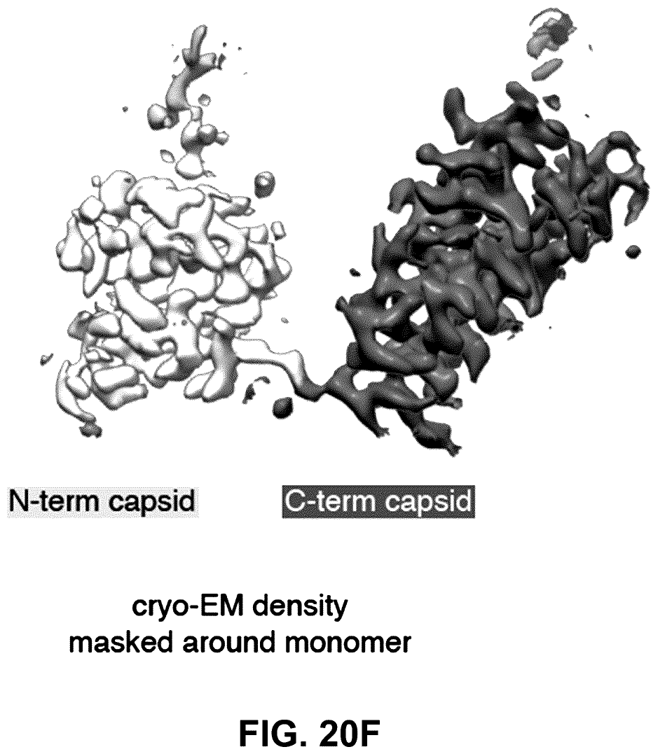

Assembled PNMA2 capsids were diluted to 1.5 mg/mL in PBS, and 4 pL was applied to a freshly glow-discharged (60 s at 25 mA) Cu300 R1.2/1.3 holey carbon grid (Quantifoil) mounted in the chamber of a Vitrobot Mark IV (Thermo Fisher Scientific) maintained at 4° C. and 100% humidity. The grid was blotted with Ø55 grade 595 filter paper (Ted Pella) for 4 seconds after a wait time of 0 seconds at a blot force of +10, and after a drain time of 1 second was plunged into liquid ethane. Cryo-EM data were collected using the Thermo Scientific Titan Krios G3i at MIT.nano using a K3 detector (Gatan) operating in super-resolution mode with 2-fold binning and an energy filter with slit width of 20 eV. Micrographs were collected using EPU in AFIS mode, yielding 17,600 movies at 130,000× magnification with a real pixel size of 0.6788 Å, a defocus range from −1 to −2.6 μm, an exposure time of 0.6 seconds fractionated into 24 frames, a flux of 23.6 c/pix/s and a total fluence per micrograph of 30.7 e⁻/Å². Cryo-EM data were processed using RELION 4.0 (35). Movies were corrected for motion using the RELION implementation of MotionCor2, with 4×4 patches and dose-weighting, and CTF parameters were estimated using CTFFIND-4.1 (36). Particles were picked using Topaz and a general model (37), yielding 722,571 particles which were extracted with a 512 pixel box, binned to 128 pixels, and classified using the VDAM 2D classification algorithm (FIGS. 20A-20B). 229,149 particles with high-quality 21) averages were re-extracted with a 512 pixel box binned to 360 pixels. 3D refinement with I4 symmetry, using an initial model generated by RELION from screening data on a Talos Arctica microscope, gave a 3.4 Å reconstruction, however the map showed radial blurring suggesting individual capsids had slightly different radii (FIG. 20C). 3D classification with regularization parameter T=15 allowed isolation of 88,320 capsids that were slightly smaller than average and had more well-defined density; these refined to 3.3 Å resolution after CTF refinement and Bayesian particle polishing but still showed some radial blurring. To improve the density, individual capsid particle images were converted to 12 sub-particles corresponding to individual pentons (FIG. 20C). This was done by 14 symmetry expansion in RELION to convert each particle to 60 subparticles, then only keeping the 1, 2, 3, 4, 5, 6, 10, 12, 20, 28, 29 and 38th subparticles, then performing particle subtraction with a mask around one of the z-axis-aligned pentons, and finally correcting for the local defocus of the subparticle based on its projected distance to the capsid center. Subparticles were then refined with C5 symmetry and 0.9° local angular sampling, producing a 3.1 Å reconstruction of an individual PNMA2 penton. Resolution is reported using the gold-standard Fourier Shell Correlation with 0.143 cutoff. The AlphaFold2 model of PNMA2 was docked into the penton cryo-EM density and adjusted using Coot (38). The model was duplicated around the 2—, 3- and 5-fold axes to produce all interfaces, then refined using ISOLDE (39). The extra monomers were then deleted, and the original monomer was duplicated with 14 symmetry and refined using PHENIX real_space_refine (40) into the 14-symmetric overall map using the starting model as a reference (sigma=0.1), one macrocycle of global minimization and ADP refinement, and a nonbonded weight of 2000. Structural figures were generated using UCSF ChimeraX (41). See also FIGS. 20D-20F.

### In Vitro Assembly and Disassembly of PNMA 2 Capsids

Purified PNMA2 protein was pH adjusted to 5, 6, 7, 8, 9, 10, 11 and 12 and NaCl concentration adjusted to 25 mM and 1M. Divalent ions were screened with addition of 10 mM MgCl2, 10 mM CaCl₂) or 100 uM ZnCl2 into 50 mM Tris pH 8 with varying concentrations of NaCl. Co-addition of 10 mM CaCl₂) and 100 uM ZnCl2 were tested with addition of 10 mM CaCl₂), 100 uM ZnCl2 or 10 mM CaCl₂) and 100 uM ZnCl2 into 50 mM Tris pH 8 with varying concentrations of NaCl.

PNMA2(340)-CCMV(30) was treated with 0 M NaCl or 1 M NaCl in addition to 0 M Urea, 1 M Urea or 6 M Urea. PNMA2(340)-CCMV(30) concentration was kept the same for all conditions.

In Vitro Transcription of Cre mRNA

A plasmid encoding Cre mRNA was digested with PsiI for 1 hour at 37° C. The digested product was PCR cleaned up using a Qiagen PCR clean up kit. mRNA was synthesized using a Hiscribe T7 ARCA mRNA kit. The pellet was dissolved in 20 μl of water. mRNA quality was checked by running a 1% E-gel. mRNA concentration was measured by nanodrop.

### Transduction Assays

VLP samples or a naked mRNA control were normalized to the same mRNA concentration, and treated with 10 μg/mL RNAse A (Qiagen) for 30 minutes at room temperature to degrade un-encapsidated genomes. A second mRNA sample was prepared at the same concentration without RNAse A treatment as a positive control. Neuro-2a-loxP-GFP reporter cells were seeded at 1.5e4 cells/well in a 96-well format. The following day, positive control mRNA, RNAse treated mRNA, and VLP samples were mixed with LAH4 cell penetrating peptide (Genscript RP20096) in Optimem (Gibco) and added to cells at a final concentration of 10 μg/mL. Media was changed the day after VLP and mRNA treatment. Ninety-six hours post treatment, cells were washed with 1×PBS, trypsinized, resuspended in full media, and spun at 1000 g for 3 minutes in a 96-well V-bottom plate. Cells were washed with FACS buffer (lx PBS with 2% FBS and 2 mM EDTA), spun at 1000 g for 3 minutes, and then resuspended in FACS buffer with DAPI at 0.01 mg/mL. Following an additional FACS buffer wash, cell fluorescence was read out on a CytoFlex S Flow Cytometer (Beckman Coulter).

### REFERENCES FOR EXAMPLE 3

1. J. A. Kulkarni, et al., The current landscape of nucleic acid therapeutics. Nat. Nanotechnol. 16, 630-643 (2021).
2. M. Campillos, T. Doerks, P. K. Shah, P. Bork, Computational characterization of multiple Gag-like human proteins. Trends Genet. 22, 585-589 (2006).
3. J. Ashley, et al., Retrovirus-like Gag Protein Arc1 Binds RNA and Traffics across Synaptic Boutons. Cell 172, 262-274.e11 (2018).
4. E. D. Pastuzyn, et al., The Neuronal Gene Arc Encodes a Repurposed Retrotransposon Gag Protein that Mediates Intercellular RNA Transfer. Cell 172, 275-288.e18 (2018).
5. M. Abed, et al., The Gag protein PEG10 binds to RNA and regulates trophoblast stem cell lineage specification. PLoS One 14, e0214110 (2019).
6. M. Segel, et al., Mammalian retrovirus-like protein PEG10 packages its own mRNA and can be pseudotyped for mRNA delivery. Science 373, 882-889 (2021).
7. S. Iwasaki, et al., Identification of a novel PNMA-MS1 gene in marsupials suggests the LTR retrotransposon-derived PNMA genes evolved differently in marsupials and eutherians. DNA Res. 20, 425-436 (2013).
8. J. Xu, et al., PNMA2 forms non-enveloped virus-like capsids that trigger paraneoplastic neurological syndrome. bioRxiv (2023) https:/doi.org/10.1101/2023.02.09.527862.
9. D. Chalopin, D. Galiana, J.-N. Volff, Genetic innovation in vertebrates: gypsy integrase genes and other genes derived from transposable elements. Int. J. Evol. Biol. 2012, 724519 (2012).
10. S. Sandmeyer, K. Patterson, V. Bilanchone, Ty3, a Position-specific Retrotransposon in Budding Yeast. Microbiol Spectr 3, MDNA3-0057-2014 (2015).
11. Q. Xie, et al., The atomic structure of adeno-associated virus (AAV-2), a vector for human gene therapy. Proc. Natl. Acad. Sci. U.S.A 99, 10405-10410 (2002).
12. R. F. Garmann, C. M. Knobler, W. M. Gelbart, Protocol for Efficient Cell-Free Synthesis of Cowpea Chlorotic Mottle Virus-Like Particles Containing Heterologous RNAs. Methods Mol. Biol. 1776, 249-265 (2018).
13. M. Comas-Garcia, R. D. Cadena-Nava, A. L. N. Rao, C. M. Knobler, W. M. Gelbart, In vitro quantification of the relative packaging efficiencies of single-stranded RNA molecules by viral capsid protein. J. Virol. 86, 12271-12282 (2012).
14. A. Kichler, A. J. Mason, B. Bechinger, Cationic amphipathic histidine-rich peptides for gene delivery. Biochim. Biophys. Acta 1758, 301-307 (2006).
15. N. B. Y. Tsui, E. K. O. Ng, Y. M. D. Lo, Stability of endogenous and added RNA in blood specimens, serum, and plasma. Clin. Chem. 48, 1647-1653 (2002).
16. T. C. Roberts, R. Langer, M. J. A. Wood, Advances in oligonucleotide drug delivery. Nat. Rev. Drug Discov. 19, 673-694 (2020).
17. P. Crespo-Escobar, et al., The role of gluten consumption at an early age in celiac disease development: a further analysis of the prospective PreventCD cohort study. Am. J. Clin. Nutr. 105, 890-896 (2017).
18. W. Shi, J. S. Bartlett, RGD inclusion in VP3 provides adeno-associated virus type 2 (AAV2)-based vectors with a heparan sulfate-independent cell entry mechanism. Mol. Ther. 7, 515-525 (2003).
19. J. Jumper, et al., Highly accurate protein structure prediction with AlphaFold. Nature 596, 583-589 (2021).
20. M. Mirdita, et al., ColabFold: making protein folding accessible to all. Nat. Methods 19, 679-682 (2022).
21. G. Faure, I. Callebaut, Comprehensive repertoire of foldable regions within whole genomes. PLoS Comput. Biol. 9, e1003280 (2013).
22. I. Callebaut, et al., Deciphering protein sequence information through hydrophobic cluster analysis (HCA): current status and perspectives. Cell. Mol. Life Sci. 53, 621-645 (1997).
23. L. Holm, Dali server: structural unification of protein families. Nucleic Acids Res. 50, W210-W215 (2022).
24. L. Holm, Using Dali for Protein Structure Comparison. Methods Mol. Biol. 2112, 29-42 (2020).
25. S. Guindon, et al., New algorithms and methods to estimate maximum-likelihood phylogenies: assessing the performance of PhyML 3.0. Syst. Biol. 59, 307-321 (2010).
26. F. Gabler, et al., Protein Sequence Analysis Using the MPI Bioinformatics Toolkit. Curr. Protoc. Bioinformatics 72, e108 (2020).
27. I. Letunic, P. Bork, Interactive Tree Of Life (iTOL) v5: an online tool for phylogenetic tree display and annotation. Nucleic Acids Res. 49, W293-W296 (2021).
28. R. Evans, et al., Protein complex prediction with AlphaFold-Multimer. bioRxiv, 2021.10.04.463034 (2022).
29. A. M. Bolger, M. Lohse, B. Usadel, Trimmomatic: a flexible trimmer for Illumina sequence data. Bioinformatics 30, 2114-2120 (2014).
30. Babraham bioinformatics—FastQC A quality control tool for high throughput sequence data (Mar. 28, 2023).
31. A. Dobin, et al., STAR: ultrafast universal RNA-seq aligner. Bioinformatics 29, 15-21 (2013).
32. H. Li, et al., The Sequence Alignment/Map format and SAMtools. Bioinformatics 25, 2078-2079 (2009).
33. S. Anders, P. T. Pyl, W. Huber, HTSeq-a Python framework to work with high-throughput sequencing data. Bioinformatics 31, 166-169 (2015).
34. M. I. Love, W. Huber, S. Anders, Moderated estimation of fold change and dispersion for RNA-seq data with DESeq2. Genome Biol. 15, 550 (2014).
35. D. Kimanius, L. Dong, G. Sharov, T. Nakane, S. H. W. Scheres, New tools for automated cryo-EM single-particle analysis in RELION-4.0. Biochem. J 478, 4169-4185 (2021).
36. A. Rohou, N. Grigorieff, CTFFIND4: Fast and accurate defocus estimation from electron micrographs. J. Struct. Biol. 192, 216-221 (2015).
37. T. Bepler, et al., Positive-unlabeled convolutional neural networks for particle picking in cryo-electron micrographs. Nat. Methods 16, 1153-1160 (2019).
38. A. Casanal, B. Lohkamp, P. Emsley, Current developments in Coot for macromolecular model building of Electron Cryo-microscopy and Crystallographic Data. Protein Sci. 29, 1069-1078 (2020).
39. T. I. Croll, ISOLDE: a physically realistic environment for model building into low-resolution electron-density maps. Acta Crystallogr D Struct Biol 74, 519-530 (2018).
40. P. V. Afonine, et al., Real-space refinement in PHENIX for cryo-EM and crystallography. Acta Crystallogr D Struct Biol 74, 531-544 (2018).
41. E. F. Pettersen, et al., UCSF ChimeraX: Structure visualization for researchers, educators, and developers. Protein Sci. 30, 70-82 (2021).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. An engineered paraneoplastic Ma protein (PNMA) capable of forming a capsid and comprising one or more modifications that enhance binding or loading of a cargo into the capsid, one or more modifications that modify cell-specificity of the capsid, one or more modifications that enhance intracellular delivery of the capsid, or any combination thereof.
2. The engineered PNMA of aspect 1, wherein the one or more modifications that enhance binding or loading of the cargo comprise:
   a. addition of a peptide comprising charged residues;
   b. addition of a polynucleotide binding domain;
   c. addition of a polypeptide binding domain; or
   d. any combination thereof.
3. The engineered PNMA of aspect 2, wherein the peptide comprising charged residues is:
   a. inserted between any two consecutive amino acids in a loop domain of the PNMA;
   b. an addition to the C- or N-terminus of the PNMA;
   c. an addition to a C- or N-terminally truncated PNMA; or
   d. or (a) combined with (b) or (c).
4. The engineered PNMA of any one of aspects 2-3, wherein the peptide comprising charged residues is inserted into a loop domain at amino acids 170-180, amino acids 256-263, or amino acids 302-305 of PNMA2, or a position in another PNMA corresponding thereto.
5. The engineered PNMA of any one of aspects 2-4, wherein the peptide comprising charged residues is inserted between amino acids 175 and 176, 261 and 262, 303 and 304 of PNMA2, or at a position of another PNMA corresponding thereto.
6. The engineered PNMA of any one of aspects 3-5, wherein the size of the C-terminus truncation of the C-terminally truncated PNMA is 1 to 31 amino acids.
7. The engineered PNMA protein of any one of aspects 3-6, wherein the peptide comprising charged residues is about 20 to about 150 amino acids in size.
8. The engineered PNMA of any one of aspects 3-7, wherein the peptide comprising charged residues comprises an arginine, lysine, and/or proline rich motif.
9. The engineered PNMA of any one of aspects 3-8, wherein the peptide comprising charged residues comprises two or more RKK repeats or two or more RRLRRP (SEQ ID NO: 6) repeats.
10. The engineered PNMA of any one of aspects 8-9, wherein the peptide comprising charged residues is RRKRRKRRKRRK (SEQ ID NO: 7).
11. The engineered PNMA of any one of aspects 8-9, wherein the peptide comprising charged residues is RRLRRPRRLRRPRRPR (SEQ ID NO: 8).
12. The engineered PNMA of any one of aspects 2-11, wherein polynucleotide binding domain is
   a. inserted between any two consecutive amino acids in a loop domain of the PNMA;
   b. inserted in place of at least a portion of a zinc finger region;
   c. an extension of a C- or N-terminus of the PNMA;
   d. an extension of a C- or N-terminally truncated PNMA;
   e. or (a) combined with (b);
   f. or (a) and (b) combined with (c) or (d);
   g. or (b) combined with (c) or (d).
13. The engineered PNMA of aspect 12, wherein the polynucleotide binding domain is inserted between amino acids 256-263 or 302-305 of PNMA2 or at a position corresponding thereto.
14. The engineered PNMA of any one of aspects 12-13, wherein the polynucleotide binding domain is inserted between amino acids 261 and 262, or amino acid 303 and 304 of PNMA2 or an amino acid position in another PNMA corresponding thereto.
15. The engineered PNMA of aspect 12, wherein the polynucleotide binding domain replaces amino acids 412 to 429 of PNMA3 or an amino acid position of another PNMA corresponding thereto.
16. The engineered PNMA of aspect 12, wherein the polynucleotide binding domain is inserted between M1 and P2 of the N-terminus of PNMA3 or a position in another PNMA corresponding thereto.
17. The engineered PNMA of any one of aspects 12-16 wherein the polynucleotide binding domain comprises or consists of a PNMA RNA recognition motif, a λN polypeptide, a P22N polypeptide, a MS2 polypeptide, an R17 polypeptide, a retroviral or lentiviral Rev polypeptide, polynucleotide binding domain of a nuclease, a Zinc Finger domain, a 14-3-3 polypeptide, a STAR-family polypeptide, a toll-like receptor polypeptide, CCMV N-terminal sequence, or any combination thereof.
18. The engineered PNMA of aspect 17, wherein the λN polypeptide comprises SEQ ID NO: 1 or 2.
19. The engineered PNMA of any one of aspects 17-18, wherein the P22N comprises SEQ ID NO: 3.
20. The engineered PNMA any one of aspects 17-19, wherein the Rev polypeptide comprises SEQ ID NO: 4.
21. The engineered PNMA of any one of aspects 2-20, wherein the protein binding domain is added to a C- or N-terminus of the PNMA.
22. The engineered PNMA of aspect 21, wherein the protein binding domain is a dimerization domain, optionally a leucine zipper.
23. The engineered PNMA of any one of aspects 1-22, wherein the one or more modifications that modify cell-specificity comprise insertion of a cell surface binding peptide, cell penetrating peptide, monobody, nanobody, or antibody or fragment thereof, in the N-terminus of the PNMA, optionally wherein the one or more modifications are inserted between amino acid residues P27-E31, G125 and S138, P196 and T198, D224 and S229, G319 and S323, or any combination thereof with reference to PNMA2 or PNMA3 or a position in another PNMA corresponding thereto.
24. The engineered PNMA of aspect 23, wherein the cell surface binding peptide is an integrin binding peptide, a VEGFR-1 ligand, an EGF peptide, a human transferrin receptor binding peptide, a hepatocellular carcinoma targeting peptide, a monobody capable of specifically binding a cell surface or molecule thereon, or a nanobody capable of specifically binding a cell surface or molecule thereon.
25. The engineered PNMA of any one of aspects 1-24, wherein the one or more modifications that enhance intracellular delivery are capable of enhancing cell entry, endosomal escape or both, and optionally wherein the one or more modifications comprise or consist of endosomal escape peptides.
26. The engineered PNMA of aspect 25, wherein the endosomal escape peptides are selected from the group consisting of: pVI, H5WYG, HIV tat, and R5.
27. A polynucleotide encoding the engineered PNMA of anyone of aspects 1 to 26.
28. A vector encoding the engineered PNMA of anyone of aspects 1 to 26.
29. A delivery system comprising:
   a capsid comprising the engineered PNMA of any one of aspects 1 to 26; and
   a cargo captured by, or packaged within, the capsid.
30. A method for cellular delivery of cargoes, comprising:
   delivering the delivery system of aspect 29 to a cell or population of cells in vitro or in vivo.
31. A method of in vitro packaging a cargo in a capsid comprising one or more engineered paraneoplastic Ma proteins (PNMAs) of aspect 1 comprising:
combining a cargo and a plurality of engineered PNMA monomers according to aspect 1 in an assembly solution comprising an amount of a salt and an amount of calcium chloride thereby promoting assembly of the capsid and packaging of the cargo in the capsid.
32. The method of aspect 31, wherein the amount of salt in the assembly solution is about 100 mM to about 600 mM, wherein the amount of calcium chloride in the assembly solution is about 5 to about 100 mM, or both.
33. The method of any one of aspects 31-32, wherein the amount of salt in the assembly solution is about 500 mM, wherein the amount of calcium chloride in the assembly solution is about 10 mM, or both.
34. The method of any one of aspects 31-33, further comprising generating the plurality of engineered PNMA monomers prior to combining, wherein generating the plurality of engineered PNMA monomers comprises disassembling one or more capsids comprising a plurality of engineered PNMAs by exposing the capsid comprising one or more engineered PNMAs to a disassembly solution thereby generating the plurality of PNMA monomers.
35. The method of aspect 34, wherein the disassembly solution comprises an amount of a salt or an amount of urea effective to disassembly the one or more capsids comprising a plurality of engineered PNMAs.
37. The method of any one of aspects 34-35, wherein the disassembly solution comprises an amount of salt or an amount of urea effective to promote disassembly of the one or more capsids.
38. The method of any one of aspects 34-37, wherein the disassembly solution comprises about 5 mM to about 50 mM salt or about 6 M urea.
39. The method of any one of aspects 34-38, wherein the disassembly solution does not contain calcium chloride.

What is claimed is:

1. An engineered paraneoplastic Ma protein (PNMA) capable of forming a capsid and comprising one or more modifications that enhance binding or loading of a cargo into the capsid, one or more modifications that modify cell-specificity of the capsid, one or more modifications that enhance intracellular delivery of the capsid, or any combination thereof.

2. The engineered PNMA of claim 1, wherein the one or more modifications that enhance binding or loading of the cargo comprise:
a. addition of a peptide comprising charged residues;
b. addition of a polynucleotide binding domain;
c. addition of a polypeptide binding domain; or
d. any combination thereof.

3. The engineered PNMA of claim 2, wherein the peptide comprising charged residues is:
a. inserted between any two consecutive amino acids in a loop domain of the PNMA;
b. an addition to the C- or N-terminus of the PNMA;
c. an addition to a C- or N-terminally truncated PNMA; or
d. or (a) combined with (b) or (c).

4. The engineered PNMA of claim 3, wherein the peptide comprising charged residues is inserted into a loop domain at amino acids 170-180, amino acids 256-263, or amino acids 302-305 of PNMA2, or a position in another PNMA corresponding thereto.

5. The engineered PNMA of claim 4, wherein the peptide comprising charged residues is inserted between amino acids 175 and 176, 261 and 262, 303 and 304 of PNMA2, or at a position of another PNMA corresponding thereto.

6. The engineered PNMA of claim 3, wherein the size of the C-terminus truncation of the C-terminally truncated PNMA is 1 to 31 amino acids.

7. The engineered PNMA protein of claim 3, wherein the peptide comprising charged residues is about 20 to about 150 amino acids in size.

8. The engineered PNMA of claim 3, wherein the peptide comprising charged residues comprises an arginine, lysine, and/or proline rich motif.

9. The engineered PNMA of claim 3, wherein the peptide comprising charged residues comprises two or more RKK repeats or two or more RRLRRP (SEQ ID NO: 6) repeats.

10. The engineered PNMA of claim 8, wherein the peptide comprising charged residues is RRKRRKRRKRRK (SEQ ID NO: 7).

11. The engineered PNMA of claim 8, wherein the peptide comprising charged residues is RRLRRPRRLRRPRRPR (SEQ ID NO: 8).

12. The engineered PNMA of claim 2, wherein polynucleotide binding domain is
a. inserted between any two consecutive amino acids in a loop domain of the PNMA;
b. inserted in place of at least a portion of a zinc finger region;
c. an extension of a C- or N-terminus of the PNMA;
d. an extension of a C- or N-terminally truncated PNMA;
e. or (a) combined with (b);
f. or (a) and (b) combined with (c) or (d);
g. or (b) combined with (c) or (d).

13. The engineered PNMA of claim 12, wherein the polynucleotide binding domain is inserted between amino acids 256-263 or 302-305 of PNMA2 or at a position corresponding thereto.

14. The engineered PNMA of claim 13, wherein the polynucleotide binding domain is inserted between amino acids 261 and 262, or amino acid 303 and 304 of PNMA2 or an amino acid position in another PNMA corresponding thereto.

15. The engineered PNMA of claim 12, wherein the polynucleotide binding domain replaces amino acids 412 to 429 of PNMA3 or an amino acid position of another PNMA corresponding thereto.

16. The engineered PNMA of claim 12, wherein the polynucleotide binding domain is inserted between M1 and P2 of the N-terminus of PNMA3 or a position in another PNMA corresponding thereto.

17. The engineered PNMA of claim 13 or 14, wherein the polynucleotide binding domain comprises or consists of a PNMA RNA recognition motif, a λN polypeptide, a P22N polypeptide, a MS2 polypeptide, an R17 polypeptide, a retroviral or lentiviral Rev polypeptide, polynucleotide binding domain of a nuclease, a Zinc Finger domain, a 14-3-3 polypeptide, a STAR-family polypeptide, a toll-like receptor polypeptide, CCMV N-terminal sequence, a arginine, lysine, and/or proline rich motif, or any combination thereof.

18. The engineered PNMA of claim 17, wherein the λN polypeptide comprises SEQ ID NO: 1 or 2.

19. The engineered PNMA of claim 17, wherein the P22N comprises SEQ ID NO:
3.

20. The engineered PNMA of claim 17, wherein the Rev polypeptide comprises SEQ ID NO: 4.

21. The engineered PNMA of claim 2, wherein the protein binding domain is added to a C- or N-terminus of the PNMA.

22. The engineered PNMA of claim 21, wherein the protein binding domain is a dimerization domain, optionally a leucine zipper.

23. The engineered PNMA of claim 1, wherein the one or more modifications that modify cell-specificity comprise insertion of a cell surface binding peptide, cell penetrating peptide, monobody, nanobody, or antibody or fragment thereof, in the N-terminus of the PNMA, optionally wherein the one or more modifications are inserted between amino acid residues P27-E31, G125 and S138, P196 and T198, D224 and S229, G319 and S323, or any combination thereof with reference to PNMA2 or PNMA3 or a position in another PNMA corresponding thereto.

24. The engineered PNMA of claim 23, wherein the cell surface binding peptide is an integrin binding peptide, a VEGFR-1 ligand, an EGF peptide, a human transferrin receptor binding peptide, a hepatocellular carcinoma targeting peptide, a monobody capable of specifically binding a cell surface or molecule thereon, or a nanobody capable of specifically binding a cell surface or molecule thereon.

25. The engineered PNMA of claim 1, wherein the one or more modifications that enhance intracellular delivery are capable of enhancing cell entry, endosomal escape or both, and optionally wherein the one or more modifications comprise or consist of endosomal escape peptides.

26. The engineered PNMA of claim 25, wherein the endosomal escape peptides are selected from the group consisting of: pVI, H5WYG, HIV tat, R5, and LAH4.

27. A polynucleotide encoding the engineered PNMA of anyone of claims 1 to 26.

28. A vector encoding the engineered PNMA of anyone of claims 1 to 26.

29. A delivery system comprising:
a capsid comprising the engineered PNMA of any one of claims 1 to 26; and
a cargo captured by, or packaged within, the capsid.

30. A method for cellular delivery of cargoes, comprising:
delivering the delivery system of claim 29 to a cell or population of cells in vitro or in vivo.

31. A method of in vitro packaging a cargo in a capsid comprising one or more engineered paraneoplastic Ma proteins (PNMAs) of claim 1 comprising:
combining a cargo and a plurality of engineered PNMA monomers according to claim 1 in an assembly solution comprising an amount of a salt and an amount of calcium chloride thereby promoting assembly of the capsid and packaging of the cargo in the capsid.

32. The method of claim 31, wherein the amount of salt in the assembly solution is about 100 mM to about 600 mM, wherein the amount of calcium chloride in the assembly solution is about 5 to about 100 mM, or both.

33. The method of claim 32, wherein the amount of salt in the assembly solution is about 500 mM, wherein the amount of calcium chloride in the assembly solution is about 10 mM, or both.

34. The method of claim 31, further comprising generating the plurality of engineered PNMA monomers prior to combining, wherein generating the plurality of engineered PNMA monomers comprises disassembling one or more capsids comprising a plurality of engineered PNMAs by exposing the capsid comprising one or more engineered PNMAs to a disassembly solution thereby generating the plurality of PNMA monomers.

35. The method of claim 34, wherein the disassembly solution comprises an amount of a salt or an amount of urea effective to disassembly the one or more capsids comprising a plurality of engineered PNMAs.

37. The method of claim 35, wherein the disassembly solution comprises an amount of salt or an amount of urea effective to promote disassembly of the one or more capsids.

38. The method of claim 37, wherein the disassembly solution comprises about 5 mM to about 50 mM salt or about 6 M urea.

39. The method of claim 38, wherein the disassembly solution does not contain calcium chloride.
